(19) United States
(12) Patent Application Publication
(10) Pub. No.: US 2022/0402925 A1
(43) Pub. Date: Dec. 22, 2022

(54) COMPOUNDS AND METHODS FOR THE TREATMENT OF CYSTIC FIBROSIS

(71) Applicant: Flatley Discovery Lab, LLC, Canton, MA (US)

(72) Inventors: Feng Li, Winchester, MA (US); Christopher Oalmann, Watertown, MA (US); Bridget Cole, Quincy, MA (US); Andrew Kolodziej, Winchester, MA (US); Richard Fitzpatrick, Marblehead, MA (US)

(21) Appl. No.: 17/831,145

(22) Filed: Jun. 2, 2022

Related U.S. Application Data
(63) Continuation of application No. PCT/US2020/063535, filed on Dec. 7, 2020.
(60) Provisional application No. 62/944,208, filed on Dec. 5, 2019.

(51) Int. Cl. C07D 487/04; C07D 491/048; C07D 405/12

(52) U.S. Cl. CPC C07D 487/04; C07D 491/048; C07D 405/12

(57) ABSTRACT

The invention relates to a compound of Formula I, pharmaceutical compositions comprising a compound of Formula I, and methods of treating cystic fibrosis comprising the step of administering a therapeutically effective amount of a compound of Formula I to a subject in need thereof.

### RELATED APPLICATIONS

This application is a continuation of International Application No. PCT/US2020/063535, which designated the United States and was filed on Dec. 7, 2020, published in English, which claims the benefit of U.S. Provisional Application No. 62/944,208, filed on Dec. 5, 2019. The entire teachings of the above applications are incorporated herein by reference.

### BACKGROUND

Cystic fibrosis (CF) is a lethal, recessive, genetic disease affecting approximately 1 in 2500 live births among Caucasians. (Cohen-Cymberknoh, M. et al., Am. J Respir. Crit. Care Med. 1463-1471, 2011; Boat et al., The Metabolic Basis of Inherited Disease, 6th ed., pp 2649-2680, McGraw Hill, NY (1989)). Approximately 1 in 25 persons are carriers of the disease. The major symptoms of cystic fibrosis include chronic pulmonary disease, pancreatic exocrine insufficiency, and elevated sweat electrolyte levels. The symptoms are consistent with cystic fibrosis being an exocrine disorder. (Hantash F: U.S. Patent Application No. 20060057593).

The CF gene codes for a cAMP/PKA-dependent, ATP-requiring, membrane chloride ion channel, generally found in the apical membranes of many secreting epithelia and is known as CFTR (cystic fibrosis transmembrane conductance regulator). There are currently over 1900 known mutations affecting CFTR, many of which give rise to a disease phenotype. Around 75% of CF alleles contain the ΔF508 mutation in which a triplet codon has been lost, leading to a missing phenylalanine at position 508 in the protein. This altered protein fails to be trafficked to the correct location in the cell and is generally destroyed by the proteasome. The small amount that does reach the correct location functions poorly. (Cuthbert A W, British Journal of Pharmacology, 163(1), 173-183, 2011).

Mutations in the CFTR gene result in absence or dysfunction of the protein that regulates ion transport across the apical membrane at the surface of certain epithelia. Although CFTR functions mainly as a chloride channel, it has many other roles, including inhibition of sodium transport through the epithelial sodium channel, regulation of the outwardly rectifying chloride channel, ATP channels, intracellular vesicle transport, and inhibition of endogenous calcium-activated chloride channels. CFTR is also involved in bicarbonate-chloride exchange. A deficiency in bicarbonate secretion leads to poor solubility and aggregation of luminal mucins. Obstruction of intrapancreatic ducts with thickened secretions causes autolysis of pancreatic tissue with replacement of the body of the pancreas with fat, leading to pancreatic insufficiency with subsequent malnutrition. In the lungs, CFTR dysfunction leads to airway surface liquid (ASL) depletion and thickened and viscous mucus that adheres to airway surfaces. The result is decreased mucociliary clearance (MCC) and impaired host defenses. Dehydrated, thickened secretions lead to endobronchial infection with a limited spectrum of distinctive bacteria, mainly Staphylococcus aureus and Pseudomonas aeruginosa, and an exaggerated inflammatory response leading to development of bronchiectasis and progressive obstructive airways disease. Pulmonary insufficiency is responsible for most CF-related deaths. (Cohen-Cymberknoh, M et al., Am. J. Respir. Crit. Care Med. 1463-1471, 2011).

The prognosis for the treatment of CF has improved over the last 40 years. This was achieved by improving pancreatic enzyme supplements, drugs designed to treat pulmonary infection, reduce inflammation and enhance mucociliary clearance. Currently the therapeutic challenges are to correct the biochemical defect of CF and to identify effective treatments for chronic respiratory infection. (Frerichs C. et al., Expert Opin Pharmacother. 10(7), 1191-202, 2009).

### SUMMARY OF THE INVENTION

In one embodiment, the invention relates to a compound of Formula (I)

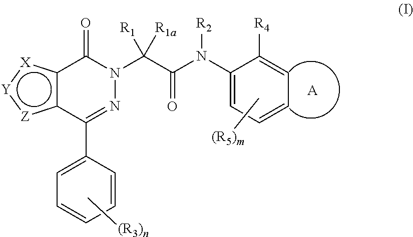

or a pharmaceutically acceptable salt thereof, wherein:
Ring A is a 4- to 6-membered optionally substituted carbocyclic or heterocyclic;
X is O, S or NR₇; Y is N or CR₆; and Z is N or CR₆; or
X is N or CR₆; Y is N or CR₆ and Z is O, S or NR₇; or
X is N or CR₆; Y is O, S or NR₇ and Z is N or CR₆;
preferably at least one of X, Y and Z is CR₆;
n is 0, 1, 2 or 3; preferably n is 1 or 2;
m is 0, 1 or 2; preferably m is 0 or 1;
R₁ and R₁ₐ are independently hydrogen, optionally substituted alkyl, or halogen; preferably, R₁ and R₁ₐ are independently hydrogen or methyl; more preferably, R₁ and R₁ₐ are both hydrogen;
R₂ is hydrogen, optionally substituted alkyl, optionally substituted cycloalkyl or optionally substituted heterocyclyl; preferably R₂ is C₁-C₄-alkyl, halo-C₁-C₄-alkyl, C₃-C₆-cycloalkyl or saturated 5- or 6-membered heterocyclyl; more preferably, R₂ is methyl, ethyl or cyclopropyl; Each R₃ is independently CN, hydroxyl, halogen, optionally substituted C₁-C₆-alkyl, optionally substituted C₁-C₆-alkoxy, optionally substituted aryl, R₈OC(O)—, (R₈)(R₉)NC(O)—, (R₈)(R₉)N—, (R₈)(R₉)NS(O)₂—; preferably, each R₃ is independently CN, F, Cl, hydroxyl, C₁-C₄-alkyl, halo-C₁-C₄-alkyl, C₁-C₄-alkoxy, or halo-C₁-C₄-alkoxy; more preferably, each R₃ is independently CN, F, Cl, methyl, t-butyl, trifluoromethyl, difluoromethyl, methoxy, t-butoxy or trifluoromethoxy;
Alternatively, two adjacent R₃ groups, together with the carbon atoms to which they are attached, form an optionally substituted carbocyclic or heterocyclic;
R₄ is hydrogen or substituted or unsubstituted C₁-C₆-alkyl;
or R₂ and R₄ together form optionally substituted C₂-C₃-alkylene;
each R₅ is independently optionally substituted C₁-C₄-alkyl;
Each R₆ is hydrogen, halogen, hydroxyl, optionally substituted C₁-C₆-alkyl, optionally substituted C₁-C₆-alkoxy, optionally substituted C₃-C₆-cycloalkyl, (R₈)(R₉)NC(O)—, R₈OC(O)— and R₁₀C(O)NH—; preferably each R₆ is hydrogen;
Each R₇ is hydrogen or optionally substituted C₁-C₄-alkyl; preferably each R₇ is hydrogen;
R₈ and R₉ are each independently hydrogen, optionally substituted C₁-C₆-alkyl, optionally substituted C₁-C₆-alkoxy or optionally substituted C₃-C₆-cycloalkyl; alternatively, R₈ and R₉, together with the nitrogen atom to which they are attached, form an optionally substituted 3 to 8-membered heterocyclyl; and
R₁₀ is optionally substituted C₁-C₆-alkyl.

In another embodiment, the present invention relates to a pharmaceutical composition comprising a compound of Formula (I), or a pharmaceutically acceptable salt thereof, and a pharmaceutically acceptable carrier or excipient.

In another embodiment, the present invention relates to a method of treating a CFTR-mediated disease or disorder, such as cystic fibrosis, in a subject in need thereof, comprising administering to the subject a therapeutically effective amount of a compound of Formula (I) or a pharmaceutically acceptable salt thereof.

### DETAILED DESCRIPTION OF THE INVENTION

The present invention relates to compounds of Formula (I) and pharmaceutically salts thereof, pharmaceutical compositions comprising such compounds and methods of using such compounds for treating a CFTR-mediated disease or condition in a subject in need thereof.

In certain embodiments of the compounds of Formula (I), the group

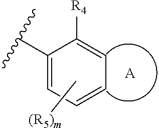

is selected from

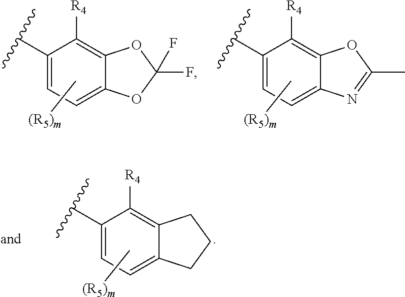

In certain embodiments, the compounds of the invention are represented by Formulas (IIa), (IIb), (IIc), (IId), (IIe), (IIf), (IIg), (IIh), (IIi), (IIj), (IIIa), (IIIb), (IIIc), (IIId), (IIIe), (IIIf), (IIIg), (IIIh), (IIIi) and (IIIj), and pharmaceutically acceptable salts thereof.

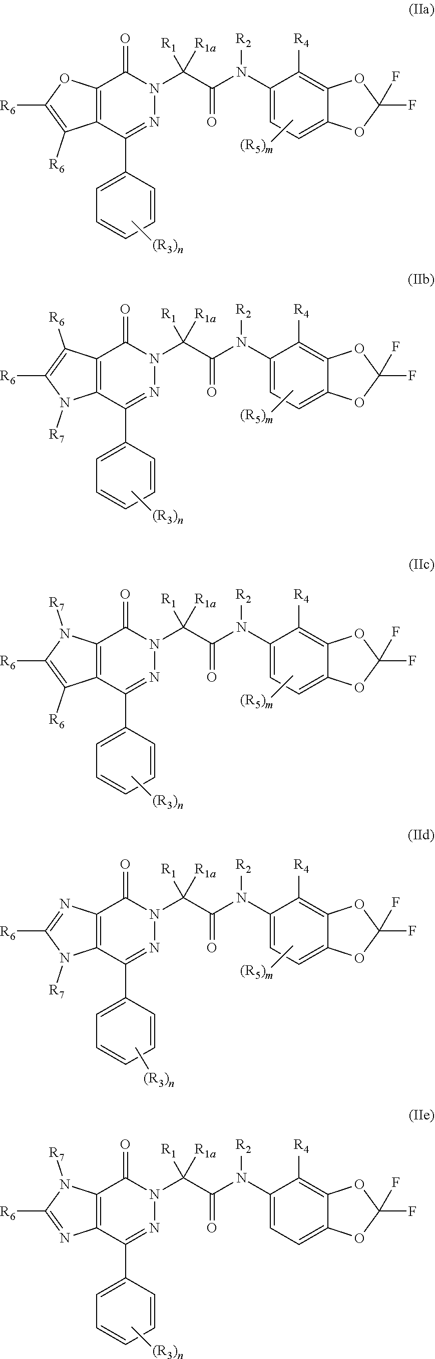

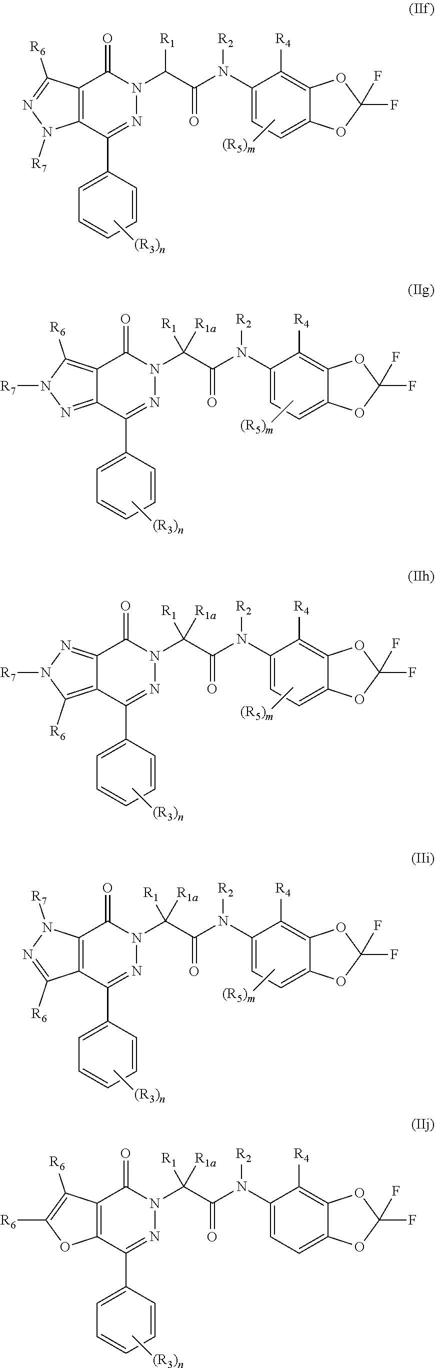

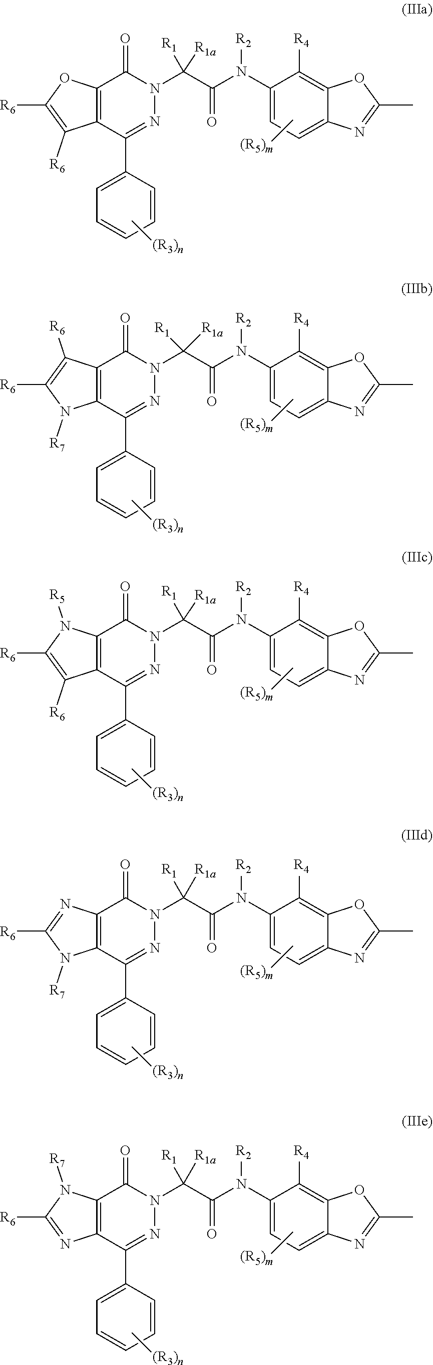

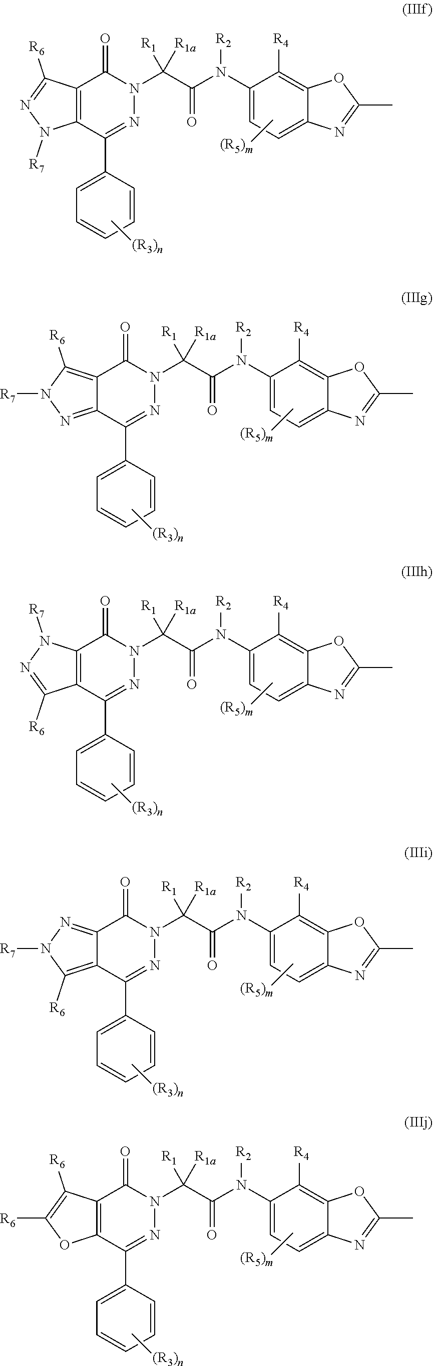

In certain embodiments of the compounds of the invention, each R₆ is hydrogen. In certain embodiments of compounds in which two R₆ groups are present, at least one R₆ is hydrogen. In certain embodiments, at least one R₆ is selected from C₁-C₄-alkyl, such as methyl and t-butyl; fluoro-substituted C₁-C₄-alkyl, such as CF₃ and CHF₂; hydroxyl-C₁-C₄-alkyl, such as 2,3-dihydroxypropyl; optionally substituted C₃- or C₄-cycloalkyl, such as cyclopropyl, cyclobutyl, 1-methylcyclopropyl and 1-trifluoromethylcyclopropyl; R₈R₉NC(O)—, where R₈, R₉ and the nitrogen atom together form a 5- or 6-membered heterocyclyl, such as a pyrrolidine, piperidine or morpholine ring; and R₈OC(O)—, where R₈ is optionally substituted hydrogen or C₁-C₄-alkyl, such as t-butyl.

In certain embodiments of the compounds of the invention,

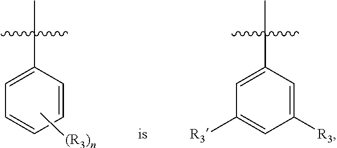

where R₃′ is R₃ or hydrogen. In certain embodiments, R₃′ is hydrogen, and R₃ is selected from CN, F, Cl, methyl, t-butyl, trifluoromethyl, difluoromethyl, methoxy, t-butoxy and trifluoromethoxy. In certain embodiments, R₃′ is hydrogen, and R₃ is selected from CN, F, and Cl.

The compounds of the invention are useful as modulators of CFTR and treating diseases or disorders mediated by CFTR. The present invention, thus, provides methods of treating a disease or disorder mediated by CFTR in a subject in need thereof, comprising administering to the subject a therapeutically effective amount of a compound of the invention. Diseases or disorders mediated by CFTR include cystic fibrosis, Asthma, Constipation, Pancreatitis, Gastrointestinal diseases or disorders, Infertility, Hereditary emphysema, Hereditary hemochromatosis, Coagulation-Fibrinolysis deficiencies, such as Protein C deficiency, Type 1 hereditary angioedema, Lipid processing deficiencies, such as Familial hypercholesterolemia, Type 1 chylomicronemia, Abetalipoproteinemia, Lysosomal storage diseases, such as I-cell disease/Pseudo-Hurler, Mucopolysaccharidoses, Sandhof/Tay-Sachs, Crigler-Najjar type II, Polyendocrinopathy/Hyperinsulemia, Diabetes mellitus, Laron dwarfism, Myeloperoxidase deficiency, Primary hypoparathyroidism, Melanoma, Glycanosis CDG type 1, Hereditary emphysema, Congenital hyperthyroidism, Osteogenesis imperfecta, Hereditary hypofibrinogenemia, ACT deficiency, Diabetes insipidus (DI), Neurophyseal DI, Neprogenic DI, Charcot-Marie Tooth syndrome, Perlizaeus-Merzbacher disease, neurodegenerative diseases such as Alzheimer's disease, Parkinson's disease, Amyotrophic lateral sclerosis, Progressive supranuclear palsy, Pick's disease, several polyglutamine neurological disorders such as Huntington's disease, Spinocerebullar ataxia type I, Spinal and bulbar muscular atrophy, Dentororubal pallidoluysian, and Myotonic dystrophy, as well as spongiform encephalopathies such as Hereditary Creutzfeldt-Jakob disease, Fabry disease, and Straussler-Scheinker disease; secretory diarrhea, polycystic kidney disease, chronic obstructive pulmonary disease (COPD), dry eye disease, Sjogren's Syndrome, congenital bilateral absence of vas deferens (CBAVD), disseminated bronchiectasis, allergic pulmonary aspergillosis, chronic sinusitis, protein C deficiency, A-lipoproteinemia, mild pulmonary disease, lipid processing deficiencies, coagulation fibrinolyis, CFTR-related metabolic syndrome, chronic bronchitis, constipation, pancreatic insufficiency, melanoma, glycanosis CDG type 1, ACT deficiency, allergic pulmonary aspergillosis; celiac disease; vascular inflammation-atherosclerotic disease, increased glucagon production, cholestatic liver disease (e.g. Primary biliary cirrhosis (PBC) and primary sclerosing cholangitis (PSC)).

In certain embodiments, the disease or disorder mediated by CFTR is selected from congenital bilateral absence of vas deferens; acute, recurrent or chronic pancreatitis; disseminated bronchiectasis; asthma; allergic pulmonary aspergillosis; smoking related lung disease (e.g., chronic obstructive pulmonary disease, COPD); dry eye disease; Sjogren's syndrome; chronic sinusitis; cholestatic liver disease, such as primary biliary cirrhosis and primary sclerosing cholangitis; and polycystic kidney disease (autosomal dominant).

In certain embodiments, the disease or disorder mediated by CFTR is selected from celiac disease; vascular inflammation-atherosclerotic disease; dry eye (keratoconjunctivitis sicca) with or without associated autoimmune disease; polycystic kidney disease; cystic fibrosis-related diabetes mellitus; increased glucagon production; non-atopic asthma; non-CF bronchiectasis; and constipation.

The compounds of the invention can be administered in combination with one or more additional therapeutic agents, such as antibiotics, anti-inflammatory medicines, bronchodilators, or mucus-thinning medicines. In particular, antibiotics for the treatment of bacteria mucoid Pseudomonas can be used in combination with compounds of the invention. Inhaled antibiotics such as tobramycin, colistin, and aztreonam can be used in combination with treatment with compounds of the invention. Anti-inflammatory medicines can also be used in combination with compounds of the invention to treat CFTR related diseases. Bronchodilators can be used in combination with compounds of the invention to treat CFTR related diseases. In certain embodiments, the compound of the invention is administered in combination with a second compound which is a CFTR modulator.

In one embodiment, the invention provides a method of treating cystic fibrosis or a symptom thereof, in a subject in need thereof, comprising administering to the subject a therapeutically effective amount of a compound of the invention. The compound of the invention is optionally administered in combination with one or more additional pharmaceutical agents useful for the treatment of cystic fibrosis, such as compounds which are CFTR modulators. In a preferred embodiment, the additional pharmaceutical agent is the aminoglycoside gentamicin. In a preferred embodiment, the additional pharmaceutical agent is a CFTR modulator, such as ataluren, ivacaftor (KALYDECO™), VX-445, VX-659, or lumacaftor or tezacaftor or combinations of two or more thereof, PTI-428, PTI-801, PTI-808, GLPG1837, GLPG2222, GLPG2737 or other modulators of CFTR expression, activity and/or function. In another embodiment, a compound of the invention is administered in combination with a second compound selected from FDL169 and FDL176. In one embodiment, the compound of the invention is administered in combination with both FDL169 and FDL176. In one embodiment, the invention relates to a pharmaceutical composition comprising a compound of the invention and a pharmaceutically acceptable excipient or carrier. The compositions can include one or more compounds of the invention, and a pharmaceutically acceptable carrier, adjuvant or vehicle. In certain embodiments, these compositions further comprise one or more additional therapeutic agents useful for the treatment of CFTR mediated diseases or disorders.

### Pharmaceutical Compositions

The pharmaceutical compositions of the present invention comprise a compound of the present invention formulated together with one or more pharmaceutically acceptable carriers or excipients.

As used herein, the term “pharmaceutically acceptable carrier or excipient” means a non-toxic, inert solid, semi-solid, gel or liquid filler, diluent, encapsulating material or formulation auxiliary of any type. Some examples of materials which can serve as pharmaceutically acceptable carriers are sugars such as lactose, glucose and sucrose; cyclodextrins such as alpha- (α), beta- (β) and gamma- (γ) cyclodextrins; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention can be administered orally, parenterally, by inhalation spray, topically, rectally, nasally, buccally, vaginally or via an implanted reservoir. In a preferred embodiment, administration is oral administration. Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

The pharmaceutical compositions of this invention can contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

In another embodiment, administration is parenteral administration by injection. Injectable preparations, for example, sterile injectable aqueous or oleaginous suspensions, may be formulated according to the known art using suitable dispersing or wetting agents and suspending agents. The sterile injectable preparation may also be a sterile injectable suspension or emulsion, such as INTRALIPID®, LIPOSYN® or OMEGAVEN®, or solution, in a nontoxic parenterally acceptable diluent or solvent, for example, as a solution in 1,3-butanediol. INTRALIPID® is an intravenous fat emulsion containing 10-30% soybean oil, 1-10% egg yolk phospholipids, 1-10% glycerin and water. LIPOSYN® is also an intravenous fat emulsion containing 2-15% safflower oil, 2-15% soybean oil, 0.5-5% egg phosphatides 1-10% glycerin and water. OMEGAVEN® is an emulsion for infusion containing about 5-25% fish oil, 0.5-10% egg phosphatides, 1-10% glycerin and water. Among the acceptable vehicles and solvents that may be employed are water, Ringer's solution, USP and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics is known in the art (see, for example U.S. Pat. No. 5,767,068 to Van Devanter et al., U.S. Pat. No. 5,508,269 to Smith et al., and WO 98/43650 by Montgomery).

The compositions described herein can be formulated in a unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. The amount of the active compound in a unit dosage form will vary depending upon, for example, the host treated, and the particular mode of administration. In one embodiment, the unit dosage form can have one of the compounds of the invention as an active ingredient in an amount of about 10 mg, 20 mg, 30 mg, 40 mg, 50 mg, 100 mg, 150 mg, 200 mg, 250 mg, 300 mg, 400 mg, 500 mg, 600 mg, 700 mg, 750 mg, 800 mg, 900 mg, 1000 mg, or 1,250 mg.

In some embodiments, the compounds of the invention can be administered in a dose of at least about 10 mg/day to at least about 1500 mg/day. In some embodiments, the compounds of the invention are administered in a dose of at least about 300 mg (e.g., at least about 450 mg, at least about 500 mg, at least about 750 mg, at least about 1,000 mg, at least about 1250 mg, or at least about 1500 mg).

Dose adjustments can be made for patients with mild, moderate or severe hepatic impairment (Child-Pugh Class A). Furthermore, dosage adjustments can be made for patients taking one or more Cytochrome P450 inhibitors and inducers, in particular CYP3A4, CYP2D6, CYP2C9, CYP2C19 and CYP2B6 inhibitors and inducers. Dose adjustments can also be made for patients with impaired Cytochrome P450 function such as poor, intermediate, extensive and ultra-rapid metabolizers.

### Definitions

Listed below are definitions of various terms used to describe this invention. These definitions apply to the terms as they are used throughout this specification and claims, unless otherwise limited in specific instances, either individually or as part of a larger group.

The term “alkyl” is intended to include both branched and straight chain, substituted or unsubstituted saturated aliphatic hydrocarbon radicals/groups having the specified number of carbons. Preferred alkyl groups comprise about 1 to about 24 carbon atoms (“C₁-C₂₄”). Other preferred alkyl groups comprise at about 1 to about 8 carbon atoms (“C₁-C₈”) such as about 1 to about 6 carbon atoms (“C₁-C₆”), or such as about 1 to about 3 carbon atoms (“C₁-C₃”). Examples of C₁-C₆ alkyl radicals include, but are not limited to, methyl, ethyl, propyl, isopropyl, n-butyl, tert-butyl, n-pentyl, neopentyl and n-hexyl radicals.

The term “alkenyl” refers to linear or branched radicals having at least one carbon-carbon double bond. Such radicals preferably contain from about two to about twenty-four carbon atoms (“C₂-C₂₄”). Other preferred alkenyl radicals are “lower alkenyl” radicals having two to about ten carbon atoms (“C₂-C₁₀”) such as ethenyl, allyl, propenyl, butenyl and 4-methylbutenyl. Preferred lower alkenyl radicals include 2 to about 6 carbon atoms (“C₂-C₆”). The terms “alkenyl”, and “lower alkenyl”, embrace radicals having “cis” and “trans” orientations, or alternatively, “E” and “Z” orientations.

The term “alkynyl” refers to linear or branched radicals having at least one carbon-carbon triple bond. Such radicals preferably contain from about two to about twenty-four carbon atoms (“C₂-C₂₄”). Other preferred alkynyl radicals are “lower alkynyl” radicals having two to about ten carbon atoms such as propargyl, 1-propynyl, 2-propynyl, 1-butyne, 2-butynyl and 1-pentynyl. Preferred lower alkynyl radicals include 2 to about 6 carbon atoms (“C₂-C₆”).

The term “aryl,” as used herein, refers to a mono- or polycyclic carbocyclic ring system comprising at least one aromatic ring, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof.

As used herein, the term “arylalkyl” means a functional group wherein an alkylene chain is attached to an aryl group, e.g., —CH₂CH₂-phenyl. The term “substituted arylalkyl” means an arylalkyl functional group in which the aryl group is substituted. Similarly, the term “heteroarylalkyl” means a functional group wherein an alkylene chain is attached to a heteroaryl group. The term “substituted heteroarylalkyl” means a heteroarylalkyl functional group in which the heteroaryl group is substituted.

As used herein, the term “alkoxy” employed alone or in combination with other terms means, unless otherwise stated, an alkyl group having the designated number of carbon atoms connected to the rest of the molecule via an oxygen atom, such as, for example, methoxy, ethoxy, 1-propoxy, 2-propoxy (isopropoxy) and the higher homologs and isomers. Preferred alkoxy are (C₁-C₃) alkoxy.

The term “cycloalkyl” refers to saturated carbocyclic radicals having three to about twelve carbon atoms (“C₃-C₁₂”). The term “cycloalkyl” embraces saturated carbocyclic radicals having three to about twelve carbon atoms. Examples of such radicals include cyclopropyl, cyclobutyl, cyclopentyl and cyclohexyl.

The term “alkoxy” is intended to refer to an alkyl-O— radical.

The term “cycloalkenyl” refers to partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The terms “heterocyclyl”, “heterocycle” “heterocyclic” or “heterocyclo” refer to saturated, partially unsaturated and unsaturated heteroatom-containing ring-shaped radicals, which can also be called “heterocyclyl”, “heterocycloalkenyl” and “heteroaryl” correspondingly, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The terms “halogen” or “halo” as used herein, refers to an atom selected from fluorine, chlorine, bromine and iodine. Preferred halogens are fluorine and chlorine.

The term “haloalkyl” refers to an alkyl group which includes one or more halogen substituents.

The term “haloalkoxy” refers to an alkoxy group which includes one or more halogen substituents.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)— heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —CO₂—C₁-C₁₂ alkyl, —CO₂—C₂-C₈ alkenyl, —CO₂—C₂-C₈ alkynyl, CO₂—C₃-C₁₂-cycloalkyl, —CO₂-aryl, CO₂-heteroaryl, CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocyclo-alkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocyclo-alkyl, —NHCO₂—C₁-C₁₂-alkyl, —NHCO₂—C₂-C₈-alkenyl, —NHCO₂— C₂-C₈-alkynyl, —NHCO₂—C₃-C₁₂-cycloalkyl, —NHCO₂-aryl, —NHCO₂-heteroaryl, —NHCO₂-heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH— heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)— heterocycloalkyl, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH— heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl, —SO₂NH₂, —SO₂NH—C₁-C₁₂-alkyl, —SO₂NH—C₂-C₈-alkenyl, —SO₂NH—C₂-C₈-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthio-methyl. In certain embodiments, the substituents are independently selected from halo, preferably C₁ and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy, —CN; —OH; NH₂; C₁-C₄-alkylamino; di(C₁-C₄-alkyl)amino; and NO₂. It is understood that the aryls, heteroaryls, alkyls, and the like can be further substituted. In some cases, each substituent in a substituted moiety is additionally optionally substituted when possible with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group, such as a substituted methyl group, is substituted with one or more halogen atoms, more preferably one or more fluorine or chlorine atoms.

The term “optionally substituted”, as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The compounds of the invention can occur in various forms, including salt forms, particularly pharmaceutically acceptable salts, co-crystals, solvates, hydrates, polymorphs, enantiomers, diastereoisomers, racemates and the like of the compounds having a formula as set forth herein. In certain embodiments, the compounds of the invention occur as a racemic mixture, for example of stereoisomers having the stereochemistry of Formulas (Ia), (IIa), (IIIa), and (IVa) and Formulas (Ib), (IIb), (IIIb), and (IVb). In other embodiments, the compounds exist as mixtures of two enantiomers, with an enantiomeric excess of one enantiomer. In still other embodiments, the compounds exist as substantially pure single enantiomers, for example with an enatiomeric excess of one enantiomer of at least 90%, 95%, 98% or 99%.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include salts of an acid drug with nontoxic ammonium, quaternary ammonium, and amine cations.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in T. H. Greene and P. G. M. Wuts, Protective Groups in Organic Synthesis, 3rd edition, John Wiley & Sons, New York (1999). Examples of hydroxyl protecting groups include benzyloxycarbonyl, 4-methoxybenzyloxycarbonyl, tert-butoxycarbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in T. H. Greene and P. G. M. Wuts, Protective Groups in Organic Synthesis, 3rd edition, John Wiley & Sons, New York (1999). Examples of amino protecting groups include, but are not limited to, methoxycarbonyl, t-butoxycarbonyl, 9-fluorenyl-methoxycarbonyl, benzyloxycarbonyl, and the like.

The term “protected amino,” as used herein, refers to an amino group protected with an amino protecting group as defined above.

The present invention includes all pharmaceutically acceptable isotopically-labeled or enriched compounds of the invention. These compounds include at one or more positions an isotopic abundance or the indicated element which differs from the natural isotopic distribution for that element. For example, a position at which a hydrogen atom is depicted can include deuterium at a higher abundance than the natural abundance of deuterium.

Examples of isotopes suitable for inclusion in the compounds of the invention comprises isotopes of hydrogen, such as ²H and ³H, carbon, such as ¹¹C, ¹³C and ¹⁴C, nitrogen, such as ¹³N and ¹⁵N, oxygen, such as ¹⁵O, ¹⁷O and ¹⁸O, chlorine, such as ³⁶Cl, fluorine, such as ¹⁸F, iodine, ¹²³I and ¹²⁵I, phosphorus, such as ³²P, and sulfur, such as ³⁵S.

Substituents indicated as attached through variable points of attachments can be attached to any available position on the ring structure.

As used herein, the term “therapeutically effective amount of the subject compounds,” with respect to the subject method of treatment, refers to an amount of the subject compound which, when delivered as part of desired dose regimen, brings about management of the disease or disorder to clinically acceptable standards.

“Treatment” or “treating” refers to an approach for obtaining beneficial or desired clinical results in a patient. For purposes of this invention, beneficial or desired clinical results include, but are not limited to, one or more of the following: alleviation of symptoms, diminishment of extent of a disease, stabilization (i.e., not worsening) of a state of disease, preventing spread (i.e., metastasis) of disease, preventing occurrence or recurrence of disease, delay or slowing of disease progression, amelioration of the disease state, and remission (whether partial or total).

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### EXAMPLES

List of Abbreviations:
All temperatures are in degrees Centigrade
BF₃.Et₂O—boron trifluoride etherate
CDCl₃—deuterated chloroform
CF—cystic fibrosis
CFTR—cystic fibrosis transmembrane conductance regulator
CH₃CN—acetonitrile
CH₃NO₂—nitromethane
CH₂Cl₂—methylene chloride
DIPEA—N,N-diisopropylethylamine
DMF—dimethylformamide
DMSO-d₆—deuterated dimethylsulfoxide
ENaC—epithelial sodium channel
Et₂O—diethyl ether
EtOAc—ethyl acetate
H₂O—water
HATU—(1-[Bis(dimethylamino)methylene]-1H-1,2,3-triazolo[4,5-b]pyridinium 3-oxid hexafluorophosphate)
HBS—Hepes-buffered saline
HCl—hydrochloric acid
HCOOH—formic acid
HPLC—high pressure liquid chromatography
hr—hours
HTS—high throughput screen
ms—milliseconds
Na₂SO₄—sodium sulfate
NaBH₄—sodium borohydride
NaOH—sodium hydroxide
NaHCO₃—sodium bicarbonate
NAUC—normalized area under the curve
NH₄OAc—ammonium acetate
NMR—nuclear magnetic resonance
Pet. Ether—petroleum ether
PBS—Phosphate buffered saline
Pd(PPh₃)₄—palladium tetrakis
s—seconds
rt—room temperature
TFA—trifluoroacetic acid
THF—tetrahydrofuran
YFP—yellow fluorescent protein

### EXAMPLES

### Synthesis of Intermediates

### (1) N-ethyl-2,2-difluorobenzo[d][1,3]dioxol-5-amine

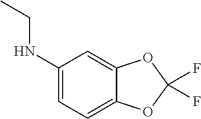

To a solution of 2,2-difluorobenzo[d][1,3]dioxol-5-amine (7.45 g, 43.0 mmol) in DMF (60 mL) was added K₂CO₃ (17.8 g, 129 mmol), and the reaction mixture was stirred at rt for 1 hr. Iodoethane (3.52 mL, 43.0 mmol) was added dropwise at 0° C. and mixture was stirring overnight at rt. The reaction mixture was diluted with water (500 mL) and the product extracted with EtOAc (3×100 mL). The combined organics were washed with brine (200 mL), dried over Na₂S₄, and concentrated. The crude product was purified by chromatography (0-10% EtOAc/hexane) to yield N-ethyl-2,2-difluorobenzo[d][1,3]dioxol-5-amine (5.98 g). (MS: ESI +ve, 202 [M+H]).

### 2,2-difluoro-N-methylbenzo[d][1,3]dioxol-5-aminene

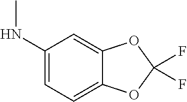

2,2-difluoro-N-methylbenzo[d][1,3]dioxol-5-aminene was synthesized in a similar fashion as N-ethyl-2,2-difluorobenzo[d][1,3]dioxol-5-amine.

### (2) N-cyclopropyl-2,2-difluorobenzo[d][1,3]dioxol-5-amine

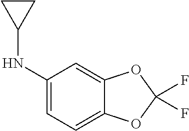 [SMILES: FC1(F)Oc2ccc(NC3CC3)cc2O1]

To a stirred solution of 2,2-difluorobenzo[d][1,3]dioxol-5-amine (10 g, 0.067 mmol) in methanol (70 mL) was added acetic acid (2 mL) at room temperature followed by sodium cynoborohydride (21.64 g, 0.344 mmol), (1-ethoxycyclopropoxy)trimethylsilane (12.9 g, 0.074 mmol). The reaction mixture was stirred at 70° C. for 16 h. and then was quenched with ice water, extracted with ethyl acetate. The organic layers were dried over sodium sulfate and concentrated. The crude product was purified by column chromatography (2-5% ethyl acetate in hexane) to obtain N-cyclopropyl-2,2-difluorobenzo[d][1,3]dioxol-5-amine (8.0 g, 67%). (MS ESI +ve, 214. [M+1]).

### (3) N-cyclobutyl-2,2-difluorobenzo[d][1,3]dioxol-5-amine

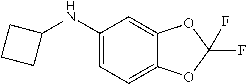 [SMILES: FC1(F)Oc2ccc(NC3CCC3)cc2O1]

A mixture of 2,2-difluorobenzo[d][1,3]dioxol-5-amine (0.6 g, 3.46 mmol), cyclobutanone (0.76 mL g, 10.39 mmol) and AcOH (0.3 mL, 5.19 mmol) in dichloromethane (10 mL) was stirred at room temperature for 1 h. The mixture was cooled to 0° C. by ice-water and sodium triacetoxy borohydride (1.09 g, 5.19 mmol) was added. The resulting solution was allowed to warm up to room temperature and stirred for 16 h. Solvent were removed under vacuum and the residue was purified by column chromatography (2-5% Ethyl acetate in hexane) to give N-cyclobutyl-2,2-difluorobenzo[d][1,3]dioxol-5-amine (0.650 g, 82%) as colorless liquid. (MS ESI +ve, 228.2 [M+H]).

### (4) N,2-dimethylbenzo[d]oxazol-6-amine

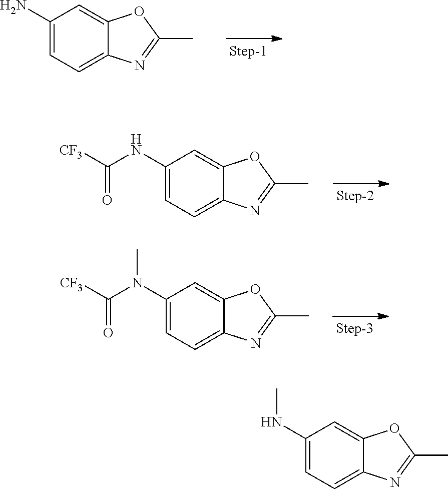 [SMILES: Cc1nc2ccc(N)cc2o1 ; Cc1nc2ccc(NC(=O)C(F)(F)F)cc2o1 ; Cc1nc2ccc(N(C)C(=O)C(F)(F)F)cc2o1 ; CNc1ccc2nc(C)oc2c1]

Step-1: A solution of 2-methylbenzo[d]oxazol-6-amine (8.4 g, 56.7 mmol) in pyridine (80 mL) at 0° C. was treated with TFAA (19.8 mL, 141.0 mmol) and stirred at rt for 4 h. The reaction mixture was diluted with water (100 mL) and the product extracted with EtOAc (3×100 mL). The organics were washed with brine, dried over Na₂SO₄ and concentrated to obtain the crude 2,2,2-trifluoro-N-(2-methylbenzo[d]oxazol-6-yl)acetamide (15.0 g), (MS: ESI +ve, 245.20 [M+H]); ¹H NMR: (400 MHz, DMSO) δ: 2.61 (s, 3H), 7.61-7.56 (m, 1H), 7.69-7.67 (d, J=8.4, 1H), 8.04-8.04 (d, J=2, 1H), 11.45 (s, 1H).

Step-2: A solution of 2,2,2-trifluoro-N-(2-methylbenzo[d]oxazol-6-yl)acetamide (15.0 g, 61.2 mmol) in DMF (100 mL) was treated with K₂CO₃ (8.45 g, 61.2 mmol) and the reaction mixture was stirred at rt for 1 h, then cooled to 0° C. Iodomethane (3.9 mL, 64.2 mmol) was added dropwise and stirring continued at rt overnight. The reaction mixture was diluted with water (100 mL) and extracted with EtOAc (3×100 mL). The organics were washed with brine, dried over Na₂SO₄ and concentrated to obtain crude 2,2,2-trifluoro-N-methyl-N-(2-methylbenzo[d]oxazol-6-yl)acetamide (16.5 g), (MS: ESI +ve, 259.26 [M+H]).

Step-3: To a solution of crude 2,2,2-trifluoro-N-methyl-N-(2-methylbenzo[d]oxazol-6-yl)acetamide (16.5 g, 63.0 mmol) in MeOH (440 mL) and water (73 mL) was added K₂CO₃ (35.3 g, 25.5 mmol). The reaction mixture was stirred at reflux for 3 h, then concentrated under vacuum, diluted with water (50 mL), extracted with EtOAc (2×50 mL). The organics were washed with brine, dried over Na₂SO₄, and concentrated to obtain crude N,2-dimethylbenzo[d]oxazol-6-amine (8.3 g), (MS: ESI +ve, 163.12 [M+H]); ¹H NMR: (400 MHz, DMSO) δ: 2.51 (s, 3H), 2.56 (s, 3H), 5.87-5.85 (m, 1H), 6.57-6.53 (m, 1H), 6.65-6.64 (d, J=2.4, 1H), 7.31-7.28 (t, 1H).

### N-ethyl-2-methylbenzo[d]oxazol-6-amine

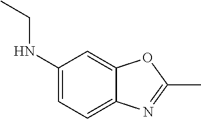

N-ethyl-2-methylbenzo[d]oxazol-6-amine was synthesized in a similar fashion as N,2-dimethylbenzo[d]oxazol-6-amine

### (5) 2,2-difluoro-6,7,8,9-tetrahydro-[1,3]dioxolo[4,5-f]quinoline

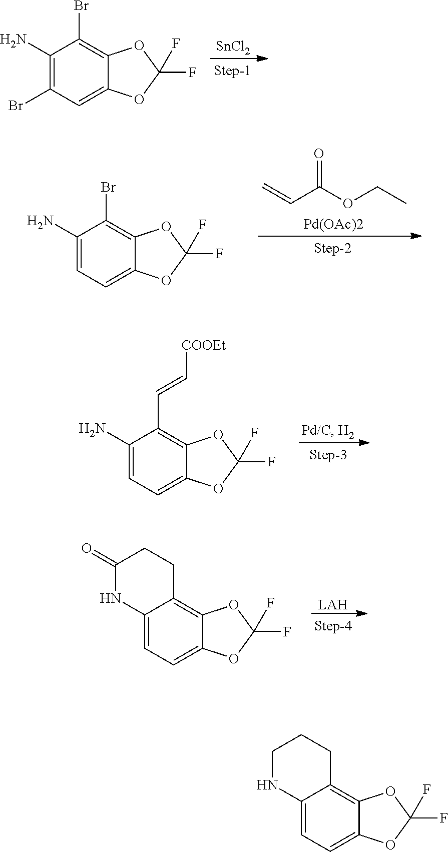

Step-1: A mixture of 4,6-dibromo-2,2-difluorobenzo[d][1,3]dioxol-5-amine (12.3 g, 37.2 mmol), tin chloride (9.2 g, 4.88 mmol) in acetic acid (60 mL) and hydrochloric acid (50 mL) was heated at 110-120° C. for 30 min. Acetic acid was removed by evaporation and the residue was basified with 1N Sodium hydroxide. The mixture was extracted with Dichloromethane (3×100 mL). The combined organic layer was washed with brine (2×100 mL), dried over sodium sulphate. The solvent were removed under vacuum to give 4-bromo-2,2-difluorobenzo[d][1,3]dioxol-5-amine (7.5 g, 80%) as white solid. 1H NMR (400 MHz, DMSO-d6): 5.48 (s, 2H), 6.53-6.55 (d, J=8.6, 1H), 7.09-7.11 (d, J=8.6, 1H).

Step-2: To a solution of 4-bromo-2,2-difluorobenzo[d][1,3]dioxol-5-amine (2.0 g, 7.93 mmol) in acetonitrile (20.0 mL) were added ethyl acrylate (1.1 g, 11.0 mmol) and triethyl amine (2 mL, 15.8 mmol). The mixture was degassed using Nitrogen balloon for 15 minutes and Pd(OAc)₂ (0.17 g, 0.79 mmol), Tri(o-tolyl)phosphine (0.72 g, 2.37 mmol) were added and the mixture was further degassed for 15 minutes. The resulting solution was heated at 120° C. for 24 h, and was diluted with water, extracted with ethyl acetate (3×100 mL). The combined organic layer was washed with brine and dried over sodium sulphate. The volatiles were removed under vacuum to provide the crude mixture which was purified by silica gel column chromatography using 15% Ethyl acetate in hexane to give ethyl (E)-3-(5-amino-2,2-difluorobenzo[d][1,3]dioxol-4-yl)acrylate as yellow solid (1.0 g, 46%). (MS ESI +ve, 272.2 [M+H]).

Step-3: To a mixture of ethyl (E)-3-(5-amino-2,2-difluorobenzo[d][1,3]dioxol-4-yl)acrylate (1.8 g, 6.63 mmol) in methanol (50 mL) was added Palladium on charcoal (1.0 g). The mixture was stirred at rt for 16 h under hydrogen atmosphere (400 psi) in autoclave. The mixture was filtered through celite bed and the volatiles were removed under vacuum to provide the crude mixture which was purified by silica gel column chromatography using 25% Ethyl acetate in hexane as eluent to give 2,2-difluoro-8,9-dihydro-[1,3]dioxolo[4,5-f]quinolin-7(6H)-one (0.9 g, 59%) as white solid. (MS: ESI +ve, 228.3 [M+H]).

Step-4: A solution of 2,2-difluoro-8,9-dihydro-[1,3]dioxolo[4,5-f]quinolin-7(6H)-one (0.850 g, 3.74 mmol) in THF (20 mL) was treated with LAH solution (1M in THF, 4.86 mL, 4.86 mmol). The resultant mixture was allowed warm to rt and stirred for 16 h. The reaction was quenched with ice-cold water. The aqueous layer was extracted with ethyl acetate (3×50 mL), combined organic layer was washed with brine and dried over sodium sulphate. The solvent were removed under vacuum to give 2,2-difluoro-6,7,8,9-tetrahydro-[1,3]dioxolo[4,5-f]quinolone (0.70 g, 87%) as white solid. (MS ESI +ve, 214.2 [M+H]).

### (6) 2,2-difluoro-N,4-dimethylbenzo[d][1,3]dioxol-5-amine

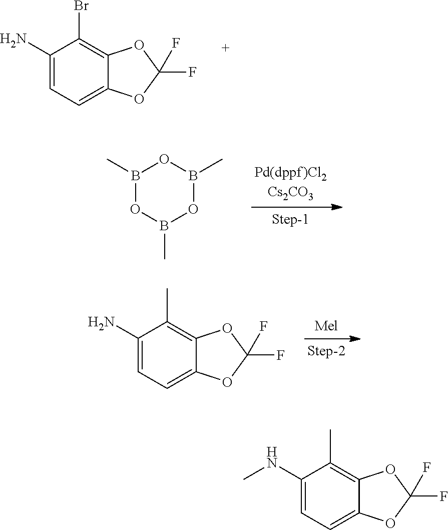

Step-1: To a solution of 4-bromo-2,2-difluorobenzo[d][1,3]dioxol-5-amine (8.0 g, 31.7 mmol) in dioxane (50.0 mL) were added trimethylboroxine (9.5 g, 38.9 mmol), aqueous cesium carbonate (20.6 g, 63.6 mmol). The mixture was degassed using nitrogen balloon for 15 minutes and PdCl₂(dppf) (0.371 g, 0.507 mmol) was added. The mixture was further degassed for 15 minutes and heated at 90° C. for 16 h. The mixture was diluted with water and the aqueous layer was extracted with ethyl acetate (3×100 mL). The combined organic layer was washed with brine and dried over sodium sulphate. The volatiles were removed under vacuum to provide the crude mixture which was purified by silica gel column chromatography using 15% Ethyl acetate in hexane as eluent to give 2,2-difluoro-4-methylbenzo[d][1,3]dioxol-5-amine (5.5 g, 92%) as colorless liquid. (MS ESI +ve, 188.0 [M+H]).

Step-2: To a mixture of 2,2-difluoro-4-methylbenzo[d][1,3]dioxol-5-amine (0.240 g, 1.28 mmol) in DMF (4.0 mL), K₂CO₃ (0.354 g, 2.56 mmol) was added methyl iodide (0.2 g, 1.41 mmol) dropwise at 0° C. The mixture was warmed up to rt and stirred for 16 h. The reaction mixture was diluted with ice-water and the aqueous layer was extracted with ethyl acetate (3×50 mL). The combined organic layer was washed with brine and dried over sodium sulphate. The volatiles were removed under vacuum and resulting material was purified by column chromatography (100-200 mesh silica eluent=5-10% Ethyl acetate in hexane) to provide 2,2-difluoro-N,4-dimethylbenzo[d][1,3]dioxol-5-amine (0.08 g, 74%) as colorless liquid. (MS ESI +ve, 202.0 [M+H]).

The following compounds were synthesized in a similar fashion:

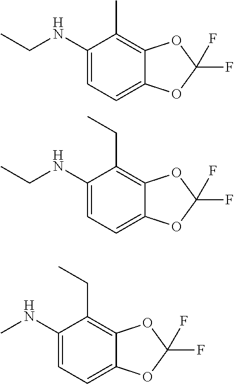

### (7) 6-ethyl-2,2-difluoro-N-methylbenzo[d][1,3]dioxol-5-amine

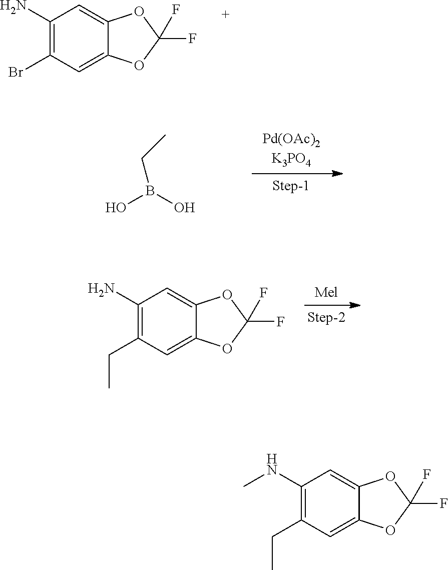

Step-1: To a mixture of 6-bromo-2,2-difluorobenzo[d][1,3]dioxol-5-amine (1.0 g, 4.0 mmol), ethyl boronic acid (1.18 g, 16.0 mmol) and potassium phosphate (3.39 g, 16.0 mmol) in toluene/water (10 mL, 1/1) was added Pd(OAc)₂ (0.18 g, 0.8 mmol) and cataCXium (0.042 g, 0.12 mmol). After purging with nitrogen for 15 min, the reaction mixture was heated at 80° C. for 12 h. The mixture was diluted with water and extracted with ethyl acetate. The combined organic layers were washed with brine, dried over Sodium sulphate and concentrated in vacuum. The product was purified by flash column to obtained 6-ethyl-2,2-difluorobenzo[d][1,3]dioxol-5-amine brown solid (0.65 g, 81%). (MS ESI +Ve: 202.0 [M+1]).

Step-2: To a mixture of 6-ethyl-2,2-difluorobenzo[d][1,3]dioxol-5-amine (0.65 g, 3.22 mmol) in DMF (10 mL), was added potassium carbonate (0.89 g, 6.43 mmol) at 0° C. The mixture was stirred for 15 min, followed by addition of methyl iodide (0.438 g, 3.54 mmol) dropwise. The reaction mixture was stirred at room temperature for 12 h and was diluted with water, extracted with ethyl acetate. Combined organic layer was washed with cold water and brine, dried over sodium sulphate and evaporated. The product was purified by column chromatography (0-20% ethyl acetate in Hexane) to give 6-ethyl-2,2-difluoro-N-methylbenzo[d][1,3]dioxol-5-amine as brown liquid (0.60 g, 86%). (MS ESI +Ve: 216.0 [M+1]).

The following compounds were synthesized in a similar fashion:

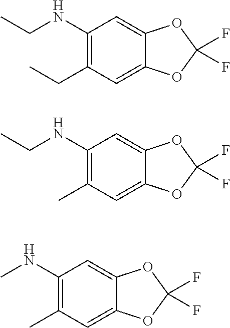 [SMILES: CCNc1cc2c(cc1CC)OC(F)(F)O2 ; CCNc1cc2c(cc1C)OC(F)(F)O2 ; CNc1cc2c(cc1C)OC(F)(F)O2]

### (8) 2′,2′-difluoro-6′,7′-dihydrospiro[cyclopropane-1,8′-[1,3]dioxolo[4,5-e]indole]

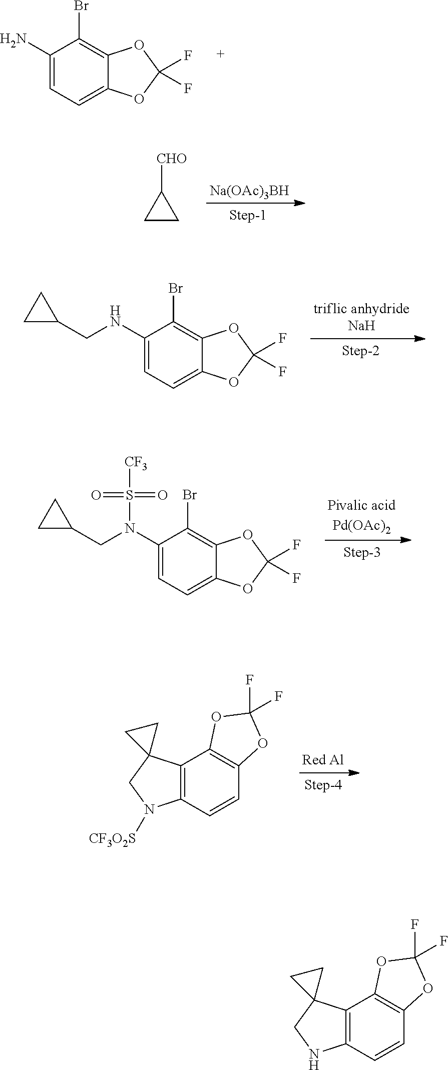

Step-1: A mixture of 4-bromo-2,2-difluorobenzo[d][1,3]dioxol-5-amine (2.0 g, 7.93 mmol), Cyclopropanecarboxaldehyde (0.61 g, 8.79 mmol) and acetic acid (1.3 mL, 23.8 mmol) in DCM (30.0 mL) was stirred at rt for 1 h. The mixture was cooled to 0° C. before addition of sodium triacetoxyborohydride (5.04 g, 23.8 mmol). The resultant mixture was warmed up to rt and stirred for 16 h. Upon completion of the reaction; reaction mixture was diluted by DCM (100.0 mL) and washed by water and brine solution (2×100 mL). Organic layer was dried over sodium sulphate, solvent were removed under vacuum and resulting material was purified by column chromatography (2-5% ethyl acetate in Hexane) to give 4-bromo-N-(cyclopropylmethyl)-2,2-difluorobenzo[d][1,3]dioxol-5-amine (2.0 g, 82%) as colorless liquid. (MS ESI +ve, 306.2 & 308.2 [M+H]).

Step-2: To a solution of 4-bromo-N-(cyclopropylmethyl)-2,2-difluorobenzo[d][1,3]dioxol-5-amine (1.5 g, 4.90 mmol) in 1,2 Dichloroethane (10 mL) was added sodium hydride (0.49 g, 12.3 mmol) portion wise at 0° C. The mixture was stirring for 2 h, followed by addition of triflic anhydride (1.65 mL, 9.80 mmol). The reaction mixture was warmed to room temperature and stirred for 16 h. The reaction was quenched with saturated sodium bicarbonate solution, extracted with dichloromethane (3×100 mL). The combined organic layer was washed with brine (2×100 mL), dried over sodium sulphate. The solvent were removed under vacuum and resulting material was purified by column chromatography (5-10% ethyl acetate in Hexane) to give N-(4-bromo-2,2-difluorobenzo[d][1,3]dioxol-5-yl)-N-(cyclopropylmethyl)-1,1,1-trifluoromethanesulfonamide (1.5 g, 69%) as colorless liquid. ¹H NMR: (400 MHz, CDCl₃): 0.56-0.60 (m, 2H), 0.94-1.02 (m, 2H), 1.24-1.29 (m, 1H), 1.24-1.29 (m, 1H), 3.60 (dd, J=14.4, 7.6 Hz, 1H), 3.77 (dd, J=14.4, 7.6 Hz, 1H), 7.12 (d, J=8.4 Hz, 1H), 7.26 (d, J=8.4 Hz, 1H).

Step-3: A mixture of N-(4-bromo-2,2-difluorobenzo[d][1,3]dioxol-5-yl)-N-(cyclopropylmethyl)-1,1,1-trifluoromethanesulfonamide (1.5 g, 3.42 mmol) in Toluene (10 mL), pivalic acid (0.28 g, 1.71 mmol), cesium carbonate (2.2 g, 6.84 mmol) and tricyclohexylphosphine tetrafluoroborate (0.19 g, 0.51 mmol) was degassed using argon for 15 minutes. Palladium acetate (0.038 g, 0.17 mmol) was added and the mixture was further degassed for 15 minutes. The resulting solution was heated at 110° C. for 16 h and was diluted with ethyl acetate (100 mL), passed through a celite bed. Filtrate was washed with brine and dried over sodium sulphate. The volatiles were removed under vacuum and the residue was purified by silica gel column chromatography (2-5% ethyl acetate in hexane) to give 2′,2′-difluoro-6′-((trifluoromethyl)sulfonyl)-6′,7′-dihydrospiro[cyclopropane-1,8′-[1,3]dioxolo[4,5-e]indole] (0.1 g, 81%) as white solid. ¹H NMR: (400 MHz, CDCl₃): 1.36-1.39 (m, 2H), 1.56-2.09 (m, 2H), 4.21 (s, 2H), 6.87-6.91 (d, J=7.6, 1H), 7.15-7.18 (d, J=7.6, 1H).

Step-4: To the solution of 2′,2′-difluoro-6′-((trifluoromethyl)sulfonyl)-6′,7′-dihydrospiro[cyclopropane-1,8′-[1,3]dioxolo[4,5-e]indole] (0.90 g, 2.52 mmol) in toluene (50 mL) was added sodium bis(2-methoxyethoxy)aluminium hydride (8.4 g, 25.2 mmol). The resultant solution was stirred at room temperature for 30 min and then heated at 50° C. for 16 h. The reaction mixture was quenched with saturated solution of Rochelle salt, extracted with DCM (3×50 mL). The combined organic layer was washed with brine and dried over sodium sulphate. The solvent were removed under vacuum and product was purified by column chromatography (20-30% Ethyl acetate in hexane) to give 2′,2′-difluoro-6′,7′-dihydrospiro[cyclopropane-1,8′-[1,3]dioxolo[4,5-e]indole] (0.24 g, 63%) as colorless liquid. (MS ESI +ve, 226.0 [M+H]).

### (9) 2′,2′-difluoro-5′,6′-dihydrospiro[cyclopropane-1,7′-[1,3]dioxolo[4,5-f]indole]

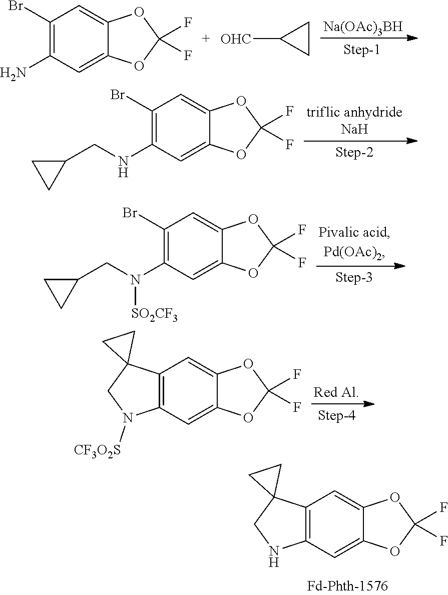

2′,2′-difluoro-5′,6′-dihydrospiro[cyclopropane-1,7′-[1,3]dioxolo[4,5-f]indole] was synthesizes in a similar fashion as colorless liquid (MS ESI +ve, 226.0 [M+H]).

### (10) 2,2-difluoro-N-(tetrahydrofuran-3-yl)benzo[d][1,3]dioxol-5-amine

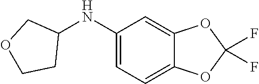 [SMILES: FC1(F)Oc2ccc(NC3CCOC3)cc2O1]

To a mixture of 2,2-difluorobenzo[d][1,3]dioxol-5-amine (3.2 g, 18.5 mmol) and dihydrofuran-3(2H)-one (2.0 mg, 23.2 mmol) in DCM (50 mL) at 0° C. was added acetic acid (3.9 mL, 69.6 mmol) followed by Na(OEt)₃BH (9.8 g, 46.4 mmol). The mixture was stirred at room temperature overnight and was quenched with water, extracted with ethyl acetate. The combined organic layers were washed with brine, dried over Na₂SO₄, and concentrated in vacuo to afford the crude product. The crude product was purified by combiflash chromatography (0-50% ethyl acetate in hexanes) to afford 2,2-difluoro-N-(tetrahydrofuran-3-yl)benzo[d][1,3]dioxol-5-amine (3.72 g, 82%). (MS ESI +Ve 244 [M+1]). ¹H NMR (400 MHz, DMSO) δ: 1.69-1.76 (m, 1H), 2.13-2.22 (m, 1H), 3.48-3.51 (m, 1H), 3.69-3.75 (m, 1H), 3.78-3.88 (m, 2H), 3.89-3.96 (m, 1H), 6.04 (d, 1H, J=6), 6.31-6.34 (dd, 1H, J=2.4), 6.61 (d, 1H, J=2), 7.08-7.10 (d, 1H, J=8.8).

The following compounds were synthesized in a similar fashion:

### 2-methyl-N-(oxetan-3-yl)benzo[d]oxazol-6-amine

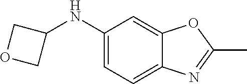

¹H NMR: (400 MHz, DMSO-d6) 2.58 (s, 3H), 4.55-4.58 (t, J=6.0, 2H), 4.62-4.67 (q, J=6.0, 1H), 5.03-5.06 (t, J=6.4, 2H), 6.53-6.55 (m, 2H), 7.43-7.44 (m, 1H).

### N-cyclopropyl-2-methylbenzo[d]oxazol-6-amine

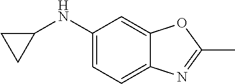

MS ESI +ve, 189.2 [M+1]

### N-cyclopentyl-2,2-difluorobenzo[d][1,3]dioxol-5-amine

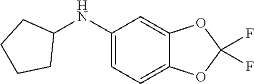 [SMILES: FC1(F)Oc2ccc(NC3CCCC3)cc2O1]

MS ESI +ve, 242.0 [M+H]

### (11) 7-methoxy-3,4-dihydro-2H-benzo[b][1,4]oxazine

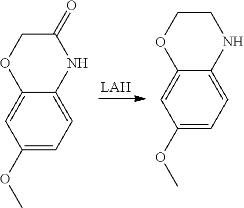

To a mixture of 7-methoxy-2H-benzo[b][1,4]oxazin-3(4H)-one (2 g, 11.2 mmol) in tetrahydrofuran (100 mL) was added lithium aluminium hydride (23.4 mL, 23.45 mmol) at 0° C. The reaction was heated at 70° C. for 7 h and was diluted with ethyl acetate, followed by dropwise addition of 15% sodium hydroxide solution. The mixture was stirred 30 min and then filtered, dried over anhydrous sodium sulphate. Solvent was evaporated to give 7-methoxy-3,4-dihydro-2H-benzo[b][1,4] oxazine (2.3 g, 89%). (MS ESI +Ve 166.21 [M+1]).

### (12) 7-(3-fluorophenyl)furo[2,3-d]pyridazin-4(5H)-one

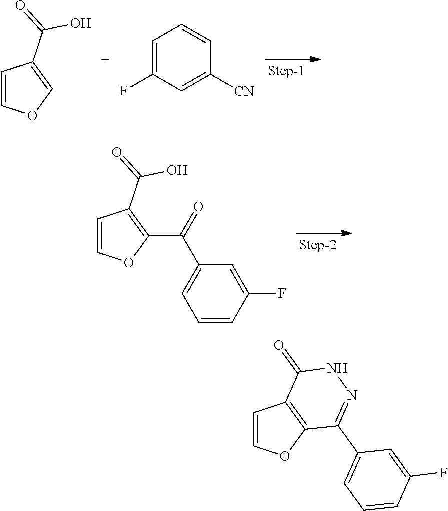 [SMILES: O=C(O)c1ccoc1 ; N#Cc1cccc(F)c1 ; O=C(O)c1ccoc1C(=O)c1cccc(F)c1 ; O=c1[nH]nc(-c2cccc(F)c2)c2occc12]

Step 1: To a solution of furan-3-carboxylic acid (3.0 g, 26.7 mmol) in THF (60 mL) was added dropwise 1.6 M solution of n-BuLi in THF (36.2 mL, 58.0 mmol) at −78° C. The reaction mixture was stirred for 1 h at −78° C., then added 3-fluorobenzonitrile (3.24 g, 26.7 mmol) in THF (20 mL) at −78° C. The reaction mixture was stirred at rt overnight, diluted with ice water (200 mL), acidified with 1N HCl (10 mL), and extracted with EtOAc (200 mL×2). The organic layer was dried over sodium sulfate and concentrated to give 2-(3-fluorobenzoyl) furan-3-carboxylic acid (4.2 g, 66.8%) crude product as white solid which was carried forward for next step without purification. (MS ESI +ve, 235.12 [M+]).

Step 2: To a solution of 2-(3-fluorobenzoyl) furan-3-carboxylic acid (4.2 g, 17.9 mmol) in ethanol (61 mL), hydrazine hydrate (0.9 mL, 19.7 mmol) was added drop wise. The reaction mixture was stirred at 90° C. overnight. The reaction mixture was concentrated, and then diluted with cooled water (250 mL). Solid was filtered out, washed with water and dried to give 7-(3-fluorophenyl)furo[2,3-d]pyridazin-4(5H)-one (1.2 g, 29%) as off white solid. MS ESI +ve, 231.10 [M+H]; ¹H NMR: (400 MHz, DMSO) δ: 7.12-7.13 (d, J=2.0 Hz, 1H), 7.24-7.27 (m, 1H), 7.48-7.52 (m, 1H), 7.67-7.70 (m, 1H), 7.79-7.81 (d, J=8.0 Hz, 1H), 8.22-8.22 (d, J=2.0 Hz, 1H), 13.10 (s, 1H).

The following compounds were synthesized in a similar fashion:

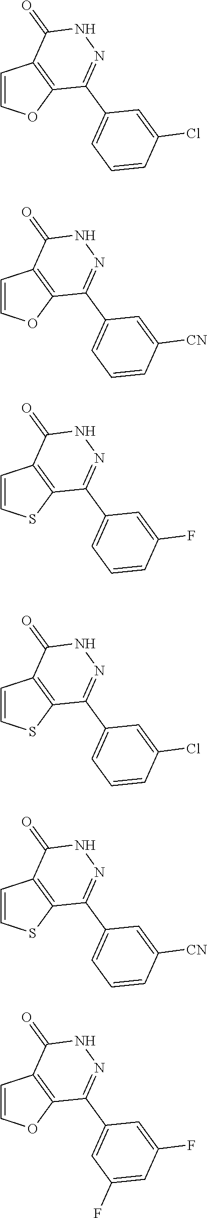 [SMILES: O=c1[nH]nc(-c2cccc(Cl)c2)c2occc12 ; N#Cc1cccc(-c2n[nH]c(=O)c3ccoc23)c1 ; O=c1[nH]nc(-c2cccc(F)c2)c2sccc12 ; O=c1[nH]nc(-c2cccc(Cl)c2)c2sccc12 ; N#Cc1cccc(-c2n[nH]c(=O)c3ccsc23)c1 ; O=c1[nH]nc(-c2cc(F)cc(F)c2)c2occc12]

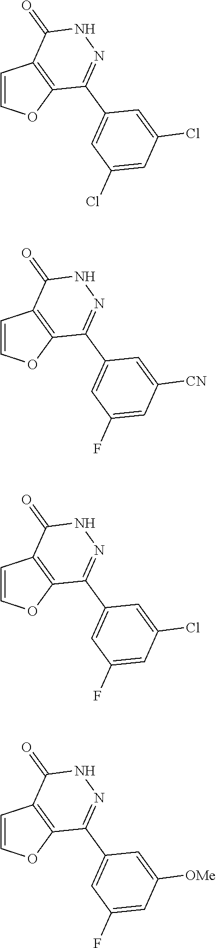 [SMILES: O=c1[nH]nc(-c2cc(Cl)cc(Cl)c2)c2occc12 ; N#Cc1cc(F)cc(-c2n[nH]c(=O)c3ccoc23)c1 ; O=c1[nH]nc(-c2cc(F)cc(Cl)c2)c2occc12 ; COc1cc(F)cc(-c2n[nH]c(=O)c3ccoc23)c1]

### (13) 3-(2-chloro-4-oxo-4,5-dihydrofuro[2,3-d]pyridazin-7-yl)benzonitrile

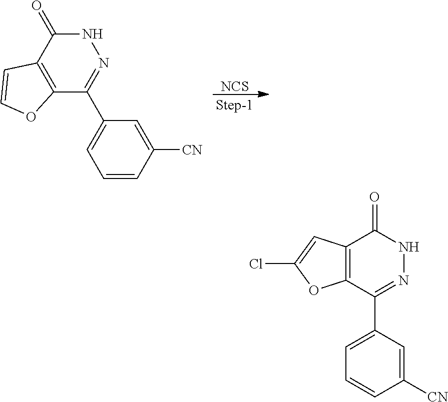 [SMILES: N#Cc1cccc(-c2n[nH]c(=O)c3ccoc23)c1 ; "NCS" ; N#Cc1cccc(-c2n[nH]c(=O)c3cc(Cl)oc23)c1]

To a solution 3-(4-oxo-4,5-dihydrofuro[2,3-d]pyridazin-7-yl)benzonitrile (1.0 g, 4.22 mmol) in water (8 mL) and chloroform (8 mL) was added NCS (0.28 g, 4.22 mmol). The reaction mixture was heated to 70° C. for 15 h. The reaction mixture was treated with aqueous sodium bicarbonate and extracted with DCM. Organic layer was dried over sodium sulfate, concentrated under reduced pressure. The product was purified by column chromatography to afford the pure product 3-(2-chloro-4-oxo-4,5-dihydrofuro[2,3-d]pyridazin-7-yl)benzonitrile (0.14 g, 12%). (MS ESI −Ve, 270[M−1]); ¹H NMR: (400 MHz, DMSO) δ: 7.42 (s, 1H), 7.78 (t, 1H), 8.00-8.02 (d, 1H, J=8), 8.13-8.15 (d, 1H, J=8), 8.18 (s, 1H), 13.8 (s, 1H).

### (14) 7-(3-fluorophenyl)-3,5-dihydrofuro[2,3-d]pyridazin-4(2H)-one

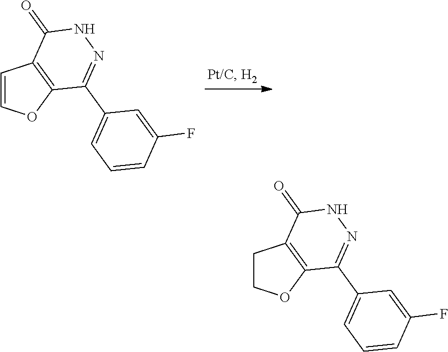 [SMILES: O=c1[nH]nc(-c2cccc(F)c2)c2occc12 ; O=c1[nH]nc(-c2cccc(F)c2)c2c1CCO2]

To a solution of 7-(3-fluorophenyl)furo[2,3-d]pyridazin-4(5H)-one (0.5 g, 2.16 mmol) in DCM (5.0 mL) and EtOAc (5.0 mL) was added 5% Pt/C (0.5 g). The mixture was hydrogenated with a hydrogen balloon for 24 h. Then reaction mixture was filtered through celite and concentrate to give 7-(3-fluorophenyl)-3,5-dihydrofuro[2,3-d]pyridazin-4(2H)-one (0.16 g, 31%) as white solid. (MS ESI +ve: 233 [M+H]).

### (15) 7-(3-fluorophenyl)-3-methylisoxazolo[4,5-d]pyridazin-4(5H)-one

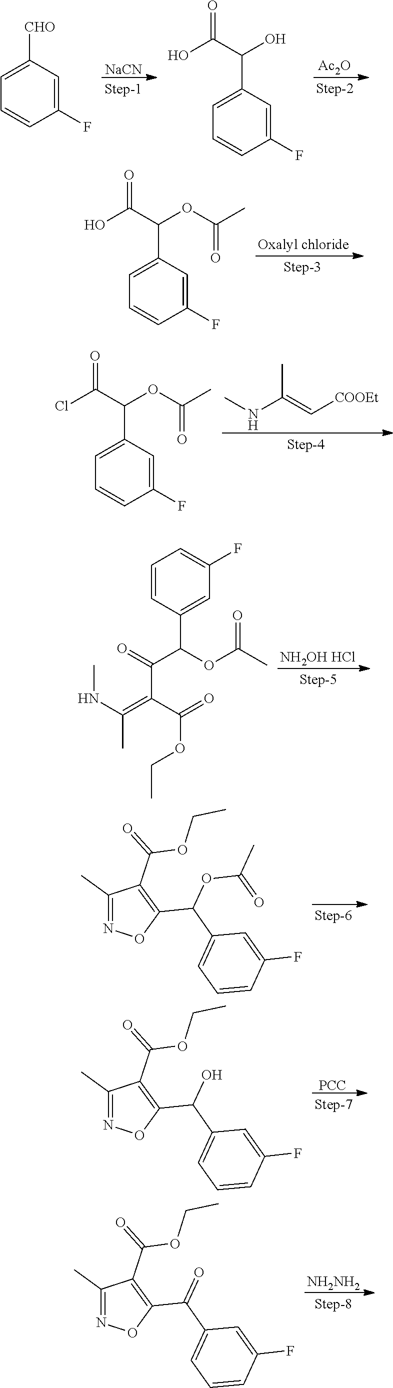

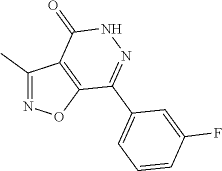 [SMILES: Cc1noc2c(-c3cccc(F)c3)n[nH]c(=O)c12]

Step-1: To a mixture of 3-fluorobenzaldehyde (10.0 g, 8.06 mmol), benzyl trimethyl ammonium chloride (20 mg) in diethyl ether (50 mL) was added sodium cyanide (5.13 g, 10.5 mmol) at 0° C. The mixture was stirred for 5 min and conc. HCl (10 mL) was added dropwise. The mixture was stirred for 16 h at rt and then was extracted with diethyl ether (200 mL×2). The organic solution was treated with 2 M NaOH solution (50 mL). Layers were separated and the inorganic layer was washed with diethyl ether, acidified with 1N aq. HCl (100 mL), extracted with diethyl ether (200 mL). The organic layer was dried, evaporated to give 2-(3-fluorophenyl)-2-hydroxyacetic acid (10.5 g, 76%) as yellowish gum. (MS ESI −ve, 169.07 [M−H]). 1H NMR: (400 MHz, DMSO) δ: 5.08 (s, 1H), 6.03 (s, 1H), 7.07-7.80 (m, 4H), 12.8 (s, 1H).

Step-2: To a solution of 2-(3-fluorophenyl)-2-hydroxyacetic acid (8.0 g, 47.0 mmol) in diethyl ether (70 mL), DMAP (0.287 g, 2.35 mmol) and pyridine (0.1 mL) was added acetic anhydride (5.27 g, 51.7 mmol) at 0° C. The mixture was stirred at rt for 16 h and the reaction was quenched with 1 N HCl (200 mL), extracted with diethyl ether (200 mL×3). The organic layer was dried and evaporated to obtain 2-acetoxy-2-(3-fluorophenyl)acetic acid (9.0 g, 90%) as yellowish gum. ¹H NMR: (400 MHz, DMSO) δ: 2.17 (s, 3H), 5.87 (s, 1H), 7.23-7.50 (m, 4H), 12.3 (s, 1H).

Step-3: To a solution of 2-acetoxy-2-(3-fluorophenyl)acetic acid (12.9 g, 60.8 mmol) in DCM (100 mL) and DMF (1.0 mL) was added dropwise oxalyl chloride (8.08 mL, 91.2 mmol) at 0° C. The mixture was stirred for 2 h at room temperature, and then solvent was evaporated to give 2-chloro-1-(3-fluorophenyl)-2-oxoethyl acetate as yellowish gum (13.0 g, 92%) for the next step.

Step-4: To a solution of ethyl (E)-3-(methylamino)but-2-enoate (8.09 g, 55.9 mmol) in DCM (50 mL) was added pyridine (9.06 mL, 112 mmol) at 0° C. A solution of 2-chloro-1-(3-fluorophenyl)-2-oxoethyl acetate (12.9 g, 55.9 mmol) in DCM (50 mL) was added dropwise to the above reaction mixture. The resulting mixture was stirred at room temperature for 16 h and diluted with water (200 mL), extracted with DCM (200 mL×3). The organic layer was dried, evaporated to give ethyl (E)-2-(2-acetoxy-2-(3-fluorophenyl)acetyl)-3-(methylamino)but-2-enoate as yellowish gum (13.6 g, 72%). (MS ESI +ve, 338.2 [M+H]); ¹H NMR: (400 MHz, DMSO) δ: 1.13-1.17 (d, 3H), 2.07 (s, 3H), 2.49 (s, 3H), 2.99 (s, 3H), 3.34-3.55 (m, 2H), 6.55 (s, 1H), 7.11-7.19 (m, 3H), 7.37-7.42 (m, 1H), 11.89-11.90 (d, J=4.8, 1H).

Step-5: To a solution of ethyl (E)-2-(2-acetoxy-2-(3-fluorophenyl)acetyl)-3-(methylamino)but-2-enoate (13.6 g, 40.3 mmol) in acetic acid (100 mL) was added hydroxyl amine hydrochloride (2.80 g, 40.3 mmol). The mixture was heated to 100° C. for 2 h and solvent was removed by evaporation. The residue was diluted with ice water (150 mL) and extracted with ethyl acetate (100 mL×3). The organic layer was washed with aq. NaHCO₃ (300 mL) and water (200 mL), dried and evaporated to give ethyl 5-(acetoxy(3-fluorophenyl)methyl)-3-methylisoxazole-4-carboxylate (12.0 g, 92%) as yellow gum. (MS ESI +ve, 322.24 [M+H]); ¹H NMR: (400 MHz, DMSO) δ: 1.27-1.32 (m, 3H), 2.15 (s, 3H), 2.33-2.38 (t, 3H), 4.27-4.32 (m, 3H), 7.22-7.32 (m, 3H), 7.45-7.51 (m, 1H).

Step-6: To a solution of ethyl 5-(acetoxy(3-fluorophenyl)methyl)-3-methylisoxazole-4-carboxylate (12.0 g, 37.3 mmol) in ethanol (120 mL) was added HCl (12.0 mL). The mixture was heated to 90° C. for 2 h. After evaporation, the residue was diluted with saturated sodium bicarbonate (150 mL). Solid was collected by filtration, and dried to give ethyl 5-((3-fluorophenyl)(hydroxy)methyl)-3-methylisoxazole-4-carboxylate as yellow gum (10.4 g, 99%). (MS ESI +ve, 280.23 [M+H]); ¹H NMR: (400 MHz, DMSO) δ: 1.11-1.32 (m, 3H), 2.50 (s, 3H), 4.27-4.34 (m, 2H), 6.20-6.21 (d, J=5.6, 1H), 6.42-6.44 (d, J=6.8, 1H), 7.12-7.44 (m, 4H).

Step-7: To a solution of ethyl 5-((3-fluorophenyl)(hydroxy)methyl)-3-methylisoxazole-4-carboxylate (10.5 g, 57.6 mmol) in DCM (100 mL) was added PCC (121 g, 56.4 mmol). The mixture was stirred at room temperature for 16 h and diluted with DCM (250 mL), water (250 mL). The mixture was filtered through celite and organic solution was evaporated to give ethyl 5-(3-fluorobenzoyl)-3-methylisoxazole-4-carboxylate (7.0 g, 67%) as yellow gum. (MS ESI +ve, 278.23 [M+H]); ¹H NMR: (400 MHz, DMSO) δ:1.07-1.10 (t, 3H), 2.59 (s, 3H), 4.14-4.20 (q, 2H), 7.28 (s, 1H), 7.38-7.55 (m, 1H), 7.61-7.63 (d, J=7.2, 2H).

Step-8: To a solution of ethyl 5-(3-fluorobenzoyl)-3-methylisoxazole-4-carboxylate (7.3 g, 26.3 mmol) in ethanol (70 mL) was added hydrazine hydrate (1.31 mL, 26.3 mmol). The mixture was heated to 90° C. for 16 h and the product was collected by filtration to give 7-(3-fluorophenyl)-3-methylisoxazolo[4,5-d]pyridazin-4(5H)-one (5.5 g, 85%) as yellow solid. (MS ESI +ve, 246.18 [M+H]); ¹H NMR: (400 MHz, DMSO) δ: 2.61 (s, 3H), 7.38-7.42 (t, 1H), 7.47-7.67 (q, 1H), 7.74-7.76 (d, J=10.4, 1H), 7.88-7.90 (d, J=7.6, 1H), 13.5 (s, 1H).

### 7-(3-chlorophenyl)-3-methylisoxazolo[4,5-d]pyridazin-4(5H)-one

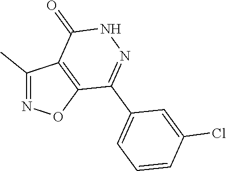 [SMILES: Cc1noc2c(-c3cccc(Cl)c3)n[nH]c(=O)c12]

7-(3-chlorophenyl)-3-methylisoxazolo[4,5-d]pyridazin-4(5H)-one was synthesized in similar fashion.

### (16) 7-(3-chlorophenyl)-2-methylthiazolo[4,5-d]pyridazin-4(5H)-one

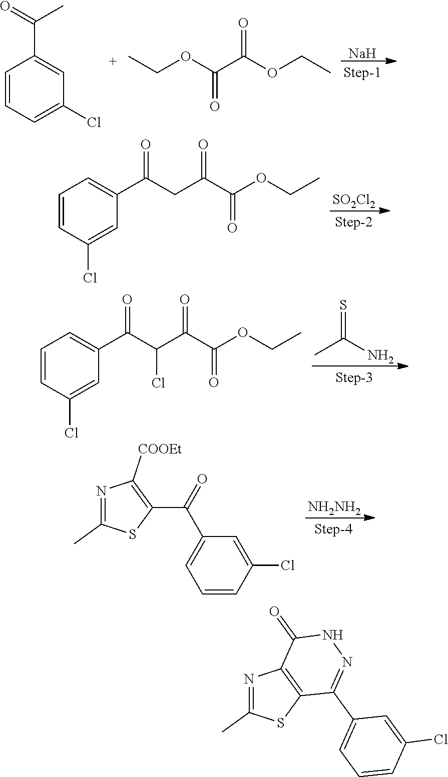

Step-1: To a mixture of 1-(3-chlorophenyl)ethan-1-one (10 g, 64.7 mmol) in THF (50 mL) was added NaH (60%, 3.1 g, 129 mmol) at 0° C. The mixture was stirring for 1 h and diethyl carbonate (14.2 g, 97.0 mmol) was added dropwise at 0° C. The mixture was stirred at room temperature for 6 h and was quenched with HCl (1N, 200 mL), extract with ethyl acetate (100 mL*3). The organic solution was dried and evaporated. The residue was distilled under vacuum to obtain ethyl 4-(3-chlorophenyl)-2,4-dioxobutanoate (13 g, 79%). (MS ESI −ve, 253 M−H]).

Step-2: To a solution of ethyl 4-(3-chlorophenyl)-2,4-dioxobutanoate (5.0 g, 19.6 mmol) in chloroform (50 mL) was added thionyl chloride (13.7 mL, 39.2 mmol) dropwise at room temperature. The reaction mixture was stirred for 4 h and solvent was removed by evaporation. The residue was diluted with water (50 mL) and extract with DCM (25 mL*3). The organic layers were dried, evaporated. The residue was distilled under vacuum to obtain ethyl 3-chloro-4-(3-chlorophenyl)-2,4-dioxobutanoate (4.0 g, 70%). (MS ESI −ve, 287 M−H]).

Step-3: To a solution of ethyl 3-chloro-4-(3-chlorophenyl)-2,4-dioxobutanoate (4.0 g, 14.8 mmol) in diethyl ether (60 mL) was added ethanethioamide (1.0 g, 14.8 mmol). The mixture was stirred for 2 h at room temperature. Solvent was evaporated and the residue was heated at 80° C. for 2 h. The reaction mixture was diluted with water (20 mL) and extract with ethyl acetate (20 mL*2). Organic layer was dried over sodium sulfate and evaporated. The product was purified by combiflash (40% ethyl acetate in hexane) to give ethyl 5-(3-chlorobenzoyl)-2-methylthiazole-4-carboxylate (2.3 g, 53%) as brown solid. (MS ESI +ve, 310 M+H]).

Step-4: To a solution of ethyl 5-(3-chlorobenzoyl)-2-methylthiazole-4-carboxylate (2.3 g, 7.43 mmol) in ethanol (20 mL) was added hydrazine hydrate (0.4 g, 8.16 mmol). The mixture was stirred at 80° C. for 16 h and was cooled down to rt. The product was collected by filtration and dried under vacuum to give 7-(3-chlorophenyl)-2-methylthiazolo[4,5-d]pyridazin-4(5H)-one (1.3 g, 63%) as white solid. (MS ESI +ve, 278 M+H]); ¹H NMR: (400 MHz, DMSO) δ: 2.88 (s, 3H), 7.59-7.64 (m, 2H), 7.75-7.79 (m, 1H), 7.82 (s, 1H).

### 7-(3-fluorophenyl)-2-methylthiazolo[4,5-d]pyridazin-4(5H)-one

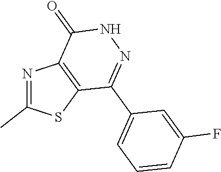 [SMILES: Cc1nc2c(=O)[nH]nc(-c3cccc(F)c3)c2s1]

7-(3-fluorophenyl)-2-methylthiazolo[4,5-d]pyridazin-4(5H)-one was synthesized in a similar fashion.

### (17) 2-bromo-N-methyl-N-(2-methylbenzo[d]oxazol-6-yl)acetamide

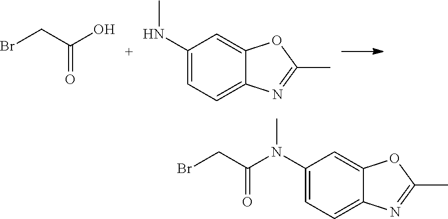

To a solution of N-methylbenzo[d]oxazol-6-amine (9.4 g, 58.0 mmol) in DCM (100 mL) was added EDC (26.6 g, 139 mmol), DMAP (0.35 g, 2.9 mmol) and bromoacetic acid (18.5 g, 133 mmol) at 0° C. under N₂. The reaction mixture was stirred at rt overnight, then diluted with water (200 mL). The product was extracted with DCM (3×100 mL) and the organics were washed with brine, dried over Na₂SO₄, concentrated to obtain 2-bromo-N-methyl-N-(2-methylbenzo[d]oxazol-6-yl)acetamide (10.0 g, 61%). (MS ESI +ve, 283.1 [M+H]); ¹HNMR (400 MHz, DMSO): 2.59 (s, 3H), 3.23 (s, 3H), 4.04 (s, 2H), 6.76-6.74 (d, J=8.8 Hz, 1H), 7.30-7.38 (dd, J=8.8 Hz, 1H), 7.71-7.73 (d, J=8.0 Hz, 1H).

### (18) tert-butyl 2-(3-benzyl-7-chloro-4-oxo-3,4-dihydro-5H-imidazo[4,5-d]pyridazin-5-yl)acetate

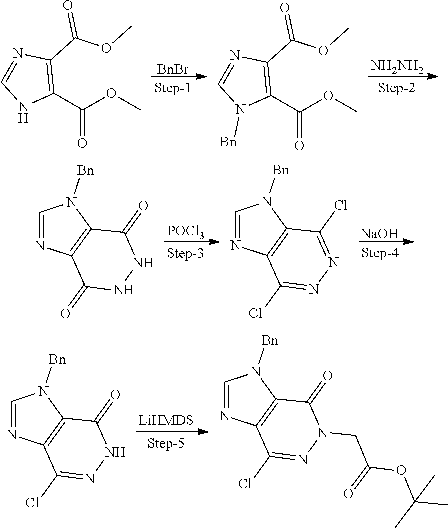

Step-1: To a suspension of NaH (2.82 g, 70.6 mmol) in DMF (20 mL) was added dimethyl 1H-imidazole-4,5-dicarboxylate (10 g, 54.3 mmol) portion wise at 0° C. The resultant mixture was stirred at 0° C. for 1 h and then benzyl bromide (7.3 mL, 59.8 mmol) was added. The mixture was stirred at room temperature for 1.5 h and diluted with ice-water (500 mL), extracted with ethyl acetate (3×500 mL). The combined organic layer was washed with brine and dried over sodium sulfate. Solvent was removed under vacuum to provide dimethyl 1-benzyl-1H-imidazole-4,5-dicarboxylate (13.4 g, 90%) as yellow liquid. (MS ESI +ve, 275.2 [M+H]).

Step-2: To a solution of ethyl 3-phenyl-1H-pyrazole-5-carboxylate (11 g, 40.1 mmol) in ethanol (80 mL) was added hydrazine hydrate (8.4 g, 168 mmol). The resultant mixture was heated to reflux at 100° C. for 16 h. The solid precipitate was filtered and washed with water, dried under vacuum to provide 1-benzyl-5,6-dihydro-1H-imidazo[4,5-d]pyridazine-4,7-dione (6.5 g, 67%) as white solid. (MS ESI +ve, 243.0 [M+H]).

Step-3: A mixture of 1-benzyl-5,6-dihydro-1H-imidazo[4,5-d]pyridazine-4,7-dione (6.5 g, 26.8 mmol) and phosphorus oxychloride (30 mL) was heated at 110° C. in a sealed tube for 16 h. The reaction mixture was slowly quenched with ice-cold saturated NaHCO₃ solution and extracted with ethyl acetate (3×250 mL). The combined organic layer was washed with brine and dried over sodium sulfate. The volatiles were removed under vacuum to provide 1-benzyl-4,7-dichloro-1H-imidazo[4,5-d]pyridazine (5.5 g, 73%) as yellow solid. (MS ESI +ve, 281.0 [M+H]).

Step-4: To a stirred solution of 1-benzyl-4,7-dichloro-1H-imidazo[4,5-d]pyridazine (5.5 g, 19.6 mmol) in 1,4-dioxane (30 mL) was added NaOH (1.6 g, 39.2 mmol) in water (30 mL). The resultant solution was heated at 110° C. for 16 h. The reaction was slowly quenched with ice-cold water and acidified with acetic acid. The aqueous layer was extracted with EtOAc (3×250 mL). The combined organic layer was washed with brine and dried over sodium sulfate. The volatiles were removed under vacuum to provide 3-benzyl-7-chloro-3,5-dihydro-4H-imidazo[4,5-d]pyridazin-4-one (4.1 g, 80%) as off-white solid. (MS ESI +ve, 261.0 [M+H]); ¹H NMR: (400 MHz, DMSO-d6) δ: 3.35 (s, 3H), 4.85 (s, 2H), 7.40-7.73 (m, 5H), 8.38-8.48 (m, 2H), 7.56-7.60 (m, 2H), 8.54 (s, 1H), 13.7 (s, 1H).

Step-5: To a stirred solution of 3-benzyl-7-chloro-3,5-dihydro-4H-imidazo[4,5-d]pyridazin-4-one (4.1 g, 15.7 mmol) in THF (15 mL) was added LiHMDS (1M in THF, 23.5 mL, 23.5 mmol) at −10° C. The mixture allowed to stir at 0° C. for 30 min. A solution of ethyl bromoacetate (52.5 mg, 17.2 mmol) in THF (10 mL) was added dropwise to the above mixture while maintaining the temperature at 0° C. The resulting mixture was stirring at room temperature for 16 h and was diluted with ice-water, extracted with ethyl acetate (3×250 mL). The combined organic layer was washed with brine and dried over sodium sulfate. The volatiles were removed under vacuum and the residue was purified by flash chromatography (0-40% EtOAc in hexane) to afford tert-butyl 2-(3-benzyl-7-chloro-4-oxo-3,4-dihydro-5H-imidazo[4,5-d]pyridazin-5-yl)acetate (3.1 g, 53%) as yellow solid. (MS ESI +ve, 375.2 [M+H]).

### (19) 2-(7-(3-fluorophenyl)-4-oxo-3,4-dihydro-5H-imidazo[4,5-d]pyridazin-5-yl)acetic acid

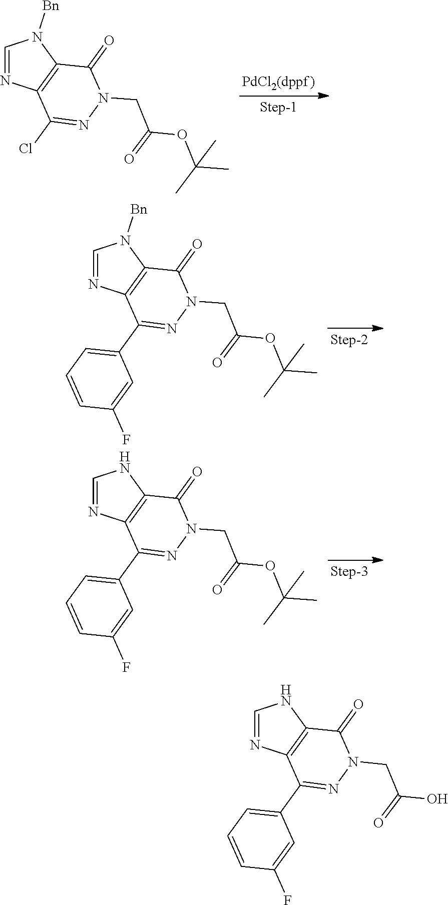

Step-1: A mixture of tert-butyl 2-(3-benzyl-7-chloro-4-oxo-3,4-dihydro-5H-imidazo[4,5-d]pyridazin-5-yl)acetate (1.0 g, 2.7 mmol) in 1,2-dimethoxyethane (6 mL), 3-fluorophenylboronic acid (448 mg, 3.2 mmol), cesium fluoride (853 mg, 5.4 mmol) in water (1.5 mL) was degassed. PdCl₂(dppf) (39.5 mg, 0.054 mmol) was added. The mixture was further degassed for 15 minutes and then heated at 110° C. for 16 h. The reaction mixture was diluted with water and extracted with ethyl acetate (3×20 mL). The combined organic layer was washed with brine and dried over sodium sulphate. The volatiles were removed under vacuum and the residue was purified by silica gel column chromatography (25% EtOAc in hexane) to afford tert-butyl 2-(3-benzyl-7-(3-fluorophenyl)-4-oxo-3,4-dihydro-5H-imidazo[4,5-d]pyridazin-5-yl)acetate (0.9 g, 77%) as yellow solid. (MS ESI +ve, 435.2 [M+H]).

Step-2: To a solution of tert-butyl 2-(3-benzyl-7-(3-fluorophenyl)-4-oxo-3,4-dihydro-5H-imidazo[4,5-d]pyridazin-5-yl)acetate (0.9 g, 2.06 mmol) in DCM (5 mL) was added palladium on charcoal (800 mg) in MeOH (15 mL). The resultant mixture was hydrogenated at room temperature for 16 h with a H₂ balloon. The catalyst was filtered off through a celite bed. The volatiles were removed under vacuum to provide tert-butyl 2-(7-(3-fluorophenyl)-4-oxo-3,4-dihydro-5H-imidazo[4,5-d]pyridazin-5-yl)acetate (610 mg, 86%) as white solid. (MS ESI +ve, 345.6 [M+H]).

Step-3: A solution of tert-butyl 2-(7-(3-chlorophenyl)-4-oxo-3,4-dihydro-5H-imidazo[4,5-d]pyridazin-5-yl)acetate (0.7 g, 2.06 mmol) in DCM (5 mL) was cooled to 0° C. and trifluoroacetic acid (2.5 mL) in DCM (2.5 mL) was added dropwise. The mixture allowed to stir at room temperature for 16 h. The volatiles were removed under vacuum. Azeotropic distillation was performed by adding DCM several times to completely remove TFA to afford 2-(7-(3-chlorophenyl)-4-oxo-3,4-dihydro-5H-imidazo[4,5-d]pyridazin-5-yl)acetic acid (570 mg, 96%) as light brown solid. (MS ESI +ve, 289.0 [M+H]).

2-(7-(3-cyanophenyl)-4-oxo-3,4-dihydro-5H-imidazo[4,5-d]pyridazin-5-yl)acetic acid and 2-(7-(3-chlorophenyl)-4-oxo-3,4-dihydro-5H-imidazo[4,5-d]pyridazin-5-yl)acetic acid were synthesized in a similar fashion:

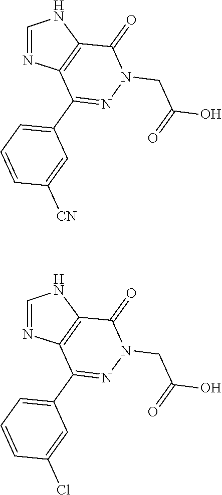 [SMILES: N#Cc1cccc(-c2nn(CC(=O)O)c(=O)c3[nH]cnc23)c1 ; O=C(O)Cn1nc(-c2cccc(Cl)c2)c2nc[nH]c2c1=O]

### (20) 2-(7-(3-chlorophenyl)-4-oxofuro[2,3-d]pyridazin-5(4H)-yl)-2-methylpropanoic acid

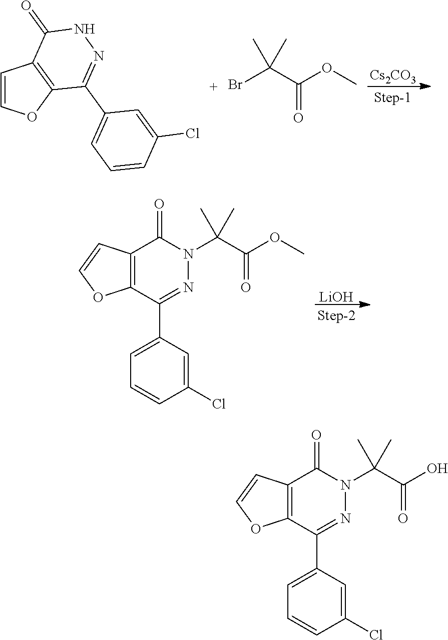

Step-1: To a solution of 7-(3-chlorophenyl)furo[2,3-d]pyridazin-4(5H)-one (0.5 g, 2.03 mmol) in DMF (10 mL) was added methyl 2-bromo-2-methylpropanoate (0.44 g, 2.44 mmol), potassium iodide (0.02 g), cesium carbonate (0.93 g, 2.84 mmol). The mixture was heated to 90° C. for 16 h and quenched with ice water and (50 mL), extracted with ethyl acetate (50 mL×3). The organic layer by drying over sodium sulfate and the product was purified by combiflash (50% ethyl acetate in hexane) to obtain methyl 2-(7-(3-chlorophenyl)-4-oxofuro[2,3-d]pyridazin-5(4H)-yl)-2-methylpropanoate as yellow solid (0.27 g, 38%). (MS ESI +ve, 347.2 [M+H]).

Step-2: To a solution of methyl 2-(7-(3-chlorophenyl)-4-oxofuro[2,3-d]pyridazin-5(4H)-yl)-2-methylpropanoate (0.25 g, 0.72 mmol) in THF (5 mL) was added lithium hydroxide (0.09 g, 2.17 mmol) in water (5 mL) at 0° C. The mixture was stirring at rt for 3 h and then concentrated under vacuum. The residue was diluted with HC (1N, 10 mL). The precipitated was filtered and dried to give 2-(7-(3-chlorophenyl)-4-oxofuro[2,3-d]pyridazin-5(4H)-yl)-2-methylpropanoic acid (0.22 g, 91%) as yellow solid. (MS ESI +ve: 333.25 [M+H]); ¹H NMR: (400 MHz, DMSO) δ:1.01-1.03 (m, 3H), 4.30-4.48 (m, 3H), 7.27-7.28 (d, J=2.4, 1H), 7.61-7.64 (m, 2H), 8.03-8.12 (m, 2H), 8.36-8.38 (t, 1H).

### (21) 2-(7-(3-cyanophenyl)-4-oxofuro[2,3-d]pyridazin-5(4H)-yl)-2-methylpropanoic acid

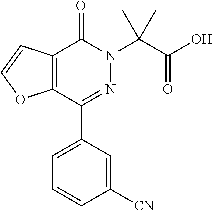 [SMILES: CC(C)(C(=O)O)n1nc(-c2cccc(C#N)c2)c2occc2c1=O]

2-(7-(3-cyanophenyl)-4-oxofuro[2,3-d]pyridazin-5(4H)-yl)-2-methylpropanoic acid was synthesized in a similar fashion.

### (22) 4,7-dichlorofuro[2,3-d]pyridazine-2-carboxylic acid

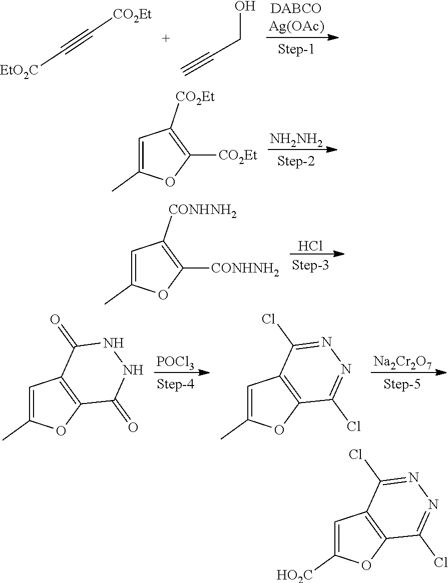

Step 1: To a solution of ethyl 3-(propionyloxy)propiolate (25 g, 146 mmol), prop-2-yn-1-ol (8.23 g, 146 mmol) in DCM (250 mL) was added DABCO (1.64 g, 14.6 mmol). The mixture was stirred for 30 minutes at rt and then concentrated. Toluene (250 mL), AgOAc (1.22 g, 7.34 mmol), PPh3 (3.85 g, 14.6 mmol) were added and the mixture was stirred overnight at 55° C. The mixture was diluted with ice cold water (300 mL), extracted with EtOAc (200 mL×2). The organic layer was dried over sodium sulfate and concentrated. The product was purified by column chromatography (20% EtOAc in hexane) to give diethyl 5-methylfuran-2,3-dicarboxylate (27 g, 80%) as an oil. ¹H NMR: (400 MHz, CDCl3) δ: 1.36-1.41 (m, 6H), 2.40 (s, 3H), 4.33-4.45 (m, 4H), 6.39 (s, 1H).

Step-2: To a solution of diethyl 5-methylfuran-2,3-dicarboxylate (27 g, 101.4 mmol) in methanol was added hydrazine hydrate (15.2 g, 304 mmol). The mixture was stirred for 16 h at 60° C. The solvent was evaporated to give 5-methylfuran-2,3-dicarbohydrazide (30 g) for next step without further purification. (MS ESI +ve, 198.95 [M+H]).

Step-3: A solution of 5-methylfuran-2,3-dicarbohydrazide (30 g, 150 mmol) in hydrazine hydrate (100 mL) was stirred at 100° C. for 16 h. The reaction mixture was concentrated to obtain solid which was dissolved in hot water (500 mL). pH was adjusted by using HCl. Solid product was collected by filtration to give 2-methyl-5,6-dihydrofuro[2,3-d]pyridazine-4,7-dione (12 g, 44%). (MS ESI +ve, 166.93[M+H]).

Step-4: To a solution of 2-methyl-5,6-dihydrofuro[2,3-d]pyridazine-4,7-dione (12.0 g, 72.2 mmol) in POCl₃ (108 mL) was added pyridine (7.2 mL). The reaction mixture was stirred for 2 h at 130° C. The solvent was removed under vacuum and the residue was poured to ice water. The solid was collected by filtration to give 4,7-dichloro-2-methylfuro[2,3-d]pyridazine (10 g, 68%). 1H NMR: (400 MHz, DMSO) δ: 2.64-2.64 (d, 3H), 7.07-7.07 (d, 1H).

Step-5: Na₂Cr₂O₇ (6.6 g) in water (10 mL) was added dropwise to cooled conc. H₂SO₄ (35 mL). 4,7-dichloro-2-methylfuro[2,3-d]pyridazine (4 g, 24.0 mmol) in con H₂SO₄ (10 mL) was added to the above mixture dropwise at 5-10° C. The mixture was stirred for 3 h at rt and poured onto ice. Solid was collected by filtration. The solid was dissolved with aq Na₂CO₃ and filtered. The filtrate was acidified with HCl and the participate was filtered to give 4,7-dichlorofuro[2,3-d]pyridazine-2-carboxylic acid (0.8 g, 17%). (MS ESI +ve, 233.15 [M+H]); ¹H NMR: (400 MHz, DMSO) δ: 8.02 (s, 1H), 14.2 (s, 1H).

### (23) methyl 4,7-dichlorofuro[2,3-d]pyridazine-2-carboxylate

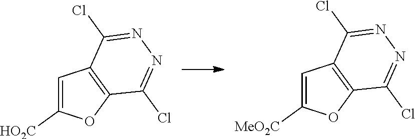 [SMILES: O=C(O)c1cc2c(Cl)nnc(Cl)c2o1 ; COC(=O)c1cc2c(Cl)nnc(Cl)c2o1]

To a solution of 4,7-dichlorofuro[2,3-d]pyridazine-2-carboxylic acid (4.4 g, 18 mmol) in DCM (30 mL) and DMF (2.0 mL) was added oxalyl chloride (2.4 mL, 28.3 mmol) dropwise at 0° C. The mixture was stirring for 2 h at rt, followed by addition of MeOH (50 mL). The reaction mixture was stirred for an additional 0.5 h. Solvents were evaporated and the residue was washed with hexane to give methyl 4,7-dichlorofuro[2,3-d]pyridazine-2-carboxylate (4.2 g, 94%).

### (24) tert-butyl 2-(7-chloro-2-formyl-4-oxofuro[2,3-d]pyridazin-5(4H)-yl)acetate

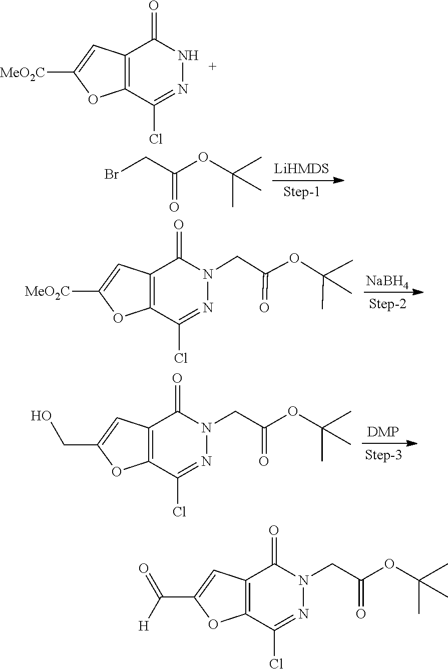

Step-1: To a solution of methyl 7-chloro-4-oxo-4,5-dihydrofuro[2,3-d]pyridazine-2-carboxylate (1.9 g, 8.40 mmol) in DMF (20 mL) was added lithium bis(trimethylsilyl)amide (12.6 mL, 12.6 mmol) at −10° C. The mixture was stirring at 0° C. for 30 min and a solution of tert-butyl 2-bromoacetate (1.34 g, 9.2 mmol) in DMF (10 mL) was added dropwise at 0° C. The mixture was stirring at rt for 16 h and diluted with ice-water, extracted with ethyl acetate (3×250 mL). The combined organic layer was washed with brine and dried over sodium sulphate. The volatiles were removed under vacuum and the residue was purified by flash chromatography (0-40% ethyl acetate in hexane) to afford methyl 5-(2-(tert-butoxy)-2-oxoethyl)-7-chloro-4-oxo-4,5-dihydrofuro[2,3-d]pyridazine-2-carboxylate (1.5 g, 52%) for next step.

Step-2: To solution of methyl 5-(2-(tert-butoxy)-2-oxoethyl)-7-chloro-4-oxo-4,5-dihydrofuro[2,3-d]pyridazine-2-carboxylate (1.0 g, 2.3 mmol) in MeOH (10 mL) was added sodium borohydride (0.45 g, 12.0 mmol) portion-wise at 0° C. The mixture was stirred at room temperature for 16 h and solvent was removed under vacuum. The residue was diluted with water, extracted with ethyl acetate (100 mL). The organic layer was washed with water, brine and dried over sodium sulfate. The solvent were removed under vacuum to give tert-butyl 2-(7-chloro-2-(hydroxymethyl)-4-oxofuro[2,3-d]pyridazin-5(4H)-yl)acetate (0.7 g, 76%) for next step.

Step-3: To a solution of tert-butyl 2-(7-chloro-2-(hydroxymethyl)-4-oxofuro[2,3-d]pyridazin-5(4H)-yl)acetate (0.65 g, 2.07 mmol) in dichloromethane (25 mL) was added Dess-Martin periodinane (2.6 g, 6.21 mmol) portion-wise at 0° C. The mixture was stirred at rt for 16 h and diluted with saturated sodium bicarbonate, extracted with dichloromethane. The combined organic layer were washed with brine, dried over anhydrous sodium sulphate, and concentrated in vacuum to give tert-butyl 2-(7-chloro-2-formyl-4-oxofuro[2,3-d]pyridazin-5(4H)-yl)acetate (0.6 g, 92%).

### (25) 2-(7-(3-cyanophenyl)-4-oxo-2-(piperidin-1-ylmethyl)furo[2,3-d]pyridazin-5(4H)-yl)acetic acid

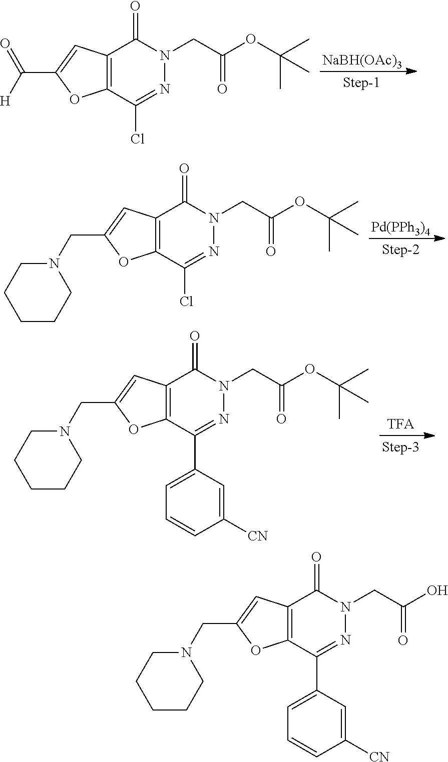

Step-1: To a solution of tert-butyl 2-(7-chloro-2-formyl-4-oxofuro[2,3-d]pyridazin-5(4H)-yl)acetate (0.70 g, 2.24 mmol) in dichloromethane (10 mL) was added piperidine (0.154 g, 1.79 mmol) and acetic acid (0.40 g, 6.72 mmol) at 0° C. The mixture was stirred at 0° C. for 30 min and then sodium triacetoxyborohydride (0.95 g, 4.48 mmol) was added. The mixture was stirred at room temperature for 12 h. The reaction was quenched with water and extracted with dichloromethane. The combined organic layer were washed with brine, dried over anhydrous sodium sulfate, and concentrated in vacuum to give tert-butyl 2-(7-chloro-4-oxo-2-(piperidin-1-ylmethyl)furo[2,3-d]pyridazin-5(4H)-yl)acetate (0.6 g, 70%).

Step-2: A mixture of tert-butyl 2-(7-chloro-4-oxo-2-(piperidin-1-ylmethyl)furo[2,3-d]pyridazin-5(4H)-yl)acetate (0.70 g, 1.83 mmol), (3-cyanophenyl)boronic acid (0.34 g, 2.20 mmol), sodium carbonate (0.29 g, 2.70 mmol) in toluene (3 mL) and water (0.5 mL) was degassed. Pd(PPh₃)₄ (0.21 g, 0.018 mmol) was added and the mixture was degassed again. The reaction mixture was heated at 80° C. overnight. Solvent was evaporated, and the crude product was purified by column chromatography (2% methanol in dichloromethane) to give tert-butyl 2-(7-(3-cyanophenyl)-4-oxo-2-(piperidin-1-ylmethyl)furo[2,3-d]pyridazin-5(4H)-yl)acetate (0.40 g, 48%).

Step-3: To a solution of tert-butyl 2-(7-(3-cyanophenyl)-4-oxo-2-(piperidin-1-ylmethyl)furo[2,3-d]pyridazin-5(4H)-yl)acetate (0.60 g, 1.33 mmol) in dichloromethane (3 mL) was added trifluoroacetic acid at 0° C. The mixture was stirred at room temperature for 3 h, then additional trifluoroacetic acid (1 mL) was added. The mixture was stirred at room temperature for 12 h. The reaction mixture was evaporated and the residue was washed with dichloromethane to give 2-(7-(3-cyanophenyl)-4-oxo-2-(piperidin-1-ylmethyl)furo[2,3-d]pyridazin-5(4H)-yl)acetic acid (0.50 g, 95%).

The following compounds were synthesized in a similar fashion:

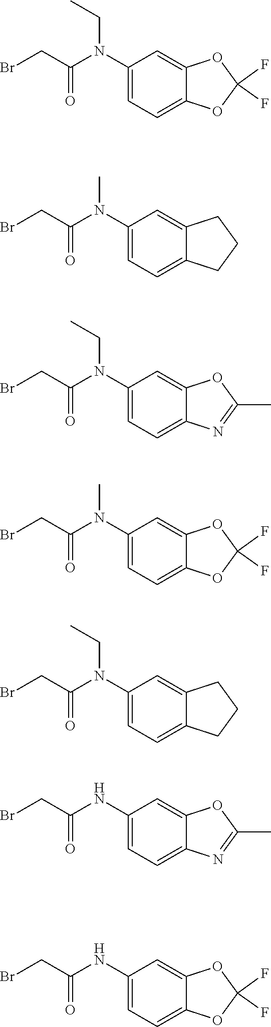

### (26) 2-chloro-N-cyclopropyl-N-(2-methylbenzo[d]oxazol-6-yl)acetamide

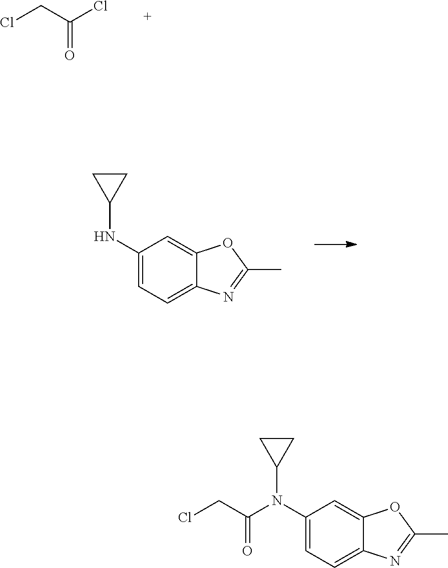 [SMILES: O=C(Cl)CCl ; Cc1nc2ccc(NC3CC3)cc2o1 ; Cc1nc2ccc(N(C(=O)CCl)C3CC3)cc2o1]

To a solution of N-cyclopropyl-2-methylbenzo[d]oxazol-6-amine (0.5 g, 2.66 mmol) in THF (15 mL) was added sodium bicarbonate (0.6 g, 7.44 mmol) at rt, followed by addition of chloroacetylchloride (0.23 mL, 2.92 mmol) at 0° C. The mixture was stirred at 0° C. for 3 h and quenched with ice water (25 mL), extracted with ethyl acetate (15 mL*3). The organic layer was dried over sodium sulfate and concentrated. The product was purified by column chromatography (50-60% DCM in hexane) to give 2-chloro-N-cyclopropyl-N-(2-methylbenzo[d]oxazol-6-yl)acetamide (0.2 g, 28%). (MS ESI +ve, 265.2 [M+1]).

The following compounds were synthesized in similar fashion:

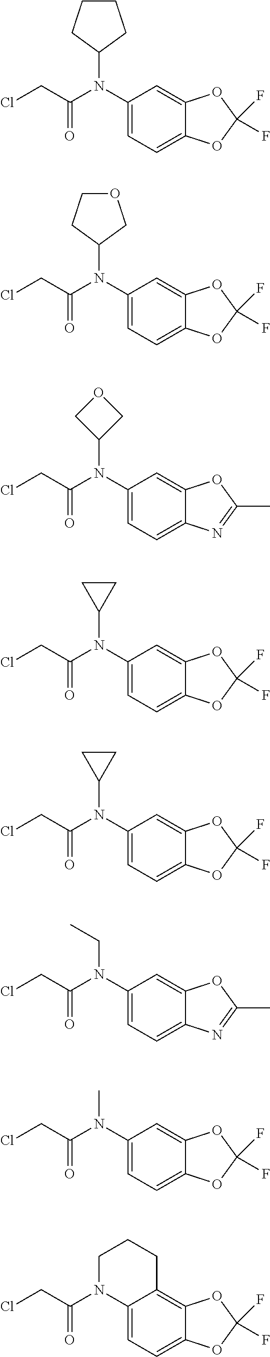

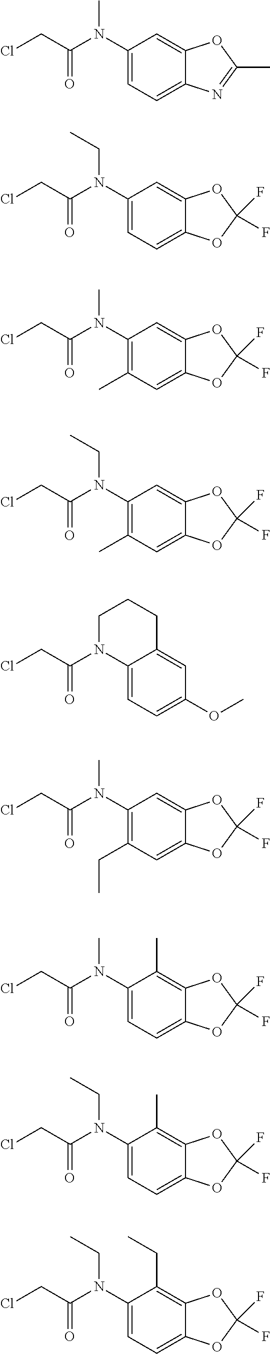

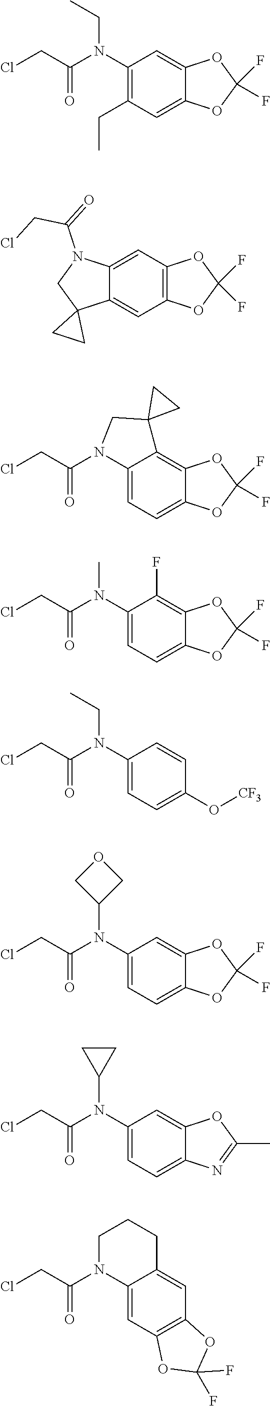

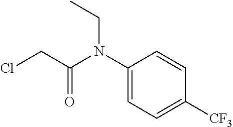 [SMILES: CCN(C(=O)CCl)c1ccc(C(F)(F)F)cc1]

### (27) diethyl 4-(trifluoromethyl)furan-2,3-dicarboxylate

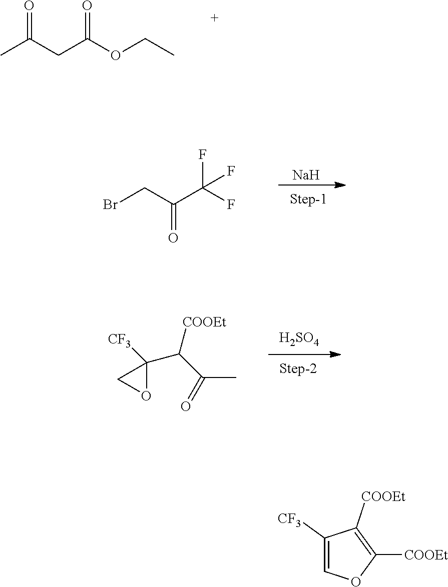

Step 1: A solution of ethyl 3-oxobutanoate (5.0 g, 38.5 mmol) in THF (50 mL) was treated with sodium hydride (60%, 1.57 g, 39.2 mmol) at 0° C. for 15 min, then 3-bromo-1,1,1-trifluoropropan-2-one (4.8 g, 25.1 mmol) was added. The reaction mixture was stirred at 25° C. for 2 h and was poured into ice cold water (100 mL), extracted with ethyl acetate. The combined organic layer was washed with brine, dried over sodium sulfate, and concentrated in vacuum. The product was purified by Combiflash (7% ethyl acetate in hexane) to afford ethyl 3-oxo-2-(2-(trifluoromethyl)oxiran-2-yl)butanoate (1.3 g, 14%). (MS ESI +Ve, 241.0 [M+1]).

Step 2: H₂SO₄ (0.5 mL) was added dropwise to ethyl 3-oxo-2-(2-(trifluoromethyl)oxiran-2-yl)butanoate (5 g, 20.8 mmol) while stirring. The mixture was stirred at room temperature for 12 h and was poured into ice cooled sodium bicarbonate solution (100 mL), extracted with ethyl acetate. The combined organic layer was washed with brine, dried, and concentrated in vacuum. The product was purified by Combiflash (3% ethyl acetate in hexane) to afford diethyl 4-(trifluoromethyl)furan-2,3-dicarboxylate (2.0 g, 34%). (MS ESI +Ve, 281.0 [M+1]).

### (28) diethyl 4,5-dimethylfuran-2,3-dicarboxylate

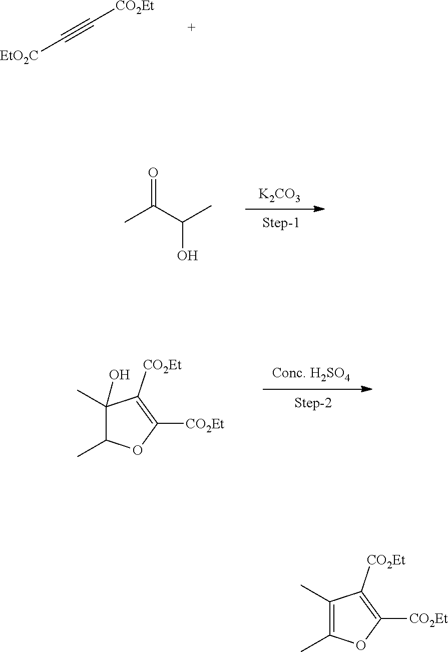

Step-1: To a solution of diethyl but-2-ynedioate (1.0 g, 5.8 mmol) and 3-hydroxybutan-2-one (0.51 g, 5.18 mmol) in acetone (10 mL) was added K₂CO₃ (0.81 g, 5.8 mmol). The mixture was heat at 60° C. for 16 h and diluted with water (10 mL), extracted with ethyl acetate (15 mL*3). The organic layer was dried over sodium sulfate and concentrated. The product was purified by combiflash (8% ethyl acetate in hexane) to give diethyl 4-hydroxy-4,5-dimethyl-4,5-dihydrofuran-2,3-dicarboxylate (0.5 g, 32%) as liquid. (MS ESI +Ve, 259[M+1]).

Step-2: To a solution of diethyl 4-hydroxy-4,5-dimethyl-4,5-dihydrofuran-2,3-dicarboxylate (0.2 g, 0.77 mmol) in methanol (2 mL) was added a drop of H₂SO₄. The mixture was heated at 60° C. for 16 h and solvent was evaporated. The residue was extracted with ethyl acetate (10 mL*2), dried, evaporated to give diethyl 4-hydroxy-4,5-dimethyl-4,5-dihydrofuran-2,3-dicarboxylate (0.13 g, 69%) as liquid. (MS ESI +ve, 241 [M+1]).

### (29) dimethyl 5-(tert-butyl)furan-2,3-dicarboxylate

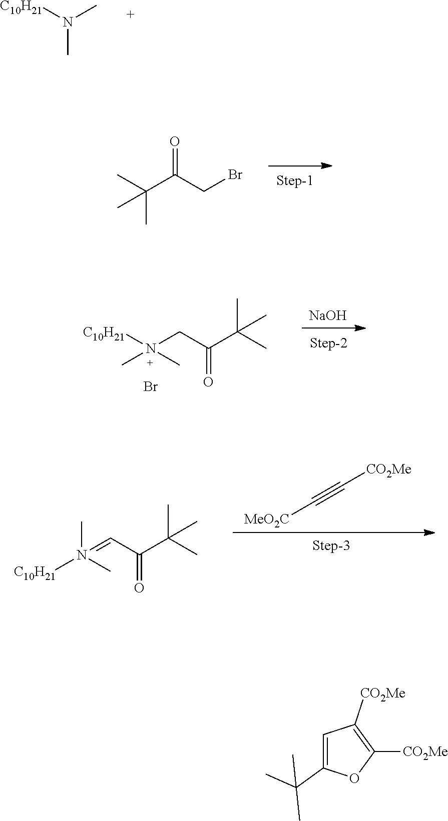

Step-1: A solution of N,N-dimethyldecan-1-amine (6.0 g, 3.23 mmol) and 1-bromo-3,3-dimethylbutan-2-one (5.79 g, 3.23 mmol) in acetonitrile (60 mL) was stirred at 80° C. for 15 min and then at room temperature overnight. The reaction mixture was evaporated to give N-(3,3-dimethyl-2-oxobutyl)-N,N-dimethyldecan-1-aminium bromide (13.4 g) as brown gum for the next step.

Step-2: To a solution of N-(3,3-dimethyl-2-oxobutyl)-N,N-dimethyldecan-1-aminium bromide (13.4 g, 36.7 mmol) in water (134 mL) was added 10% aqueous NaOH (134 mL) at 0° C. The mixture was stirred at 0° C. for 5 h and then the solid was filtered, dried to obtain (N-decyl-N,N,3,3-tetramethyl-2-oxobutan-1-ide-1-aminium) (10 g) as off white solid for next step.

Step-3: To a solution of N-decyl-N,N,3,3-tetramethyl-2-oxobutan-1-ide-1-aminium (10.6 g, 37.2 mmol) in THF (95 mL) was added diethyl but-2-ynedioate (5.23 g, 30.7 mmol) in THF (10 mL) drop wise at 0° C. for 2 h and the mixture was stirred at room temperature overnight. The reaction mixture was diluted with water (250 mL), extracted with ethyl acetate (200 mL×2), evaporated. The crude product obtained was purified by column (0 to 2% ethyl acetate in hexane) to obtain (diethyl 5-(tert-butyl)furan-2,3-dicarboxylate) as off white gum (6.2 g, 62%). (MS ESI +ve, 269.0 [M+1]). ¹H NMR: (400 MHz, DMSO) δ: 1.22-1.33 (m, 15H), 4.18-4.28 (m, 4H), 8.28 (s, 1H).

### (30) dimethyl benzofuran-2,3-dicarboxylate

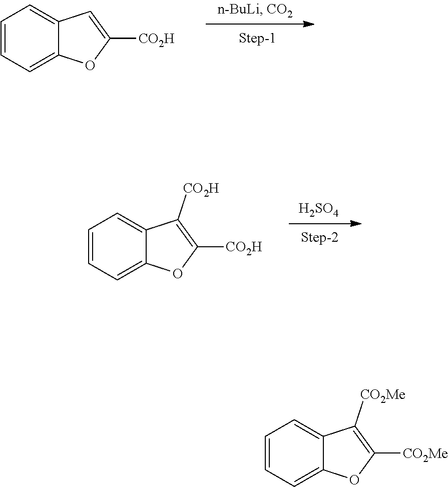 [SMILES: O=C(O)c1cc2ccccc2o1 ; O=C(O)c1oc2ccccc2c1C(=O)O ; "H2SO4" ; COC(=O)c1oc2ccccc2c1C(=O)OC]

Step-1: To a solution of diisopropyl amine (62.7 mL, 444 mmol) in THF (620 mL) was added n-BuLi (2.5 M in hexane, 185 mL, 444 mmol) dropwise at −78° C. The above solution was added to a solution of benzofuran-2-carboxylic acid (24.0 g, 148 mmol) in tetrahydrofuran (500 mL) dropwise at −78° C. The resulting mixture was stirred at −78° C. for 1 h and dry ice (100 g) was added portion wise. The mixture was stirred at room temperature overnight and quenched with ice, diluted with 1 N hydrochloric acid (700 mL), extracted with ethyl acetate (700 mL×3). The organic layer was dried and evaporated to give benzofuran-2,3-dicarboxylic acid (26.0 g, 85%) as white solid. (MS ESI −ve, 205.03 [M−H]).

Step-2: To a solution of benzofuran-2,3-dicarboxylic acid (26.0 g, 126 mmol) in methanol (260 mL) was added dropwise conc. H₂SO₄ (13 mL). The mixture was refluxing overnight, and solvent was evaporated. The residue was treated with aq. bicarbonate (100 mL), extracted with ethyl acetate (300 mL×2). The organic layer was dried, evaporated. The product was purified by column (0 to 20% ethyl acetate in hexane) to obtain dimethyl benzofuran-2,3-dicarboxylate as off-white gum (8.33 g, 26%). (MS ESI +ve, 235.25 [M+1]); ¹H NMR: (400 MHz, DMSO) δ: 3.99 (s, 6H), 7.38-7.47 (m, 1H), 7.52-7.54 (m, 1H), 7.73-7.79 (m, 2H).

### (31) 2-(tert-butyl)-7-chlorofuro[2,3-d]pyridazin-4(5H)-one

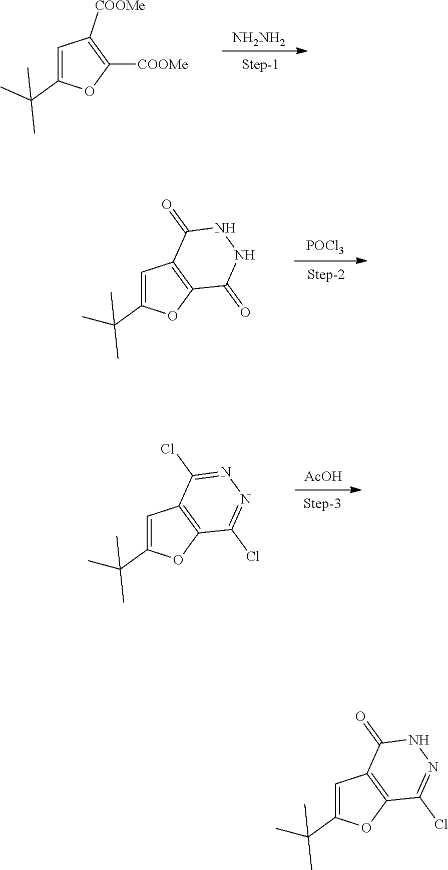

Step-1: To a solution of diethyl 5-(tert-butyl)furan-2,3-dicarboxylate 1.0 g, 3.72 mmol) in methanol (10 mL) was added hydrazine hydrate (0.9 mL, 18.6 mmol). The mixture was heated at 60° C. for 48 h and the solvent was evaporated to give (2-(tert-butyl)-5,6-dihydrofuro[2,3-d]pyridazine-4,7-dione) as yellow solid (0.45 g, 57%). (MS ESI +ve, 209.0 [M+1]); ¹H NMR: (400 MHz, DMSO) δ: 1.46 (s, 9H), 8.31 (s, 1H), 11.0 (s, 2H).

Step-2: A mixture of 2-(tert-butyl)-5,6-dihydrofuro[2,3-d]pyridazine-4,7-dione (0.4 g, 1.92 mmol), phosphorus oxychloride (3.6 mL) and pyridine (0.24 mL) was heated to 130° C. for 2 h. The mixture was poured into ice water (50 mL) and extracted to ethyl acetate (50 mL×3). The organic layer was dried, evaporated to obtain (2-(tert-butyl)-4,7-dichlorofuro[2,3-d]pyridazine) as yellow solid (0.45 g, 95%). (MS ESI +ve, 227.0 [M+1]).

Step-3: A solution of 2-(tert-butyl)-4,7-dichlorofuro[2,3-d]pyridazine (0.45 g, 1.83 mmol) in AcOH (6.25 mL) was heated to 130° C. for 3 h. Solvent was evaporated, and the reside was diluted with saturated sodium bicarbonate (50 mL), extracted with ethyl acetate (50 mL×3). The organic layer was dried, evaporated and the reside was purified column (10% ethyl acetate in hexane) to obtain (2-(tert-butyl)-7-chlorofuro[2,3-d]pyridazin-4(5H)-one) as yellow solid (0.2 g, 48%). (MS ESI +ve, 227.30 [M+1]); ¹H NMR: (400 MHz, DMSO) δ:1.47 (s, 9H), 8.45 (s, 1H), 11.9 (s, 1H).

In a similar fashion, the following compounds were synthesized:

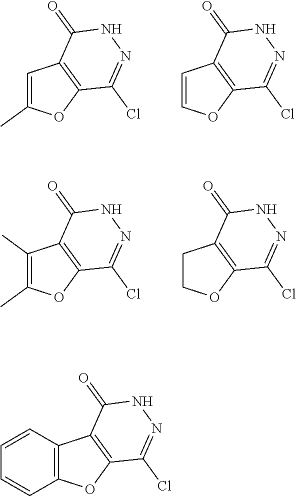

### (32) 7-chloro-N-isopropyl-4-oxo-4,5-dihydrofuro[2,3-d]pyridazine-2-carboxamide

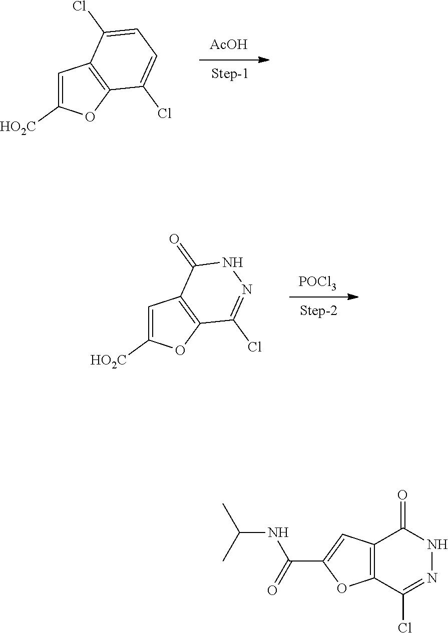

Step-1: A solution of 4,7-dichlorofuro[2,3-d]pyridazine-2-carboxylic acid (1.8 g, 7.72 mmol) in acetic acid (25 mL) was stirred for 3 h at 130° C. The reaction mixture was cooled to rt and solid was collected by filtration, washed with hexane to give pure 7-chloro-4-oxo-4,5-dihydrofuro[2,3-d]pyridazine-2-carboxylic acid (0.8 g, 48%) as a white solid. (MS ESI +ve, 214.84 [M+H]).

Step-2: To a solution of 7-chloro-4-oxo-4,5-dihydrofuro[2,3-d]pyridazine-2-carboxylic acid (0.5 g, 2.33 mmol) in DCM (20 mL), pyridine (2.5 mL) was added POCl₃ (2 mL) at 0° C. The mixture was stirred at 0° C. for 15 min before the addition of propan-2-amine (0.28 g, 4.66 mmol). The mixture was stirred at rt for 2 h and poured onto ice, neutralized by NaHCO₃ solution. The product was extracted with DCM (100 mL×2). The organic layer was dried, concentrated to give 7-chloro-N-isopropyl-4-oxo-4,5-dihydrofuro[2,3-d]pyridazine-2-carboxamide (0.3 g, 50%). (MS ESI +ve, 256.58 [M+H]); ¹H NMR (400 MHz, DMSO) δ:1.19 (d, 6H), 4.06-4.14 (m, 1H), 8.67-8.69 (d, 1H), 13.2 (s, 1H).

In a similar fashion, the following compounds were synthesized:

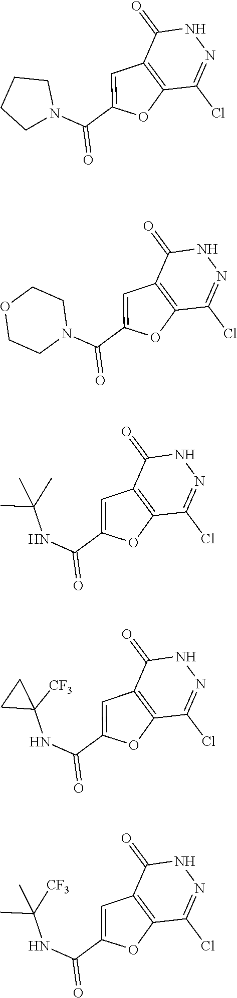 [SMILES: O=C(c1cc2c(=O)[nH]nc(Cl)c2o1)N1CCCC1 ; O=C(c1cc2c(=O)[nH]nc(Cl)c2o1)N1CCOCC1 ; CC(C)(C)NC(=O)c1cc2c(=O)[nH]nc(Cl)c2o1 ; O=C(NC1(C(F)(F)F)CC1)c1cc2c(=O)[nH]nc(Cl)c2o1 ; CC(C)(NC(=O)c1cc2c(=O)[nH]nc(Cl)c2o1)C(F)(F)F]

### (33) 2-(7-chloro-2-formyl-4-oxofuro[2,3-d]pyridazin-5(4H)-yl)-N-(2,2-difluorobenzo[d][1,3]dioxol-5-yl)-N-methylacetamide

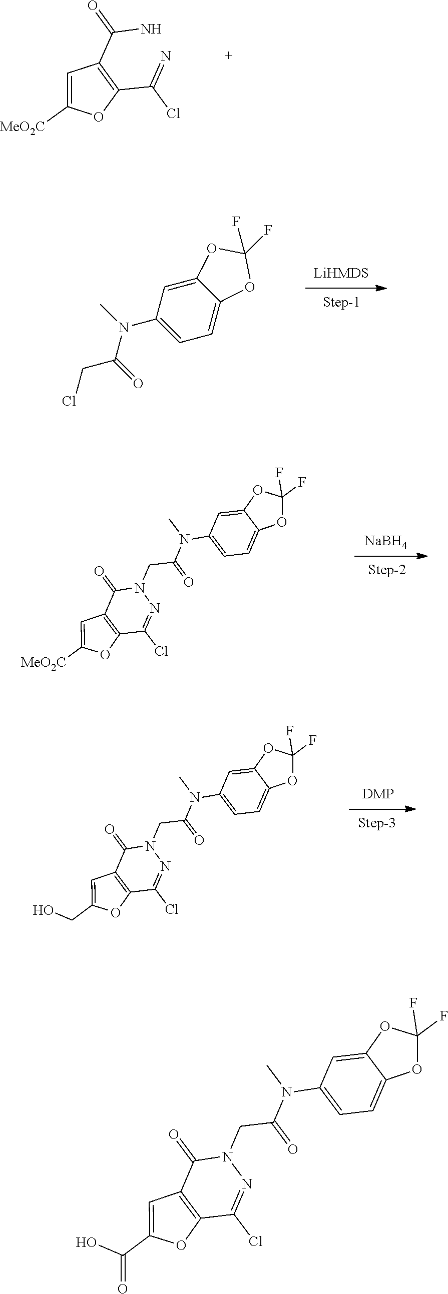 [SMILES: COC(=O)c1cc(C(N)=O)c(C(=N)Cl)o1 ; CN(C(=O)CCl)c1ccc2c(c1)OC(F)(F)O2 ; COC(=O)c1cc2c(=O)n(CC(=O)N(C)c3ccc4c(c3)OC(F)(F)O4)nc(Cl)c2o1 ; "NaBH4" ; CN(C(=O)Cn1nc(Cl)c2oc(CO)cc2c1=O)c1ccc2c(c1)OC(F)(F)O2 ; CN(C(=O)Cn1nc(Cl)c2oc(C(=O)O)cc2c1=O)c1ccc2c(c1)OC(F)(F)O2]

Step-1: To a solution of methyl 7-chloro-4-oxo-4,5-dihydrofuro[2,3-d]pyridazine-2-carboxylate (2.0 g, 8.74 mmol) in DMF (20 mL) was added lithium bis(trimethylsilyl)amide (13.1 mL, 13.1 mmol, 1M in THF) at 0° C. The resultant solution was stirred at 0° C. for 20 min and then a solution of 2-chloro-N-(2,2-difluorobenzo[d][1,3]dioxol-5-yl)-N-methylacetamide (2.7 g, 10.5 mmol) in DMF (10 mL) was added drop wise. The reaction mixture allowed to warm up to rt and stirred for 16 h. The mixture was diluted with water (50 mL) and extracted with ethyl acetate (200 mL). The organic layer ware washed with water, brine and dried over sodium sulfate. The solvent were removed under vacuum and the residue was purified by column chromatography (40% ethyl acetate in hexane) to give methyl 7-chloro-5-(2-((2,2-difluorobenzo[d][1,3]dioxol-5-yl)(methyl)amino)-2-oxoethyl)-4-oxo-4,5-dihydrofuro[2,3-d]pyridazine-2-carboxylate (1.0 g, 25%) as white solid. (MS ESI +ve, 456.0 [M+H]).

Step-2: To a solution of methyl 7-chloro-5-(2-((2,2-difluorobenzo[d][1,3]dioxol-5-yl)(methyl)amino)-2-oxoethyl)-4-oxo-4,5-dihydrofuro[2,3-d]pyridazine-2-carboxylate (1.0 g, 2.19 mmol) in MeOH (20 mL) was added sodium borohydride (0.249 g, 6.58 mmol) portion-wise at 0° C. The mixture was stirred at rt for 16 h and methanol were removed under vacuum. The residue was diluted with water, extracted with ethyl acetate (200 mL). The organic layer was washed with water, brine and dried over sodium sulfate. The solvent were removed under vacuum to give the 2-(7-chloro-2-(hydroxymethyl)-4-oxofuro[2,3-d]pyridazin-5(4H)-yl)-N-(2,2-difluorobenzo[d][1,3]dioxol-5-yl)-N-methylacetamide (0.90 g, 95%). (MS ESI +ve, 428.3 [M+H]).

Step-3: To a solution of 2-(7-chloro-2-(hydroxymethyl)-4-oxofuro[2,3-d]pyridazin-5(4H)-yl)-N-(2,2-difluorobenzo[d][1,3]dioxol-5-yl)-N-methylacetamide (0.90 g, 2.10 mmol) in DCM (30 mL) was added Dess-Martin periodinane (2.67 g, 6.31 mmol) portion-wise at 0° C. The mixture was stirred at rt for 16 h and diluted with aq. sodium bicarbonate, extracted with DCM. The combined organic layer was washed with brine, dried over sodium sulfate, and concentrated. The product was purified using column chromatography (0-40% ethyl acetate in hexanes) to obtain 2-(7-chloro-2-formyl-4-oxofuro[2,3-d]pyridazin-5(4H)-yl)-N-(2,2-difluorobenzo[d][1,3]dioxol-5-yl)-N-methylacetamide (0.7 g, 78%) as white solid. (MS ESI +ve, 426.0 [M+H]).

### (34) 2-cyclopropyl-4-oxo-4,5-dihydrofuro[2,3-d]pyridazin-7-yl 4-methylbenzenesulfonate

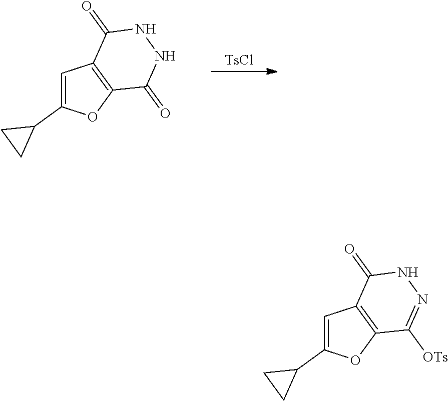 [SMILES: O=c1[nH][nH]c(=O)c2oc(C3CC3)cc12 ; "TsCl" ; Cc1ccc(S(=O)(=O)Oc2n[nH]c(=O)c3cc(C4CC4)oc23)cc1]

To a mixture of 2-cyclopropyl-5,6-dihydrofuro[2,3-d]pyridazine-4,7-dione (0.16 g, 0.83 mmol) in tetrahydrofuran (5.0 mL), triethylamine (0.002 mL, 1.2 mmol) was added TsCl (158 mg, 0.83 mmol) at 0° C. The reaction mixture was stirred at rt overnight and diluted with water, extracted with ethyl acetate. The organic layer was evaporated under vacuum. The residue was purified by column (25-30% ethyl acetate in hexane) to give 2-cyclopropyl-4-oxo-4,5-dihydrofuro[2,3-d]pyridazin-7-yl 4-methylbenzenesulfonate (0.10 g, 34%). (MS ESI +ve, 347.0 [M+H]).

### 2-cyclobutyl-4-oxo-4,5-dihydrofuro[2,3-d]pyridazin-7-yl 4-methylbenzenesulfonate

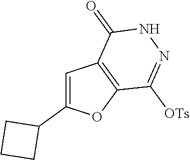 [SMILES: Cc1ccc(S(=O)(=O)Oc2n[nH]c(=O)c3cc(C4CCC4)oc23)cc1]

2-cyclobutyl-4-oxo-4,5-dihydrofuro[2,3-d]pyridazin-7-yl 4-methylbenzenesulfonate was synthesized in a similar fashion.

### (35) 1-methyl-4-oxo-4,5-dihydro-1H-pyrazolo[3,4-d]pyridazin-7-yl 4-methylbenzenesulfonate and 1-methyl-7-oxo-6,7-dihydro-1H-pyrazolo[3,4-d]pyridazin-4-yl 4-methylbenzenesulfonate

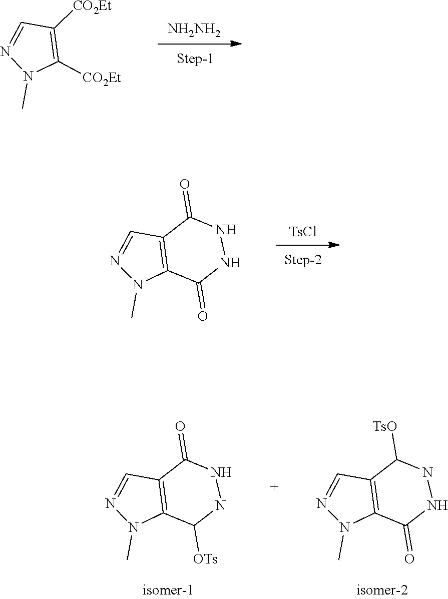 [SMILES: CCOC(=O)c1cnn(C)c1C(=O)OCC ; "NH2NH2" ; Cn1ncc2c(=O)[nH][nH]c(=O)c21 ; "TsCl" ; Cc1ccc(S(=O)(=O)OC2NNC(=O)c3cnn(C)c32)cc1 ; Cc1ccc(S(=O)(=O)OC2NNC(=O)c3c2cnn3C)cc1]

Step-1: A mixture of diethyl 1-methyl-1H-pyrazole-4,5-dicarboxylate (3.0 g, 14 mmol), hydrazine hydrate (1.3 g, 28 mmol) in ethanol (20 mL) was stirred at 80° C. overnight. The reaction mixture was concentrated under vacuum to give 1-methyl-5,6-dihydro-1H-pyrazolo[3,4-d]pyridazine-4,7-dione (3.0 g, 90%). (MS ESI +Ve 166[M+1]).

Step-2: To a mixture of 1-methyl-5,6-dihydro-1H-pyrazolo[3,4-d]pyridazine-4,7-dione (3.0 g, 18 mmol) in THF (30 mL), TEA (0.4 g, 54 mmol) was added TsCl (4.1 g, 21 mmol) at 0° C. The mixture was stirred at room temperature overnight and was diluted with water, extracted with ethyl acetate. The organic layer was dried over Na₂SO₄ and evaporated under vacuum. The product was separated by column chromatography (50-70% ethyl acetate in hexane) to give 1-methyl-4-oxo-4,5-dihydro-1H-pyrazolo[3,4-d]pyridazin-7-yl 4-methylbenzenesulfonate (isomer-1)(1.0 g, 17%); (MS ESI +Ve 320[M+1]) and 1-methyl-7-oxo-6,7-dihydro-1H-pyrazolo[3,4-d]pyridazin-4-yl 4-methylbenzenesulfonate (isomer-2); (MS ESI +Ve 320[M+1]).

### (36) 5-(2-((2,2-difluorobenzo[d][1,3]dioxol-5-yl)(methyl)amino)-2-oxoethyl)-4-oxo-4,5-dihydro-1H-imidazo[4,5-d]pyridazin-7-yl 4-methylbenzenesulfonate

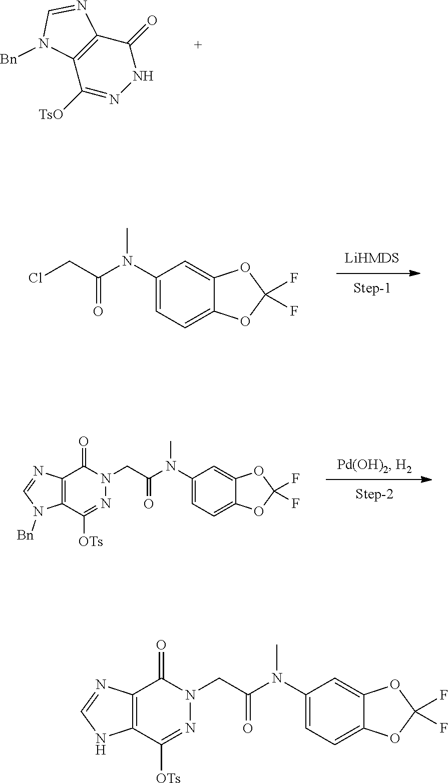 [SMILES: Cc1ccc(S(=O)(=O)Oc2n[nH]c(=O)c3ncn(Cc4ccccc4)c23)cc1 ; CN(C(=O)CCl)c1ccc2c(c1)OC(F)(F)O2 ; Cc1ccc(S(=O)(=O)Oc2nn(CC(=O)N(C)c3ccc4c(c3)OC(F)(F)O4)c(=O)c3ncn(Cc4ccccc4)c23)cc1 ; Cc1ccc(S(=O)(=O)Oc2nn(CC(=O)N(C)c3ccc4c(c3)OC(F)(F)O4)c(=O)c3nc[nH]c23)cc1]

Step-1: To a mixture of 1-benzyl-4-oxo-4,5-dihydro-1H-imidazo[4,5-d]pyridazin-7-yl 4-methylbenzenesulfonate (1.5 g, 3.78 mmol) in DMF (10 mL) was added lithium bis(trimethylsilyl)amide (4.9 mL, 4.91 mmol) at 0° C. The mixture was stirred for 5 min followed by addition of 2-chloro-N-(2,2-difluorobenzo[d][1,3]dioxol-5-yl)-N-methylacetamideide (1.1 g, 4.54 mmol). The reaction mixture was stirred overnight at rt and was quenched with water, extracted with ethyl acetate. The organic layer was washed with brine, dried and evaporated. The product was purified by CombiFlash (3% methanol in Dichloromethane) to give 1-benzyl-5-(2-((2,2-difluorobenzo[d][1,3]dioxol-5-yl)(methyl)amino)-2-oxoethyl)-4-oxo-4,5-dihydro-1H-imidazo[4,5-d]pyridazin-7-yl 4-methylbenzenesulfonate (1.6 g, 67%). (MS ESI +Ve 625[M+1]).

Step-2: To a mixture of 1-benzyl-5-(2-((2,2-difluorobenzo[d][1,3]dioxol-5-yl)(methyl)amino)-2-oxoethyl)-4-oxo-4,5-dihydro-1H-imidazo[4,5-d]pyridazin-7-yl 4-methylbenzenesulfonate (0.65 g, 2.0 mmol) in methanol (40 mL) was added Pd(OH)₂ (0.60 g, 0.87 mmol). The reaction mixture was stirred under hydrogen atmosphere at rt for 16 h. The catalyst was filtered off through a celite bed and the solution was concentrated to give 5-(2-((2,2-difluorobenzo[d][1,3]dioxol-5-yl)(methyl)amino)-2-oxoethyl)-4-oxo-4,5-dihydro-1H-imidazo[4,5-d]pyridazin-7-yl 4-methylbenzenesulfonate (0.25 g, 91%). (MS ESI +Ve 534 [M+1]).

The following compounds were synthesized in similar fashion:

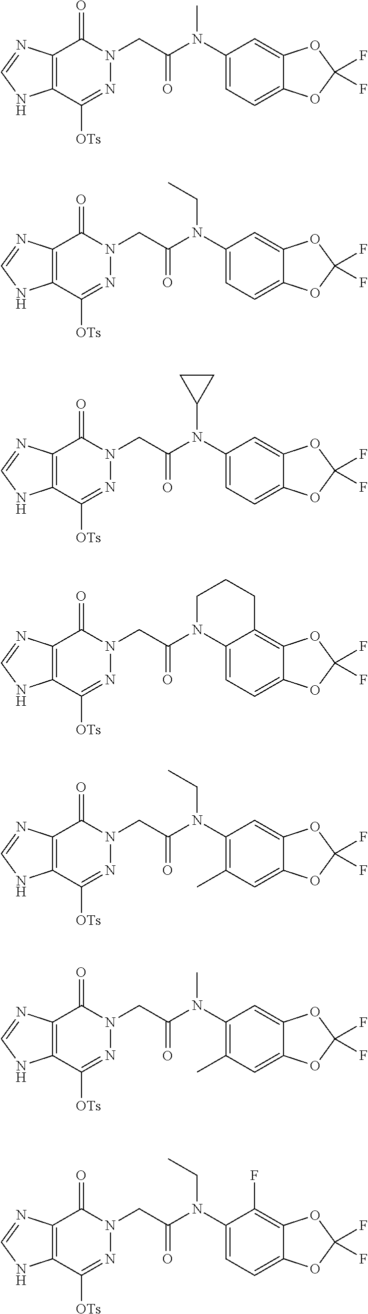

### (37) 4,6-dichloropyridazin-3(2H)-one and 5,6-dichloropyridazin-3(2H)-one

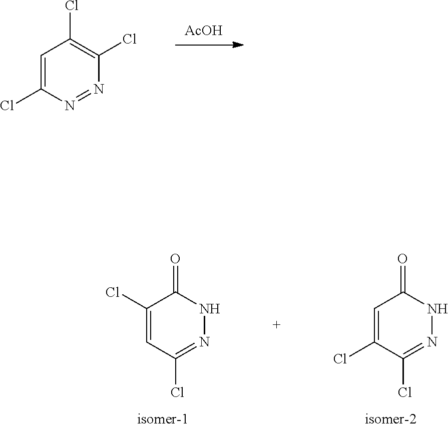 [SMILES: "AcOH" ; Clc1cc(Cl)c(Cl)nn1 ; O=c1[nH]nc(Cl)cc1Cl ; O=c1cc(Cl)c(Cl)n[nH]1]

3,4,6-trichloropyridazine (10 g, 56 mmol) was refluxed in acetic acid (20 mL) overnight at 110° C. The reaction mixture was concentrated and then the residue was diluted with water. The solid was filtered and separated by column chromatography (15-22% ethyl acetate in hexane) to give 5,6-dichloropyridazin-3(2H)-one (isomer-2) (3.5 g, 38%) and 4,6-dichloropyridazin-3(2H)-one (isomer-1) (3.5 g, 38%).

### (38) 7-chloro-1-(phenylsulfonyl)-1,5-dihydro-4H-pyrrolo[2,3-d]pyridazin-4-one and 4-chloro-1-(phenylsulfonyl)-1,6-dihydro-7H-pyrrolo[2,3-d]pyridazin-7-one

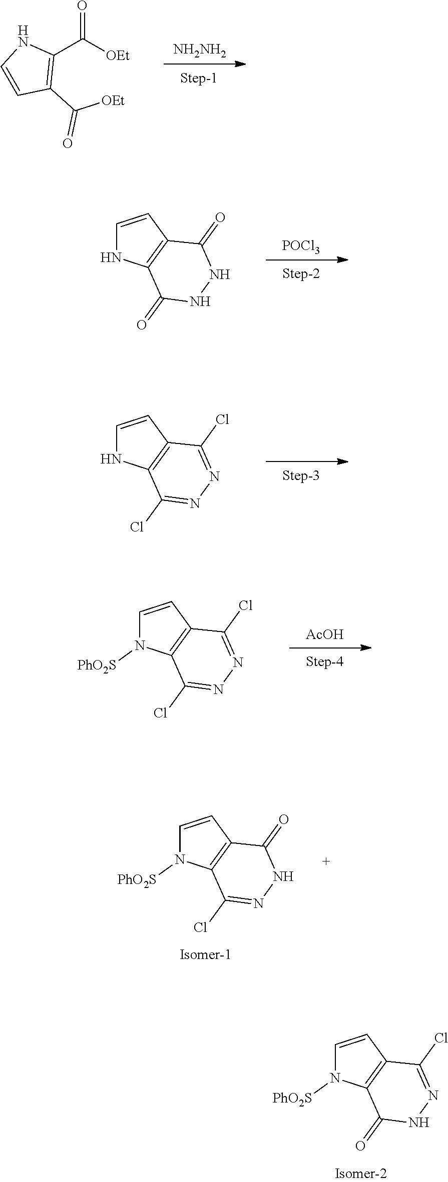

Step-1: A solution of diethyl 1H-pyrrole-2,3-dicarboxylate (5.0 g, 23.7 mmol) in ethanol (80 mL), was refluxing in hydrazine hydrate (20 mL) overnight. Solvent was evaporated and the crude product (5.0 g) was used for next step.

Step-2: a solution of above 5,6-dihydro-1H-pyrrolo[2,3-d]pyridazine-4,7-dione (1.0 g) in POCl₃ (20 mL) was refluxing overnight. The solvent was removed under vacuum and residue was poured onto ice water. The resulting mixture was extracted with AcOEt. The organic layers were evaporated to give 4,7-dichloro-1H-pyrrolo[2,3-d]pyridazine (0.7 g).

Step-3: To a solution of 4,7-dichloro-1H-pyrrolo[2,3-d]pyridazine (0.3 g, 1.6 mmol) in THF (10 mL) was added NaH (98 mg, 2.4 mmol) at 0° C. The mixture was stirring for 30 min before the addition of benzenesulfonyl chloride (0.31 mL, 1.9 mmol). The mixture was stirring at room temperature for 3 h and then quenched with ice water, extracted with AcOEt. The product was purified by combiflash (AcOEt/Hex) to give 4,7-dichloro-1-(phenylsulfonyl)-1H-pyrrolo[2,3-d]pyridazine (0.25 g, 48%).

Step-4: 4,7-dichloro-1-(phenylsulfonyl)-1H-pyrrolo[2,3-d]pyridazine (2.0 g, 3.81 mmol) was refluxed in acetic acid (10 mL) overnight. The reaction mixture was concentrated and then quenched with ice water. The solid was filtered and separated by column chromatography (15-20% ethyl acetate in hexane) to give 7-chloro-1-(phenylsulfonyl)-1,5-dihydro-4H-pyrrolo[2,3-d]pyridazin-4-one (isomer-1, 0.9 g) and 4-chloro-1-(phenylsulfonyl)-1,6-dihydro-7H-pyrrolo[2,3-d]pyridazin-7-one (1.0 g).

### Example-1 2-(7-(3-Fluorophenyl)-4-oxothieno[2,3-d]pyridazin-5(4H)-yl)-N-methyl-N-(2-methylbenzo[d]oxazol-6-yl)acetamide

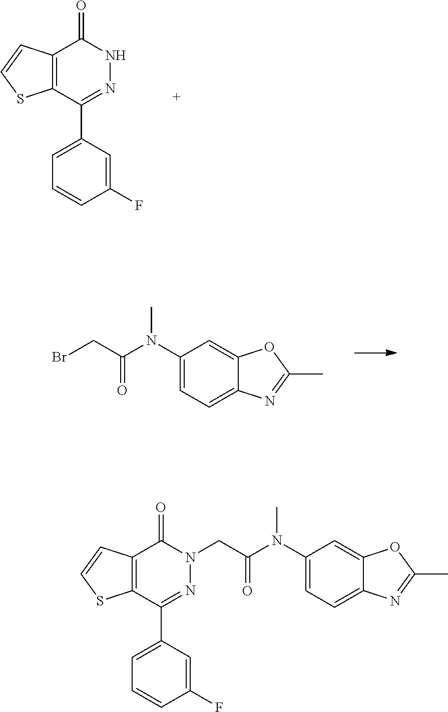 [SMILES: O=c1[nH]nc(-c2cccc(F)c2)c2sccc12 ; Cc1nc2ccc(N(C)C(=O)CBr)cc2o1 ; Cc1nc2ccc(N(C)C(=O)Cn3nc(-c4cccc(F)c4)c4sccc4c3=O)cc2o1]

To a solution of 7-(3-fluorophenyl)thieno[2,3-d]pyridazin-4(5H)-one (0.20 g, 0.81 mmol) in THF (10 mL), was added LiHMDS (1.2 mL, 1.22 mmol, 1.0 M in THF) dropwise at 0° C. The reaction mixture was stirred for 30 min, then 2-bromo-N-methyl-N-(2-methylbenzo[d]oxazol-6-yl)acetamide (0.23 g, 0.81 mmol) in THF (5 mL) was added. The mixture was stirred at rt overnight and was diluted with ice cold water (50 mL), extracted with EtOAc (2×50 mL). The organic layer was dried over Na₂SO₄ and concentrated to give crude product which was purified by column chromatography (40-50% EtOAc in hexane) to give 2-(7-(3-fluorophenyl)-4-oxothieno[2,3-d]pyridazin-5(4H)-yl)-N-methyl-N-(2-methylbenzo[d]oxazol-6-yl)acetamide (0.11 g, 29%) as white solid. (MS ESI +ve, 449.26 [M+]); ¹H NMR (400 MHz, DMSO) δ: 2.63 (s, 3H), 3.25 (s, 3H), 4.79 (s, 2H), 7.40-7.45 (m, 2H), 7.59-7.83 (m, 5H), 7.94 (s, 1H), 8.19-8.20 (d, J=5.6 Hz, 1H).

The following examples were synthesized in a similar fashion:

| Example# | Structure | m/z | ¹H NMR |
|---|---|---|---|
| 2 | 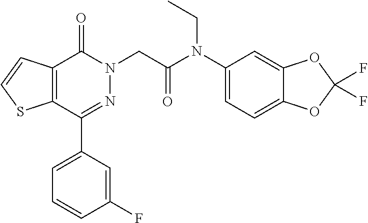 [SMILES: CCN(C(=O)Cn1nc(-c2cccc(F)c2)c2sccc2c1=O)c1ccc2c(c1)OC(F)(F)O2] | 488.33 [M + H] | (400 MHz, DMSO) δ: 1.03 (t, J = 6.8 Hz, 3H), 3.68 (q, J = 6.8 Hz, 2H), 4.77 (s, 2H), 7.32 (d, J = 8.4 Hz, 1H), 7.41-7.46 (m, 1H), 7.53 (d, J = 8.4 Hz, 1H), 7.61-7.72 (m, 5H), 8.21 (d, J = 5.2 Hz, 1H) |
| 3 | 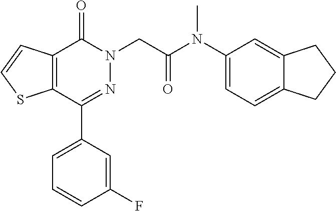 [SMILES: CN(C(=O)Cn1nc(-c2cccc(F)c2)c2sccc2c1=O)c1ccc2c(c1)CCC2] | 434.32 [M + H] | (400 MHz, DMSO) δ: 1.99-2.07 (m, 2H), 2.84-2.89 (m, 4H), 3.18 (s, 3H), 4.77 (s, 2H), 7.21-7.23 (d, J = 8.0 Hz, 1H), 7.32-7.34 (m, 2H), 7.41-7.46 (m, 1H), 7.61-7.72 (m, 4H), 8.19-8.20 (d, J = 5.6 Hz, 1H) |
| 4 | 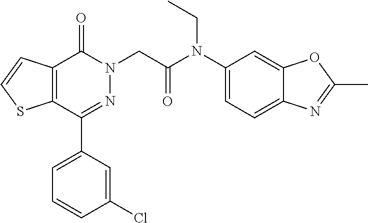 [SMILES: CCN(C(=O)Cn1nc(-c2cccc(Cl)c2)c2sccc2c1=O)c1ccc2nc(C)oc2c1] | 479.33 [M + 1] | (400 MHz, DMSO) δ: 1.05 (t, H = 6.8 Hz, 3H), 2.64 (s, 3H), 3.73 (q, J = 6.8 Hz, 2H), 4.74 (s, 2H), 7.40 (d, J = 8.0 Hz, 1H), 7.61 (m, 2H), 7.64 (d, J = 6.0 Hz, 2H), 7.70 (d, J = 5.2 Hz, 1H), 7.82 (s, 2H), 7.89 (s, 1H), 8.19 (d, J = 5.2 Hz, 1H) |
| 5 | 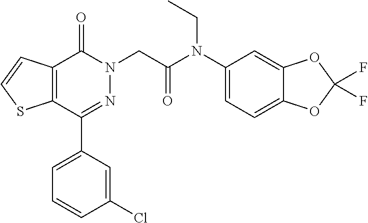 [SMILES: CCN(C(=O)Cn1nc(-c2cccc(Cl)c2)c2sccc2c1=O)c1ccc2c(c1)OC(F)(F)O2] | 504.22 [M + H] | (400 MHz, DMSO) δ: 1.03 (t, J = 7.2 Hz, 3H), 3.68 (q, J = 7.2 Hz, 2H), 4.77 (s, 2H), 7.33 (d, J = 9.2 Hz, 1H), 7.53 (d, J = 8.4 Hz, 1H), 7.63- 7.72 (m, 4H), 7.82-7.85 (m, 2H), 8.20 (d, J = 5.2 Hz, 1H) |
| 6 | 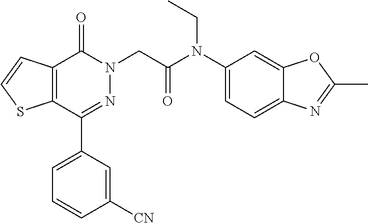 | 456.38 [M + H] | (400 MHz, DMSO) δ: 2.64 (s, 3H), 3.25 (s, 3H), 4.81 (s, 2H), 7.46 (d, J = 8.4 Hz, 1H), 7.71-7.76 (m, 2H), 7.83 (t, J = 8.0 Hz, 1H), 7.95 (s, 1H), 8.06 (d, J = 8.0 Hz, 1H), 8.18 (d, J = 8.0 Hz, 1H), 8.21-8.24 (m, 2H) |
| 7 | 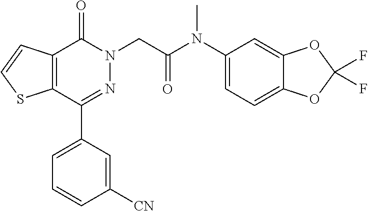 [SMILES: CN(C(=O)Cn1nc(-c2cccc(C#N)c2)c2sccc2c1=O)c1ccc2c(c1)OC(F)(F)O2] | 481.33 [M + H] | (400 MHz, DMSO) δ: 3.20 (s, 3H), 4.84 (s, 2H), 7.37-7.38 (d, J = 7.2 Hz, 1H), 7.51-7.53 (d, J = 8.4 Hz, 1H), 7.72-7.74 (m, 2H), 7.83 (t, J = 8.0 Hz, 1H), 8.06 (d, J = 7.6 Hz, 1H), 8.18- 8.32 (m, 3H) |
| 8 | 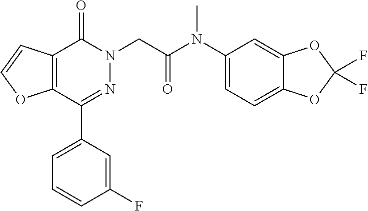 [SMILES: CN(C(=O)Cn1nc(-c2cccc(F)c2)c2occc2c1=O)c1ccc2c(c1)OC(F)(F)O2] | 458.45 [M + H] | (400 MHz, DMSO) δ: 3.19 (s, 3H), 4.83 (s, 2H), 7.24 (s, 1H), 131-1.42 (m, 2H), 7.53 (d, J = 8.0 Hz, 1H), 7.61-7.67 (m, 1H), 7.72 (s, 1H), 7.82 (d, J = 10.8 Hz, 1H), 7.92 (d, J = 8.0 Hz, 1H), 8.37 (d, J = 1.6 Hz, 1H) |
| 9 | 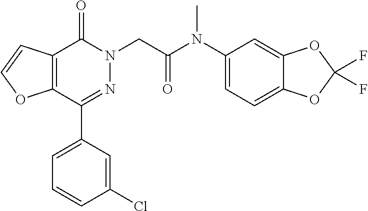 [SMILES: CN(C(=O)Cn1nc(-c2cccc(Cl)c2)c2occc2c1=O)c1ccc2c(c1)OC(F)(F)O2] | 474.28 [M + H] | (400 MHz, DMSO) δ: 3.20 (s, 3H), 4.84 (s, 2H), 7.24 (s, 1H), 7.38-7.40 (d, J = 6.8 Hz, 1H), 7.52-7.54 (d, J = 7.6 Hz, 1H), 7.61-7.65 (m, 2H), 7.72 (s, 1H), 8.02-8.06 (m, 2H), 8.37-8.38 (d, J = 2.0 Hz, 1H) |
| 10 | 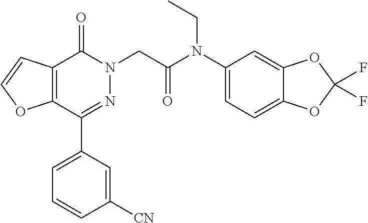 [SMILES: CCN(C(=O)Cn1nc(-c2cccc(C#N)c2)c2occc2c1=O)c1ccc2c(c1)OC(F)(F)O2] | 479.47 [M + H] | (400 MHz, DMSO) δ: 1.04 (t, J = 6.8 Hz, 3H), 3.69 (q, J = 6.8 Hz, 2H), 4.81 (s, 2H), 7.27 (s, 1H), 7.35 (d, J = 8.0 Hz, 1H), 7.56 (d, J = 8.4 Hz, 1H), 7.69 (s, 1H), 7.83 (t, J = 8.0 Hz, 1H), 8.04 (d, J = 7.6 Hz, 1H), 8.36-8.40 (m, 2H), 8.44 (s, 1H) |
| 11 | 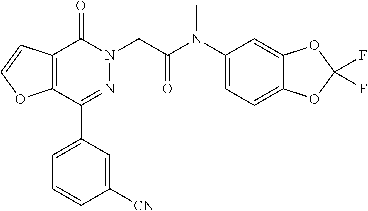 [SMILES: CN(C(=O)Cn1nc(-c2cccc(C#N)c2)c2occc2c1=O)c1ccc2c(c1)OC(F)(F)O2] | 465.28 [M + H] | (400 MHz, DMSO) δ: 3.20 (s, 3H), 4.85 (s, 2H), 7.26 (s, 1H), 7.39 (d, J = 8.4 Hz, 1H), 7.54 (d, J = 8.4 Hz, 1H), 7.73 (s, 1H), 7.82 (t, J = 8.0 Hz, 1H), 8.03 (d, J = 7.6 Hz, 1H), 8.36- 8.39 (m, 2H), 8.43 (s, 1H) |
| 12 | 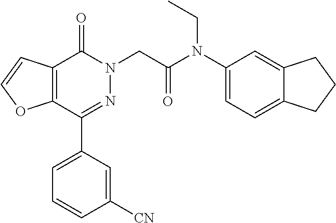 [SMILES: CCN(C(=O)Cn1nc(-c2cccc(C#N)c2)c2occc2c1=O)c1ccc2c(c1)CCC2] | 439.37 [M + H] | (400 MHz, DMSO) δ: 1.02 (t, J = 7.2 Hz, 3H), 2.01-2.07 (m, 2H), 2.83-2.88 (m, 4H), 3.65 (q, J = 7.2 Hz, 2H), 4.74 (s, 2H), 7.18 (d, J = 8.0 Hz, 1H), 7.24-7.28 (m, 2H), 7.34 (d, J = 8.0 Hz, 1H), 7.81 (t, J = 8.0 Hz, 1H), 8.02 (dt, J = 8.0, 1.2 Hz, 1H), 8.35-8.42 (m, 3H) |
| 13 | 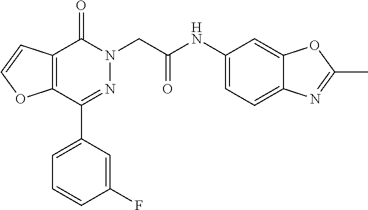 [SMILES: Cc1nc2ccc(NC(=O)Cn3nc(-c4cccc(F)c4)c4occc4c3=O)cc2o1] | 419.40 [M + H] | (400 MHz, DMSO) δ: 2.58 (s, 3H), 5.16 (s, 2H), 7.31 (s, 1H), 7.36-7.44 (m, 2H), 7.61 (d, J = 8.8 Hz, 1H), 7.65 (m, 1H), 7.87 (d, J = 10.0 Hz, 1H), 7.96 (d, J = 7.6 Hz, 1H), 8.09 (s, 1H), 8.41 (s, 1H), 10.62 (s, 1H) |
| 14 | 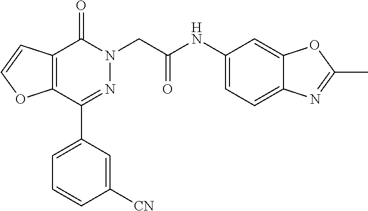 [SMILES: Cc1nc2ccc(NC(=O)Cn3nc(-c4cccc(C#N)c4)c4occc4c3=O)cc2o1] | 426.4 [M + H] | (400 MHz, DMSO) δ: 2.64 (s, 3H), 5.17 (s, 2H), 7.33 (s, 1H), 7.37 (d, J = 9.6 Hz, 1H), 7.61 (d, J = 8.8 Hz, 1H), 7.82 (t, J = 7.6 Hz, 1H), 8.04 (d, J = 7.2 Hz, 1H), 8.09 (s, 1H), 8.40-8.43 (m, 2H), 8.49 (s, 1H), 10.64 (s, 1H) |
| 15 | 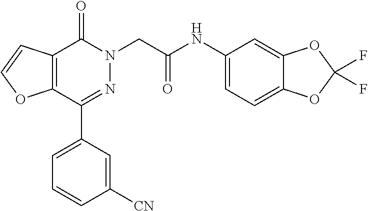 [SMILES: N#Cc1cccc(-c2nn(CC(=O)Nc3ccc4c(c3)OC(F)(F)O4)c(=O)c3ccoc23)c1] | 451.10 [M + 1] | (400 MHz, DMSO) δ: 5.14 (s, 2H), 7.27 (dd, J = 8.8, 1.6 Hz, 1H), 7.32 (d, J = 2.0 Hz, 1H), 7.39 (d, J = 8.8 Hz, 1H), 7.75 (d, J = 1.6 Hz, 1H), 7.82 (t, J = 8.0 Hz, 1H), 8.04 (d, J = 7.6 Hz, 1H), 8.39-8.42 (m, 2H), 8.48 (s, 1H), 10.65 (s, 1H) |
| 16 | 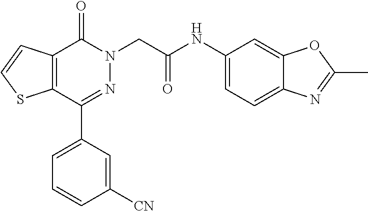 [SMILES: Cc1nc2ccc(NC(=O)Cn3nc(-c4cccc(C#N)c4)c4sccc4c3=O)cc2o1] | 442.36 [M + H] | (400 MHz, DMSO) δ: 2.54 (s, 3H), 5.15 (s, 2H), 7.37 (dd, J = 8.8, 2.0 Hz, 1H), 7.61 (d, J = 8.4 Hz, 1H), 7.79 (m, 1H), 7.85 (d, J = 8.0 Hz, 1H), 8.05-8.09 (m, 2H), 8.21-8.32 (m, 1H), 10.63 (s, 1H) |
| 17 | 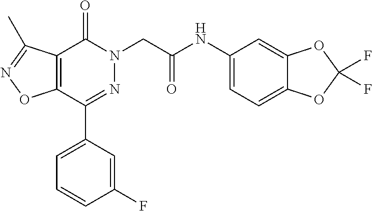 [SMILES: Cc1noc2c(-c3cccc(F)c3)nn(CC(=O)Nc3ccc4c(c3)OC(F)(F)O4)c(=O)c12] | 457.0 [M − H] | (400 MHz, DMSO) δ: 2.63 (s, 3H), 5.14 (s, 2H), 7.27 (dd, J = 8.8, 2.0 Hz, 1H), 7.39 (d, J = 8.8 Hz, 1H), 7.44 (td, J = 8.8, 1.6 Hz, 1H), 7.68 (m, 1H), 7.75 (d, J = 2.0 Hz, 1H), 7.84 (d, J = 10.0 Hz, 1H), 7.95 (d, J = 7.6 Hz, 1H), 10.66 (s, 1H) |
| 18 | 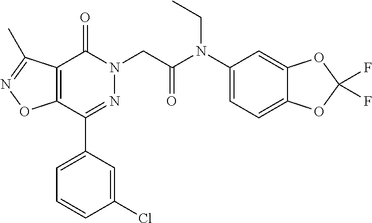 [SMILES: CCN(C(=O)Cn1nc(-c2cccc(Cl)c2)c2onc(C)c2c1=O)c1ccc2c(c1)OC(F)(F)O2] | 503.1 [M+] | (400 MHz, DMSO) δ: 1.03 (t, J = 6.8 Hz, 3H), 2.60 (s, 3H), 3.69 (q, J = 6.8 Hz, 2H), 4.80 (s, 2H), 7.34 (dd, J = 8.4, 1.6 Hz, 1H), 7.56 (d, J = 8.8 Hz, 1H), 7.63-7.69 (m, 3H), 8.01-8.03 (m, 2H) |
| 19 | 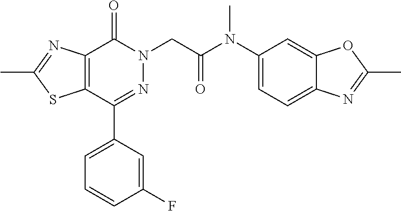 | 464.2 [M + H] | (400 MHz, DMSO) δ: 2.63 (s, 3H), 2.89 (s, 3H), 3.25 (s, 3H), 4.82 (s, 2H), 7.41-7.47 (m, 2H), 7.57-7.66 (m, 3H), 7.75 (d, J = 8.4 Hz, 1H), 7.96 (s, 1H) |
| 20 | 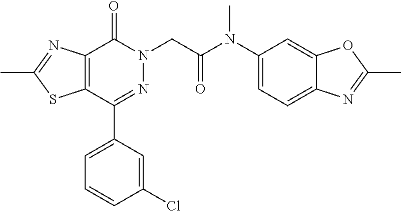 | 480.3 M + H] | (400 MHz, DMSO) δ: 2.64 (m, 3H), 2.89 (s, 3H), 3.26 (s, 3H), 4.83 (s, 2H), 7.46 (d, J = 8.4 Hz, 1H), 7.63-7.66 (m, 2H), 7.74-7.77 (m, 2H), 7.81 (s, 1H), 7.97 (s, 1H) |

### Example 21

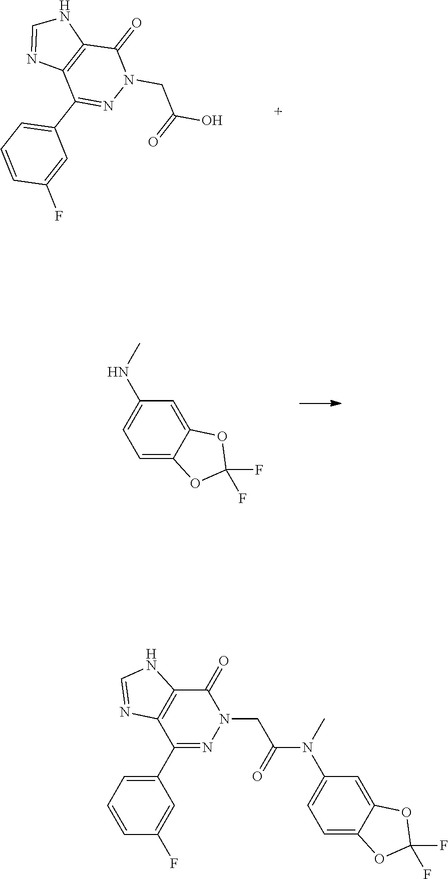 [SMILES: O=C(O)Cn1nc(-c2cccc(F)c2)c2nc[nH]c2c1=O ; CNc1ccc2c(c1)OC(F)(F)O2 ; CN(C(=O)Cn1nc(-c2cccc(F)c2)c2nc[nH]c2c1=O)c1ccc2c(c1)OC(F)(F)O2]

A mixture of 2-(7-(3-fluorophenyl)-4-oxo-3,4-dihydro-5H-imidazo[4,5-d]pyridazin-5-yl)acetic acid (0.29 g, 1.0 mmol) in DCM (2 mL), 2,2-difluoro-N-methylbenzo[d][1,3]dioxol-5-amine (0.21 g, 1.1 mmol) and 4-dimethylaminopyridine (0.024 g) was stirred at room temperature for 5 min and then N-(3-dimethylaminopropyl)-N-ethylcarbodiimide hydrochloride (0.287 g, 1.5 mmol) was added. The resultant mixture was stirring at room temperature for 16 h and diluted with water, extracted with DCM (3×30 mL). The combined organic layer was washed with brine and dried over sodium sulfate. The volatiles were removed under vacuum and the residue was purified by neutral alumina chromatography (0-10% methanol in Dichloromethane) to afford 2-(7-(3-chlorophenyl)-4-oxo-3,4-dihydro-5H-imidazo[4,5-d]pyridazin-5-yl)-N-(2,2-difluorobenzo[d][1,3]dioxol-5-yl)-N-methylacetamide (0.12 g, 260%) as off-white solid. (MS ESI +ve, 458.2 [M+H]); ¹H NMR: (400 MHz, DMSO-d6) δ: 3.20 (s, 3H), 4.85 (s, 2H), 7.31-7.40 (m, 2H), 7.51-7.61 (m, 2H), 7.79 (s, 1H), 8.27 (m, 2H), 8.52 (s, 1H), 14.19 (br, 1H).

The following compounds were synthesized in a similar fashion:

| Example# | Stucture | m/z | HNMR |
|---|---|---|---|
| 22 | 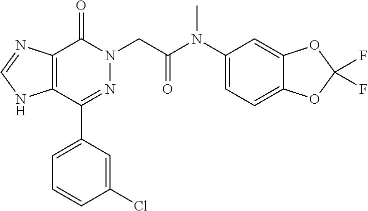 [SMILES: CN(C(=O)Cn1nc(-c2cccc(Cl)c2)c2[nH]cnc2c1=O)c1ccc2c(c1)OC(F)(F)O2] | 474.2 [M + H] | (400 MHz, DMSO-d₆) δ: 3.34 (s, 3H), 4.85 (s, 2H), 7.40 (m, 1H), 7.52-7.60 (m, 3H), 7.73 (s, 1H), 8.38 (m, 1H), 8.48 (s, 1H), 8.53 (s, 1H), 13.61 (br, 1H) |
| 23 | 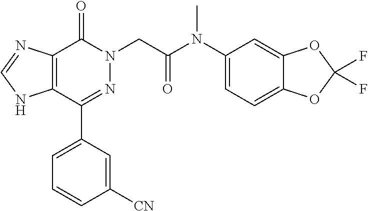 [SMILES: CN(C(=O)Cn1nc(-c2cccc(C#N)c2)c2[nH]cnc2c1=O)c1ccc2c(c1)OC(F)(F)O2] | 465.4 [M + H] | (400 MHz, DMSO-d₆) δ: 3.18 (s, 3H), 4.84 (s, 2H), 7.38 (d, J = 7.6 Hz, 1H), 7.51 (d, J = 8.0 Hz, 1H), 7.71-7.77 (m, 2H), 7.95 (d, J = 7.6 Hz, 1H), 8.54 (s, 1H), 8.69 (s, 1H), 8.79 (s, 1H), 14.26 (br, 1H) |
| 24 | 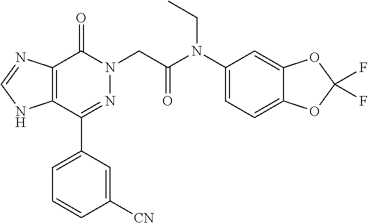 [SMILES: CCN(C(=O)Cn1nc(-c2cccc(C#N)c2)c2[nH]cnc2c1=O)c1ccc2c(c1)OC(F)(F)O2] | 479.0 [M + H] | (400 MHz, DMSO-d₆) 1.03 (t, J = 6.8 Hz, 3H), 3.68 (q, J = 6.8 Hz, 2H), 4.81 (s, 2H), 7.33 (d, J = 8.0 Hz, 1H), 7.53 (d, J = 8.4 Hz, 1H), 7.67 (s, 1H), 7.74-7.78 (m, 2H), 7.96 (d, J = 7.6 Hz, 1H), 8.56 (s, 1H), 8.72 (d, J = 7.6 Hz, 1H), 8.83 (s, 1H), 14.29 (br, 1H) |
| 25 | 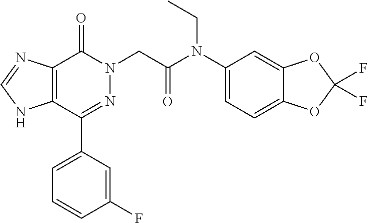 [SMILES: CCN(C(=O)Cn1nc(-c2cccc(F)c2)c2[nH]cnc2c1=O)c1ccc2c(c1)OC(F)(F)O2] | 472.2 [M + H] | (400 MHz, DMSO-d₆) δ: 1.04 (m, 3H), 3.68 (q, J = 7.2 Hz, 2H), 4.79 (s, 2H), 7.34 (m, 2H), 7.52-7.60 (m, 2H), 7.66 (s, 1H), 8.26 (m, 3H), 8.53 (s, 1H), 14.22 (br, 1H) |
| 26 | 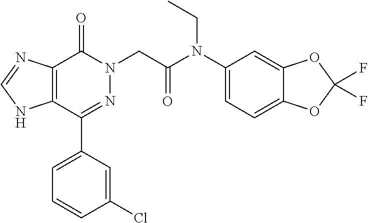 [SMILES: CCN(C(=O)Cn1nc(-c2cccc(Cl)c2)c2[nH]cnc2c1=O)c1ccc2c(c1)OC(F)(F)O2] | 488.4 [M + H] | (400 MHz, DMSO-d₆) δ: 1.04 (t, J = 7.2 Hz, 3H), 3.68 (q, J = 7.2 Hz, 2H), 4.79 (s, 2H), 7.34 (d, J = 8.4 Hz, 1H), 7.52-7.60 (m, 3H), 7.67 (s, 1H), 8.37 (m, 1H), 8.48-8.52 (m, 2H), 14.08 (br, 1H) |
| 27 | 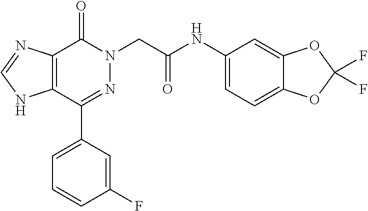 [SMILES: O=C(Cn1nc(-c2cccc(F)c2)c2[nH]cnc2c1=O)Nc1ccc2c(c1)OC(F)(F)O2] | 444.0 [M + H] | (400 MHz, DMSO-d₆) δ: 5.17 (s, 2H), 7.31- 7.38 (m, 3H), 7.55-7.61 (m, 1H), 7.81 (s, 1H), 8.28 (br, 2H), 8.55 (s, 1H), 11.09 (s, 1H), 14.28 (s, 1H) |
| 28 | 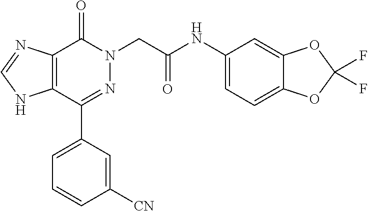 [SMILES: N#Cc1cccc(-c2nn(CC(=O)Nc3ccc4c(c3)OC(F)(F)O4)c(=O)c3nc[nH]c23)c1] | 451.0 [M + H] | (400 MHz, DMSO-d₆) δ: 5.10 (s, 2H), 7.25- 7.28 (dd, J = 1.6, 1H), 7.35-7.37 (d, J = 8.4, 1H), 7.70-7.74 (m, 2H), 7.90-7.92 (d, J = 7.6, 1H), 8.33 (s, 1H), 8.75-8.77 (d, J = 8.4, 1H), 8.92 (s, 1H), 10.57 (s, 1H) |
| 29 | 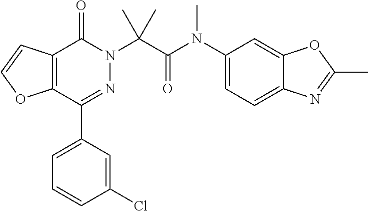 [SMILES: Cc1nc2ccc(N(C)C(=O)C(C)(C)n3nc(-c4cccc(Cl)c4)c4occc4c3=O)cc2o1] | 477.2 [M + H] | (400 MHz, DMSO) δ: 1.04-1.05 (d, 3H), 2.56 (s, 3H), 3.08-3.15 (m, 4H), 4.16-4.21 (m, 1H), 4.34-4.39 (q, 1H), 7.04 (m, 1H), 7.23-7.23 (d, J = 2, 1H), 7.36 (m, 1H), 7.51- 7.64 (m, 3H), 7.85-7.92 (m, 2H), 8.39-8.39 (d, J = 5, 1H) |
| 30 | 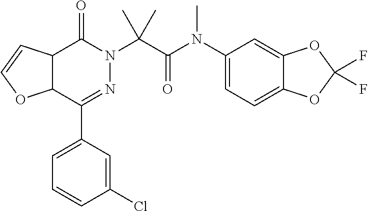 [SMILES: CN(C(=O)C(C)(C)N1N=C(c2cccc(Cl)c2)C2OC=CC2C1=O)c1ccc2c(c1)OC(F)(F)O2] | 502.0 [M + H] | (400 MHz, DMSO) δ: 1.03-1.05 (d, 3H), 3.10 (s, 3H), 4.22-4.38 (m, 2H), 6.98-7.00 (d, J = 5, 1H), 7.24 (s, 2H), 7.32-7.34 (d, J = 8.4, 1H), 7.59-7.63 (t, 2H), 7.92-7.96 (t, 2H), 7.38-7.38 (d, J = 1.6, 1H) |
| 31 | 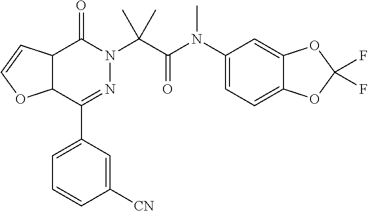 [SMILES: CN(C(=O)C(C)(C)N1N=C(c2cccc(C#N)c2)C2OC=CC2C1=O)c1ccc2c(c1)OC(F)(F)O2] | 493.2 [M + H] | (400 MHz, DMSO) δ: 1.04-1.06 (d, 3H), 3.09 (s, 3H), 4.18-4.39 (m, 2H), 6.99 (s, 1H), 7.25 (s, 2H), 7.32-7.34 (d, J = 8.8, 1H), 7.79-7.83 (t, 1H), 8.04-8.05 (d, J = 7.6, 1H), 8.24-8.26 (d, J = 8, 1H), 8.34(s, 1H), 8.39- 8.40 (d, J = 1.6, 1H) |
| 32 | 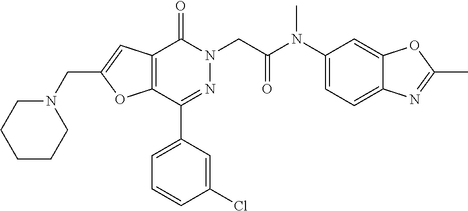 | 537.2 [M + 1] | (400 MHz, DMSO) δ: 1.37 (m, 2H); 1.51 (m, J = 5.2 Hz, 4H); 2.64 (m, 4H); 3.25 (s, 3H); 3.78 (s, 2H); 4.82 (s, 2H); 7.02 (s, 1H), 7.46 (m, 1H), 7.75 (d, J = 8.4 Hz, 1H), 7.82 (t, J = 8.0 Hz, 1H), 7.95 (s, 1H), 8.02 (d, J = 7.6 Hz, 1H), 8.34 (d, J = 8.0 Hz, 1H), 8.38 (s, 1H) |

### Example 33

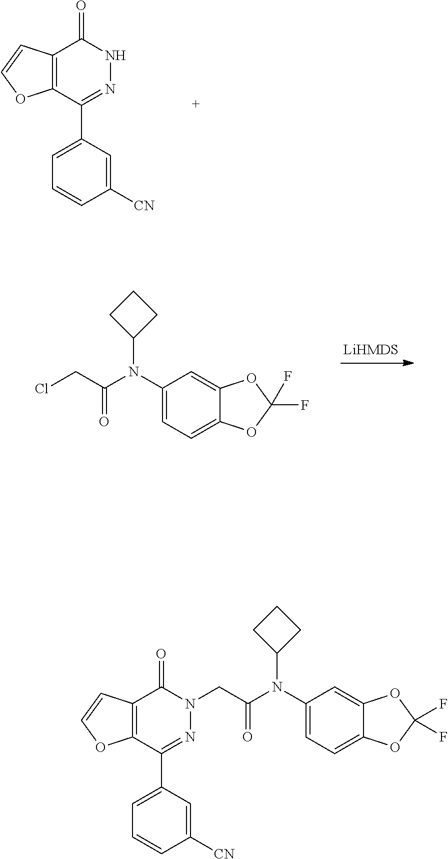 [SMILES: N#Cc1cccc(-c2n[nH]c(=O)c3ccoc23)c1 ; "+" ; O=C(CCl)N(c1ccc2c(c1)OC(F)(F)O2)C1CCC1 ; N#Cc1cccc(-c2nn(CC(=O)N(c3ccc4c(c3)OC(F)(F)O4)C3CCC3)c(=O)c3ccoc23)c1]

To a solution of 3-(4-oxo-4,5-dihydrofuro[2,3-d]pyridazin-7-yl)benzonitrile (0.15 g, 0.63 mmol) in DMF (10 mL) was added lithium bis(trimethylsilyl)amide (0.95 mL, 0.94 mmol, 1M in THF) at 0° C. The resultant solution was stirred at 0° C. for 20 min and then 2-chloro-N-cyclobutyl-N-(2,2-difluorobenzo[d][1,3]dioxol-5-yl)acetamide (0.25 g, 0.76 mmol) in DMF (2 mL) was added drop wise at 0° C. The reaction mixture allowed to warm rt and stirred for 16 h. The reaction was quenched with ice-cold water, extracted with ethyl acetate (3×50 mL). The combined organic layer was washed with brine and dried over sodium sulfate. The solvent were removed under vacuum and resulting material was purified by column chromatography (25-30% ethyl acetate in hexane) to give 2-(7-(3-cyanophenyl)-4-oxofuro[2,3-d]pyridazin-5(4H)-yl)-N-cyclobutyl-N-(2,2-difluorobenzo[d][1,3]dioxol-5-yl)acetamide (0.27 g, 84%) as white solid. (MS ESI +ve, 505.2 [M+H]) ¹H NMR: (400 MHz, DMSO-d6): 1.24-1.27 (m, 1H), 1.47-1.62 (m, 2H), 1.76-192 (m, 2H), 2.03 (m, 2H), 4.71 (s, 2H), 4.90 (m, 1H), 7.26 (m, 1H), 7.52-7.55 (m, 2H), 7.82 (t, J=8.0 Hz, 1H), 8.04 (d, J=7.6 Hz, 1H), 8.36-8.44 (m, 3H).

Examples synthesized in a similar fashion:

| Example# | Stucture | m/z | ¹H NMR |
|---|---|---|---|
| 34 | 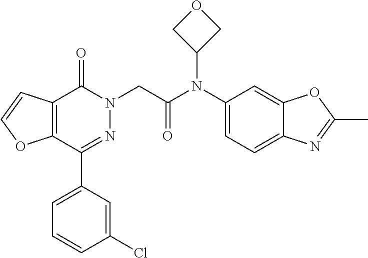 [SMILES: Cc1nc2ccc(N(C(=O)Cn3nc(-c4cccc(Cl)c4)c4occc4c3=O)C3COC3)cc2o1] | 492.2 [M + H] | (400 MHz, DMSO-d₆) 2.66 (s, 3H), 4.41 (t, J = 6.8 Hz, 2H), 4.61 (t, J = 6.8 Hz, 2H), 4.77 (s, 2H), 5.45 (m, 1H), 7.24 (d, J = 2.0 Hz, 1H), 7.49 (d, J = 7.6 Hz, 1H), 7.62-7.65 (m, 2H), 7.82 (d, J = 8.4 Hz, 1H), 8.00-8.03 (m, 3H), 8.37 (d, J = 2.0 Hz, 1H) |
| 35 | 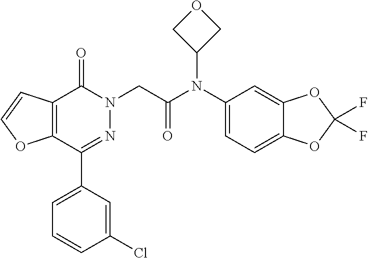 [SMILES: O=C(Cn1nc(-c2cccc(Cl)c2)c2occc2c1=O)N(c1ccc2c(c1)OC(F)(F)O2)C1COC1] | 507 [M + 1] | (400 MHz, DMSO) δ: 4.41 (t, J = 6.8 Hz, 2H), 4.61 (t, J = 6.8 Hz, 2H), 4.81 (s, 2H), 5.40 (m, 1H), 7.27 (d, J = 1.6 Hz, 1H), 7.44 (d, J = 8.0 Hz, 1H), 7.61 (d, J = 8.4 Hz, 1H), 7.78-7.84 (m, 2H), 8.04 (d, J = 7.6 Hz, 1H), 8.35-8.43 (m, 3H) |
| 36 | 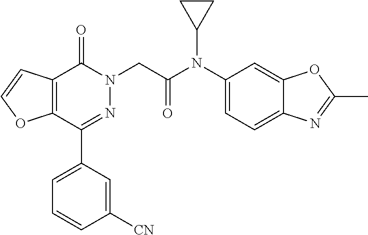 [SMILES: Cc1nc2ccc(N(C(=O)Cn3nc(-c4cccc(C#N)c4)c4occc4c3=O)C3CC3)cc2o1] | 466.2 [M + 1] | (400 MHz, DMSO) δ: 0.63 (m, 2H), 0.87 (d, J = 5.6 Hz, 2H), 2.67 (s, 3H), 3.35 (m, 1H), 5.13 (s, 2H), 7.21 (d, J = 2.0 Hz, 1H), 7.26 (dd, J = 8.4, 1.6 Hz, 1H), 7.63-7.67 (m, 2H), 7.80 (t, J = 8.0 Hz, 1H), 7.97 (d, J = 7.6 Hz, 1H), 8.30 (d, J = 2.0 Hz, 1H), 8.38 (d, J = 8.0 Hz, 1H), 8.41 (s, 1H) |
| 37 | 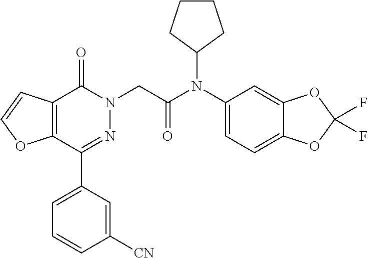 [SMILES: N#Cc1cccc(-c2nn(CC(=O)N(c3ccc4c(c3)OC(F)(F)O4)C3CCCC3)c(=O)c3ccoc23)c1] | 519.2 [M + H] | (400 MHz, DMSO): 1.18-1.24 (m, 2H), 1.31-1.35 (m, 4H), 1.84 (m, 2H), 4.69 (s, 2H), 4.72 (m, 1H), 7.26-7.30 (m, 2H), 7.58 (d, J = 8.8 Hz, 1H), 7.63 (s, 1H), 7.82 (dd, J = 8.0, 7.6 Hz, 1H), 8.04 (d, J = 8.0 Hz, 1H), 8.36-8.40 (m, 2H), 8.44 (s, 1H) |
| 38 | 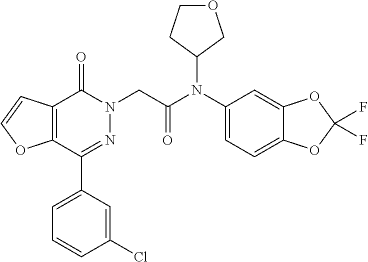 [SMILES: O=C(Cn1nc(-c2cccc(Cl)c2)c2occc2c1=O)N(c1ccc2c(c1)OC(F)(F)O2)C1CCOC1] | 530.0 [M + 1] | (400 MHz, DMSO) δ: 1.79-1.84 (m, 1H), 2.09-2.18 (m, 1H), 3.51-3.57 (m, 2H), 3.70-3.82 (m, 2H), 4.69 (s, 2H), 5.01 (m, 1H), 7.26 (d, J = 1.6 Hz, 1H), 7.37 (d, J = 7.2 Hz, 1H), 7.57 (d, J = 8.4 Hz, 1H), 7.63 (d, J = 4.0 Hz, 1H), 7.68 (s, 1H), 8.03 (d, J = 8.4 Hz, 1H), 8.06 (d, J = 4.0 Hz, 1H), 8.39 (d, J = 2.0 Hz, 1H) |
| 39 | 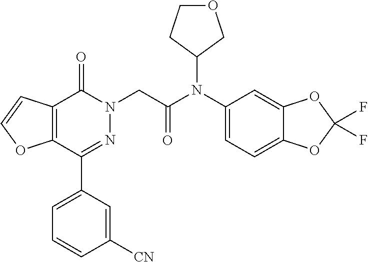 [SMILES: N#Cc1cccc(-c2nn(CC(=O)N(c3ccc4c(c3)OC(F)(F)O4)C3CCOC3)c(=O)c3ccoc23)c1] | 521 [M + 1] | (400 MHz, DMSO) δ: 1.83 (m, 1H), 2.12 (m, 1H), 3.27-3.47 (m, 1H), 3.51-3.57 (m, 1H), 3.69-3.82 (m, 2H), 4.71 (s, 2H), 5.01 (m, 1H), 7.27 (d, J = 2.0 Hz, 1H), 7.37 (d, J = 7.6 Hz, 1H), 7.57 (d, J = 8.4 Hz, 1H), 7.69 (s, 1H), 7.82 (dd, J = 8.0, 7.6 Hz, 1H), 8.04 (d, J = 7.6 Hz, 1H), 8.36-8.43 (m, 3H) |
| 40 | 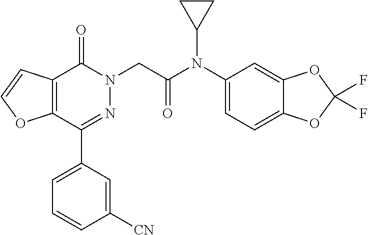 [SMILES: N#Cc1cccc(-c2nn(CC(=O)N(c3ccc4c(c3)OC(F)(F)O4)C3CC3)c(=O)c3ccoc23)c1] | 491.5. [M + 1] | (400 MHz, DMSO) δ: 0.67 (s, 2H), 0.91 (d, J = 6.0 Hz, 2H), 3.31 (m, 1H), 5.19 (s, 2H), 7.15 (d, J = 8.4 Hz, 1H), 7.21 (s, 1H), 7.38- 7.41 (m, 2H), 7.80 (t, J = 8.0 Hz, 1H), 7.97 (d, J = 7.6 Hz, 1H), 8.30 (s, 1H), 8.39 (d, J = 8.0 Hz, 1H), 8.42 (s, 1H) |
| 41 | 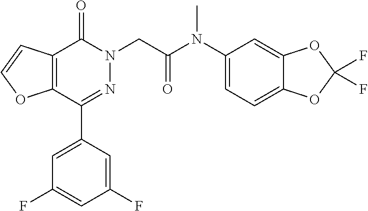 [SMILES: CN(C(=O)Cn1nc(-c2cc(F)cc(F)c2)c2occc2c1=O)c1ccc2c(c1)OC(F)(F)O2] | 476.0 [M + 1] | (400 MHz, DMSO) δ: 3.21 (s, 3H), 4.86 (s, 2H), 7.27 (s, 1H), 7.40 (d, J = 8.0 Hz, 1H), 7.47-7.56 (m, 2H), 7.74-7.77 (m, 3H), 8.39 (d, J = 2.0 Hz, 1H) |
| 42 | 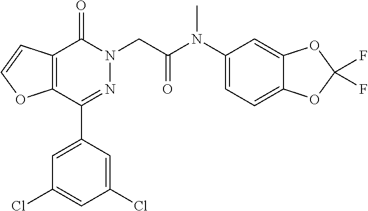 [SMILES: CN(C(=O)Cn1nc(-c2cc(Cl)cc(Cl)c2)c2occc2c1=O)c1ccc2c(c1)OC(F)(F)O2] | 508 [M + 1] | (400 MHz, DMSO) δ: 3.21 (s, 3H), 4.86 (s, 2H), 7.26 (s, 1H), 7.40 (d, J = 7.6 Hz, 1H), 7.55 (d, J = 8.4 Hz, 1H), 7.74 (s, 1H), 7.83 (t, J = 2.0 Hz, 1H), 8.07 (m 2H), 8.401- 8.40 (d, J = 2.0 Hz, 1H) |
| 43 | 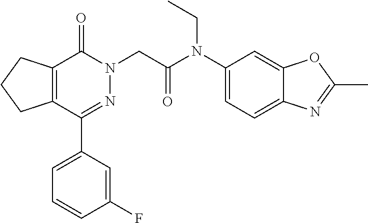 [SMILES: CCN(C(=O)Cn1nc(-c2cccc(F)c2)c2c(c1=O)CCC2)c1ccc2nc(C)oc2c1] | 447.2 [M + 1] | (400 MHz, DMSO) δ 1.04 (t, J = 6.4 Hz, 3H), 2.06 (t, J = 7.2 Hz, 2H), 2.59 (s, 3H), 2.75 (s, 2H), 3.05 (s, 2H), 3.73 (m, 2H), 4.65 (s, 2H), 7.30-7.39 (m, 4H), 7.56 (t, J = 6.8 Hz, 1H), 7.77 (d, J = 8.0 Hz, 1H), 7.88 (s, 1H) |
| 44 | 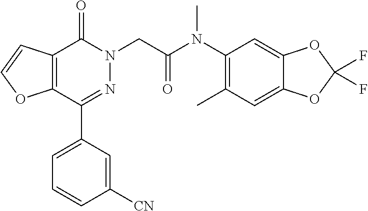 [SMILES: Cc1cc2c(cc1N(C)C(=O)Cn1nc(-c3cccc(C#N)c3)c3occc3c1=O)OC(F)(F)O2] | 479.2 [M + 1] | (400 MHz, DMSO) δ: 400; 2.38 (s, 3H), 3.12 (s, 3H), 4.63 (d, J = 16.0 Hz, 1H), 4.77 (d, J = 16.0 Hz, 1H), 7.28 (d, J = 2.0 Hz, 1H), 7.54 (s, 1H), 7.69 (s, 1H), 7.83 (t, J = 8.0 Hz, 1H), 8.04 (d, J = 7.6 Hz, 1H), 8.36-8.44 (m, 3H) |
| 45 | 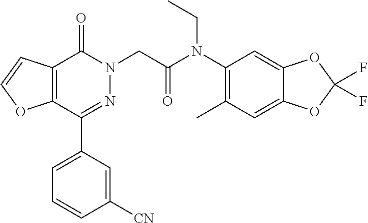 [SMILES: CCN(C(=O)Cn1nc(-c2cccc(C#N)c2)c2occc2c1=O)c1cc2c(cc1C)OC(F)(F)O2] | 493.2 [M + 1] | (400 MHz, DMSO) δ: 1.06 (t, J = 6.8 Hz, 3H), 2.38 (s, 3H), 3.21 (q, J = 6.8 Hz, 1H), 3.99 (q, J = 6.8 Hz, 1H), 4.58 (d, J = 16.0 Hz, 1H), 4.75 (d, J = 16.0 Hz, 1H), 7.27 (d, J = 1.6 Hz, 1H), 7.55 (s, 1H), 7.63 (s, 1H), 7.82 (m, 1H), 8.03 (d, J = 7.6 Hz, 1H), 8.36- 8.44 (m, 3H) |
| 46 | 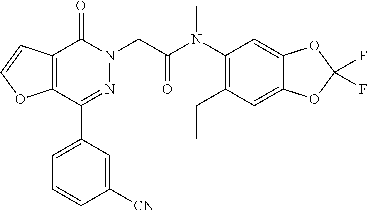 [SMILES: CCc1cc2c(cc1N(C)C(=O)Cn1nc(-c3cccc(C#N)c3)c3occc3c1=O)OC(F)(F)O2] | 492 [M + 1] | (400 MHz, DMSO) δ: 1.31 (m, 3H), 2.76 (m, 1H), 2.91 (m, 1H), 3.15 (m, 3H), 4.63 (d, J = 16.0 Hz, 1H), 4.83 (d, J = 16.0 Hz, 1H), 7.21 (s, 1H), 7.45 (s, 1H), 7.56 (s, 1H), 7.80 (t, J = 8.0 Hz, 1H), 7.97 (d, J = 7.6 Hz, 1H), 8.29 (d, J = 1.6 Hz, 1H), 8.36-8.40 (m, 2H) |
| 47 | 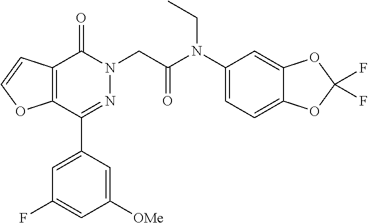 [SMILES: CCN(C(=O)Cn1nc(-c2cc(F)cc(OC)c2)c2occc2c1=O)c1ccc2c(c1)OC(F)(F)O2] | 502.15 [M + 1] | (400 MHz, DMSO) δ: 1.04 (t, J = 7.2 Hz, 3H), 3.69 (q, J = 7.2 Hz, 2H), 3.88 (s, 3H), 4.78 (s, 2H), 7.06 (dd, J = 10.8, J = 2.4 Hz, 1H), 7.24 (s, 1H), 7.34 (d, J = 8.4 Hz, 1H), 7.42 (d, J = 9.6 Hz, 1H), 7.47 (s, 1H), 7.55 (d, J = 8.4 Hz, 1H), 7.67 (s, 1H), 8.37 (d, J = 2.0 Hz, 1H) |
| 48 | 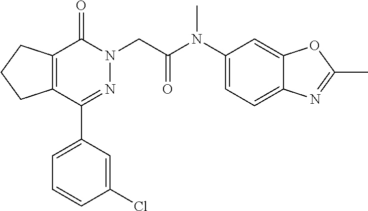 [SMILES: Cc1nc2ccc(N(C)C(=O)Cn3nc(-c4cccc(Cl)c4)c4c(c3=O)CCC4)cc2o1] | 449.2 [M + 1] | (400 MHz, DMSO-d₆) δ 2.08 (m, 2H), 2.56 (m, 2H), 3.05 (m, 2H), 3.25 (s, 3H), 4.72 (s, 2H), 7.44 (d, J = 6.8 Hz, 1H), 7.51- 7.65 (m, 3H), 7.77 (d, J = 8.8 Hz, 1H), 7.82 (m, 1H), 7.93 (s, 1H) |
| 49 | 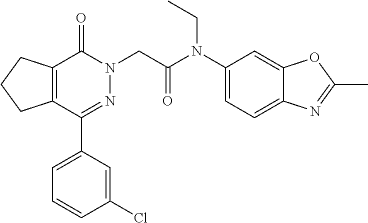 [SMILES: CCN(C(=O)Cn1nc(-c2cccc(Cl)c2)c2c(c1=O)CCC2)c1ccc2nc(C)oc2c1] | 463.0 [M + 1] | (400 MHz, DMSO-d₆) δ : 1.06 (t, J = 7.2 Hz , 3H), 2.05 (m, 2H), 2.65 (s, 3H), 2.75 (m, 2H), 3.04 (m, 2H), 3.73 (q, J = 7.2 Hz, 2H), 4.65 (s, 2H), 7.39 (d, J = 8.4 Hz, 1H), 7.54 (s, 2H), 7.59 (d, J = 7.6 Hz, 1H), 7.65 (s, 1H), 7.77 (d, J = 8.4 Hz, 1H), 7.88 (s, 1H) |
| 50 | 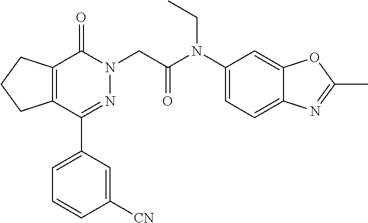 [SMILES: CCN(C(=O)Cn1nc(-c2cccc(C#N)c2)c2c(c1=O)CCC2)c1ccc2nc(C)oc2c1] | 454.30 [M + 1] | (400 MHz, DMSO-d₆) δ 1.04 (t, J = 7.2 Hz, 3H), 2.06 (m, 2H), 2.65 (s, 3H), 2.76 (t, J = 7.2 Hz, 2H), 3.05 (t, J = 6.8 Hz, 2H), 3.7 (q, J = 6.8 Hz, 2H), 4.66 (s, 2H), 7.39 (d, J = 8.0 Hz, 1H), 7.70-7.79 (m, 2H), 7.89 (s, 1H), 7.96 (m, 2H), 8.06 (s, 1H) |
| 51 | 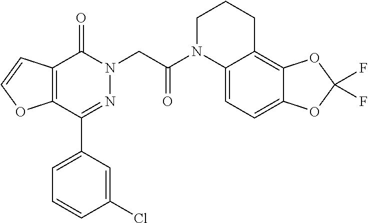 [SMILES: O=C(Cn1nc(-c2cccc(Cl)c2)c2occc2c1=O)N1CCCc2c1ccc1c2OC(F)(F)O1] | 500.0 [M + H] | (400 MHz, DMS0-d₆): 2.00 (m, 2H), 2.84 (t, J = 6.8 Hz, 2H), 3.83 (m, 2H), 5.35 (s, 2H), 7.24 (d, H-8.4 Hz, 1H), 7.29 (s, H), 7.53 (d, J = 8.0 Hz, 1H), 7.67-7.63 (m, 2H), 8.08-8.05 (m, 2H), 8.40 (s, 1H) |
| 52 | 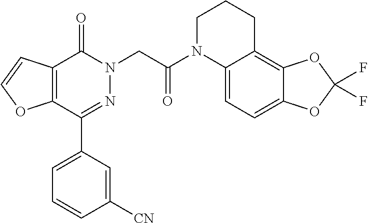 [SMILES: N#Cc1cccc(-c2nn(CC(=O)N3CCCc4c3ccc3c4OC(F)(F)O3)c(=O)c3ccoc23)c1] | 491.0 [M + H] | (400 MHz, DMSO-d₆): 2.00 (m, 2H), 2.81- 2.85 (t, J = 7.6 Hz, 2H), 3.83 (m, 2H), 5.36 (s, 2H), 7.25 (d, J = 8.4 Hz, 1H), 7.30 (s, 1H), 7.53 (d, J = 6.8 Hz, 1H), 7.83 (t, J = 8.0 Hz, 1H), 8.04 (d, J = 8.0 Hz, 1H), 8.41-8.39 (m, 2H), 8.46 (s, 1H) |
| 53 | 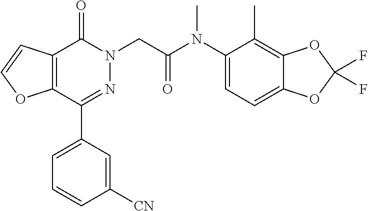 [SMILES: Cc1c(N(C)C(=O)Cn2nc(-c3cccc(C#N)c3)c3occc3c2=O)ccc2c1OC(F)(F)O2] | 479.0 [M + H] | (400 MHz, DMSO-d₆): 2.34 (s, 3H), 3.14 (s, 3H), 4.63-4.77 (m, 2H), 7.28(d, J = 2.0 Hz, 1H), 7.40 (s, 2H), 7.80-7.84 (dd, J = 8.0, 7.6 Hz, 2H), 8.04 (d, J = 7.6, 1H), 8.40-8.36 (m, 2H), 8.44 (s, 1H) |
| 54 | 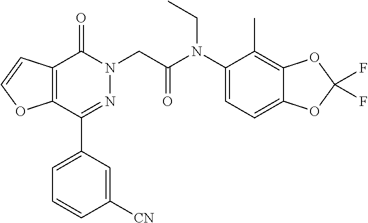 [SMILES: CCN(C(=O)Cn1nc(-c2cccc(C#N)c2)c2occc2c1=O)c1ccc2c(c1C)OC(F)(F)O2] | 493 [M + H] | (400 MHz, DMSO-d₆): 1.05 (s, J = 6.8 Hz, 3H), 2.34 (s, 3H), 3.24 (q, J = 6.8 Hz, 1H), 4.01 (q, J = 6.8 Hz, 1H), 4.62 (d, J = 16 Hz, 1H), 4.71 (d, J = 16 Hz, 1H), 7.27 (s, 1H), 7.34-7.41 (m, 2H), 7.82 (t, J = 8.0 Hz, 1H), 8.03 (d, J = 7.2 Hz, 1H), 8.36-8.44 (m, 3H) |
| 55 | 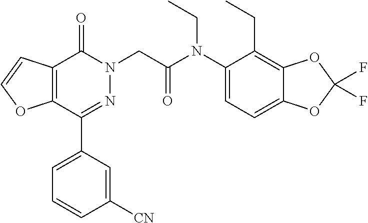 [SMILES: CCc1c(N(CC)C(=O)Cn2nc(-c3cccc(C#N)c3)c3occc3c2=O)ccc2c1OC(F)(F)O2] | 507.4 [M + H] | (400 MHz, DMSO-d₆): 1.11 (t, J = 6.8 Hz, 3H), 1.34 (t, J = 7.2 Hz, 3H), 2.77 (q, J = 7.2 Hz, 2H), 3.14 (q, J = 6.8 Hz, 1H), 4.08 (q, J = 6.8 Hz, 1H), 4.58 (d, J = 16.0 Hz, 1H), 4.77 (d, J = 16.0 Hz, 1H), 7.29 (s, 1H), 7.37 (d, J = 8.4 Hz, 1H), 7.43 (d, J = 8.4 Hz, 1H), 7.82 (t, J = 8.0 Hz, 1H), 8.04 (d, J = 7.6 Hz, 1H), 8.36-8.43 (m, 3H) |
| 56 | 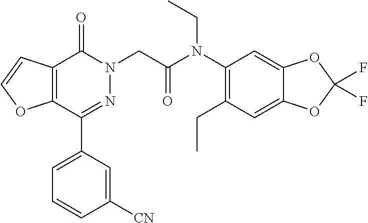 [SMILES: CCc1cc2c(cc1N(CC)C(=O)Cn1nc(-c3cccc(C#N)c3)c3occc3c1=O)OC(F)(F)O2] | 507.2 [M + 1] | (400 MHz, DMSO) δ: 1.06 (t, J = 6.8 Hz, 3H); 1.30 (t, J = 7.2 Hz, 3H); 2.64-2.77 (m, 2H); 3.16 (q, J = 6.8 Hz, 1H); 4.03 (q, J = 6.8 Hz, 1H); 4.54 (d, J = 16.0 Hz, 1H); 4.79 (d, J = 16.0 Hz, 1H); 7.28 (t, J = 6.0 Hz, 1H); 7.58 (s, 1H); 7.64 (s, 1H); 7.82 (t, J = 8.0 Hz, 1H); 8.03 (d, J = 8.0 Hz, 1H); 8.35-8.42 (m, 3H) |
| 57 | 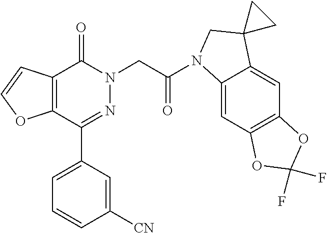 [SMILES: N#Cc1cccc(-c2nn(CC(=O)N3CC4(CC4)c4cc5c(cc43)OC(F)(F)O5)c(=O)c3ccoc23)c1] | 503.83 [M + 1] | (400 MHz, DMSO) 1.16-1.19 (m, 4H), 4.40 (s, 2H), 5.32 (m, 2H), 7.07 (s, 1H), 7.35 (d, J = 1.6 Hz, 1H), 7.83 (t, J = 8.0 Hz, 1H), 7.94 (s, 1H), 8.04 (d, J = 7.6 Hz, 1H), 8.42 (d, J = 8.0 Hz, 1H), 8.44 (d, J = 1.6 Hz, 1H), 8.47(s, 1H) |
| 58 | 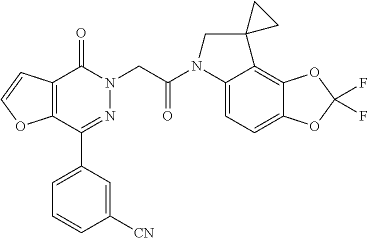 [SMILES: N#Cc1cccc(-c2nn(CC(=O)N3CC4(CC4)c4c3ccc3c4OC(F)(F)O3)c(=O)c3ccoc23)c1] | 503.0 [M + H] | (400 MHz, DMSO): 1.24-1.28 (m, 2H), 1.39 (s, 2H), 4.43 (s, 2H), 5.31 (s, 2H), 7.16 (d, J = 8.4 Hz, 1H), 7.35 (d, J = 2.0 Hz, 1H), 7.85-7.81 (m, 2H), 8.05 (d, J = 7.6 Hz, 1H), 8.40-8.48 (m, 3H) |
| 59 | 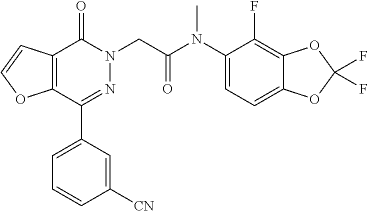 [SMILES: CN(C(=O)Cn1nc(-c2cccc(C#N)c2)c2occc2c1=O)c1ccc2c(c1F)OC(F)(F)O2] | 483.0 [M + H] | (400 MHz, DMSO): 3.17 (s, 3H), 4.83 (d, J = 16.0 Hz, 1H), 4.92 (d, J = 16.0 Hz, 1H), 7.28 (d, J = 2.0 Hz, 1H), 7.80-7.85 (m, 2H), 7.92 (d, J = 6.8 Hz, 1H), 8.04 (d, J = 7.6 Hz, 1H), 8.36 (d, J = 8.4 Hz, 1H), 8.40-8.46 (m, 2H) |
| 60 | 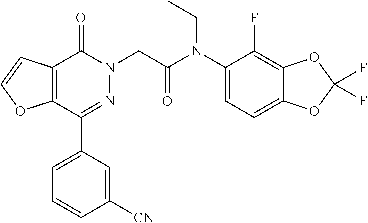 [SMILES: CCN(C(=O)Cn1nc(-c2cccc(C#N)c2)c2occc2c1=O)c1ccc2c(c1F)OC(F)(F)O2] | 497.4 [M + H] | (400 MHz, DMSO): 1.04 (t, J = 6.4 Hz, 3H), 3.66 (m, 2H), 4.78 (d, J = 16.0 Hz, 1H), 4.87 (d, J = 16.0 Hz, 1H), 7.28 (s, 1H), 7.84-7.87 (m, 3H), 8.05 (d, J = 7.6 Hz, 1H), 8.35-8.44 (m, 3H) |
| 61 | 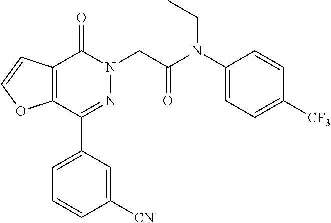 [SMILES: CCN(C(=O)Cn1nc(-c2cccc(C#N)c2)c2occc2c1=O)c1ccc(C(F)(F)F)cc1] | 467 [M + 1] | (400 MHz, DMSO) δ: 1.06 (m, 3H), 3.76 (m, 2H), 4.82 (s, 2H), 7.26 (d, J = 1.6 Hz, 1H), 7.72 (m, 2H), 7.82 (t, J = 8.0 Hz, 1H), 7.88 (d, J = 7.6 Hz, 2H), 8.03 (d, J = 7.6 Hz, 1H), 8.37 (d, J = 8.0 Hz, 1H), 8.39 (d, J = 2.0 Hz, 1H), 8.43 (s, 1H) |
| 62 | 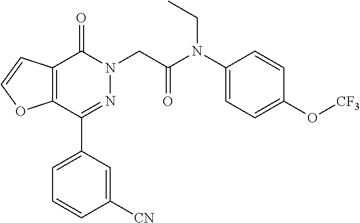 [SMILES: CCN(C(=O)Cn1nc(-c2cccc(C#N)c2)c2occc2c1=O)c1ccc(OC(F)(F)F)cc1] | 483 M + 1] | (400 MHz, DMSO-d₆) δ: 1.09 (t, J = 6.8 Hz, 3H), 3.71 (m, 2H), 4.77 (s, 2H), 7.27 (s, 1H), 7.55 (m, 2H), 7.64 (m, 2H), 7.82 (t, J = 7.6 Hz, 1H), 8.04 (d, J = 7.6 Hz, 1H), 8.37-8.40 (m, 2H), 8.45 (s, 1H) |
| 63 | 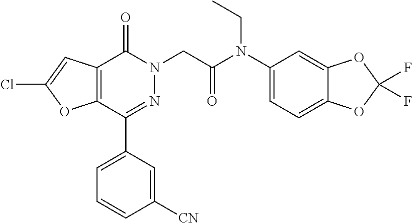 [SMILES: CCN(C(=O)Cn1nc(-c2cccc(C#N)c2)c2oc(Cl)cc2c1=O)c1ccc2c(c1)OC(F)(F)O2] | 513 [M + 1] | (400 MHz, DMSO) δ: 1.04 (t, J = 6.8 Hz, 3H), 3.68 (q, J = 6.8 Hz, 2H), 4.78 (s, 2H), 7.35 (d, J = 8.0 Hz, 1H), 7.57 (d, J = 8.4 Hz, 1H), 7.69 (s, 1H), 7.82 (t, J = 8.0 Hz, 1H), 8.05 (d, J = 7.6 Hz, 1H), 8.32 (d, J = 7.6 Hz, 1H), 8.39 (s, 1H), 8.66 (s, 1H) |
| 64 | 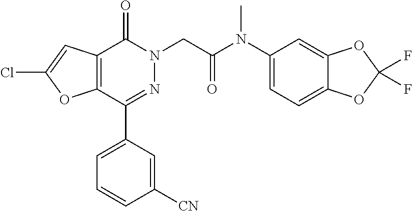 [SMILES: CN(C(=O)Cn1nc(-c2cccc(C#N)c2)c2oc(Cl)cc2c1=O)c1ccc2c(c1)OC(F)(F)O2] | 499 [M + 1] | (400 MHz, DMSO )δ: 3.36 (s, 3H), 4.85 (s, 2H), 7.42 (s, 1H), 7.56 (m, 1H), 7.75 (s, 1H), 7.80-7.86 (m, 1H), 8.05 (d, J = 8.0 Hz, 1H), 8.32 (d, J = 8.0 Hz, 1H), 8.39 (d, J = 8.8 Hz, 1H), 8.67 (s, 1H) |

### Example 65

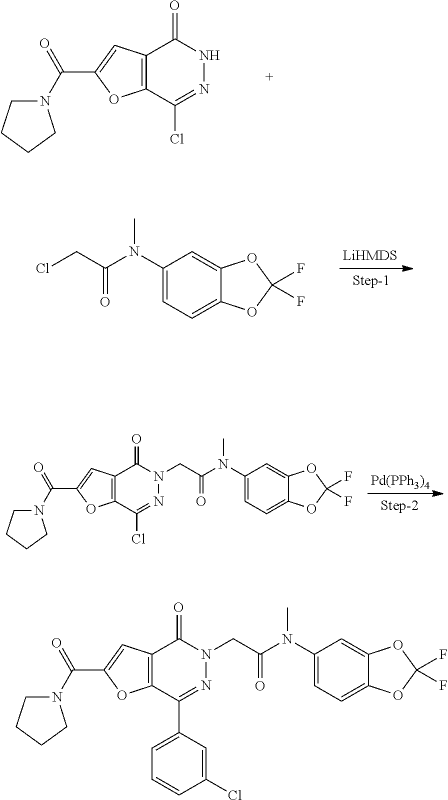 [SMILES: O=C(c1cc2c(=O)[nH]nc(Cl)c2o1)N1CCCC1 ; "+" ; CN(C(=O)CCl)c1ccc2c(c1)OC(F)(F)O2 ; CN(C(=O)Cn1nc(Cl)c2oc(C(=O)N3CCCC3)cc2c1=O)c1ccc2c(c1)OC(F)(F)O2 ; CN(C(=O)Cn1nc(-c2cccc(Cl)c2)c2oc(C(=O)N3CCCC3)cc2c1=O)c1ccc2c(c1)OC(F)(F)O2 ; "Pd(PPh3)4"]

Step-1: To a stirred solution of 7-chloro-2-(pyrrolidine-1-carbonyl)furo[2,3-d]pyridazin-4(5H)-one (0.2 g, 0.75 mmol) in tetrahydrofuran (10 mL) was added LiHMDS (1 M in THF, 1.12 mL, 1.12 mmol) drop wise at 0° C. The reaction mixture was stirred for 30 min at 0° C. and 2-chloro-N-(2,2-difluorobenzo[d][1,3]dioxol-5-yl)-N-methylacetamide (0.22 g, 0.82 mmol) in tetrahydrofuran (3 mL) was added drop wise at 0° C. The reaction mixture was stirred at 25° C. for 16 h and was diluted with water (50 mL). The product was extracted with ethyl acetate (3×50 mL), washed with brine (40 mL), dried over anhydrous sodium sulfate and concentrated. The crude product was purified by combiflash flash (5% methanol in dichloromethane) to give 2-(7-chloro-4-oxo-2-(pyrrolidine-1-carbonyl)furo[2,3-d]pyridazin-5(4H)-yl)-N-(2,2-difluorobenzo[d][1,3]dioxol-5-yl)-N-methylacetamide (0.12 g, 32%) as brown solid. (MS ESI +ve, 495.50 [M+1]).

Step-2: A mixture of 2-(7-chloro-4-oxo-2-(pyrrolidine-1-carbonyl)furo[2,3-d]pyridazin-5(4H)-yl)-N-(2,2-difluorobenzo[d][1,3]dioxol-5-yl)-N-methylacetamide (0.12 g, 0.24 mmol), (3-chlorophenyl)boronic acid (0.057 g, 0.36 mmol) in toluene (2 mL), ethanol (2 mL), water (1 mL) and sodium carbonate (0.039 g, 0.36 mmol) was degassed, followed by addition of Pd(PPh₃)₄ (0.028 g, 0.024 mmol). The mixture was degassed again and heated at 60° C. overnight. The reaction mixture was evaporated and the residue was purified by column chromatography (5% methanol in dichloromethane) to give crude product, which was purified by reverse phase preparative HPLC to give 2-(7-(3-chlorophenyl)-4-oxo-2-(pyrrolidine-1-carbonyl)furo[2,3-d]pyridazin-5(4H)-yl)-N-(2,2-difluorobenzo[d][1,3]dioxol-5-yl)-N-methylacetamide (0.06 g, 43%) as off white solid. (MS ESI +ve, 571.2 [M+1]); ¹H NMR: (400 MHz, DMSO) δ: 1.85-2.04 (m, 4H), 3.21 (s, 3H), 3.55 (t, J=6.4 Hz, 2H), 3.91 (t, J=6.4 Hz, 2H), 4.86 (s, 2H), 7.41 (d, J=8.4 Hz, 1H), 7.56 (d, J=8.4 Hz, 1H), 7.64-7.68 (m, 3H), 7.76 (s, 1H), 8.03 (d, J=3.2 Hz, 1H), 8.11 (s, 1H).

In a similar fashion, the following examples were synthesized:

| Example# | Stucture | m/z | ¹H NMR |
|---|---|---|---|
| 66 | 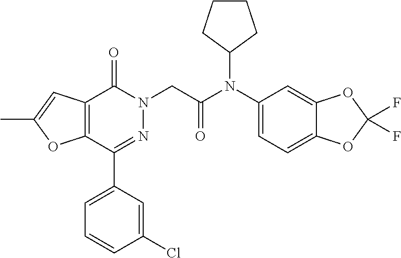 [SMILES: Cc1cc2c(=O)n(CC(=O)N(c3ccc4c(c3)OC(F)(F)O4)C3CCCC3)nc(-c3cccc(Cl)c3)c2o1] | 542.6 [M + H] | (400 MHz, DMSO-d₆): 1.32-1.35 (m, 2H), 1.47 (m, 4H), 1.84 (m, 2H), 2.57 (s, 3H), 4.65 (s, 2H), 4.72 (m, 1H), 6.87 (s, 1H), 7.28 (d, J = 8.4 Hz, 1H), 7.57 (d, J = 8.4 Hz, 1H), 7.61-7.65 (m, 3H), 8.03-8.04 (m, 2H) |
| 67 | 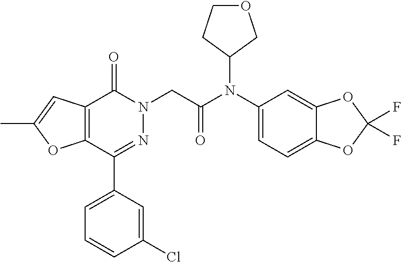 [SMILES: Cc1cc2c(=O)n(CC(=O)N(c3ccc4c(c3)OC(F)(F)O4)C3CCOC3)nc(-c3cccc(Cl)c3)c2o1] | 544 [M + 1] | (400 MHz, DMSO) δ: 1.78-1.85 (m, 1H), 2.09-2.18 (m, 1H), 2.56 (s, 3H), 3.30-3.46 (m, 1H), 3.51-3.57 (m, 1H), 3.69-3.73 (m, 1H), 3.77-3.81 (m, 1H), 4.65 (s, 2H), 5.0 (m, 1H), 6.86 (s, 1H), 7.35 (d, J = 7.6 Hz, 1H), 7.55- 7.65 (m, 3H), 7.68 (s, 1H), 8.01-8.04 (m, 2H) |
| 68 | 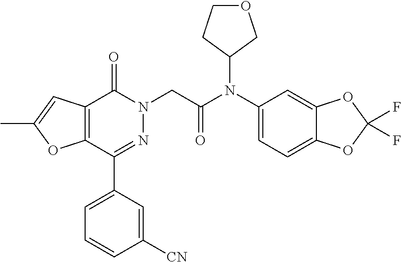 [SMILES: Cc1cc2c(=O)n(CC(=O)N(c3ccc4c(c3)OC(F)(F)O4)C3CCOC3)nc(-c3cccc(C#N)c3)c2o1] | 535.2 [M + 1] | (400 MHz, DMSO) δ: 1.78-1.82 (m, 1H), 2.10-2.15 (m, 1H), 2.56 (s, 3H), 3.44-3.54 (m, 2H), 3.68-3.80 (m, 2H), 4.66 (s, 2H), 5.00 (m, 1H), 6.87 (s, 1H), 7.35 (d, J = 7.6 Hz, 1H), 7.56 (d, J = 8.4 Hz, 1H), 7.67 (s, 1H), 7.80 (dd, J = 8.4, 8.0 Hz, 1H), 8.01 (d, J = 8.0 Hz, 1H), 8.35 (d, J = 8.0 Hz, 1H), 8.39 (s, 1H) |
| 69 | 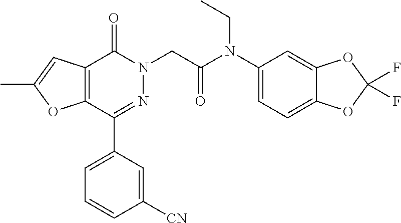 [SMILES: CCN(C(=O)Cn1nc(-c2cccc(C#N)c2)c2oc(C)cc2c1=O)c1ccc2c(c1)OC(F)(F)O2] | 493.6 [M + 1] | (400 MHz, DMSO) δ: 1.04 (t, J = 6.8 Hz, 3H), 2.58 (s, 3H) 3.69 (q, J = 6.8 Hz, 2H), 4.78 (s, 2H), 6.89 (s, 1H), 7.34 (d, J = 7.2 Hz, 1H), 7.55 (d, J = 8.4 Hz, 1H), 7.67 (s, 1H), 7.82 (t, J = 8.0 Hz, 1H), 8.03 (d, J = 8.0 Hz, 1H), 8.36-8.42 (m, 2H) |
| 70 | 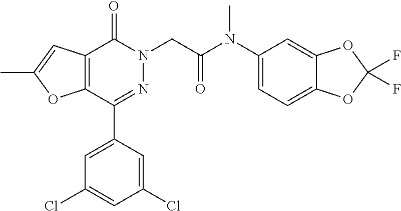 [SMILES: Cc1cc2c(=O)n(CC(=O)N(C)c3ccc4c(c3)OC(F)(F)O4)nc(-c3cc(Cl)cc(Cl)c3)c2o1] | 522.5 [M + 1] | (400 MHz, DMSO) δ: 2.58 (s, 3H); 3.20 (s, 3H),4.83 (s, 2H) 6.88 (s, 1H); 7.41 (m, 1H), 7.55 (m, 1H), 7.83 (t, J = 2.0 Hz, 1H); 8.04 (d, J = 1.2 Hz, 2H) |
| 71 | 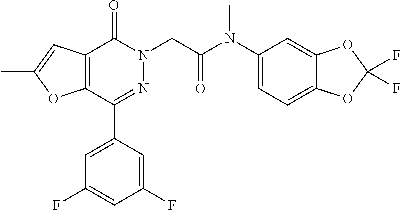 [SMILES: Cc1cc2c(=O)n(CC(=O)N(C)c3ccc4c(c3)OC(F)(F)O4)nc(-c3cc(F)cc(F)c3)c2o1] | 490.0 [M + 1] | (400 MHz, DMSO) δ: 2.58 (s, 3H); 3.20 (s, 3H), 4.83 (s, 2H), 6.88 (s, 1H), 7.38 (d, J = 7.6 Hz, 1H); 7.44- 7.55 (m, 2H), 7.73-7.75 (m, 3H) |
| 72 | 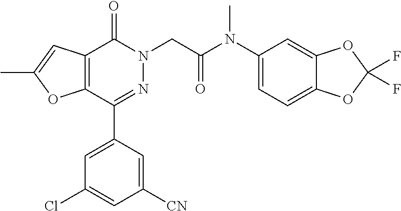 [SMILES: Cc1cc2c(=O)n(CC(=O)N(C)c3ccc4c(c3)OC(F)(F)O4)nc(-c3cc(Cl)cc(C#N)c3)c2o1] | 513 [M + 1] | (400 MHz, DMSO) δ: 2.57 (s, 3H), 3.20 (s, 3H), 4.85 (s, 2H), 6.90 (s, 1H), 7.41 (m, 1H); 7.55 (d, J = 8.0 Hz, 1H); 7.73 (s, 1H), 8.25 (d, J = 1.6 Hz, 1H); 8.37 (s, 1H), 8.42 (s, 1H) |
| 73 | 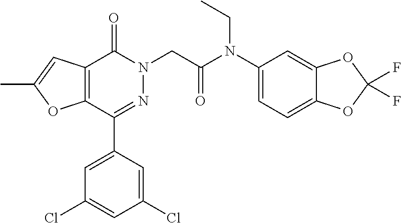 [SMILES: CCN(C(=O)Cn1nc(-c2cc(Cl)cc(Cl)c2)c2oc(C)cc2c1=O)c1ccc2c(c1)OC(F)(F)O2] | 536 [M + 1] | (400 MHz, DMSO) δ: 1.20 (t, J = 7.2 Hz, 3H), 2.61 (s, 3H); 3.83 (q, J = 7.2 Hz, 2H), 4.82 (s, 2H), 6.75 (s, 1H); 7.23 (d, J = 8.0 Hz, 1H), 7.30 (s, 1H), 7.47 (m, 1H), 7.64 (s, J = 2.0 Hz, 1H); 8.03 (m, 2H) |
| 74 | 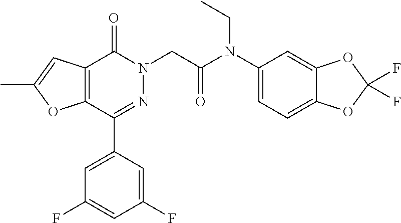 [SMILES: CCN(C(=O)Cn1nc(-c2cc(F)cc(F)c2)c2oc(C)cc2c1=O)c1ccc2c(c1)OC(F)(F)O2] | 504.0 [M + 1] | (400 MHz, DMSO) δ: 1.04 (t, J = 6.8 Hz, 3H); 2.58 (s, 3H); 3.68 (q, J = 6.8 Hz, 2H), 4.77 (s, 2H), 6.87 (s, 1H); 7.33 (d, J = 8.0 Hz, 1H), 7.47 (t, J = 9.2 Hz, 1H), 7.54 (d, J = 8.4 Hz, 1H); 7.66 (s, 1H), 7.73 (d, J = 6.8 Hz, 2H) |
| 75 | 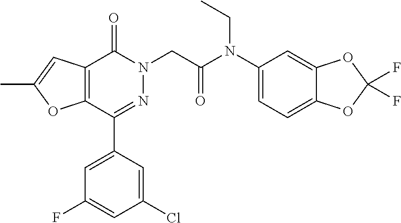 [SMILES: CCN(C(=O)Cn1nc(-c2cc(F)cc(Cl)c2)c2oc(C)cc2c1=O)c1ccc2c(c1)OC(F)(F)O2] | 520.0 [M + 1] | (400 MHz, DMSO) δ: 1.04 (t, J = 6.8 Hz, 3H); 2.58 (s, 3H); 3.68 (q, J = 6.8 Hz, 2H), 4.77 (s, 2H), 6.88 (s, 1H); 7.33 (d, J = 8.4 Hz, 1H), 7.55 (d, J = 8.4 Hz, 1H); 7.65-7.67 (m, 2H); 7.85 (d, J = 9.6 Hz, 1H), 7.93 (s, 1H) |
| 76 | 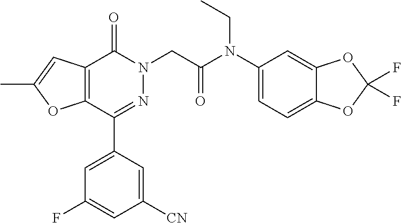 [SMILES: CCN(C(=O)Cn1nc(-c2cc(F)cc(C#N)c2)c2oc(C)cc2c1=O)c1ccc2c(c1)OC(F)(F)O2] | 511.0 [M + 1] | (400 MHz, DMSO) δ: 1.04 (t, J = 7.2 Hz, 3H); 2.59 (s, 3H); 3.69 (q, J = 7.2 Hz, 2H), 4.79 (s, 2H), 6.89 (s, 1H); 7.33 (d, J = 8.0 Hz, 1H), 7.55 (d, J = 8.4 Hz, 1H); 7.67 (s, 1H); 8.07 (d, J = 8.0 Hz, 1H); 8.19 (d, J = 9.6 Hz, 1H); 8.30 (s, 1H) |
| 77 | 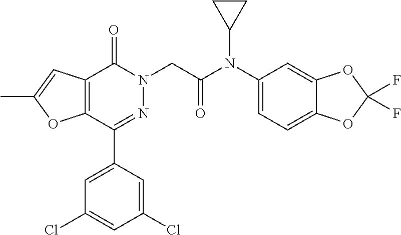 [SMILES: Cc1cc2c(=O)n(CC(=O)N(c3ccc4c(c3)OC(F)(F)O4)C3CC3)nc(-c3cc(Cl)cc(Cl)c3)c2o1] | 548.2 [M+1] | (400 MHz, DMSO) δ: 0.66 (s, 2H), 0.90 (d, J = 6.0 Hz, 2H), 2.68 (s, 3H), 3.30 (m, 1H), 5.17 (s, 2H), 6.86 (s, 1H), 7.15 (d, J = 8.4 Hz, 1H), 7.42 (m, 2H), 7.75 (s, 1H), 8.050-8.05 (d, J = 2.4 Hz, 2H) |
| 78 | 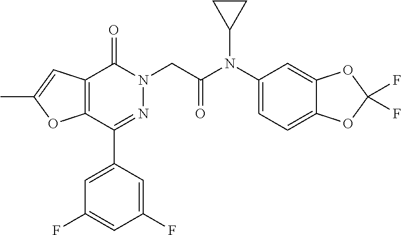 [SMILES: Cc1cc2c(=O)n(CC(=O)N(c3ccc4c(c3)OC(F)(F)O4)C3CC3)nc(-c3cc(F)cc(F)c3)c2o1] | 516.1 [M + 1] | (400 MHz, DMSO) δ: 0.66 (s, 2H), 0.90 (d, J = 6.0 Hz, 2H), 2.59 (s, 3H), 3.29 (m, 1H), 5.16 (s, 2H), 6.84 (s, 1H), 7.13 (dd, J = 8.8, 2.0 Hz, 1H), 7.33-7.41 (m, 3H), 7.71-7.76 (m, 2H) |
| 79 | 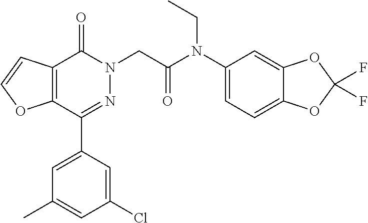 [SMILES: CCN(C(=O)Cn1nc(-c2cc(C)cc(Cl)c2)c2occc2c1=O)c1ccc2c(c1)OC(F)(F)O2] | 502.2 [M + 1] | (400 MHz, DMSO) δ: 1.04 (t, J = 6.8 Hz, 3H); 2.46 (s, 3H); 3.69 (q, J = 6.8 Hz, 2H), 4.79 (s, 2H); 7.25 (s, 1H); 7.35 (d, J = 8.0 Hz, 1H); 7.48 (s, 1H); 7.56 (d, J = 8.4 Hz, 1H); 7.69 (s, 1H); 7.84 (s, 1H), 7.87 (s, 1H), 8.39 (d, J = 1.6 Hz, 1H) |
| 80 | 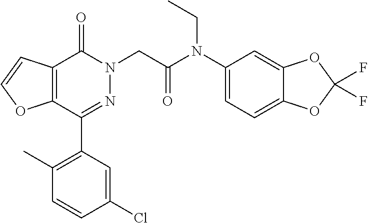 [SMILES: CCN(C(=O)Cn1nc(-c2cc(Cl)ccc2C)c2occc2c1=O)c1ccc2c(c1)OC(F)(F)O2] | 502.0 [M + 1] | (400 MHz, DMSO) δ: 1.04 (t, J = 7.2 Hz, 3H), 2.25 (s, 3H), 3.68 (q, J = 7.2 Hz, 2H), 4.74 (s, 2H), 7.23 (s, 1H), 7.34 (d, J = 7.6 Hz, 1H), 7.45 (d, J = 8.0 Hz, 1H), 7.51-7.58 (m, 3H), 7.68 (s, 1H), 8.27 (d, J = 2.0 Hz, 1H) |
| 81 | 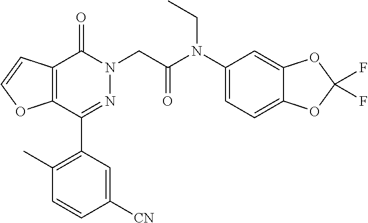 [SMILES: CCN(C(=O)Cn1nc(-c2cc(C#N)ccc2C)c2occc2c1=O)c1ccc2c(c1)OC(F)(F)O2] | 493.2 [M + 1] | (400 MHz, DMSO) δ: 1.04 (t, J = 7.2 Hz, 3H), 2.37 (s, 3H), 3.68 (q, J = 7.2 Hz, 2H), 4.75 (s, 2H), 7.24 (s, 1H), 7.34 (d, J = 7.6 Hz, 1H), 7.55 (d, J = 8.4 Hz, 1H), 7.64-7.68 (m, 2H), 7.94 (d, J = 8.0 Hz, 1H), 8.02 (s, 1H), 8.28 (d, J = 1.6 Hz, 1H) |
| 82 | 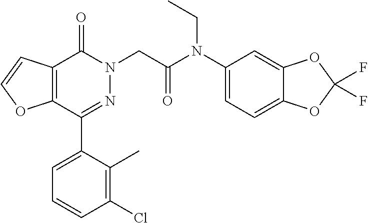 [SMILES: CCN(C(=O)Cn1nc(-c2cccc(Cl)c2C)c2occc2c1=O)c1ccc2c(c1)OC(F)(F)O2] | 502.2 [M + 1] | (400 MHz, DMSO) δ: 1.04 (t, J = 7.2 Hz, 3H), 2.25 (s, 3H), 3.68 (q, J = 7.2 Hz, 2H), 4.74 (s, 2H), 7.24 (s, 1H), 7.34 (d, J = 8.4 Hz, 1H), 7.39-7.47 (m, 2H), 7.55 (d, J = 8.4 Hz, 1H), 7.64- 7.68 (m, 2H), 8.25 (d, J = 2.0 Hz, 1H) |
| 83 | 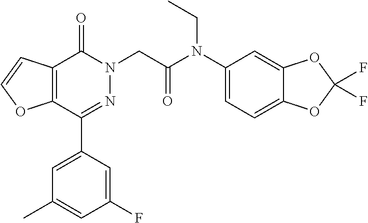 [SMILES: CCN(C(=O)Cn1nc(-c2cc(C)cc(F)c2)c2occc2c1=O)c1ccc2c(c1)OC(F)(F)O2] | 486.2 [M + 1] | (400 MHz, DMSO) δ: 1.05 (t, J = 6.8 Hz, 3H); 2.46 (s, 3H); 3.69 (q, J = 6.8 Hz, 2H); 4.78 (s, 2H); 7.24-7.27 (m, 2H); 7.34 (d, J = 7.6 Hz, 1H); 7.55 (d, J = 8.4 Hz, 1H); 7.63 (d, J = 10.0 Hz, 1H), 7.68 (s, 1H); 7.74 (s, 1H); 8.37 (d, J = 2.0 Hz, 1H) |
| 84 | 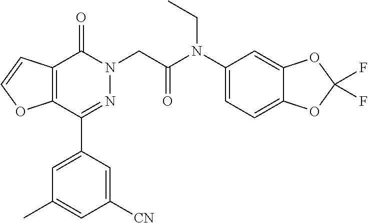 [SMILES: CCN(C(=O)Cn1nc(-c2cc(C)cc(C#N)c2)c2occc2c1=O)c1ccc2c(c1)OC(F)(F)O2] | 493.2 [M + 1] | (400 MHz, DMSO) δ: 1.04 (t, J = 6.8 Hz, 3H); 2.49 (s, 3H); 3.69 (d, J = 6.8 Hz, 2H); 4.80 (s, 2H); 7.26 (s, 1H); 7.36 (m, 1H); 7.56 (d, J = 8.4 Hz, 1H); 7.68 (s, 1H); 7.87 (s, 1H); 8.17 (s, 1H), 8.24 (s, H); 8.39 (d, J = 1.6 Hz, 1H) |
| 85 | 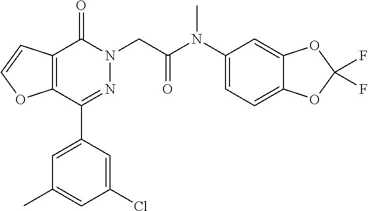 [SMILES: Cc1cc(Cl)cc(-c2nn(CC(=O)N(C)c3ccc4c(c3)OC(F)(F)O4)c(=O)c3ccoc23)c1] | 488 [M + 1] | (400 MHz, DMSO) 2.44 (s, 3H); 3.21 (s, 3H); 4.84 (s, 2H); 7.25 (s, 1H); 7.40 (d, J = 8.0 Hz, 1H); 7.47 (s, 1H); 7.55 (d, J = 8.4 Hz, 1H); 7.74 (s, 1H); 7.86 (m, 2H); 8.38 (d, J = 1.6 Hz, 1H) |
| 86 | 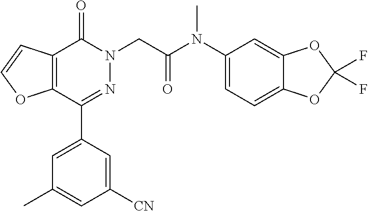 [SMILES: Cc1cc(C#N)cc(-c2nn(CC(=O)N(C)c3ccc4c(c3)OC(F)(F)O4)c(=O)c3ccoc23)c1] | 479.3 [M + 1] | (400 MHz, DMSO) δ: 2.49 (s, 3H); 3.21 (s, 3H); 4.86 (s, 2H); 7.27 (s, 1H); 7.42 (d, J = 8.4 Hz, 1H); 7.56 (d, J = 8.8 Hz, 1H); 7.75 (s, 1H); 7.88 (s, 1H); 8.19 (s, 1H); 8.25 (s, 1H); 8.40 (s, 1H) |
| 87 | 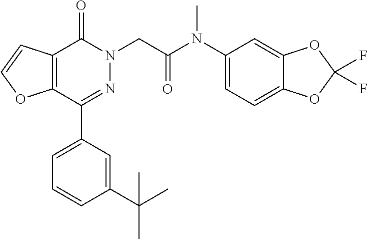 [SMILES: CN(C(=O)Cn1nc(-c2cccc(C(C)(C)C)c2)c2occc2c1=O)c1ccc2c(c1)OC(F)(F)O2] | 496.2 [M + 1] | (400 MHz, DMSO) δ: 1.37 (s, 9H), 3.21 (s, 3H), 4.83 (s, 2H), 7.23 (s, 1H), 7.39 (d, J = 7.6 Hz, 1H), 7.49- 7.55 (m, 2H), 7.60 (d, J = 7.6 Hz, 1H), 7.72 (s, 1H), 7.83 (d, J = i7.6 Hz, 1H), 8.04 (s, 1H), 8.37 (d, J = 1.6 Hz, 1H) |
| 88 | 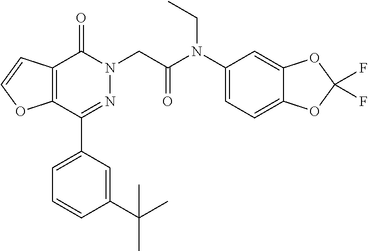 [SMILES: CCN(C(=O)Cn1nc(-c2cccc(C(C)(C)C)c2)c2occc2c1=O)c1ccc2c(c1)OC(F)(F)O2] | 510.2 [M + 1] | (400 MHz, DMSO) δ: 1.05 (t, J = 6.8 Hz, 3H), 1.37 (s, 9H), 3.69 (q, J = 6.8 Hz, 2H), 4.78 (s, 2H), 7.23 (s, 1H), 7.34 (d, J = 8.4 Hz, 1H), 7.50-7.56 (m, 2H), 7.60 (d, J = 8.0 Hz, 1H), 7.67 (s, 1H), 7.83 (d, J = 7.6 Hz, 1H)), 8.04 (s, 1H) 8.37 (d, J = 2.0 Hz, 1H) |
| 89 | 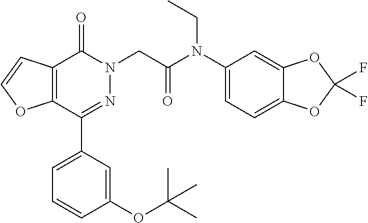 [SMILES: CCN(C(=O)Cn1nc(-c2cccc(OC(C)(C)C)c2)c2occc2c1=O)c1ccc2c(c1)OC(F)(F)O2] | 526.2 [M + 1] | (400 MHz, DMSO) δ: 1.05 (t, J = 6.8 Hz, 3H), 1.38 (s, 9H), 3.69 (q, J = 6.8 Hz, 2H), 4.78 (s, 2H), 7.18 (dd, J = 8.0, 1.6 Hz, 1H), 7.23 (s, 1H), 7.33 (d, J = 8.0 Hz, 1H), 7.48-7.56 (m, 3H), 7.63 (s, 1H), 7.66 (s, 1H), 7.75 (d, J = 8.0 Hz, 1H), 8.37 (d, J = 1.6 Hz, 1H) |
| 90 | 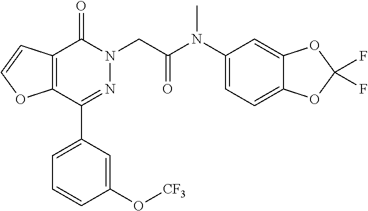 [SMILES: CN(C(=O)Cn1nc(-c2cccc(OC(F)(F)F)c2)c2occc2c1=O)c1ccc2c(c1)OC(F)(F)O2] | 524.2 [M + 1] | (400 MHz, DMSO) δ: 3.20 (s, 3H), 4.85 (s, 2H), 7.26 (s, 1H), 7.39 (d, J = 8.0 Hz, 1H), 7.53-7.59 (m, 2H), 7.73-7.77 (m, 2H), 7.97 (s, 1H), 8.12 (d, J = 7.6 Hz, 1H), 8.40 (d, J = 1.6 Hz, 1H) |
| 91 | 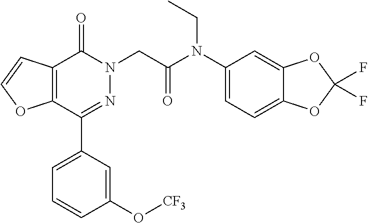 [SMILES: CCN(C(=O)Cn1nc(-c2cccc(OC(F)(F)F)c2)c2occc2c1=O)c1ccc2c(c1)OC(F)(F)O2] | 538.2 [M + 1] | (400 MHz, DMSO) δ: 1.04 (t, J = 6.8 Hz, 3H), 3.69 (q, J = 6.8 Hz, 2H), 4.80 (s, 2H), 7.26 (s, 1H), 7.34 (d, J = 8.4 Hz, 1H), 7.55 (d, J = 8.8 Hz, 1H), 7.59 (d, J = 9.2 Hz, 1H), 7.68 (s, 1H), 7.76 (t, J = 8.0 Hz, 1H), 7.97 (s, 1H), 8.12 (d, J = 8.0 Hz, 1H), 8.40 (d, J = 2.0 Hz, 1H) |
| 92 | 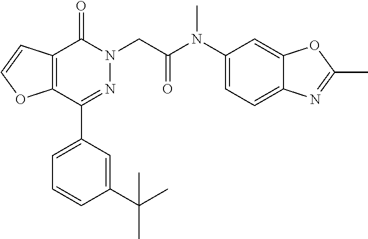 [SMILES: Cc1nc2ccc(N(C)C(=O)Cn3nc(-c4cccc(C(C)(C)C)c4)c4occc4c3=O)cc2o1] | 471.2 [M + 1] | (400 MHz, DMSO) 1.36 (s, 9H), 2.64 (s, 3H), 3.26 (s, 3H), 4.80 (s, 2H), 7.22 (s, 1H), 7.52-7.48 (m, 2H), 7.58 (d, J = 8.0 Hz, 1H), 7.82-7.77 (m, 2H), 7.94 (s, 1H), 8.03 (s, 1H), 8.35 (d, J = 2.0 Hz, 1H) |
| 93 | 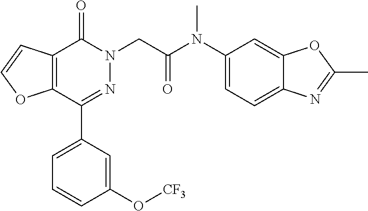 [SMILES: Cc1nc2ccc(N(C)C(=O)Cn3nc(-c4cccc(OC(F)(F)F)c4)c4occc4c3=O)cc2o1] | 499.0 [M + 1] | (400 MHz, DMSO) 2.64 (s, 3H), 3.25 (s, 3H), 4.82 (s, 2H), 7.25 (s, 1H), 7.46 (d, J = 8.0 Hz, 1H), 7.58 (d, J = 8.0 Hz, 1H), 7.76-7.72 (m, 2H), 7.96 (s, 2H), 8.10 (d, J = 7.6 Hz, 1H), 8.39 (s, 1H) |
| 94 | 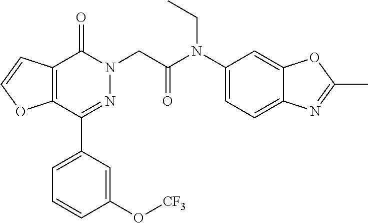 [SMILES: CCN(C(=O)Cn1nc(-c2cccc(OC(F)(F)F)c2)c2occc2c1=O)c1ccc2nc(C)oc2c1] | 513 [M + 1] | (400 MHz, DMSO): 1.06 (t, J = 7.2 Hz, 3H); 2.64 (s, 3H); 3.74 (q, J = 7.2 Hz, 2H); 4.77 (s, 2H); 7.25 (s, 1H); 7.41 (d, J = 7.6 Hz, 1H); 7.58 (d, J = 8.0 H, 1H); 7.73-7.78 (m, 2H); 7.91-7.96 (m, 2H), 8.10 (d, J = 8.0 Hz, 1H); 8.39 (d, J = 1.6 Hz, 1H) |
| 95 | 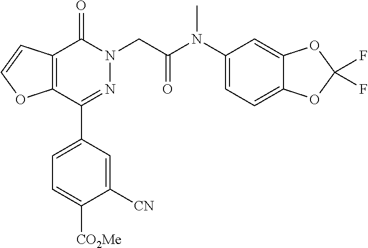 [SMILES: COC(=O)c1ccc(-c2nn(CC(=O)N(C)c3ccc4c(c3)OC(F)(F)O4)c(=O)c3ccoc23)cc1C#N] | 523.2 [M + 1] | (400 MHz, DMSO) 3.20 (s, 3H), 3.97 (s, 3H), 4.85 (s, 2H), 7.29 (s, 1H), 7.41 (d, J = 8.0 Hz, 1H), 7.55 (d, J = 8.4 Hz, 1H), 7.74 (s, 1H), 8.34 (d, J = 8.4 Hz, 1H), 8.43 (d, J = 2.0 Hz, 1H), 8.5.0 (d, J = 8.4 H, 1H), 8.60 (s, 1H) |
| 96 | 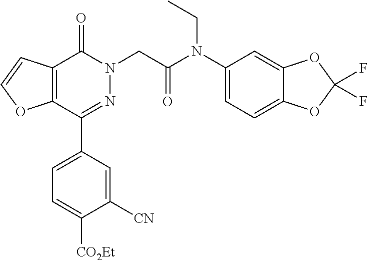 [SMILES: CCOC(=O)c1ccc(-c2nn(CC(=O)N(CC)c3ccc4c(c3)OC(F)(F)O4)c(=O)c3ccoc23)cc1C#N] | 551.1 [M + 1] | (400 MHz, DMSO-d₆) δ : 1.04 (t, J = 6.8 Hz , 3H), 1.40 (t, J = 7.2, 3H), 3.69 (q, J = 6.8 Hz, 2H), 4.44 (q, J = 7.2 Hz, 2H), 4.84 (s, 2H), 7.29 (s, 1H), 7.36 (d, J = 8.4 Hz, 1H), 7.57 (d, J = 8.8 Hz, 1H), 7.70 (s, 1H), 8.34 (d, J = 8.4 Hz, 1H), 8.44 (s, 1H), 8.51 (d, J = 8.4 Hz, 1H), 8.59 (s, 1H) |
| 97 | 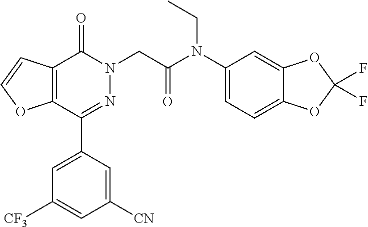 [SMILES: CCN(C(=O)Cn1nc(-c2cc(C#N)cc(C(F)(F)F)c2)c2occc2c1=O)c1ccc2c(c1)OC(F)(F)O2] | 547.2 [M + 1] | (400 MHz, DMSO) δ: 1.05 (t, J = 6.8 Hz, 3H), 3.69 (q, J = 6.8 Hz, 2H), 4.84 (s, 2H), 7.30 (s, 1H), 7.36 (d, J = 9.6 Hz, 1H), 7.57 (d, J = 8.4 Hz, 1H), 7.70 (s, 1H), 8.45 (s, 1H), 8.59 (s, 1H), 8.63 (s, 1H), 8.77 (s, 1H) |
| 98 | 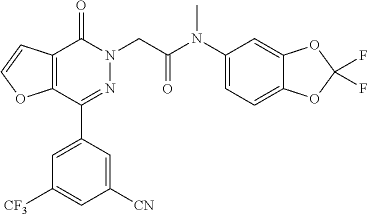 [SMILES: CN(C(=O)Cn1nc(-c2cc(C#N)cc(C(F)(F)F)c2)c2occc2c1=O)c1ccc2c(c1)OC(F)(F)O2] | 533 [M + 1] | (400 MHz, DMSO) δ: 3.21 (s, 3 H), 4.90 (s, 2H), 7.30 (s, 1H), 7.42 (s, 1H), 7.56 (d, J = 9.2 Hz, 1H), 7.75 (s, 1H), 8.45 (s, 1H), 8.59 (s, 1H), 8.64 (s, 1H), 8.77 (s, 1H) |
| 99 | 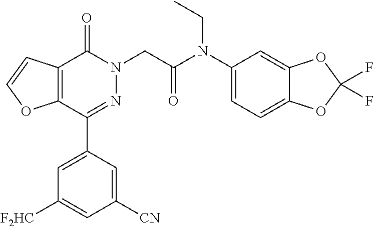 [SMILES: CCN(C(=O)Cn1nc(-c2cc(C#N)cc(C(F)F)c2)c2occc2c1=O)c1ccc2c(c1)OC(F)(F)O2] | 529.5 [M + 1] | (400 MHz, DMSO) δ: 1.03 (t, J = 6.8 Hz, 3H), 3.68 (q, J = 6.8 Hz, 2H), 4.81 (s, 2H), 7.25 (t, J = 55.2 Hz, 1H), 7.27 (m, 1H), 7.35 (d, J = 8.0 Hz, 1H), 7.56 (d, J = 8.4 Hz, 1H), 7.69 (s, 1H), 8.28 (s, 1H), 8.43 (s, 1H), 8.52 (s, 1H), 8.63 (s, 1H) |
| 100 | 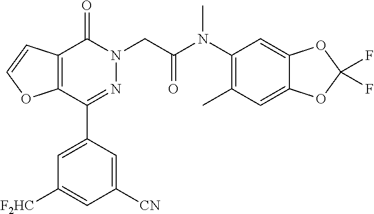 | 515.0 [M + 1] | (400 MHz, DMSO) δ: 3.21(s, 3H), 4.89 (s, 2H), 7.27 (t, J = 55.2 Hz, 1H), 7.30 (s, 1H), 7.43 (m, 1H), 7.57 (d, J = 8.4 Hz, 1H), 7.75 (s, 1H), 8.30 (s, 1H), 8.44 (d, J = 2.0 Hz, 1H), 8.54 (s, 1H), 8.65 (s, 1H) |
| 101 | 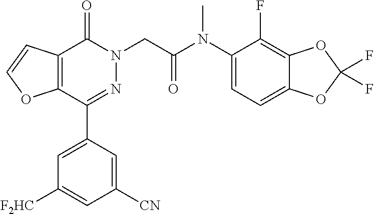 [SMILES: CN(C(=O)Cn1nc(-c2cc(C#N)cc(C(F)F)c2)c2occc2c1=O)c1ccc2c(c1F)OC(F)(F)O2] | 533 [M + 1] | (400 MHz, DMSO) δ: 3.16 (s, 3H), 4.81-4.96 (m, 2H), 7.26 (t, J = 54.8 Hz, 1H), 7.29 (s, 1H), 7.83 (d, J = 8.8 Hz, 1H), 7.94 (s, 1H), 8.29 (s, 1H), 8.44 (s, 1H), 8.52 (s, 1H), 8.66 (s, 1H) |
| 102 | 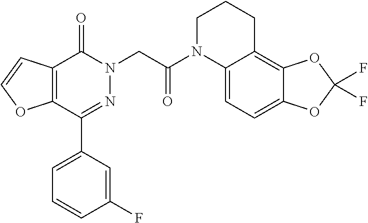 [SMILES: O=C(Cn1nc(-c2cccc(F)c2)c2occc2c1=O)N1CCCc2c1ccc1c2OC(F)(F)O1] | 484.4 [M + H] | (400 MHz, DMSO-d₆): 2.00 (m, 2H), 2.80-2.84 (t, J = 6.8 Hz, 2H), 3.82 (s, 2H), 5.34(s, 2H), 7.23-7.28 (m, 2H), 7.42 (m, 1H), 7.52 (d, J = 8.4 Hz, 1H), 7.67 (dd, J = 14.0, 8.0 Hz, 1H), 7.86 (d, J = 10.4 Hz, 1H), 7.95 (d, J = 8.0 Hz, 1H), 8.39 (d, J = 2.0 Hz, 1H) |
| 103 | 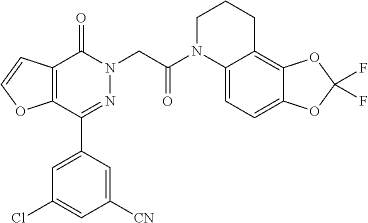 [SMILES: N#Cc1cc(Cl)cc(-c2nn(CC(=O)N3CCCc4c3ccc3c4OC(F)(F)O3)c(=O)c3ccoc23)c1] | 525.0 [M + 1] | (400 MHz, DMSO) δ: 2.01 (s, 2H), 2.84 (t, J = 6.8 Hz, 2H), 3.83 (m, 2H), 5.38 (s, 2H), 7.26 (m, 1H), 7.32 (s, 1H), 7.52 (m, 1H), 8.28 (s, 1H), 8.41-8.47 (m, 3H) |
| 104 | 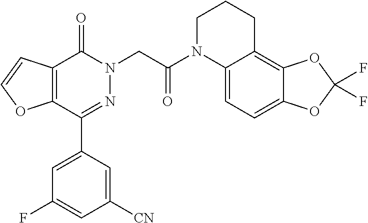 [SMILES: N#Cc1cc(F)cc(-c2nn(CC(=O)N3CCCc4c3ccc3c4OC(F)(F)O3)c(=O)c3ccoc23)c1] | 509.2 [M + 1] | (400 MHz, DMSO) δ: 2.10 (m, 2H), 2.85 (t, J = 6.8 Hz, 2H), 3.83 (m, 2H), 5.37 (s, 2H), 7.25 (d, J = 8.0 Hz, 1H), 7.32 (s, 1H), 7.51 (m, 1H), 8.10 (d, J = 8.0 Hz), 8.23 (d, J = 9.6 Hz, 1H), 8.36 (s, 1H), 8.42 (d, J = 1.6 Hz, 1H) |
| 105 | 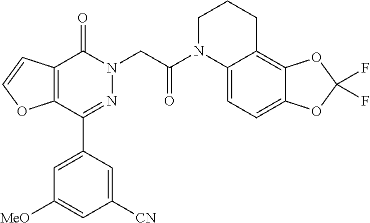 [SMILES: COc1cc(C#N)cc(-c2nn(CC(=O)N3CCCc4c3ccc3c4OC(F)(F)O3)c(=O)c3ccoc23)c1] | 521.2 [M + 1] | (400 MHz, DMSO) δ: 2.01 (m, 2H), 2.84 (t, J = 6.8 Hz, 2H), 3.83 (m, 2H), 3.93 (s, 3H), 5.36 (s, 2H), 7.25 (d, J = 7.2 Hz, 1H), 7.30 (s, 1H), 7.53 (m, 1H), 7.68 (s, 1H), 7.93 (s, 1H), 8.05 (s, 1H), 8.41 (d, J = 2.0 Hz, 1H) |
| 106 | 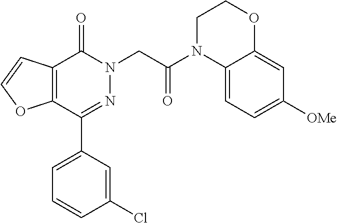 [SMILES: COc1ccc2c(c1)OCCN2C(=O)Cn1nc(-c2cccc(Cl)c2)c2occc2c1=O] | 452.05 [M + 1] | (400 MHz, DMSO-d₆) δ: 3.73 (s, 3H). 3.96 (s, 2H), 4.35 (s, 2H), 5.42 (s, 2H), 6.51 (s, 2H), 7.30 (s, 1H), 7.64 (m, 2H), 8.02-8.16 (m, 2H), 8.40 (d, J = 2.0 Hz, 1H) |
| 107 | 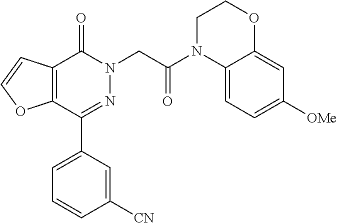 [SMILES: COc1ccc2c(c1)OCCN2C(=O)Cn1nc(-c2cccc(C#N)c2)c2occc2c1=O] | 443.1 [M + 1] | (400 MHz, DMSO-d₆) δ: 3.73 (s, 3H), 3.90-3.98 (m, 2H), 4.29-4.36 (m, 2H), 5.45 (s, 2H), 6.51 (m, 2H), 7.48 (s, 1H), 7.80 (t, J = 8.0 Hz, 1H), 7.92 (s, 1H), 8.03 (d, J = 7.8 Hz, 1H), 8.21 (d, J = 8.0 Hz, 1H), 8.27 (s, 1H), 8.51 (s, 1H) |
| 108 | 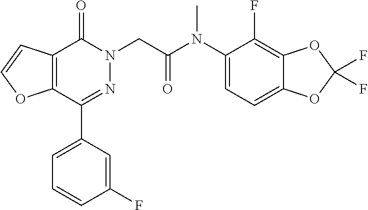 [SMILES: CN(C(=O)Cn1nc(-c2cccc(F)c2)c2occc2c1=O)c1ccc2c(c1F)OC(F)(F)O2] | 476.0 [M + H] | (400 MHz, DMSO): 3.13 (s, 3H), 4.80 (d, J = 16.0 Hz, 1H), 4.91 (d, J = 16.0 Hz, 1H), 7.26 (d, J = 2.0 Hz, 1H), 7.40-7.44 (td, J = 8.4, 2.4 Hz, 1H), 7.62-7.69 (m, 1H), 7.81 (d, J = 8.8 Hz, 2H), 7.92 (m, 2H), 8.39 (d, J = 2.0 Hz, 1H) |
| 109 | 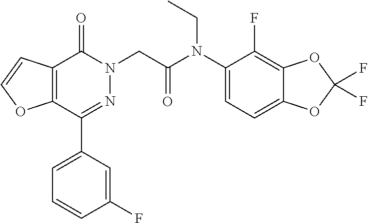 [SMILES: CCN(C(=O)Cn1nc(-c2cccc(F)c2)c2occc2c1=O)c1ccc2c(c1F)OC(F)(F)O2] | 490.3 [M + H] | (400 MHz, DMSO): 1.04 (t, J = 7.2 Hz, 3H), 3.66 (m, 2H), 4.76 (d, J = 16.0 Hz, 1H), 4.86 (d, J = 16.0 Hz, 1H), 7.26 (d, J = 2.0 Hz, 1H), 7.42 (td, J = 8.4, 2.0 Hz, 1H), 7.61-7.68 (m, 1H), 7.80-7.87( m, 3H), 7.92 (d, J = 8.0 Hz, 1H), 8.39 (d, J = 2.0 Hz, 1H) |
| 110 | 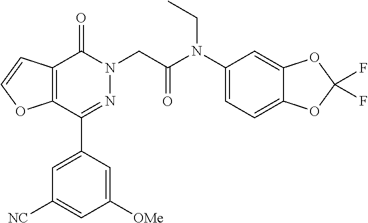 [SMILES: CCN(C(=O)Cn1nc(-c2cc(C#N)cc(OC)c2)c2occc2c1=O)c1ccc2c(c1)OC(F)(F)O2] | 509.5 [M + 1] | (400 MHz, DMSO) δ: 1.04 (t, J = 7.2 Hz, 3H); 3.69 (q, J = 7.2 Hz, 2H); 3.93 (s, 3H); 4.81 (s, 2H); 7.26 (s, 1H); 7.35 (d, J = 8.8 Hz, 1H); 7.55 (d, J = 8.4 Hz, 1H); 7.67-7.69 (m, 2H); 7.90 (s, 1H); 8.02 (s, 1H); 8.39 (d, J = 2.0 Hz, 1H) |
| 111 | 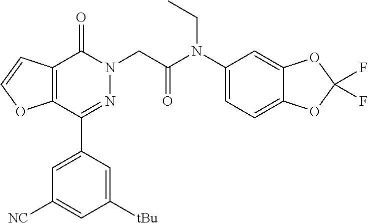 [SMILES: CCN(C(=O)Cn1nc(-c2cc(C#N)cc(C(C)(C)C)c2)c2occc2c1=O)c1ccc2c(c1)OC(F)(F)O2] | 535.2 [M + 1] | (400 MHz, DMSO) δ: 1.03 (t, J = 6.8 Hz, 3H), 1.38 (s, 9H), 3.68 (q, J = 6.8 Hz, 2H), 4.80 (s, 2H), 7.24 (d, J = 1.2 Hz, 1H), 7.33 (d, J = 7.6 Hz, 1H), 7.54 (d, J = 8.4 Hz, 1H), 7.66 (s, 1H), 8.06 (s, 1H), 8.23 (s, 1H), 8.35 (s, 1H), 8.39 (d, J = 2.0 Hz, 1H) |
| 112 | 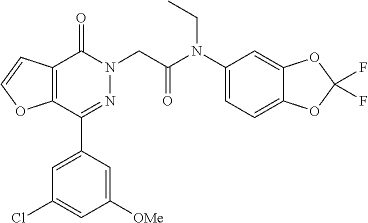 [SMILES: CCN(C(=O)Cn1nc(-c2cc(Cl)cc(OC)c2)c2occc2c1=O)c1ccc2c(c1)OC(F)(F)O2] | 518.0 [M + 1] | (400 MHz, DMSO) δ: 1.04 (t, J = 7.2 Hz, 3H); 3.69 (q, J = 7.2 Hz, 3H); 3.89 (s, 3H); 4.79 (s, 2H); 7.24-7.25 (m, 2H); 7.34 (d, J = 8.4 Hz, 1H); 7.54- 7.58 (m, 2H); 7.66-7.68 (m, 2H); 8.38 (d, J = 2.0 Hz, 1H) |
| 113 | 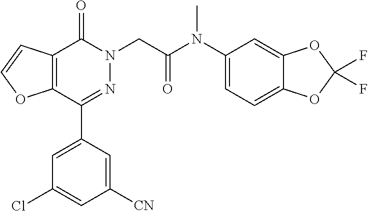 | 598.20 [M + 1] | (400 MHz, DMSO) δ: 1.42 (s, 9H), 3.21 (s, 3H), 4.88 (s, 2H), 7.42 (d, J = 8.4 Hz, 1H), 7.57 (d, J = 8.4 Hz, 1H), 7.76 (s, 1H), 7.90 (s, 1H), 8.23 (s, 1H), 8.28 (s, 1H), 8.44 (s, 1H), 8.51 (s, 1H) |
| 114 | 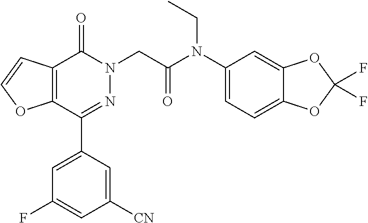 | 596.2 [M + 1] | (400 MHz, DMSO) δ: 1.04 (t, J = 6.8 Hz, 3H), 1.42 (s, 9H), 3.69 (q, J = 6.8 Hz, 2H), 4.82 (s, 2H), 7.35 (d, J = 8.4 Hz, 1H), 7.57 (d, J = 8.4 Hz, 1H), 7.71 (s, 1H), 7.89 (s, 1H), 8.12 (d, J = 7.6 Hz, 1H), 8.24-8.27 (m, 2H), 8.40 (s, 1H) |
| 115 | 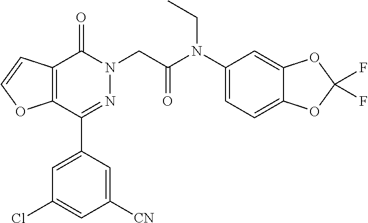 | 612.2 [M + 1] | (400 MHz, DMSO) δ: 1.04 (t, J = 7.2 Hz, 3H), 1.42 (s, 9H), 3.69 (q, J = 7.2 Hz, 2H), 4.83 (s, 2H), 7.36 (d, J = 8.4 Hz, 1H), 7.57 (d, J = 8.4 Hz, 1H), 7.71 (s, 1H), 7.89 (s, 1H), 8.23 (s, 1H), 8.28 (s, 1H), 8.44 (s, 1H), 8.50 (s, 1H) |
| 116 | 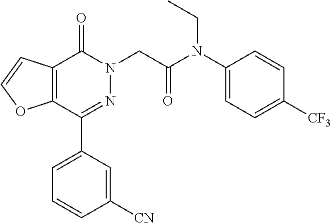 | 566 [M + 1] | (400 MHz, CDCl3) δ: 1.22 (t, J = 7.2 Hz, 3H) , 1.55 (s, 9H), 3.89 (q, J = 7.2 Hz, 2H), 4.82 (s, 2H), 6.22 (s, 1H), 7.61 (d, J = 8.0 Hz, 2H), 7.66 (s, 1H), 7.70 (t, J = 8.0 Hz, 1H), 7.80-7.85 (m, 3H), 8.33 (d, J = 8.0 Hz, 1H), 8.38 (s, 1H) |
| 117 | 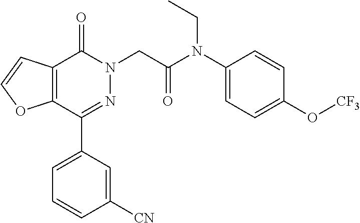 | 582 [M + 1] | (400 MHz, DMSO) δ: 1.05 (t, J = 6.8 Hz, 3H) , 1.43 (s, 9H), 3.72 (q, J = 6.8 Hz, 2H), 4.78 (s, 2H), 7.55 (d, J = 7.2 Hz, 2H), 7.66 (d, J = 8.0 Hz, 2H), 7.80-7.96 (m, 2H), 8.06 (d, J = 7.6 Hz, 1H), 8.21 (s, 1H), 8.42 (m, 1H), 8.51 (s, 1H) |
| 118 | 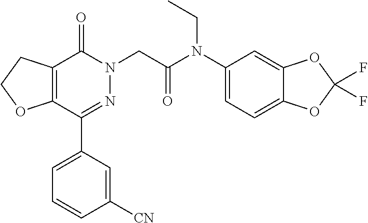 [SMILES: CCN(C(=O)Cn1nc(-c2cccc(C#N)c2)c2c(c1=O)CCO2)c1ccc2c(c1)OC(F)(F)O2] | 481.15 [M + 1] | (400 MHz, DMSO) δ: 1.03 (t, J = 6.8 Hz, 3H), 3.50 (t, J = 5.2 Hz, 2H), 3.68 (q, J = 6.8 Hz, 2H), 4.70 (s, 2H), 4.77 (t, J = 5.2 Hz, 2H), 7.32 (d, J = 8.4 Hz, 1H), 7.56 (d, J = 8.4 Hz, 1H), 7.68 (s, 1H), 7.73 (t, J = 8.0 Hz, 1H), 7.96 (d, J = 8.0 Hz, 1H), 8.02 (d, J = 8.0 Hz, 1H), 8.76 (s, 1H) |
| 119 | 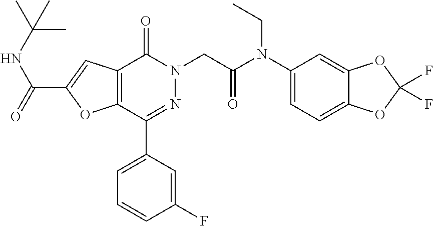 [SMILES: CCN(C(=O)Cn1nc(-c2cccc(F)c2)c2oc(C(=O)NC(C)(C)C)cc2c1=O)c1ccc2c(c1)OC(F)(F)O2] | 571 [M + 1] | (400 MHz, DMSO) δ: 1.04 (t, J = 6.8 Hz, 3H), 1.42 (s, 9H), 3.69 (q, J = 6.8 Hz, 2H), 4.81 (s, 2H), 7.34 (d, J = 6.8 Hz, 1H), 7.43 (d, J = 8.0 Hz, 1H), 7.56 (d, J = 8.4 Hz, 1H), 7.63-7.69 (m, 2H), 7.87-7.99 (m, 3H), 8.20 (s, 1H) |
| 120 | 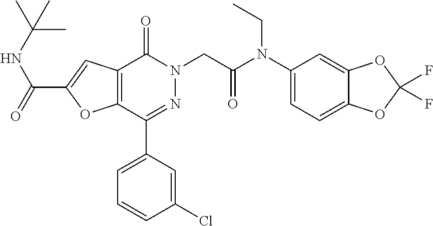 [SMILES: CCN(C(=O)Cn1nc(-c2cccc(Cl)c2)c2oc(C(=O)NC(C)(C)C)cc2c1=O)c1ccc2c(c1)OC(F)(F)O2] | 587.2 [M + 1] | (400 MHz, DMSO) δ: 1.04 (t, J = 6.8 Hz, 3H), 1.42 (s, 9H), 3.69 (q, J = 6.8 Hz, 2H), 4.82 (s, 2H), 7.37 (d, J = 7.6 Hz, 1H), 7.56 (d, J = 8.4 Hz, 1H), 7.62-7.66 (m, 2H), 7.71 (s, 1H), 7.82- 7.85 (m, 2H), 7.90 (s, 1H), 8.27 (s, 1H) |
| 121 | 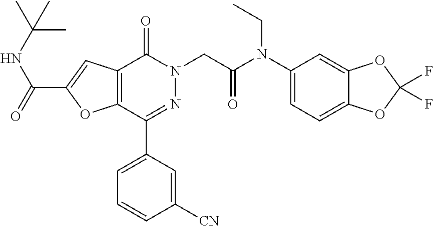 [SMILES: CCN(C(=O)Cn1nc(-c2cccc(C#N)c2)c2oc(C(=O)NC(C)(C)C)cc2c1=O)c1ccc2c(c1)OC(F)(F)O2] | 578.2 [M + 1] | (400 MHz, DMSO) δ: 1.04 (t, J = 7.2 Hz, 3H), 1.42 (s, 9H), 3.70 (q, J = 7.2 Hz, 2H), 4.82 (s, 2H), 7.35 (d, J = 8.0 Hz, 1H), 7.57 (d, J = 8.4 Hz, 1H), 7.70 (s, 1H), 7.85 (t, J = 8.0 Hz, 1H), 7.89 (s, 1H), 8.06 (d, J = 7.6 Hz, 1H), 8.22 (s, 2H), 8.50 (s, 1H) |
| 122 | 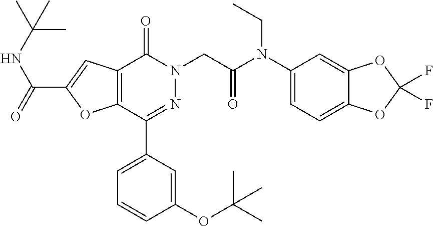 [SMILES: CCN(C(=O)Cn1nc(-c2cccc(OC(C)(C)C)c2)c2oc(C(=O)NC(C)(C)C)cc2c1=O)c1ccc2c(c1)OC(F)(F)O2] | 625 [M + 1] | (400 MHz, DMSO): 1.04 (t, J = 6.8 Hz, 3H); 1.41 (s, 9H); 3.69 (q, J = 6.8 Hz, 2H); 4.80 (s, 2H); 7.34 (d, J = 8.0 Hz, 1H); 7.56 (d, J = 8.4 Hz, 1H); 7.60 (d, J = 8.0 Hz, 1H), 7.68 (s, 1H); 7.77 (t, J = 8.0 Hz, 1H); 7.79 (s, 1H); 8.05 (s, 1H); 8.14-8.17 (m, 2H) |
| 123 | 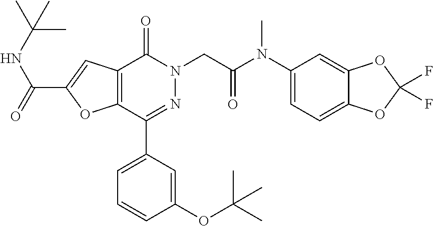 [SMILES: CN(C(=O)Cn1nc(-c2cccc(OC(C)(C)C)c2)c2oc(C(=O)NC(C)(C)C)cc2c1=O)c1ccc2c(c1)OC(F)(F)O2] | 611.0 [M − H] | (400 MHz, DMSO) δ: 1.38 (s, 9H), 1.40 (s, 9H), 3.20 (s, 3H), 4.83 (s, 2H), 7.18 (dd, J = 8.0, 1.6 Hz, 1H), 7.38 (d, J = 8.0 Hz, 1H), 7.48-7.55 (m, 2H), 7.68-7.71 (m, 2H), 7.78 (d, J = 8.0 Hz, 1H), 7.84 (s, 1H), 8.12 (s, 1H) |
| 124 | 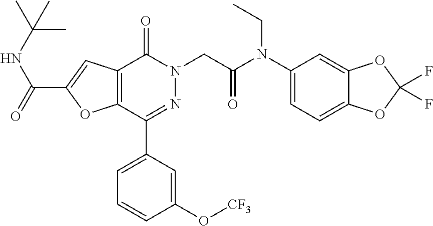 [SMILES: CCN(C(=O)Cn1nc(-c2cccc(OC(F)(F)F)c2)c2oc(C(=O)NC(C)(C)C)cc2c1=O)c1ccc2c(c1)OC(F)(F)O2] | 637 [M + 1] | (400 MHz, DMSO): 1.04 (t, J = 6.8 Hz, 3H); 1.41 (s, 9H); 3.70 (q, J = 6.8 Hz, 2H); 4.80 (s, 2H); 7.34 (d, J = 8.0 Hz, 1H); 7.54-7.61 (m, 2H); 7.68 (s, 1H); 7.77 (t, J = 8.0 Hz, 1H); 7.87 (s, 1H); 8.05 (s, 1H); 8.14-8.17 (m, 2H) |
| 125 | 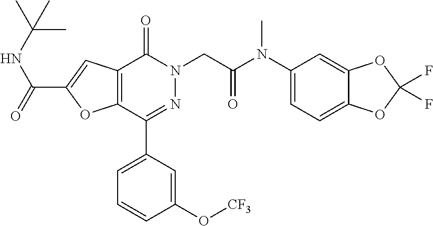 [SMILES: CN(C(=O)Cn1nc(-c2cccc(OC(F)(F)F)c2)c2oc(C(=O)NC(C)(C)C)cc2c1=O)c1ccc2c(c1)OC(F)(F)O2] | 623.0 [M + 1] | (400 MHz, DMSO) δ: 1.41 (s, 9H), 3.21 (s, 3H), 4.86 (s, 2H), 7.40 (d, J = 8.0 Hz, 1H), 7.54-7.61 (m, 2H), 7.74-7.79 (m, 2H), 7.88 (s, 1H), 8.05 (s, 1H), 8.15-8.17 (m, 2H) |
| 126 | 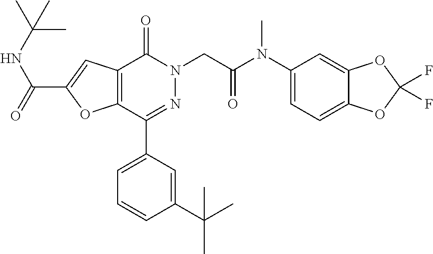 [SMILES: CN(C(=O)Cn1nc(-c2cccc(C(C)(C)C)c2)c2oc(C(=O)NC(C)(C)C)cc2c1=O)c1ccc2c(c1)OC(F)(F)O2] | 595.0 [M + 1] | (400 MHz, DMSO) δ: 1.38 (s, 9H), 1.40 (s, 9H), 3.20 (s, 3H), 4.83 (s, 2H), 7.39 (d, J = 8.0 Hz, 1H), 7.50- 7.56 (m, 2H), 7.60 (d, J = 7.6 Hz, 1H), 7.72 (s, 1H), 7.81-7.86 (m, 2H), 8.07 (s, 1H), 8.14 (s, 1H) |
| 127 | 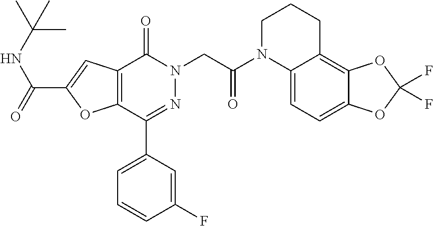 [SMILES: CC(C)(C)NC(=O)c1cc2c(=O)n(CC(=O)N3CCCc4c3ccc3c4OC(F)(F)O3)nc(-c3cccc(F)c3)c2o1] | 583.87 [M + 1] | (400 MHz, DMSO): 1.43 (s, 9H), 2.00 (m, 2H), 2.84 (t, J = 6.8 Hz, 2H), 3.83 (m, 2H), 5.39 (s, 2H), 7.25 (d, J = 8.8 Hz, 1H), 7.45(m, 1H), 7.53 (m, 1H), 7.64-7.70 (m, 2H), 7.74 (d, J = 8.0 Hz, 1H), 7.95 (s, 1H), 8.29 (s, 1H) |
| 128 | 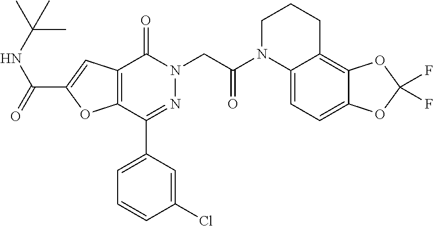 [SMILES: CC(C)(C)NC(=O)c1cc2c(=O)n(CC(=O)N3CCCc4c3ccc3c4OC(F)(F)O3)nc(-c3cccc(Cl)c3)c2o1] | 599.2 [M + H] | (400 MHz, DMSO): 1.42 (s, 9H). 2.00 (m, 2H), 2.84 (t, J = 6.4 Hz, 2H), 3.83 (m, 2H), 5.37 (s, 2H), 7.25 (d, J = 7.2 Hz, 1H), 7.53 (s, 1H), 7.66-7.67 (m, 2H), 7.92 (s, 1H), 8.11-8.19 (m, 3H) |
| 129 | 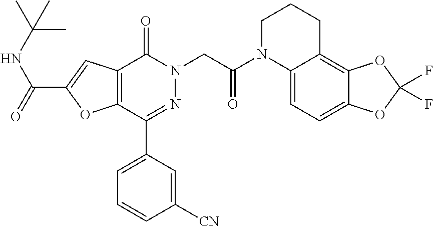 [SMILES: CC(C)(C)NC(=O)c1cc2c(=O)n(CC(=O)N3CCCc4c3ccc3c4OC(F)(F)O3)nc(-c3cccc(C#N)c3)c2o1] | 590.2 [M + H] | (400 MHz, DMSO): 1.43 (s, 9H), 2.01 (s, 2H), 2.84 (t, J = 6.4 Hz, 2H), 3.83 (m, 2H), 5.41 (s, 2H), 7.26 (m, 1H), 7.54 (m, 1H), 7.83 (t, J = 8.0 Hz, 1H), 7.98 (s, 1H), 8.06 (d, J = 7.6 Hz, 1H), 8.21 (d, J = 8.0 Hz, 1H), 8.28-8.31 (m, 2H) |
| 130 | 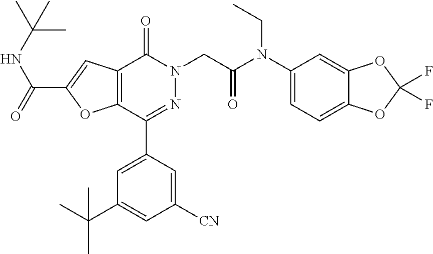 [SMILES: CCN(C(=O)Cn1nc(-c2cc(C#N)cc(C(C)(C)C)c2)c2oc(C(=O)NC(C)(C)C)cc2c1=O)c1ccc2c(c1)OC(F)(F)O2] | 634.0 [M + 1] | (400 MHz, DMSO) δ: 1.05 (t, J = 6.8 Hz, 3H), 1.41 (s, 9H), 1.42 (s, 9H), 3.70 (q, J = 6.8 Hz, 2H), 4.82 (s, 2H), 7.35 (d, J = 8.4 Hz, 1H), 7.57 (d, J = 8.4 Hz, 1H), 7.69 (s, 1H), 7.83 (s, 1H), 8.08 (s, 1H), 8.12 (s, 1H), 8.28 (s, 1H), 8.47 (s, 1H) |
| 131 | 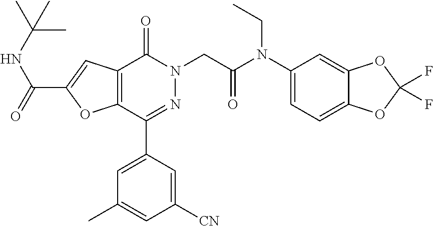 [SMILES: CCN(C(=O)Cn1nc(-c2cc(C)cc(C#N)c2)c2oc(C(=O)NC(C)(C)C)cc2c1=O)c1ccc2c(c1)OC(F)(F)O2] | 592.0 [M + 1] | (400 MHz, DMSO) δ: 1.04 (t, J = 6.8 Hz, 3H); 1.42 (s, 9H), 2.52 (s, 3H), 3.69 (q, J = 6.8 Hz, 2H); 4.83 (s, 2H); 7.36 (d, J = 10.0 Hz, 1H), 7.56 (d, J = 8.4 Hz, 1H); 7.71 (s, 1H); 7.89 (s, 1H), 7.94 (s, 1H); 7.97 (s, 1H), 8.07 (s, 1H), 8.27 (s, 1H) |
| 132 | 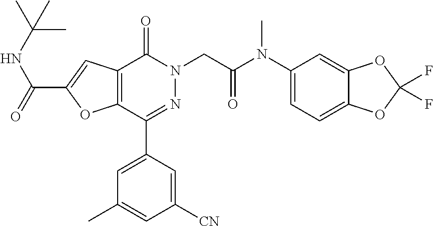 [SMILES: Cc1cc(C#N)cc(-c2nn(CC(=O)N(C)c3ccc4c(c3)OC(F)(F)O4)c(=O)c3cc(C(=O)NC(C)(C)C)oc23)c1] | 578.0 [M + 1] | (400 MHz, DMSO) δ: 1.41 (s, 9H); 2.51 (s, 3H), 3.20 (s, 3H), 4.85 (s, 2H), 7.41 (m, 1H); 7.56 (m, 1H); 7.74 (s, 1H), 7.88 (s, 2H); 8.19-8.23 (m, 2H); 8.31 (s, 1H) |
| 133 | 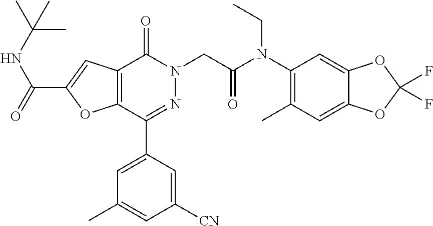 [SMILES: CCN(C(=O)Cn1nc(-c2cc(C)cc(C#N)c2)c2oc(C(=O)NC(C)(C)C)cc2c1=O)c1cc2c(cc1C)OC(F)(F)O2] | 606.2 [M + 1] | (400 MHz, DMSO-d₆) δ: 1.06 (m, 3H), 1.42 (s, 9H), 2.38 (s, 3H), 2.51 (s, 3H), 3.22 (m, 1H), 4.00 (m, 1H), 4.58 (d, J = 15.2 Hz, 1H), 4.75 (d, J = 15.2 Hz, 1H), 7.55 (s, 1H), 7.64 (s, 1H), 7.85 (s, 1H), 7.89 (s, 1H), 8.21- 8.24 (m, 2H), 8.32 (s, 1H) |
| 134 | 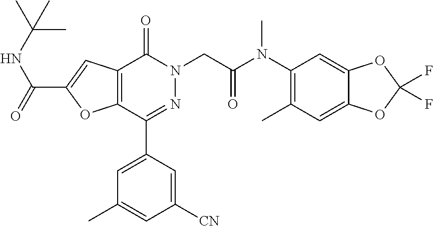 [SMILES: Cc1cc(C#N)cc(-c2nn(CC(=O)N(C)c3cc4c(cc3C)OC(F)(F)O4)c(=O)c3cc(C(=O)NC(C)(C)C)oc23)c1] | 592.2 [M + H] | (400 MHz, DMSO) δ: 1.42 (s, 9H), 2.37 (s, 3H), 2.48 (s, 3H), 3.11 (s, 3H), 4.62 (d, J = 16.0 Hz, 1H), 4.77 (d, J = 16.0 Hz, 1H), 7.53 (s, 1H), 7.69 (s, 1H), 7.85 (s, 1H), 7.88 (s, 1H), 8.20- 8.23 (m, 2H), 8.31 (s, 1H) |
| 135 | 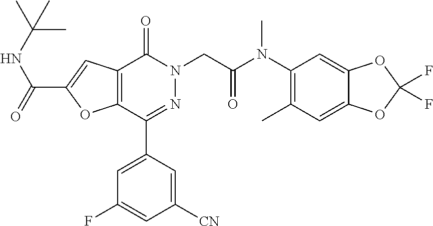 [SMILES: Cc1cc2c(cc1N(C)C(=O)Cn1nc(-c3cc(F)cc(C#N)c3)c3oc(C(=O)NC(C)(C)C)cc3c1=O)OC(F)(F)O2] | 596.2 [M + 1] | (400 MHz, DMSO) 1.43 (s, 9H), 2.37 (s, 3H), 3.12 (s, 3H), 4.64 (d, J = 16.0 Hz, 1H), 4.79 (d, J = 16.0 Hz, 1H), 7.54 (s, 1H), 7.69 (s, 1H), 7.86 (s, 1H), 8.11 (d, J = 8.4 Hz, 1H), 8.24- 8.27 (m, 2H), 8.39 (s, 1H) |
| 136 | 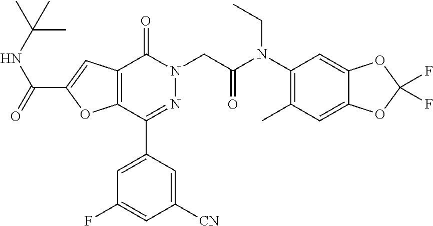 [SMILES: CCN(C(=O)Cn1nc(-c2cc(F)cc(C#N)c2)c2oc(C(=O)NC(C)(C)C)cc2c1=O)c1cc2c(cc1C)OC(F)(F)O2] | 610.2 [M + 1] | (400 MHz, DMSO) 1.07 (t, J = 6.8 Hz, 3H), 1.43 (s, 9H), 2.37 (s, 3H ), 3.23 (q, J = 6.8 Hz, 1H), 3.99 (q, J = 6.8 Hz, 1H), 4.60 (d, J = 16.0 Hz, 1H), 4.76 (d, J = 16.0 Hz, 1H), 7.55 (s, 1H), 7.64 (m, 1H), 7.86 (s, 1H), 8.10 (d, J = 7.6 Hz, 1H), 8.25-8.27 (m, 2H), 8.40 (s, 1H) |
| 137 | 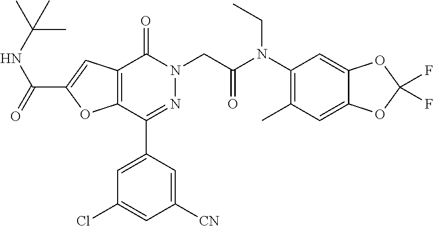 [SMILES: CCN(C(=O)Cn1nc(-c2cc(Cl)cc(C#N)c2)c2oc(C(=O)NC(C)(C)C)cc2c1=O)c1cc2c(cc1C)OC(F)(F)O2] | 626.2 [M + 1] | (400 MHz, DMSO) 1.06 (t, J = 6.8 Hz, 3H), 1.43 (s, 9H), 2.38 (s, 3H), 3.22 (q, J = 6.8 Hz, 1H), 3.99 (q, J = 6.8 Hz, 1H), 4.60 (d, J = 16.0 Hz, 1H), 4.77 (d, J = 16.0 Hz, 1H), 7.56 (s, 1H), 7.64 (s, 1H), 7.87 (s, 1H), 8.25 (s, 1H), 8.28 (t, J = 1.6 Hz, 1H), 8.44 (t, J = 1.6 Hz, 1H), 8.51 (m, 1H) |
| 138 | 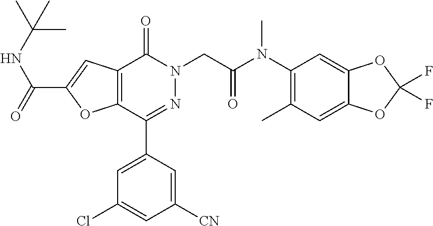 [SMILES: Cc1cc2c(cc1N(C)C(=O)Cn1nc(-c3cc(Cl)cc(C#N)c3)c3oc(C(=O)NC(C)(C)C)cc3c1=O)OC(F)(F)O2] | 612.2 [M + 1] | (400 MHz, DMSO) δ: 1.43 (s, 9H), 2.38 (s, 3H), 3.13 (s, 3H), 4.65 (d, J = 16.0 Hz, 1H), 4.80 (d, J = 16.0 Hz, 1H), 7.37 (d, J = 2.0 Hz, 1H), 7.55 (s, 1H), 7.70 (s, 1H), 7.872(s, 1H), 8.24-8.28 (m, 2H), 8.44 (s, 1H), 8.51 (s, 1H) |
| 139 | 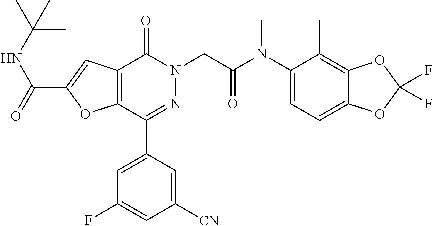 [SMILES: Cc1c(N(C)C(=O)Cn2nc(-c3cc(F)cc(C#N)c3)c3oc(C(=O)NC(C)(C)C)cc3c2=O)ccc2c1OC(F)(F)O2] | 596.2 [M + 1] | (400 MHz, DMSO) 1.43 (s, 9H), 2.35 (s, 3H), 3.14 (s, 3H), 4.68 (d, J = 16.0 Hz, 1H), 4.77 (d, J = 16.0 Hz, 1H), 7.41 (m, 2H), 7.87 (s, 1H), 8.11 (d, J = 7.6 Hz, 1H), 8.25-8.30 (m, 2H), 8.41 (s, 1H) |
| 140 | 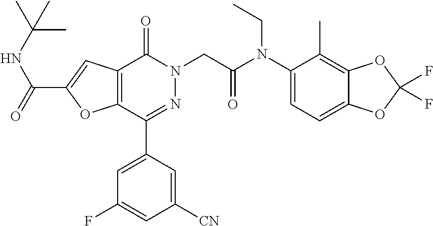 [SMILES: CCN(C(=O)Cn1nc(-c2cc(F)cc(C#N)c2)c2oc(C(=O)NC(C)(C)C)cc2c1=O)c1ccc2c(c1C)OC(F)(F)O2] | 610.6 [M + 1] | (400 MHz, DMSO) 1.06 (t, J = 7.2 Hz, 3H), 1.43 (s, 9H), 2.35 (s, 3H), 3.23 (q, J = 7.2 Hz, 1H), 4.00 (q, J = 7.2 Hz, 1H), 4.64 (d, J = 16.0 Hz, 1H), 4.74 (d, J = 16.0 Hz, 1H), 7.33 (d, J = 8.4 Hz, 1H), 7.42 (d, J = 8.4 Hz, 1H), 7.87 (s, 1H), 8.11 (d, J = 8.4 Hz, 1H), 8.25- 8.28 (m, 2H), 8.41 (s, 1H) |
| 141 | 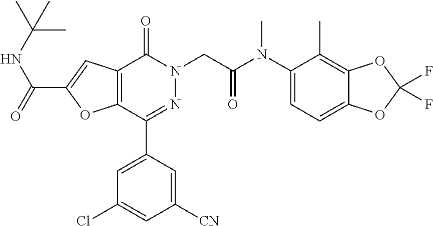 [SMILES: Cc1c(N(C)C(=O)Cn2nc(-c3cc(Cl)cc(C#N)c3)c3oc(C(=O)NC(C)(C)C)cc3c2=O)ccc2c1OC(F)(F)O2] | 612.2 [M + 1] | (400 MHz, DMSO) δ: 1.43 (s, 9H), 2.35 (s, 3H) 3.14 (s, 3H), 4.68 (d, J = 16.0 Hz, 1H), 4.78 (d, J = 16.0 Hz, 1H), 7.42 (s, 2H), 7.88 (s, 1H), 8.25- 8.29 (m, 2H), 8.45 (m, 1H), 8.52 (s, 1H) |
| 142 | 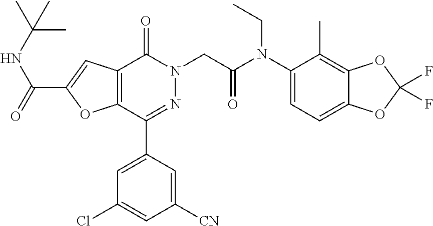 [SMILES: CCN(C(=O)Cn1nc(-c2cc(Cl)cc(C#N)c2)c2oc(C(=O)NC(C)(C)C)cc2c1=O)c1ccc2c(c1C)OC(F)(F)O2] | 626.2 [M + 1] | (400 MHz, DMSO) 1.06 (t, J = 7.2 Hz, 3H), 1.42 (s, 9H), 2.35 (s, 3H), 3.24 (m, 1H), 4.01 (q, J = 7.2 Hz, 1H), 4.63 (d, J = 16.0 Hz, 1H), 4.74 (d, J = 16.0 Hz, 1H), 7.34 (d, J = 8.4 Hz, 1H), 7.41 (d, J = 8.4 Hz, 1H), 7.86 (s, 1H), 8.24 (s, 1H), 8.27 (s, 1H), 8.44 (m, 1H), 8.51 (s, 1H), 8.53 (s, 1H) |
| 143 | 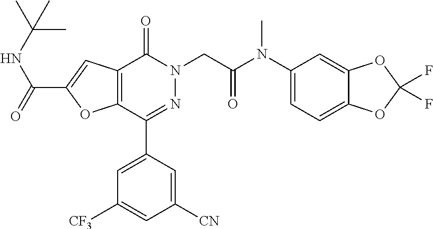 [SMILES: CN(C(=O)Cn1nc(-c2cc(C#N)cc(C(F)(F)F)c2)c2oc(C(=O)NC(C)(C)C)cc2c1=O)c1ccc2c(c1)OC(F)(F)O2] | 632.2 [M + 1] | (400 MHz, DMSO) δ: 1.42 (s, 9H), 3.21 (s, 3H), 4.9 (s, 2H), 7.42 (d, J = 8.0 Hz, 1H), 7.57 (d, J = 8.0 Hz, 1H), 7.76 (s, 1H), 7.88 (s, 1H), 8.21 (s, 1H), 8.5 9(s, 1H), 8.71 (s, 1H), 8.83 (s, 1H) |
| 144 | 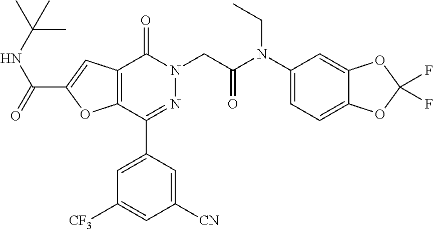 [SMILES: CCN(C(=O)Cn1nc(-c2cc(C#N)cc(C(F)(F)F)c2)c2oc(C(=O)NC(C)(C)C)cc2c1=O)c1ccc2c(c1)OC(F)(F)O2] | 646.6 [M + 1] | (400 MHz, DMSO) δ: 1.06 (t, J = 7.2 Hz, 3H), 1.42 (s, 9H), 3.69 (q, J = 7.2 Hz, 2H), 4.85 (s, 2H), 7.36 (d, J = 8.0 Hz, 1H), 7.57 (d, J = 8.0 Hz, 1H), 7.71 (s, 1H), 7.88 (s, 1H), 8.21 (s, 1H), 8.60 (s, 1H), 8.71 (s, 1H), 8.83 (s, 1H) |
| 145 | 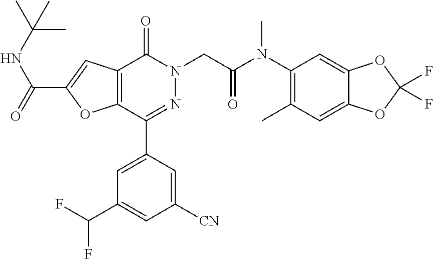 [SMILES: Cc1cc2c(cc1N(C)C(=O)Cn1nc(-c3cc(C#N)cc(C(F)F)c3)c3oc(C(=O)NC(C)(C)C)cc3c1=O)OC(F)(F)O2] | 628.2 [M + 1] | (400 MHz, DMSO) 1.05 (t, J = 6.8 Hz, 3H), 1.43 (s, 9H), 3.69 (q, J = 6.8 Hz, 2H), 4.83 (s, 2H), 7.26 (t, J = 54.8 Hz, 1H), 7.36 (d, J = 8.4 Hz, 1H), 7.57 (d, J = 8.4 Hz, 1H), 7.71 (s, 1H), 7.88 (s, 1H), 8.21 (s, 1H), 8.30 (s, 1H), 8.58 (s, 1H), 8.71 (s, 1H) |
| 146 | 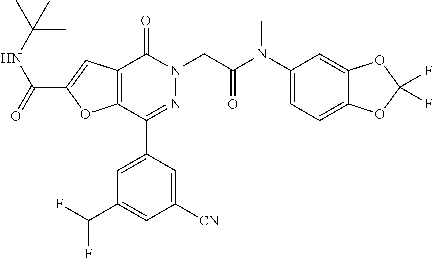 [SMILES: CN(C(=O)Cn1nc(-c2cc(C#N)cc(C(F)F)c2)c2oc(C(=O)NC(C)(C)C)cc2c1=O)c1ccc2c(c1)OC(F)(F)O2] | 614.0 [M + 1] | (400 MHz, DMSO) δ: 1.43 (s, 9H), 3.22 (s, 3H), 4.89 (s, 2H), 7.26 (t, J = 55.2 Hz, 1H), 7.42 (t, J = 8.0 Hz, 1H), 7.58 (d, J = 8.4 Hz, 1H), 7.76 (s, 1H), 7.89 (s, 1H), 8.20 (s, 1H), 8.31 (s, 1H), 8.58 (s, 1H), 8.71 (s, 1H) |
| 147 | 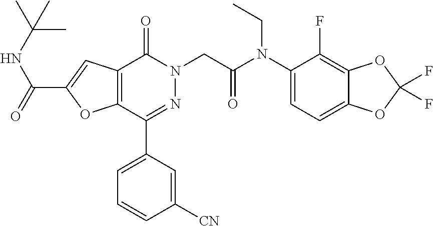 [SMILES: CCN(C(=O)Cn1nc(-c2cccc(C#N)c2)c2oc(C(=O)NC(C)(C)C)cc2c1=O)c1ccc2c(c1F)OC(F)(F)O2] | 596 [M + 1] | (400 MHz, DMSO) δ: 1.04 (t, J = 6.8 Hz, 3H), 1.43 (s, 9H), 3.66 (q, J = 6.8 Hz, 2H), 4.79 (d, J = 16.0 Hz, 1H), 4.87 (d, J = 16.0 Hz, 1H) 7.80-7.96 (m, 3H), 8.06 (d, J = 7.6 Hz, 1H), 8.27 (m, 1H), 8.40 (d, J = 8.0 Hz, 1H), 8.49 (s, 1H) |
| 148 | 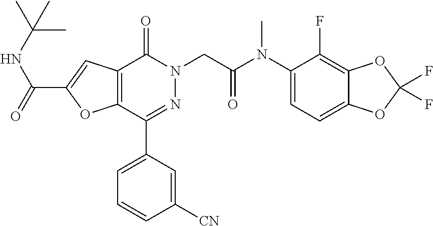 [SMILES: CN(C(=O)Cn1nc(-c2cccc(C#N)c2)c2oc(C(=O)NC(C)(C)C)cc2c1=O)c1ccc2c(c1F)OC(F)(F)O2] | 582 [M + 1] | (400 MHz, DMSO) 1.41 (s, 9H), 3.16 (s, 3H), 4.79-4.97 (m, 2H), 7.61-7.93 (m, 4H), 8.05 (d, J = 7.6 Hz, 1H), 8.14- 8.27 (m, 1H), 8.43 (d, J = 9.6 Hz, 1H), 8.51 (s, 1H) |
| 149 | 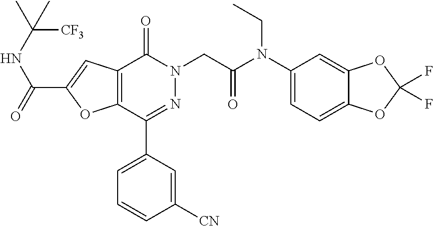 [SMILES: CCN(C(=O)Cn1nc(-c2cccc(C#N)c2)c2oc(C(=O)NC(C)(C)C(F)(F)F)cc2c1=O)c1ccc2c(c1)OC(F)(F)O2] | 630.0 [M − H] | (400 MHz, DMSO) δ: 1.04 (t, J = 6.8 Hz, 3H), 1.67 (s, 6H); 3.69 (q, J = 6.8 Hz, 2H), 4.84 (s, 2H), 131 (d, J = 6.0 Hz, 1H), 7.57 (d, J = 8.8 Hz, 1H), 7.71 (s, 1H); 7.83 (t, J = 8.0 Hz, 1H); 8.06- 8.08 (m, 2H), 8.18 (d, J = 8.0 Hz, 1H); 8.29 (s, 1H), 8.66 (s, 1H) |
| 150 | 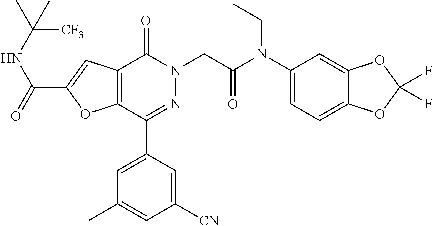 [SMILES: CCN(C(=O)Cn1nc(-c2cc(C)cc(C#N)c2)c2oc(C(=O)NC(C)(C)C(F)(F)F)cc2c1=O)c1ccc2c(c1)OC(F)(F)O2] | 646.0 [M + 1] | (400 MHz, DMSO) δ: 1.05 (t, J = 7.2 Hz, 3H); 1.68 (s, 6H); 3.69 (q, J = 6.8 Hz, 2H), 4.82 (s, 2H), 7.36 (d, J = 8.4 Hz, 1H), 7.57 (d, J = 8.4 Hz, 1H), 7.71 (s, 1H); 7.90 (s, 1H); 8.00 (s, 1H), 8.24 (s, 1H); 8.32 (s, 1H), 8.56 (s, 1H) |
| 151 | 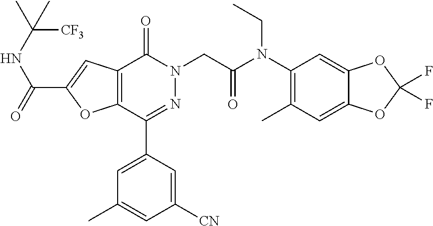 [SMILES: CCN(C(=O)Cn1nc(-c2cc(C)cc(C#N)c2)c2oc(C(=O)NC(C)(C)C(F)(F)F)cc2c1=O)c1cc2c(cc1C)OC(F)(F)O2] | 660.0 [M + 1] | (400 MHz, DMSO) δ: 1.07 (t, J = 6.8 Hz, 3H), 1.67 (s, 6H); 2.38 (s, 3H), 2.49 (s, 3H), 3.22 (q, J = 6.8 Hz, 1H); 3.99 (q, J = 6.8 Hz, 1H); 4.59 (d, J = 16.0 Hz, 1H); 4.76 (d, J = 16.0 Hz, 1H), 7.56 (s, 1H), 7.64 (s, 1H), 7.89 (s, 1H); 7.97 (s, 1H), 8.24 (s, 1H), 8.32 (s, 1H), 8.58 (s, 1H) |
| 152 | 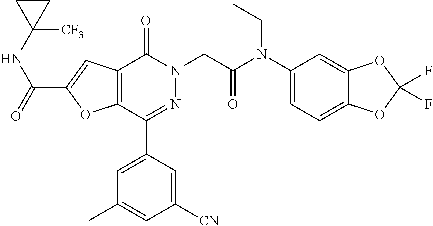 [SMILES: CCN(C(=O)Cn1nc(-c2cc(C)cc(C#N)c2)c2oc(C(=O)NC3(C(F)(F)F)CC3)cc2c1=O)c1ccc2c(c1)OC(F)(F)O2] | 644.0 [M + 1] | (400 MHz, DMSO) δ: 1.04 (t, J = 6.8 Hz, 3H), 1.24 (s, 2H), 1.41 (d, J = 8.0 Hz, 2H), 2.52 (s, 3H), 3.68 (q, J = 6.8 Hz, 2H), 4.81 (s, 2H), 7.35 (d, J = 7.2 Hz, 1H), 7.57 (d, J = 8.4 Hz, 1H), 7.69 (s, 1H), 7.87-7.90 (m, 2H), 8.01 (m, 1H), 8.19 (s, 1H), 8.29 (s, 1H) |
| 153 | 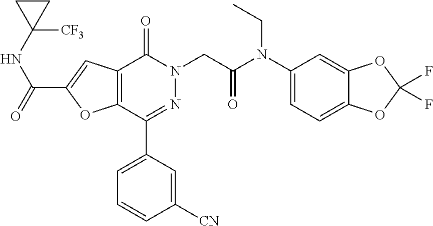 [SMILES: CCN(C(=O)Cn1nc(-c2cccc(C#N)c2)c2oc(C(=O)NC3(C(F)(F)F)CC3)cc2c1=O)c1ccc2c(c1)OC(F)(F)O2] | 630.0 [M + 1] | (400 MHz, DMSO) δ: 1.03 (t, J = 7.2 Hz, 3H), 1.21-1.24 (m, 4H), 3.68 (q, J = 7.2 Hz, 2H), 4.83 (s, 2H), 7.35 (d, J = 8.8 Hz, 1H), 7.55 (d, J = 8.4 Hz, 1H), 7.70 (s, 1H); 7.8l(t, J = 8.0 Hz, 1H); 8.02 (s, 1H), 8.05 (d, J = 8.8 Hz, 1H), 8.16 (d, J = 8.8 Hz, 1H), 8.26 (s, 1H), 9.75(s, 1H) |
| 154 | 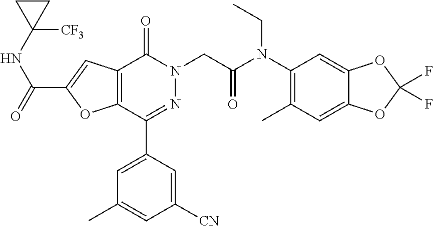 [SMILES: CCN(C(=O)Cn1nc(-c2cc(C)cc(C#N)c2)c2oc(C(=O)NC3(C(F)(F)F)CC3)cc2c1=O)c1cc2c(cc1C)OC(F)(F)O2] | 658.2 [M + 1] | (400 MHz, DMSO) 1.07 (t, J = 7.2 Hz, 3H), 1.23 (m, 2H), 1.40 (m, 2H), 2.37 (s, 3H), 3.21 (q, J = 7.2 Hz, 1H), 4.00 (q, J = 7.2 Hz, 1H), 4.59 (d, J = 16.0 Hz, 1H), 4.80 (d, J = 16.0 Hz, 1H), 7.56 (s, 1H), 7.64 (s, 1H), 7.89 (s, 1H), 7.98 (s, 1H), 8.04 (s, 1H), 8.07 (s, 1H), 9.77 (s, 1H) |
| 155 | 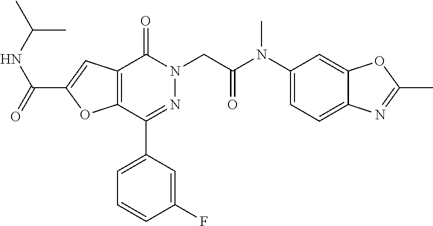 [SMILES: Cc1nc2ccc(N(C)C(=O)Cn3nc(-c4cccc(F)c4)c4oc(C(=O)NC(C)C)cc4c3=O)cc2o1] | 518.53 [M + H] | (400 MHz, DMSO) δ: 1.16-1.30 (m, 6H), 2.63 (s, 3H), 3.25 (s, 3H), 4.07- 4.13 (q, 1H), 4.82 (s, 2H), 7.38-7.45 (m, 2H), 7.63-7.69 (q, 1H), 7.73-7.77 (m, 2H), 7.82-7.85 (d, 1H), 7.94-7.96 (d, 2H), 8.65-8.68 (d, J = 7.6 Hz, 1H) |
| 156 | 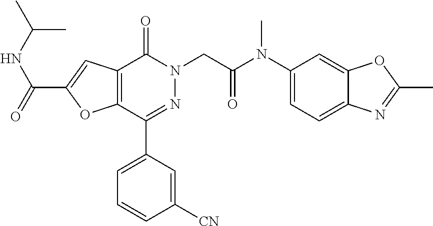 [SMILES: Cc1nc2ccc(N(C)C(=O)Cn3nc(-c4cccc(C#N)c4)c4oc(C(=O)NC(C)C)cc4c3=O)cc2o1] | 525.53 [M + H] | (400 MHz, DMSO) δ: 1.18-1.22 (t, 6H), 2.63 (s, 3H), 3.33 (s, 3H), 4.05- 4.14 (m, 1H), 4.84 (s, 2H), 7.44-7.46 (d, J = 7.6 Hz, 1H), 7.74-7.78 (m, 2H), 7.81-7.85 (t, 1H), 7.95 (s, 1H), 8.02- 8.05 (d, J = 8 Hz, 1H), 8.38-8.40 (d, J = 7.8 Hz, 1H), 8.44 (s, 1H), 8.68- 8.70 (d, J = 7.6 Hz, 1H) |
| 157 | 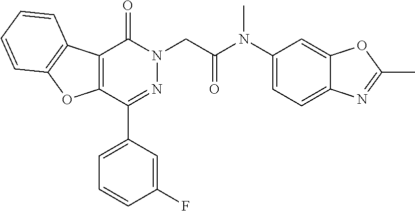 [SMILES: Cc1nc2ccc(N(C)C(=O)Cn3nc(-c4cccc(F)c4)c4oc5ccccc5c4c3=O)cc2o1] | 483.65 [M + H] | (400 MHz, DMSO) δ: 2.62 (s, 3H), 3.27 (s, 3H), 4.91 (s, 2H), 7.41-7.49 (m, 2H), 7.62 (dd, J = 8.0, 6.8 Hz, 1H), 7.65-7.77 (m, 2H), 7.89 (d, J = 8.8 Hz, 1H), 7.97-8.02 (m, 3H), 8.15 (d, J = 8.0 Hz, 1H) |
| 158 | 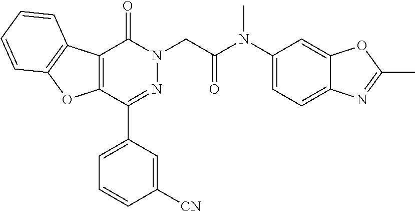 [SMILES: Cc1nc2ccc(N(C)C(=O)Cn3nc(-c4cccc(C#N)c4)c4oc5ccccc5c4c3=O)cc2o1] | 490.2 [M + H] | (400 MHz, DMSO) δ: 2.63 (s, 3H), 3.27 (s, 3H), 4.93 (s, 2H), 7.49 (d, J = 8.8 Hz, 1H), 7.61 (dd, J = 8.8, 7.2 Hz, 1H), 7.70-7.81 (m, 2H), 7.86 (t, J = 8.0 Hz, 1H), 7.98-8.07 (m, 2H), 8.15 (d, J = 8.0 Hz, 1H), 8.45 (d, J = 8.0 Hz, 1H), 8.50 (s, 1H) |
| 159 | 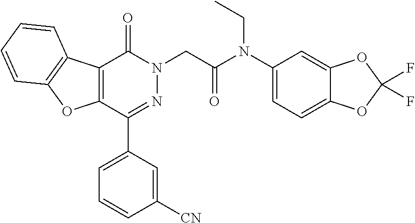 [SMILES: CCN(C(=O)Cn1nc(-c2cccc(C#N)c2)c2oc3ccccc3c2c1=O)c1ccc2c(c1)OC(F)(F)O2] | 529.70 [M + H] | (400 MHz, DMSO) δ: 1.025 (t, J = 7.2 Hz, 3H), 3.70 (q, J = 7.2 Hz, 2H), 4.88 (s, 2H), 7.36 (d, J = 1.6 Hz, 1H), 7.38- 7.62 (m, 2H), 7.70-7.75 (m, 2H), 7.85 (t, J = 8.0 Hz, 1H), 8.01-8.07 (m, 2H), 8.15 (d, J = 7.6 Hz, 1H), 8.46 (d, J = 8.0 Hz, 1H), 8.50 (s, 1H) |
| 160 | 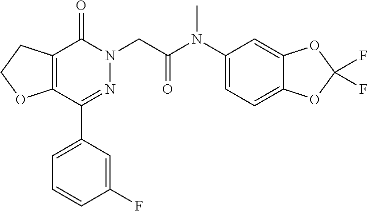 [SMILES: CN(C(=O)Cn1nc(-c2cccc(F)c2)c2c(c1=O)CCO2)c1ccc2c(c1)OC(F)(F)O2] | 460 [M + 1] | (400 MHz, DMSO) δ: 3.06 (t, J = 10.2 Hz, 2H), 3.19 (s, 3H), 4.74 (s, 2H), 4.86 (t, J = 10.2 Hz, 2H), 7.30-7.38 (m, 2H), 7.53-7.58 (m, 2H), 7.64-7.75 (m, 3H) |
| 161 | 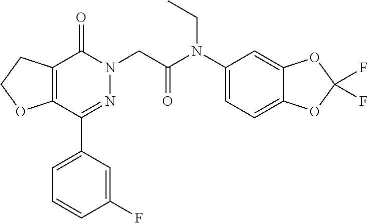 [SMILES: CCN(C(=O)Cn1nc(-c2cccc(F)c2)c2c(c1=O)CCO2)c1ccc2c(c1)OC(F)(F)O2] | 474.2 [M + 1] | (400 MHz, DMSO) δ: 1.04 (t, J = 6.8 Hz, 3H), 3.09 (t J = 10.6 Hz, 2H), 3.67 (q, J = 6.8 Hz, 2H), 4.69 (s, 2H), 4.61- 4.63 (d, J = 8, 2H), 4.86 (t, J = 10.6 Hz, 2H), 7.33 (m, 2H), 7.52-7.59 (m, 2H), 7.66-7.74 (m, 2H), 7.76 (d, J = 8.0 Hz, 1H) |
| 162 | 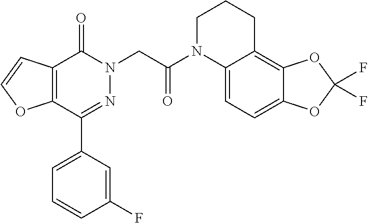 [SMILES: O=C(Cn1nc(-c2cccc(F)c2)c2occc2c1=O)N1CCCc2c1ccc1c2OC(F)(F)O1] | 484.0 [M + 1] | (400 MHz, DMSO) δ: 1.95 (m, 2H), 2.73 (m, 2H), 3.78 (m, 2H), 5.331 (m, 2H), 7.27-7.32 (m, 2H), 7.40-7.44 (td, J = 8.0, 2.4 Hz, 1H), 7.63-7.69 (m, 1H), 7.75 (s, 1H), 7.85 (d, J = 9.6 Hz, 1H), 7.95 (d, J = 8.0 Hz, 1H), 8.39 (d, J = 1.6 Hz, 1H) |
| 163 | 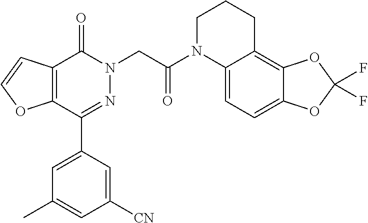 [SMILES: Cc1cc(C#N)cc(-c2nn(CC(=O)N3CCCc4c3ccc3c4OC(F)(F)O3)c(=O)c3ccoc23)c1] | 505.4 [M + 1] | (400 MHz, DMSO) δ: 2.00 (m, 2H), 2.49 (s, 3H) , 2.84 (t, J = 6.8 Hz, 2H), 3.83 (m, 2H), 5.35 (s, 2H), 7.25 (d, J = 8.4 Hz, 1H), 7.30 (s, 1H), 7.51 (s, 1H), 7.88 (s, 1H ), 8.21 (s, 1H ), 8.27 (s, 1H), 8.41 (d, J = 2.0 Hz, 1H) |
| 164 | 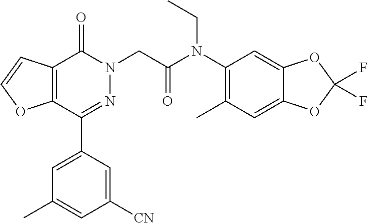 [SMILES: CCN(C(=O)Cn1nc(-c2cc(C)cc(C#N)c2)c2occc2c1=O)c1cc2c(cc1C)OC(F)(F)O2] | 507.2 [M + 1] | (400 MHz, DMSO) δ: 1.05 (t, J = 6.8 Hz, 3H), 2.38 (s, 3H), 3.20 (q, J = 6.8 Hz, 1H), 3.37 (s, 3H), 3.98 (q, J = 6.8 Hz, 1H), 4.56 (d, J = 15.6 Hz, 1H), 4.73 (d, J = 15.6 Hz, 1H), 7.27 (m, 1H), 7.55 (m, 1H), 7.63 (m, 1H), 7.87 (m, 1H), 8.19 (m, 1H), 8.25 (m, 1H), 8.40 (m, 1H) |
| 165 | 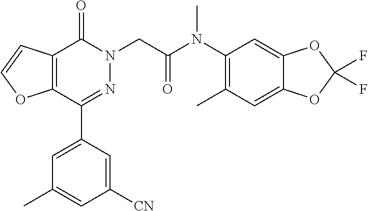 [SMILES: Cc1cc(C#N)cc(-c2nn(CC(=O)N(C)c3cc4c(cc3C)OC(F)(F)O4)c(=O)c3ccoc23)c1] | 493.0 [M + 1] | (400 MHz, DMSO) δ: 2.39 (s, 3H), 2.48 (s, 3H), 3.12 (s, 3H), 4.63 (d, J = 16.0 Hz, 1H), 4.77 (d, J = 16.0 Hz, 1H), 7.28 (d, J = 2.0 Hz, 1H), 7.54 (s, 1H), 7.69 (s, 1H), 7.88 (s, 1H), 8.18 (s, 1H), 8.25 (s, 1H), 8.40 (d, J = 2.0 Hz, 1H) |
| 166 | 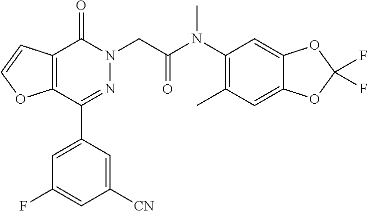 [SMILES: Cc1cc2c(cc1N(C)C(=O)Cn1nc(-c3cc(F)cc(C#N)c3)c3occc3c1=O)OC(F)(F)O2] | 497.4 [M + 1] | (400 MHz, DMSO) δ: 2.34 (s, 3H), 3.12 (s, 3H), 4.61-4.81 (m, 2H), 7.29 (d, J = 2.0 Hz, 1H), 7.54 (s, 1H), 7.68 (s, 1H), 8.09 (d, J = 8.4 Hz, 1H), 8.20 (d, J = 9.2 Hz, 1H), 8.33 (s, 1H), 8.42 (d, J = 2.0 Hz, 1H) |
| 167 | 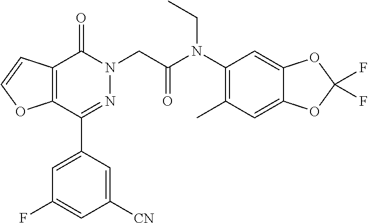 [SMILES: CCN(C(=O)Cn1nc(-c2cc(F)cc(C#N)c2)c2occc2c1=O)c1cc2c(cc1C)OC(F)(F)O2] | 511.1 [M + 1] | (400 MHz, DMSO) δ: 1.07 (t, J = 7.2 Hz, 3H), 2.15 (s, 3H), 3.22 (q, J = 7.2 Hz, 1H), 3.99 (q, J = 7.2 Hz, 1H), 4.60 (d, J = 16.0 Hz, 1H), 4.76 (d, J = 16.0 Hz, 1H), 7.29 (d, J = 1.6 Hz, 1H), 7.56 (s, 1H), 7.63 (s, 1H), 8.10 (d, J = 8.4 Hz, 1H), 8.20 (d, J = 9.2 Hz, 1H), 8.34 (s, 1H), 8.42 (d, J = 2.0 Hz, 1H) |
| 168 | 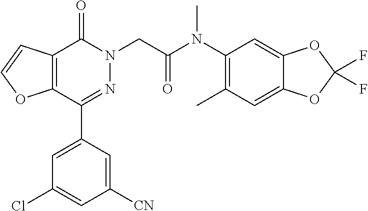 [SMILES: Cc1cc2c(cc1N(C)C(=O)Cn1nc(-c3cc(Cl)cc(C#N)c3)c3occc3c1=O)OC(F)(F)O2] | 513 [M + 1] | (400 MHz, DMSO) δ: 2.38 (s, 3H), 3.12 (s, 3H), 4.64 (d, J = 16.0 Hz, 1H), 4.80 (d, J = 16.0 Hz, 1H) 7.29 (d, J = 2.0 Hz, 1H), 7.54 (s, 1H), 7.69 (s, 1H), 8.26 (s, 1H), 8.39-8.44 (m, 3H) |
| 169 | 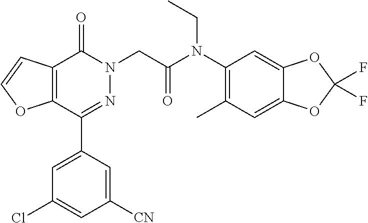 [SMILES: CCN(C(=O)Cn1nc(-c2cc(Cl)cc(C#N)c2)c2occc2c1=O)c1cc2c(cc1C)OC(F)(F)O2] | 527.10 [M + H] | (400 MHz, DMSO) δ: 1.07 (t, J = 7.2 Hz, 3H) 2.39 (s, 3H), 3.22 (q, J = 7.2 Hz, 1H), 4.00 (q, J = 7.2 Hz, 1H), 4.60 (d, J = 16.0 Hz, 1H), 4.76 (d, J = 16.0 Hz, 1H), 7.29 (d, J = 2.0 Hz, 1H), 7.56 (s, 1H), 7.63 (s, 1H), 8.27 (s, 1H), 8.39 (s, 1H), 8.43 (d, J = 2.0 Hz, 1H), 8.44 (s, 1H) |
| 170 | 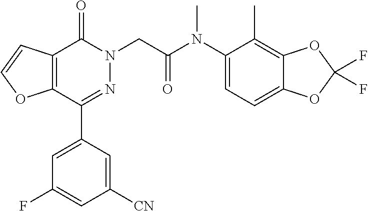 [SMILES: Cc1c(N(C)C(=O)Cn2nc(-c3cc(F)cc(C#N)c3)c3occc3c2=O)ccc2c1OC(F)(F)O2] | 497.0 [M + H] | (400 MHz, DMSO): 2.35 (s, 3H), 3.14 (s, 3H), 4.67 (d, J = 16.0 Hz, 1H), 4.76 (d, J = 16.0 Hz, 1H), 7.29 (d, J = 2.0 Hz, 1H), 7.40 (s, 2H), 8.10 (d, J = 8.4 Hz, 1H), 8.21 (d, J = 9.2 Hz, 1H), 8.34 (s, 1H), 8.42 (d, J = 2.0 Hz, 1H) |
| 171 | 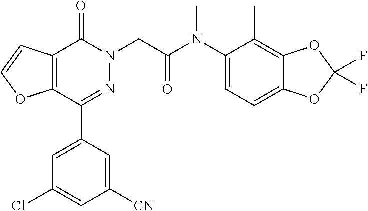 [SMILES: Cc1c(N(C)C(=O)Cn2nc(-c3cc(Cl)cc(C#N)c3)c3occc3c2=O)ccc2c1OC(F)(F)O2] | 513 [M + 1] | (400 MHz, DMSO) δ: 2.35 (s, 3H), 3.14 (s, 3H), 4.67 (d, J = 16.0 Hz, 1H), 4.76 (d, J = 16.0 Hz, 1H), 7.29 (d, J = 2.0 Hz, 1H), 7.41 (s, 2H), 8.27 (s, 1H), 8.39-8.45 (m, 3H) |
| 172 | 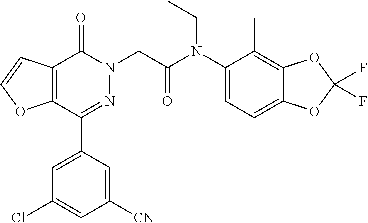 [SMILES: CCN(C(=O)Cn1nc(-c2cc(Cl)cc(C#N)c2)c2occc2c1=O)c1ccc2c(c1C)OC(F)(F)O2] | 527.4 [M + H] | (400 MHz, DMSO): 1.06 (t, J = 7.2 Hz, 3H), 2.35( s, 3H), 3.22 (m, 1H), 4.01 (q, J = 7.2 Hz, 1H), 4.64(d, J = 16.0 Hz, 1H), 4.74 (d, J = 16.0 Hz, 1H), 7.29 (d, J = 1.6 Hz, 1H), 7.35 (d, J = 8.4 Hz, 1H), 7.41 (d, J = 8.4 Hz, 1H), 8.27 (s, 1H), 8.40 (s, 1H), 8.43(d, J = 1.6 Hz, 1H), 8.44 (s, 1H) |
| 173 | 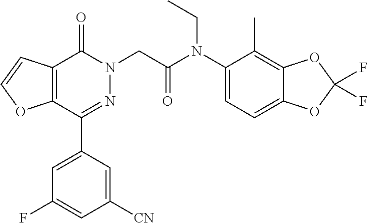 [SMILES: CCN(C(=O)Cn1nc(-c2cc(F)cc(C#N)c2)c2occc2c1=O)c1ccc2c(c1C)OC(F)(F)O2] | 511.2 [M + H] | (400 MHz, DMSO): 1.06 (t, J = 6.8 Hz, 3H), 2.35 (s, 3H), 3.17-3.26 (m, 1H), 4.00 (m, 1H), 4.63 (d, J = 16.0 Hz, 1H), 4.72 (d, J = 16.0 Hz, 1H), 7.29 (d, J = 1.6 Hz, 1H), 7.34 (d, J = 8.4 Hz, 1H), 7.41 (d, J = 8.4 Hz, 1H), 8.09 (d, J = 8.4 Hz, 1H), 8.21 (d, J = 9.6 Hz, 1H), 8.34 (s, 1H), 8.42 (d, J = 2.0 Hz, 1H) |
| 174 | 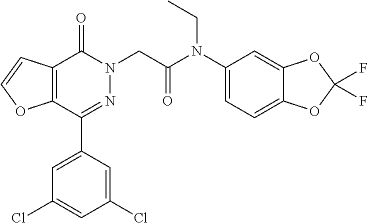 [SMILES: CCN(C(=O)Cn1nc(-c2cc(Cl)cc(Cl)c2)c2occc2c1=O)c1ccc2c(c1)OC(F)(F)O2] | 522 [M + 1] | (400 MHz, DMSO) δ: 1.04 (t, J = 6.8 Hz, 3H), 3.68 (q, J = 6.8 Hz, 2H), 4.81 (s, 2H), 7.26 (s, 1H), 7.35 (d, J = 8.4 Hz, 1H), 7.56 (d, J = 8.4 Hz, 1H), 7.69 (s, 1H), 7.84 (s, 1H), 8.07 (m, 2H), 8.41 (d, J = 2.0 Hz, 1H) |
| 175 | 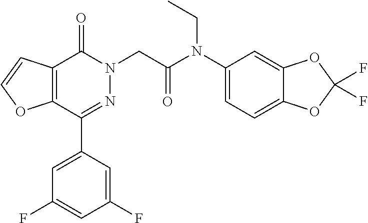 [SMILES: CCN(C(=O)Cn1nc(-c2cc(F)cc(F)c2)c2occc2c1=O)c1ccc2c(c1)OC(F)(F)O2] | 490.2 [M + 1] | (400 MHz, DMSO) δ: 1.04 (t, J = 6.8 Hz, 3H), 3.70 (q, J = 6.8 Hz, 2H), 4.80 (s, 2H), 7.25 (d, J = 1.6 Hz, 1H), 7.34 (d, J = 8.4 Hz, 1H), 7.48-7.56 (m, 2H), 7.68 (s, 1H), 7.73-7.76 (m, 2H), 8.39 (d, J = 1.6 Hz, 1H) |
| 176 | 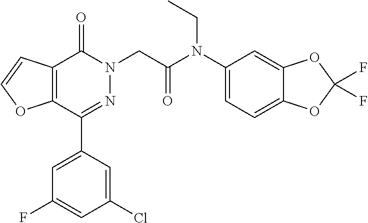 [SMILES: CCN(C(=O)Cn1nc(-c2cc(F)cc(Cl)c2)c2occc2c1=O)c1ccc2c(c1)OC(F)(F)O2] | 506.0 [M + 1] | (400 MHz, DMSO) δ: 1.04 (t, J = 6.8 Hz, 3H), 3.67 (q, H = 6.8 Hz, 2H), 4.80 (s, 2H), 7.26 (d, J = 1.2 Hz, 1H), 7.34 (d, J = 8.4 Hz, 1H), 7.55 (d, J = 8.4 Hz, 1H), 7.66-7.69 (m, 2H), 7.85 (d, J = 9.6 Hz, 1H), 7.96 (s, 1H), 8.39 (d, J = 2.0 Hz, 1H) |
| 177 | 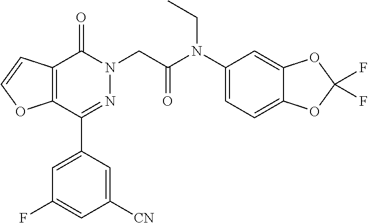 [SMILES: CCN(C(=O)Cn1nc(-c2cc(F)cc(C#N)c2)c2occc2c1=O)c1ccc2c(c1)OC(F)(F)O2] | 497.0 [M + 1] | (400 MHz, DMSO) δ: 1.03 (t, J = 6.8 Hz, 3H), 3.68 (q, J = 6.8 Hz, 2H), 4.81 (s, 2H), 7.27 (s, 1H), 7.34 (d, J = 8.4 Hz, 1H), 7.56 (d, J = 8.4 Hz, 1H), 7.68 (s, 1H), 8.09 (d, J = 8.4 Hz, 1H), 8.19 (d, J = 9.6 Hz, 1H), 8.32 (s, 1H), 8.40 (d, J = 2.0 Hz, 1H) |
| 178 | 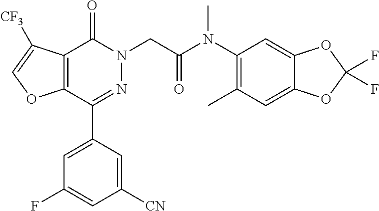 [SMILES: Cc1cc2c(cc1N(C)C(=O)Cn1nc(-c3cc(F)cc(C#N)c3)c3occ(C(F)(F)F)c3c1=O)OC(F)(F)O2] | 565.5 [M + 1] | (400 MHz, DMSO) 2.38 (s, 3H), 3.13 (s, 3H), 4.66 (d, J = 16.0 Hz, 1H), 4.81 (d, J = 16.0 Hz, 1H), 7.55 (s, 1H), 7.69 (s, 1H), 8.12 (d, J = 8.4 Hz, 1H), 8.18 (d, J = 9.6 Hz, 1H), 8.31 (s, 1H), 9.14 (s, 1H) |
| 179 | 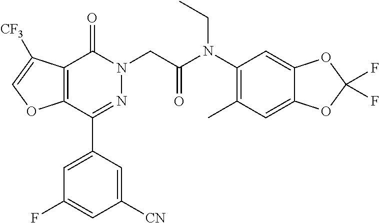 [SMILES: CCN(C(=O)Cn1nc(-c2cc(F)cc(C#N)c2)c2occ(C(F)(F)F)c2c1=O)c1cc2c(cc1C)OC(F)(F)O2] | 579.2 [M + 1] | (400 MHz, DMSO) 1.07 (t, J = 6.8 Hz, 3H), 2.37 (s, 3H), 3.24 (q, J = 6.8 Hz, 1H), 3.99 (q, J = 6.8 Hz, 1H), 4.62 (d, J = 16.0 Hz, 1H), 4.77 (d, J = 16.0 Hz, 1H), 7.56 (s, 1H), 7.63 (s, 1H), 8.12 (d, J = 8.0 Hz, 1H), 8.17 (d, J = 9.6 Hz, 1H), 8.30 (s, 1H), 9.14 (s, 1H) |

### Example 180

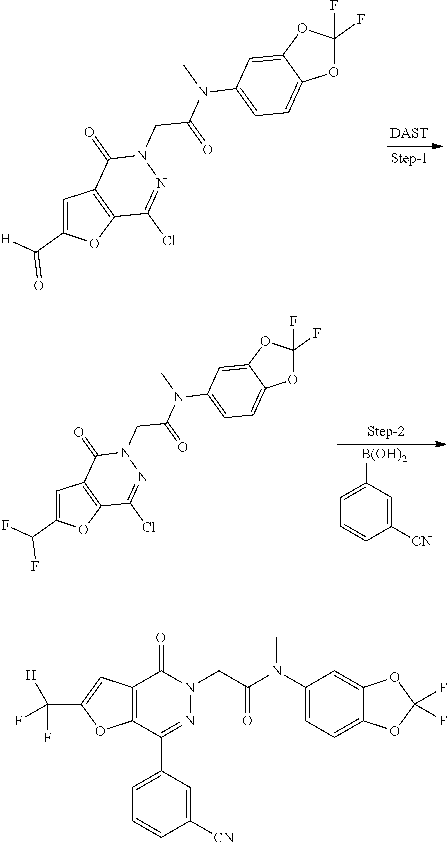 [SMILES: CN(C(=O)Cn1nc(Cl)c2oc(C=O)cc2c1=O)c1ccc2c(c1)OC(F)(F)O2 ; "DAST" ; CN(C(=O)Cn1nc(Cl)c2oc(C(F)F)cc2c1=O)c1ccc2c(c1)OC(F)(F)O2 ; N#Cc1cccc(B(O)O)c1 ; CN(C(=O)Cn1nc(-c2cccc(C#N)c2)c2oc(C(F)F)cc2c1=O)c1ccc2c(c1)OC(F)(F)O2]

Step-1: To a solution of 2-(7-chloro-2-formyl-4-oxofuro[2,3-d]pyridazin-5(4H)-yl)-N-(2,2-difluorobenzo[d][1,3]dioxol-5-yl)-N-methylacetamide (0.70 g, 1.64 mmol) in DCM (30 mL) was added diethylaminosulfur trifluoride (1.19 g, 4.43 mmol) portion-wise at 0° C. The mixture was stirred at 50° C. for 3 h and the reaction mixture was quenched with sat.NaHCO₃, extracted with DCM (3×50 mL). The combined organic layers were washed with brine, dried over sodium sulfate, concentrated. The product was purified by chromatography (0-40% ethyl acetate in hexanes) to obtain 2-(7-chloro-2-(difluoromethyl)-4-oxofuro[2,3-d]pyridazin-5(4H)-yl)-N-(2,2-difluorobenzo[d][1,3]dioxol-5-yl)-N-methylacetamide (0.70 g, 95%) as white solid. (MS ESI +ve, 448.0 [M+H]).

Step-2: A mixture of 2-(7-chloro-2-(difluoromethyl)-4-oxofuro[2,3-d]pyridazin-5(4H)-yl)-N-(2,2-difluorobenzo[d][1,3]dioxol-5-yl)-N-methylacetamide (0.40 g, 0.89 mmol) in ethanol (4 mL), toluene (4 mL), water (2 mL), (3-cyanophenyl)boronic acid (0.20 g, 1.33 mmol) and sodium carbonate (141 mg, 1.33 mmol) was degassed for 15 minutes, followed by addition of Pd(PPh₃)₄ (0.10 g, 0.089 mmol). The mixture was further degassed for 15 minutes and was heated at 70° C. for 16 h. The mixture was diluted with water and extracted with ethyl acetate (3×100 mL). The combined organic layer was washed with brine and dried over sodium sulphate. The volatiles were removed under vacuum and the product was purified by column chromatography (0-40% ethyl acetate in hexanes) to give 2-(7-(3-cyanophenyl)-2-(difluoromethyl)-4-oxofuro[2,3-d]pyridazin-5(4H)-yl)-N-(2,2-difluorobenzo[d][1,3]dioxol-5-yl)-N-methylacetamide (0.19 g, 41%) as white solid. (MS ESI +ve, 515.0 [M+H]); ¹H NMR: (400 MHz, DMSO-d6): 3.21 (s, 3H), 4.88 (s, 2H), 7.41 (m, 1H), 7.41 (s, 1H), 7.54-7.58 (m, 2H), 7.69 (s, 1H), 7.74 (s, 1H), 7.86 (t, J=8.0 Hz, 1H), 8.06 (d, J=8.0 Hz, 1H), 8.35 (d, J=7.6 Hz, 1H), 8.40 (s, 1H).

### Example 181

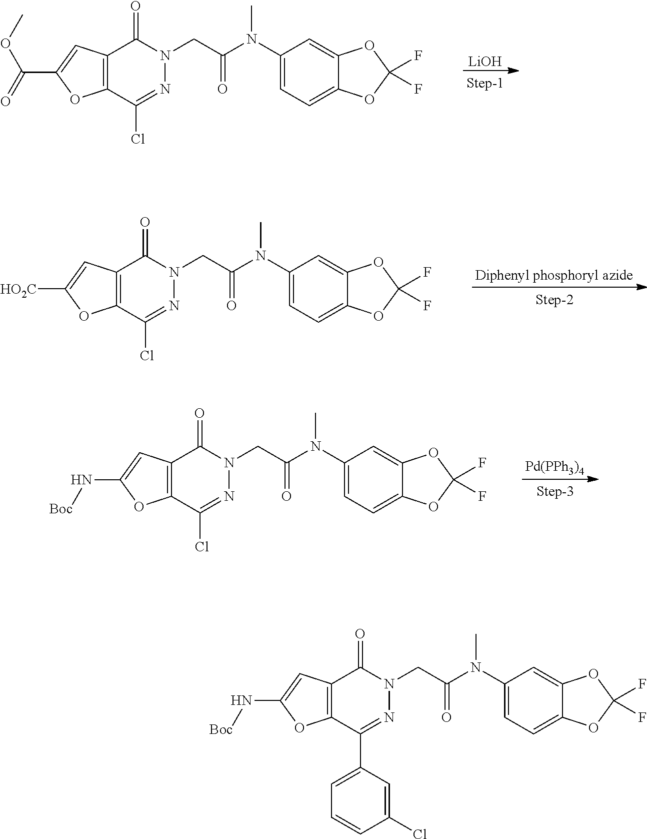

Step-1: To a solution methyl 7-chloro-5-(2-((2,2-difluorobenzo[d][1,3]dioxol-5-yl)(methyl)amino)-2-oxoethyl)-4-oxo-4,5-dihydrofuro[2,3-d]pyridazine-2-carboxylate (0.70 g, 1.54 mmol) in THF (6 mL) and water (6 mL) was added LiOH (0.16 g, 3.76 mmol). The mixture was stirred at rt overnight and was neutralized with 1N HCl. The resulting mixture was extracted with ethyl acetate. The organic layer was washed with brine and dried over sodium sulfate. Solvent was evaporated to give 7-chloro-5-(2-((2,2-difluorobenzo[d][1,3]dioxol-5-yl)(methyl)amino)-2-oxoethyl)-4-oxo-4,5-dihydrofuro[2,3-d]pyridazine-2-carboxylic acid (0.50 g, 73%) for next step.

Step-2: To a solution of 7-chloro-5-(2-((2,2-difluorobenzo[d][1,3]dioxol-5-yl)(methyl)amino)-2-oxoethyl)-4-oxo-4,5-dihydrofuro[2,3-d]pyridazine-2-carboxylic acid (0.48 g, 1.09 mmol) in toluene (15 mL) was added diphenyl phosphoryl azide (0.36 g, 1.31 mmol), triethyl amine (0.15 g, 1.44 mmol) and tert-butanol (0.11 g, 1.44 mmol). The mixture was heated at 70° C. for 6 h and ice water was added. The mixture was extracted with ethyl acetate, washed with brine, dried over anhydrous sodium sulfate. Solvent was evaporated to give 7-(3-chlorophenyl)-5-(2-((2,2-difluorobenzo[d][1,3]dioxol-5-yl)(methyl)amino)-2-oxoethyl)-4-oxo-4,5-dihydrofuro[2,3-d]pyridazine-2-carboxylic acid (0.41 g, 73%) for next step.

Step-3: A mixture of methyl tert-butyl (7-chloro-5-(2-((2,2-difluorobenzo[d][1,3]dioxol-5-yl)(methyl)amino)-2-oxoethyl)-4-oxo-4,5-dihydrofuro[2,3-d]pyridazin-2-yl)carbamate (1.0 g, 2.2 mmol), (3-chlorophenyl)boronic acid (0.40 g, 0.77 mmol) in DMF (5 mL), cesium fluoride (0.66 g, 4.3 mmol) in water (1 mL) was degassed and PdCl₂dppf (0.048 g, 0.06 mmol) was added. The mixture was degassed again and heated at 100° C. overnight. Solvent was evaporated and the product was purified by column chromatography (5% methanol in dichloromethane) to give tert-butyl (7-(3-chlorophenyl)-5-(2-((2,2-difluorobenzo[d][1,3]dioxol-5-yl)(methyl)amino)-2-oxoethyl)-4-oxo-4,5-dihydrofuro[2,3-d]pyridazin-2-yl)carbamate (0.06 g, 13%). (MS ESI +Ve, 589.2 [M+1]); ¹H NMR: (400 MHz, DMSO) δ: 1.51 (s, 9H); 3.20 (s, 3H); 4.80 (s, 2H); 6.42 (s, 1H); 7.38 (m, 1H), 7.53 (d, J=8.4 Hz, 1H); 7.60 (m, 2H), 7.70 (s, 1H), 8.02 (m, 1H), 8.08 (s, 1H), 11.35 (br, 1H).

### Example 182

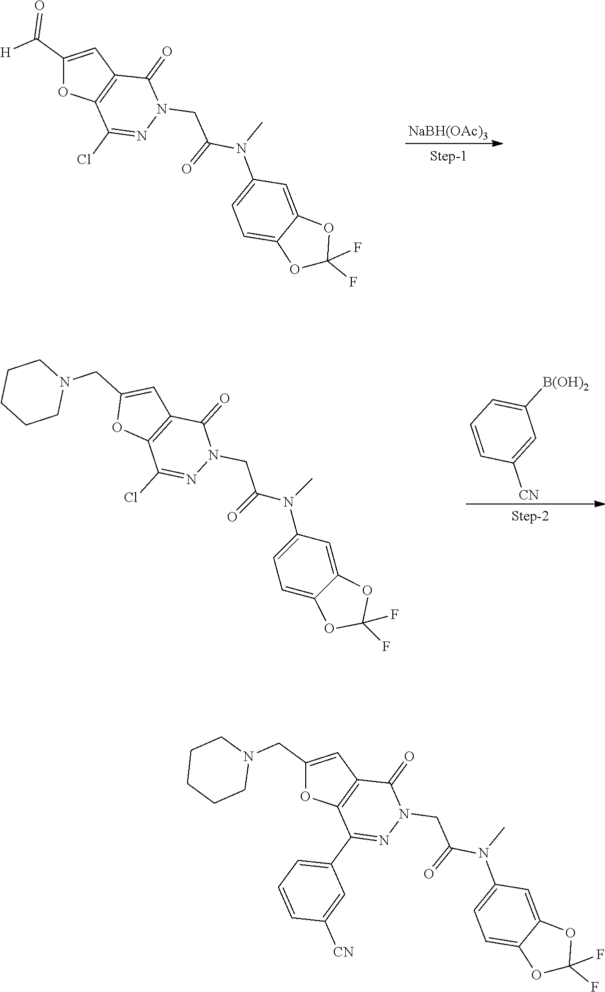 [SMILES: CN(C(=O)Cn1nc(Cl)c2oc(C=O)cc2c1=O)c1ccc2c(c1)OC(F)(F)O2 ; CN(C(=O)Cn1nc(Cl)c2oc(CN3CCCCC3)cc2c1=O)c1ccc2c(c1)OC(F)(F)O2 ; N#Cc1cccc(B(O)O)c1 ; "NaBH(OAc)3" ; CN(C(=O)Cn1nc(-c2cccc(C#N)c2)c2oc(CN3CCCCC3)cc2c1=O)c1ccc2c(c1)OC(F)(F)O2]

Step-1: A mixture of 2-(7-chloro-2-formyl-4-oxofuro[2,3-d]pyridazin-5(4H)-yl)-N-(2,2-difluorobenzo[d][1,3]dioxol-5-yl)-N-methylacetamide (0.30 g, 0.70 mmol) in dichloromethane, piperidine (0.05 g, 0.58 mmol), AcOH (0.13 g, 2.10 mmol) was stirred for 30 min before the addition of triacetoxy borohydride (0.30 g, 1.40 mmol). The mixture was stirred at room temperature for 12 h and quenched with water, extracted with dichloromethane. The combined organic layer was washed with brine, dried over anhydrous sodium sulfate, and concentrated in vacuum to give 2-(7-chloro-4-oxo-2-(piperidin-1-ylmethyl)furo[2,3-d]pyridazin-5(4H)-yl)-N-(2,2-difluorobenzo[d][1,3]dioxol-5-yl)-N-methylacetamide (0.25 g, 71%). (MS ESI +Ve, 495.2 [M+1]).

Step-2: A mixture of 2-(7-chloro-4-oxo-2-(piperidin-1-ylmethyl)furo[2,3-d]pyridazin-5(4H)-yl)-N-(2,2-difluorobenzo[d][1,3]dioxol-5-yl)-N-methylacetamide (0.20 g, 0.40 mmol), (3-cyanophenyl)boronic acid (0.07 g, 0.48 mmol) in ethanol (3 mL), toluene (3 mL), water (1.5 mL) and sodium carbonate (0.063 g, 0.60 mmol) was degassed and then Pd(PPh₃)₄ (0.046 g, 0.040 mmol) was added. The mixture was degassed again and heated at 80° C. overnight. The solvent was evaporated and the residue was purified by column chromatography pure product (4% methanol in dichloromethane) to give crude product, which was purify by reverse phase preparative HPLC (gradient ACN/H₂O, 0.1% formic acid) to give 2-(7-(3-cyanophenyl)-4-oxo-2-(piperidin-1-ylmethyl)furo[2,3-d]pyridazin-5(4H)-yl)-N-(2,2-difluorobenzo[d][1,3]dioxol-5-yl)-N-methylacetamide (0.04 g, 17%). (MS ESI +Ve, 562.2 [M+1]); ¹H NMR: (400 MHz, DMSO) δ: 1.37 (m, 2H); 1.51 (d, J=4.8 Hz, 4H); 2.46 (m, 4H); 3.20 (s, 3H); 3.78 (s, 2H); 4.84 (s, 2H); 7.03 (s, 1H); 7.39 (d, J=7.6 Hz, 1H), 7.53 (d, J=8.0 Hz, 1H), 7.72 (s, 1H), 7.83 (t, J=8.0 Hz, 1H), 8.03 (d, J=7.6 Hz, 1H), 8.35 (d, J=8.0 Hz, 1H), 8.40 (s, 1H).

### Example 183

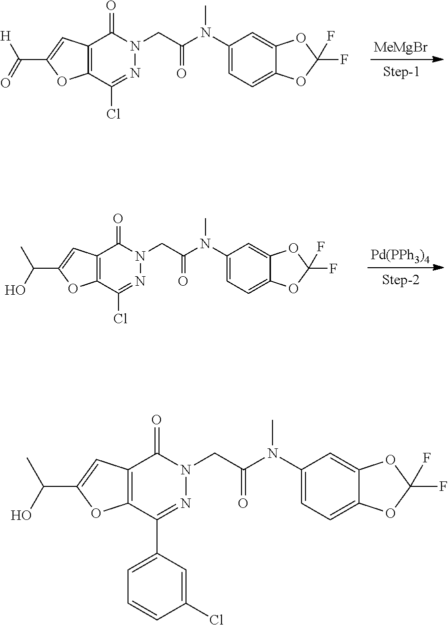 [SMILES: "MeMgBr" ; CN(C(=O)Cn1nc(Cl)c2oc(C=O)cc2c1=O)c1ccc2c(c1)OC(F)(F)O2 ; CC(O)c1cc2c(=O)n(CC(=O)N(C)c3ccc4c(c3)OC(F)(F)O4)nc(Cl)c2o1 ; CC(O)c1cc2c(=O)n(CC(=O)N(C)c3ccc4c(c3)OC(F)(F)O4)nc(-c3cccc(Cl)c3)c2o1 ; "Pd(PPh3)4"]

Step-1: To a solution of 2-(7-chloro-2-formyl-4-oxofuro[2,3-d]pyridazin-5(4H)-yl)-N-(2,2-difluorobenzo[d][1,3]dioxol-5-yl)-N-methylacetamide (0.05 g, 0.12 mmol) in THF (5 mL) was added MeMgBr (0.04 mL, 0.17 mmol) dropwise at 0° C. The mixture was stirred at room temperature for 2 h and was quenched with 1N HCl, extracted with dichloromethane. The organic layer was dried, evaporated to give 2-(7-chloro-2-(1-hydroxyethyl)-4-oxofuro[2,3-d]pyridazin-5(4H)-yl)-N-(2,2-difluorobenzo[d][1,3]dioxol-5-yl)-N-methylacetamide (0.025 g, 48%) for next step.

Step-2: A mixture of 2-(7-chloro-2-(1-hydroxyethyl)-4-oxofuro[2,3-d]pyridazin-5(4H)-yl)-N-(2,2-difluorobenzo[d][1,3]dioxol-5-yl)-N-methylacetamide (0.10 g, 0.23 mmol), (3-chlorophenyl)boronic acid (0.038 g, 0.24 mmol) in ethanol (3 mL), toluene (3 mL), water (1.5 mL), sodium carbonate (0.035 g, 0.34 mmol) was degassed and then Pd(PPh₃)₄ (0.026 g, 0.022 mmol) was added. The mixture was degassed again and was heated at 80° C. overnight. The mixture was evaporated and the residue was purified by column chromatography (4% methanol in dichloromethane) to give crude product, which was purify by reverse phase preparative HPLC purification to give 2-(7-(3-chlorophenyl)-2-(1-hydroxyethyl)-4-oxofuro[2,3-d]pyridazin-5(4H)-yl)-N-(2,2-difluorobenzo[d][1,3]dioxol-5-yl)-N-methylacetamide (0.005 g, 4.3%). (MS ESI +Ve, 518.5 [M+1]). ¹H NMR: (400 MHz, DMSO) δ: 1.51 (d, J=6.8 Hz, 3H); 3.19 (s, 3H); 4.83 (s, 2H); 4.94 (m, 1H); 5.82 (m, 2H); 6.96 (s, 1H), 7.39 (d, J=8.0 Hz, 1H), 7.54 (d, J=8.4 Hz, 1H), 7.62-7.66 (m, 2H), 8.05-8.08 (m, 2H).

### Example 184

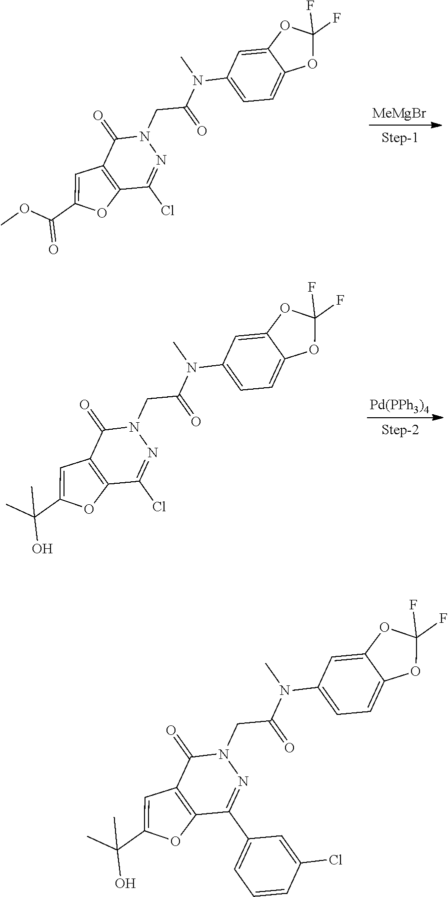 [SMILES: "MeMgBr" ; COC(=O)c1cc2c(=O)n(CC(=O)N(C)c3ccc4c(c3)OC(F)(F)O4)nc(Cl)c2o1 ; CN(C(=O)Cn1nc(Cl)c2oc(C(C)(C)O)cc2c1=O)c1ccc2c(c1)OC(F)(F)O2 ; CN(C(=O)Cn1nc(-c2cccc(Cl)c2)c2oc(C(C)(C)O)cc2c1=O)c1ccc2c(c1)OC(F)(F)O2 ; "Pd(PPh3)4"]

2-(7-(3-chlorophenyl)-2-(2-hydroxypropan-2-yl)-4-oxofuro[2,3-d]pyridazin-5(4H)-yl)-N-(2,2-difluorobenzo[d][1,3]dioxol-5-yl)-N-methylacetamide (0.015 vg, 6.4%) was synthesized in a similar fashion as showed in scheme above. (MS ESI +Ve, 532.4 [M+1]). ¹H NMR: (400 MHz, DMSO) δ: 1.57 (s, 3H), 1.62 (s, 3H); 3.20 (s, 3H); 4.83 (s, 2H); 5.71 (s, 1H); 6.92 (s, 1H); 7.40 (d, J=8.0 Hz, 1H); 7.55 (d, J=8.4 Hz, 1H); 7.60-7.67 (m, 2H); 7.73 (s, 1H), 8.06-8.09 (m, 2H).

The following compounds were synthesized in similar fashion:

| Exam- | | | |
|---|---|---|---|
| ple | | | |
| # | Stucture | m/z | ¹H NMR |
| 185 | 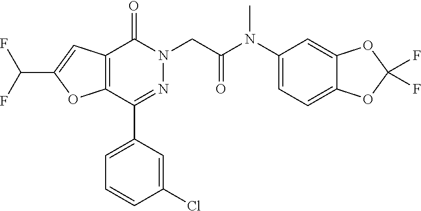 [SMILES: CN(C(=O)Cn1nc(-c2cccc(Cl)c2)c2oc(C(F)F)cc2c1=O)c1ccc2c(c1)OC(F)(F)O2] | 524.0 [M + 1] | (400 MHz, DMSO) δ: 3.21 (s, 3H), 4.87 (s, 2H), 7.40-7.42 (m, 2H), 7.53-7.57 (m, 2H), 7.66-7.69 (m, 3H), 7.74 (m, 1H), 8.00-8.03 (m, 2H) |
| 186 | 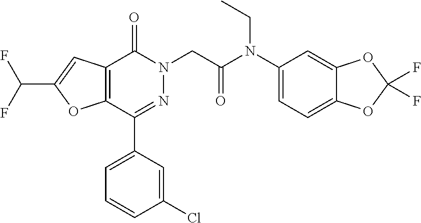 [SMILES: CCN(C(=O)Cn1nc(-c2cccc(Cl)c2)c2oc(C(F)F)cc2c1=O)c1ccc2c(c1)OC(F)(F)O2] | 538.2 [M + 1] | (400 MHz, DMSO) δ: 1.04 (t, J = 6.8 Hz, 3H); 3.68 (q, J = 6.8 Hz, 2H); 4.80 (s, 2H), 7.26-7.39 (m, 2H), 7.54 (m, 1H), 7.65-7.68 (m, 4H), 8.01 (m, 2H) |
| 187 | 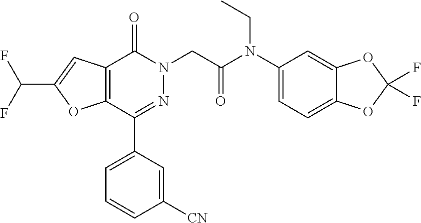 [SMILES: CCN(C(=O)Cn1nc(-c2cccc(C#N)c2)c2oc(C(F)F)cc2c1=O)c1ccc2c(c1)OC(F)(F)O2] | 529.2 [M + 1] | (400 MHz, DMSO) δ: 1.05 (t, J = 6.8 Hz, 3H); 3.69 (q, J = 6.8 Hz, 2H); 4.82 (s, 2H), 7.34-7.41 (m, 2H); 7.55 (m, 1H), 7.69 (s, 2H); 7.86 (t, J = 8.0 Hz, 1H), 8.35 (d, J = 8.0 Hz, 1H); 8.40 (s, 1H) |
| 188 | 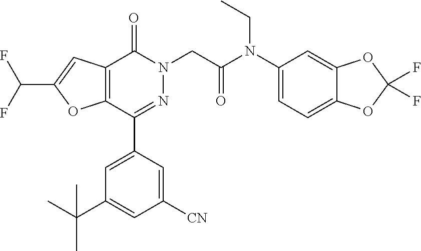 [SMILES: CCN(C(=O)Cn1nc(-c2cc(C#N)cc(C(C)(C)C)c2)c2oc(C(F)F)cc2c1=O)c1ccc2c(c1)OC(F)(F)O2] | 585.5 [M + 1] | (400 MHz, DMSO) δ: 1.05 (t, J = 6.8 Hz, 3H), 1.39 (s, 9H), 3.70 (q, J = 6.8 Hz, 2H), 4.83 (s, 2H), 7.35 (d, J = 8.4 Hz, 1H), 7.41 (CHF2) (t, J = 52.4 Hz, 1H), 7.56 (d, J = 8.4 Hz, 1H), 7.68 (s, 2H), 8.09 (s, 1H), 8.20 (s, 1H), 8.37 (s, 1H) |
| 189 | 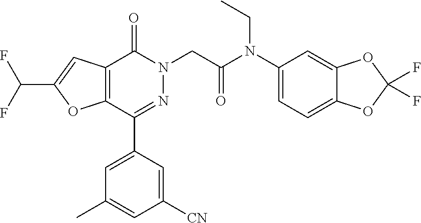 [SMILES: CCN(C(=O)Cn1nc(-c2cc(C)cc(C#N)c2)c2oc(C(F)F)cc2c1=O)c1ccc2c(c1)OC(F)(F)O2] | 543.4 [M + 1] | (400 MHz, CDCl3) δ: 1.21 (t, J = 7.2 Hz, 3H) 2.55 (s, 3H), 3.83 (q, J = 7.2 Hz, 2H), 4.84 (s, 2H), 6.87 (s, 1H), 7.20-7.25 (m, 3H), 7.38 (s, 1H), 7.61 (s, 1H), 8.15 (s, 1H), 8.21 (s, 1H) |
| 190 | 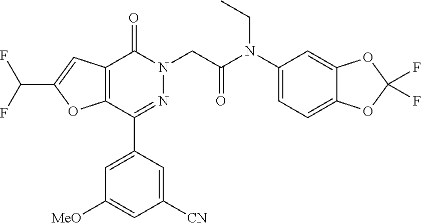 [SMILES: CCN(C(=O)Cn1nc(-c2cc(C#N)cc(OC)c2)c2oc(C(F)F)cc2c1=O)c1ccc2c(c1)OC(F)(F)O2] | 559.0 [M + 1] | (400 MHz, DMSO) δ: 1.04 (t, J = 6.8 Hz, 3H), 3.69 (q, J = 6.8 Hz, 2H), 3.93 (s, 3H), 4.82 (s, 2H), 7.35 (CHF2) (m, 1H), 7.41 (s, 1H), 7.54-7.57 (m, 1H), 7.68 (s, 3H), 7.87 (s, 1H) 7.98 (s, 1H) |
| 191 | 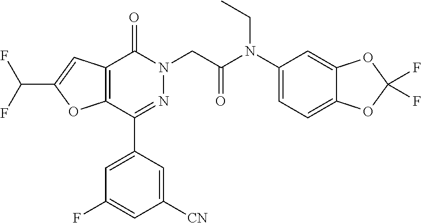 [SMILES: CCN(C(=O)Cn1nc(-c2cc(F)cc(C#N)c2)c2oc(C(F)F)cc2c1=O)c1ccc2c(c1)OC(F)(F)O2] | 547.0 [M + 1] | 400 MHz, DMSO) δ: 1.04 (t, J = 6.8 Hz, 3H), 3.68 (q, J = 6.8 Hz, 2H) 4.82 (s, 2H), 7.35 (d, J = 8.8 Hz, 1H), 7.41 (CHF2) (t, J = 52.4 Hz, 1H), 7.56 (d, J = 8.8 Hz, 1H), 7.69 (s, 2H), 8.11 (d, J = 8.4 Hz, 1H), 8.18 (d, J = 9.2 Hz, 1H), 8.28 (s, 1H) |
| 192 | 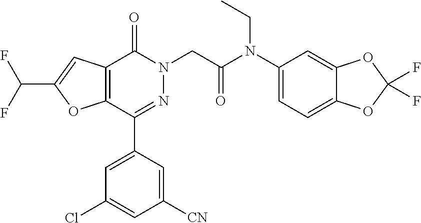 [SMILES: CCN(C(=O)Cn1nc(-c2cc(Cl)cc(C#N)c2)c2oc(C(F)F)cc2c1=O)c1ccc2c(c1)OC(F)(F)O2] | 563.4 [M + 1] | (400 MHz, CDCl3) δ: 1.21 (t, J = 7.2 Hz, 3H), 3.83 (q, J = 7.2 Hz, 2H), 4.84 (s, 2H), 6.89 (CHF2) (t, J = 52.4 Hz, 1H), 7.16-7.27 (m, 3H), 7.40 (s, 1H), 7.79 (s, 1H), 8.33-8.36 (m, 2H) |
| 193 | 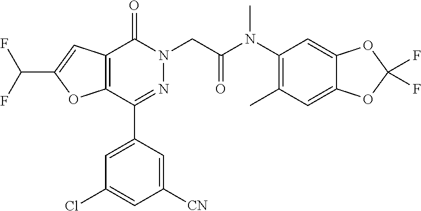 [SMILES: Cc1cc2c(cc1N(C)C(=O)Cn1nc(-c3cc(Cl)cc(C#N)c3)c3oc(C(F)F)cc3c1=O)OC(F)(F)O2] | 563.5 [M + 1] | (400 MHz, DMSO) δ: 2.39 (s, 3H), 3.12 (s, 3H), 4.65 (d, J = 16.0 Hz, 1H), 4.82 (d, J = 16.0 Hz, 1H), 7.43 (CHF2) (t, J = 52.4 Hz, 1H), 7.56 (s, 1H), 7.69 (s, 1H), 7.77 (m, 1H), 8.29 (s, 1H), 8.36 (s, 1H), 8.41 (s, 1H) |
| 194 | 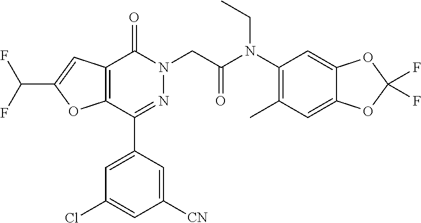 [SMILES: CCN(C(=O)Cn1nc(-c2cc(Cl)cc(C#N)c2)c2oc(C(F)F)cc2c1=O)c1cc2c(cc1C)OC(F)(F)O2] | 577.5 [M + 1] | (400 MHz, DMSO) δ: 1.07 (t, J = 6.8 Hz, 3H), 2.39 (s, 3H), 3.21 (m, 1H), 3.99 (m, 1H), 4.60 (d, J = 16.0 Hz, 1H), 4.78 (d, J = 16.0 Hz, 1H), 7.43 (CHF2) (t, J = 52.4 Hz, 1H), 7.57 (s, 1H), 7.63 (s, 1H), 7.71 (s, 1H), 8.29 (s, 1H), 8.35 (s, 1H), 8.40 (s, 1H) |
| 195 | 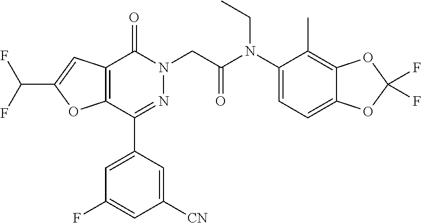 [SMILES: CCN(C(=O)Cn1nc(-c2cc(F)cc(C#N)c2)c2oc(C(F)F)cc2c1=O)c1ccc2c(c1C)OC(F)(F)O2] | 561.4 [M + 1] | (400 MHz, DMSO) δ: 1.06 (t, J = 6.8 Hz, 3H), 2.347 (s, 3H), 3.18 (m, 1H), 4.00 (m, 1H), 4.64 (d, J = 16.0 Hz, 1H), 4.74 (d, J = 16.0 Hz, 1H), 7.33 (d, J = 8.4 Hz, 1H), 7.41 (d, J = 8.4 Hz, 1H), 7.43 (CHF2) (t, J = 52.4 Hz, 1H), 7.71 (s, 1H), 8.11 (m, 1H), 8.19 (m, 1H), 8.30 (s, 1H) |
| 196 | 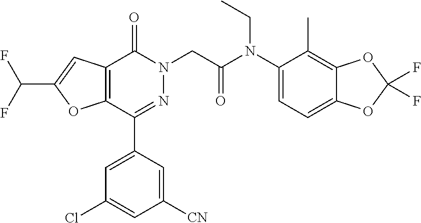 [SMILES: CCN(C(=O)Cn1nc(-c2cc(Cl)cc(C#N)c2)c2oc(C(F)F)cc2c1=O)c1ccc2c(c1C)OC(F)(F)O2] | 577.1 [M + 1] | (400 MHz, DMSO) δ: 1.06 (t, J = 6.8 Hz, 3H), 2.35 (s, 3H), 3.22 (q, J = 6.8 Hz, 1H), 4.00 (q, J = 6.8 Hz, 1H), 4.63 (d, J = 16.0 Hz, 1H), 4.74 (d, J = 16.0 Hz, 1H), 7.34 (d, J = 8.4 Hz, 1H), 7.41 (d, J = 9.6 Hz, 1H), 7.43 (t, J = 52.0 Hz, 1H), 7.70 (s, 1H), 8.27 (s, 1H), 8.34 (s, 1H), 8.39 (s, 1H) |
| 197 | 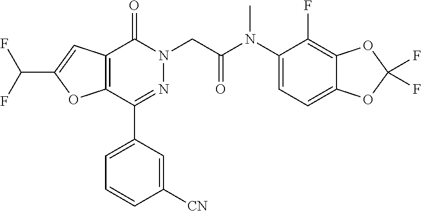 [SMILES: CN(C(=O)Cn1nc(-c2cccc(C#N)c2)c2oc(C(F)F)cc2c1=O)c1ccc2c(c1F)OC(F)(F)O2] | 533.0 [M + 1] | (400 MHz, DMSO) δ: 3.17 (s, 1H), 4.84 (d, J = 16.0 Hz, 1H), 4.94 (d, J = 16.0 Hz, 1H), 7.41 (CHF2) (t, J = 52.4 Hz, 1H), 7.70 (s, 1H), 7.79-7.88 (m, 2H), 7.93 (d, J = 6.8 Hz, 1H), 8.06 (d, J = 7.6 Hz, 1H), 8.34 (d, J = 8.4 Hz, 1H), 8.42 (s, 1H) |
| 198 | 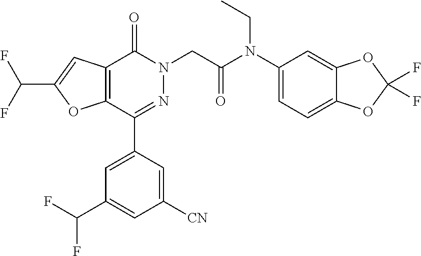 [SMILES: CCN(C(=O)Cn1nc(-c2cc(C#N)cc(C(F)F)c2)c2oc(C(F)F)cc2c1=O)c1ccc2c(c1)OC(F)(F)O2] | 579.0 [M + 1] | (400 MHz, DMSO) δ: 1.04 (t, J = 7.2 Hz, 3H), 3.69 (q, J = 7.2 Hz, 2H), 4.84 (s, 2H), 7.26 (t, J = 54.8 Hz, 1H), 7.36 (d, J = 8.8 Hz, 1H), 7.41 (t, J = 52.4 Hz, 1H), 7.57 (d, J = 8.4 Hz, 1H), 7.69 (m, 2H), 8.31 (s, 1H), 8.48 (s, 1H), 8.60 (s, 1H) |
| 199 | 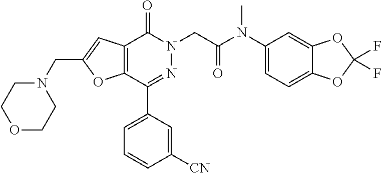 [SMILES: CN(C(=O)Cn1nc(-c2cccc(C#N)c2)c2oc(CN3CCOCC3)cc2c1=O)c1ccc2c(c1)OC(F)(F)O2] | 564.5 [M + 1] | (400 MHz, DMSO) δ: 2.49 (m, 4H); 3.18 (s, 3H); 3.59 (d, J = 4.0 Hz, 4H); 3.80 (s, 2H), 4.83 (s, 2H); 7.07 (s, 1H); 7.35 (m, 1H); 7.47 (m, 1H), 7.64 (m, 1H); 7.80 (t, J = 7.6 Hz, 1H); 7.99 (d, J = 7.6 Hz, 1H); 8.35- 8.40 (m, 2H) |
| 200 | 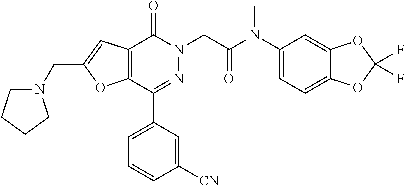 [SMILES: CN(C(=O)Cn1nc(-c2cccc(C#N)c2)c2oc(CN3CCCC3)cc2c1=O)c1ccc2c(c1)OC(F)(F)O2] | 548.0 [M + 1] | (400 MHz, DMSO) δ: 1.72 (s, 4H), 2.58 (m, 4H) 3.21 (s, 3H); 3.89 (s, 2H); 4.85 (s, 2H), 7.05 (s, 1H); 7.40 (d, J = 8.4 Hz, 1H), 7.55 (d, J = 8.0 Hz, 1H), 7.74 (s, 1H); 7.83 (t, J = 8.0 Hz, 1H), 8.04 (d, J = 7.6 Hz, 1H), 8.36 (d, J = 8.0 Hz), 8.40 (s, 1H) |
| 201 | 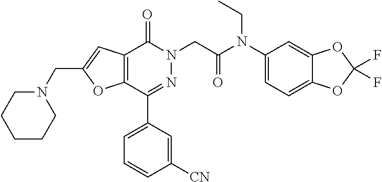 [SMILES: CCN(C(=O)Cn1nc(-c2cccc(C#N)c2)c2oc(CN3CCCCC3)cc2c1=O)c1ccc2c(c1)OC(F)(F)O2] | 576.2 [M + 1] | (400 MHz, DMSO) δ: 1.04 (t, J = 6.8 Hz, 3H); 1.38 (m, 2H); 1.52 (d, J = 4.8 Hz, 4H); 2.47 (m, 4H); 69 (q, J = 7.2 Hz, 2H); 3.79 (s, 2H); 4.79 (s, 2H); 7.03 (s, 1H); 7.34 (d, J = 8.0 Hz, 1H); 7.55 (d, J = 8.4 Hz, 1H); 7.68 (s, 1H); 7.84 (t, J = 8.0 Hz, 1H); 8.03 (d, J = 7.6 Hz, 1H); 8.36 (t, J = 8.0 Hz, 1H), 8.41 (s, 1H) |
| 202 | 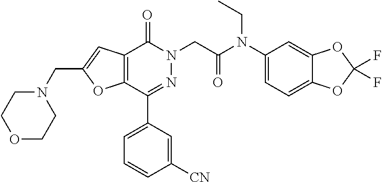 [SMILES: CCN(C(=O)Cn1nc(-c2cccc(C#N)c2)c2oc(CN3CCOCC3)cc2c1=O)c1ccc2c(c1)OC(F)(F)O2] | 578.4 [M + 1] | (400 MHz, DMSO) δ: 1.04 (t, J = 6.8 Hz, 3H); 2.51 (m, 4H); 3.60 (t, J = 4.0 Hz, 4H); 3.69 (q, J = 7.2 Hz, 2H); 3.82 (s, 2H); 4.79 (s, 2H); 7.08 (s, 1H); 7.34 (d, J = 7.6 Hz, 1H); 7.55 (d, J = 8.8 Hz, 1H); 7.68 (s, 1H); 7.84 (t, J = 8 Hz, 1H); 8.03 (d, J = 7.6 Hz, 1H); 8.36 (t, J = 8.o0 Hz, 2H), 8.40 (s, 1H) |
| 203 | 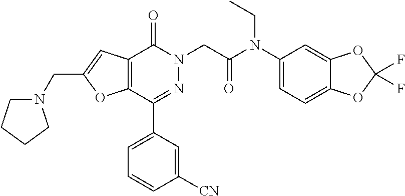 [SMILES: CCN(C(=O)Cn1nc(-c2cccc(C#N)c2)c2oc(CN3CCCC3)cc2c1=O)c1ccc2c(c1)OC(F)(F)O2] | 562.2 [M + 1] | (400 MHz, DMSO) δ: 1.04 (t, J = 7.2 Hz, 3H); 1.72 (s, 4H); 2.58 (s, 4H); 3.69 (d, J = 7.2 Hz, 2H); 3.89 (s, 2H); 4.79 (s, 2H); 7.04 (s, 1H); 7.35 (d, J = 8.0 Hz, 1H); 7.55 (d, J = 8.8 Hz, 1H); 7.69 (s, 1H); 7.84 (t, J = 8.0 Hz, 1H); 8.04 (d, J = 7.6 Hz, 1H); 8.35 (d, J = 8.0 Hz, 1H), 8.40 (s, 1H) |
| 204 | 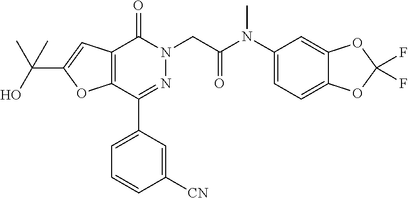 [SMILES: CN(C(=O)Cn1nc(-c2cccc(C#N)c2)c2oc(C(C)(C)O)cc2c1=O)c1ccc2c(c1)OC(F)(F)O2] | 523.4 [M + 1] | (400 MHz, DMSO) δ: 1.58 (s, 6H); 3.20 (s, 3H); 4.84 (s, 2H); 5.74 (s, 1H); 6.94 (s, 1H), 7.40 (d, J = 7.6 Hz, 1H); 7.55 (d, J = 8.8 Hz, 1H); 7.74 (s, 1H); 7.83 (t, J = 8.0 Hz, 1H); 8.02 (d, J = 7.6 Hz, 1H); 8.38-8.44 (m, 2H) |

### Example 205

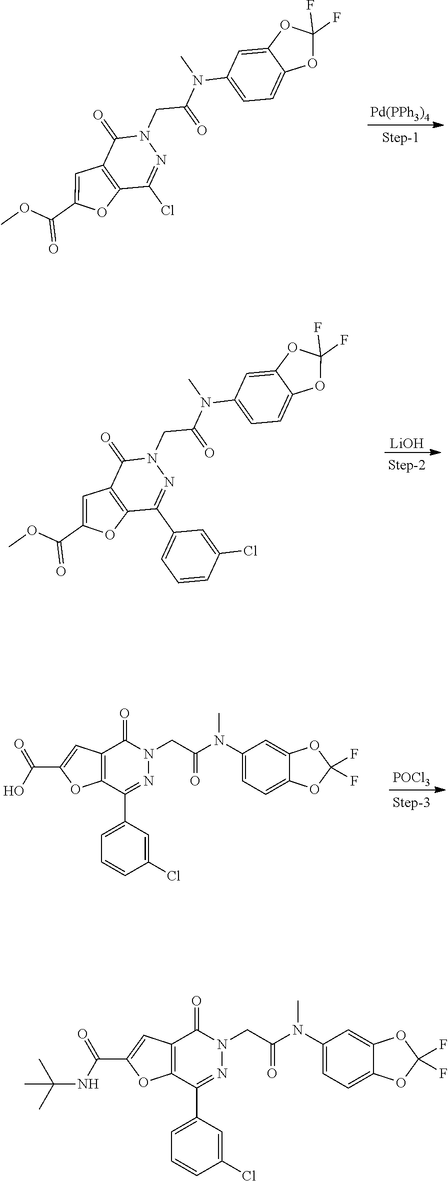

Step-1: A mixture of methyl 7-chloro-5-(2-((2,2-difluorobenzo[d][1,3]dioxol-5-yl)(methyl)amino)-2-oxoethyl)-4-oxo-4,5-dihydrofuro[2,3-d]pyridazine-2-carboxylate (1.0 g, 2.2 mmol), (3-chlorophenyl)boronic acid (0.41 g, 2.6 mmol) in dimethoxyethane (5 mL), cesium fluoride (0.66 g, 4.3 mmol), water (1 mL) was degassed and then PdCl₂dppf (0.048 g, 0.06 mmol) was added. The mixture was degassed again and was heated at 100° C. overnight. Solvent was evaporated the residue was purified by column chromatography (5% methanol in dichloromethane) to give methyl 7-(3-chlorophenyl)-5-(2-((2,2-difluorobenzo[d][1,3]dioxol-5-yl)(methyl)amino)-2-oxoethyl)-4-oxo-4,5-dihydrofuro[2,3-d]pyridazine-2-carboxylate (0.60 g, 51%). (MS ESI +ve, 532.5 [M+1]).

Step-2: To a solution of methyl 7-(3-chlorophenyl)-5-(2-((2,2-difluorobenzo[d][1,3]dioxol-5-yl)(methyl)amino)-2-oxoethyl)-4-oxo-4,5-dihydrofuro[2,3-d]pyridazine-2-carboxylate (0.70 g, 0.94 mmol) in THF (6 mL) and water (6 mL) was added LiOH (0.16 g, 3.8 mmol). The mixture was stirred at room temperature overnight and was neutralized by using 1N HCl. The resulting mixture was extracted with ethyl acetate. The organic layer was washed, dried, evaporated to give 7-(3-chlorophenyl)-5-(2-((2,2-difluorobenzo[d][1,3]dioxol-5-yl)(methyl)amino)-2-oxoethyl)-4-oxo-4,5-dihydrofuro[2,3-d]pyridazine-2-carboxylic acid (0.50 g, 730%) for next step. (MS ESI +ve, 518.4 [M+1]).

Step-3. To a solution of 7-(3-chlorophenyl)-5-(2-((2,2-difluorobenzo[d][1,3]dioxol-5-yl)(methyl)amino)-2-oxoethyl)-4-oxo-4,5-dihydrofuro[2,3-d]pyridazine-2-carboxylic acid (0.20 g, 0.38 mmol) in dichloromethane (5 mL) was added phosphoryl chloride (0.8 mL) at 0° C. The mixture was stirred for 30 min before the addition of 2-methylpropan-2-amine (0.042 g, 0.58 mmol) in dichloromethane (3 ml), pyridine (1 mL). The reaction mixture was stirred at room temperature for 12 h and was quenched with aq. sodium bicarbonate, extracted with dichloromethane. The organic layer was washed with brine, dried and evaporated. The residue was purified by combiflash (2-5% Methanol in dichloromethane) to give crude product, which was further purified by reverse phase preparative HPLC to afford N-(tert-butyl)-7-(3-chlorophenyl)-5-(2-((2,2-difluorobenzo[d][1,3]dioxol-5-yl)(methyl)amino)-2-oxoethyl)-4-oxo-4,5-dihydrofuro[2,3-d]pyridazine-2-carboxamide (0.065 g, 290). (MS ESI +Ve, 573.2 [M+1]).

¹H NMR: (400 MHz, DMSO) δ: 1.39 (s, 9H); 3.18 (s, 3H); 4.82 (s, 2H); 7.34 (d, J=8. Hz, 1H); 7.49 (d, J=8.0 Hz, 1H); 7.61-7.65 (i, 3H); 7.82 (s, 1H); 8.07 (m, 2H); 8.20 (s, 1H).

In a similar fashion, the following examples were synthesized:

| Exam- | | | |
|---|---|---|---|
| ple | | | |
| # | Stucture | m/z | ¹H NMR |
| 206 | 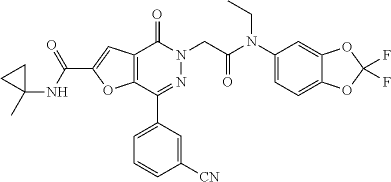 [SMILES: CCN(C(=O)Cn1nc(-c2cccc(C#N)c2)c2oc(C(=O)NC3(C)CC3)cc2c1=O)c1ccc2c(c1)OC(F)(F)O2] | 576.5 [M + 1] | (400 MHz, DMSO): 0.68-0.71 (m, 2H), 0.80 (m, 2H), 1.04 (t, J = 6.8 Hz, 3H), 1.41 (s, 1H), 3.69 (q, J = 6.8 Hz, 2H), 4.81 (s, 2H), 7.34 (d, J = 8.4 Hz, 1H), 7.56 (d, J = 8.4 Hz, 1H), 7.69 (s, 1H), 7.72 (s, 1H), 7.84 (t, J = 8.0 Hz, 1H), 8.05 (d, J = 7.6 Hz, 1H), 8.44 (d, J = 8.0 Hz, 1H), 8.47 (s, 1H), 9.17 (s, 1H) |
| 207 | 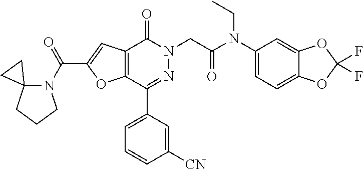 [SMILES: CCN(C(=O)Cn1nc(-c2cccc(C#N)c2)c2oc(C(=O)N3CCCC34CC4)cc2c1=O)c1ccc2c(c1)OC(F)(F)O2] | 602.2 [M + 1] | (400 MHz, DMSO) 0.62 (m, 2H), 1.05 (t, J = 6.8 Hz, 3H), 1.97 (m, 6H), 3.69 (q, J = 6.8 Hz, 2H), 4.05 (m, 2H), 4.82 (s, 2H), 7.36 (d, J = 8.0 Hz, 1H), 7.57 (d, J = 8.4 Hz, 1H), 7.61 (s, 1H), 7.71 (s, 1H), 7.85 (t, J = 8.0 Hz, 1H), 8.06 (d, J = 7.6 Hz, 1H), 8.36 (d, J = 8.0 Hz, 1H), 8.44 (s, 1H) |
| 208 | 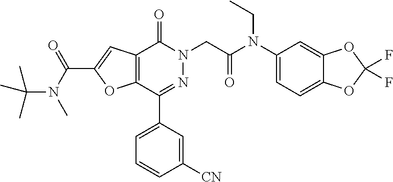 [SMILES: CCN(C(=O)Cn1nc(-c2cccc(C#N)c2)c2oc(C(=O)N(C)C(C)(C)C)cc2c1=O)c1ccc2c(c1)OC(F)(F)O2] | 592 [M + 1] | (400 MHz, DMSO) δ: 1.05 (t, J = 6.8 Hz, 3H), 1.46 (s, 9H), 3.15 (s, 3H), 3.69 (q, J = 6.8 Hz, 2H), 4.82 (s, 2H), 7.36 (d, J = 8.8 Hz, 1H), 7.57 (m, 2H), 7.70 (s, 1H), 7.86 (t, J = 8.0 Hz, 1H), 8.06 (d, J = 8.0 Hz, 1H), 8.36 (d, J = 8.0 Hz, 1H), 8.42 (s, 1H) |
| 209 | 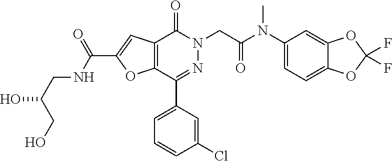 [SMILES: CN(C(=O)Cn1nc(-c2cccc(Cl)c2)c2oc(C(=O)NC[C@@H](O)CO)cc2c1=O)c1ccc2c(c1)OC(F)(F)O2] | 591.5 [M + 1] | (400 MHz, DMSO) δ: 3.30 (s, 3H); 3.35 (m, 2H); 3.40-3.45 (m, 2H); 3.65-3.68 (m, 1H), 4.65 (t, J = 5.6 Hz, 1H), 4.85 (s, 2H); 4.91 (d, J = 4.8 Hz, 1H), 7.41 (m, 1H); 7.55 (d, J = 7.6 Hz, 1H), 7.64-7.78 (m, 4H); 8.08-8.14 (m, 2H); 8.85 (t, J = 6.0 Hz, 1H) |
| 210 | 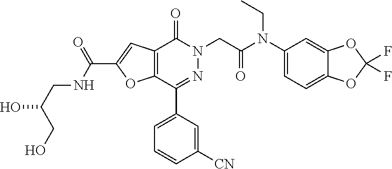 | 569.2 [M + 1] | (400 MHz, DMSO-d₆) δ 1.18 (t, J = 6.8 Hz, 3H), 3.48 (t, J = 5.6 Hz, 2H), 3.68 (q, J = 6.8 Hz, 2H), 3.84 (m, 1H), 4.02- 06 (m, 2H), 4.17 (dd, J = 10.0, 3.6 Hz, 1H), 4.75 (t, J = 5.6 Hz, H), 4.80 (s, 2H), 5.08 (d, J = 4.8 Hz, 1H), 7.25 (s, 1H), 7.34 (d, J = 8.4 Hz, 1H), 7.55 (d, J = 8.4 Hz, 1H), 7.64 (s, 1H), 7.68 (s, 1H), 7.91 (s, 1H), 8.01 (s, 1H), 8.39 (d, J = 2.0 |
| | | | Hz, 1H) |
| 211 | 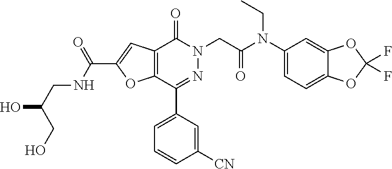 | 569.2 [M + 1] | (400 MHz, DMSO-d₆) δ 1.03 (t, J = 6.4 Hz, 3H), 3.42 (s, 2H), 3.68 (q, J = 6.4 Hz, 2H), 3.84 (m, 2H), 4.04 (m, 1H), 4.16 (m, 1H), 4.80 (s, 2H), 5.10 (m, 1H), 7.24 (s, 1H), 7.34 (d, J = 6.8 Hz, 1H), 7.54 (d, J = 8.0 Hz, 1H), 7.63-7.67 (m, H), 7.91 (s, 1H), 8.01 (s, 1H), 8.38 (s, 1H) |
| 212 | 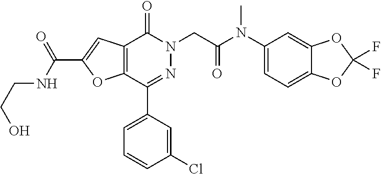 [SMILES: CN(C(=O)Cn1nc(-c2cccc(Cl)c2)c2oc(C(=O)NCCO)cc2c1=O)c1ccc2c(c1)OC(F)(F)O2] | 561.0 [M + 1] | (400 MHz, DMSO) δ: 3.20 (s, 3H), 3.36-3.38 (m, 2H), 3.54 (t, J = 5.6 Hz, 2H), 4.85 (s, 2H), 7.39 (d, J = 8.4 Hz, 1H), 7.55 (d, J = 8.4 Hz, 1H), 7.64-7.67 (m, 2H), 7.75 (d, J = 8.0 Hz, 2H), 8.08 (s, 1H), 8.13-8.14 (m, 1H), 8.92 (t, J = 5.6 Hz, 1H) |

### Example 213

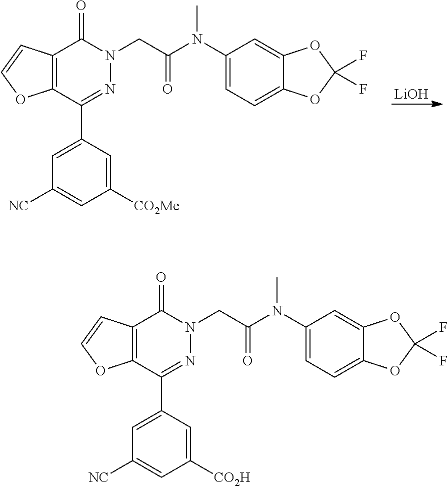

A solution of methyl 3-cyano-5-(5-(2-((2,2-difluorobenzo[d][1,3]dioxol-5-yl)(methyl)amino)-2-oxoethyl)-4-oxo-4,5-dihydrofuro[2,3-d]pyridazin-7-yl)benzoate (0.16 g, 0.30 mmol) in THF (5 mL), water (5 mL) was treated with LiOH (0.50 g, 1.22 mmol). The mixture was stirred at rt overnight and neutralized by using 1N HCl. The resulting slurry was extracted with ethyl acetate. The organic layer was washed with brine, dried, and evaporated. The residue was purified by combiflash (0-100% ethyl acetate in hexane) to give 3-cyano-5-(5-(2-((2,2-difluorobenzo[d][1,3]dioxol-5-yl)(methyl)amino)-2-oxoethyl)-4-oxo-4,5-dihydrofuro[2,3-d]pyridazin-7-yl)benzoic acid (0.036 g, 23%). (MS ESI +ve, 509.2 [M+1]). ¹H NMR: (400 MHz, DMSO) 3.20 (s, 3H), 4.88 (s, 2H), 7.28 (s, 1H), 7.41 (d, J=8.0 Hz, 1H), 7.54 (d, J=8.8 Hz, 1H), 7.74 (s, 1H), 8.45 (s, 2H), 8.69 (s, 1H), 8.88 (s, 1H), 13.87 (br, 1H).

### Example 214

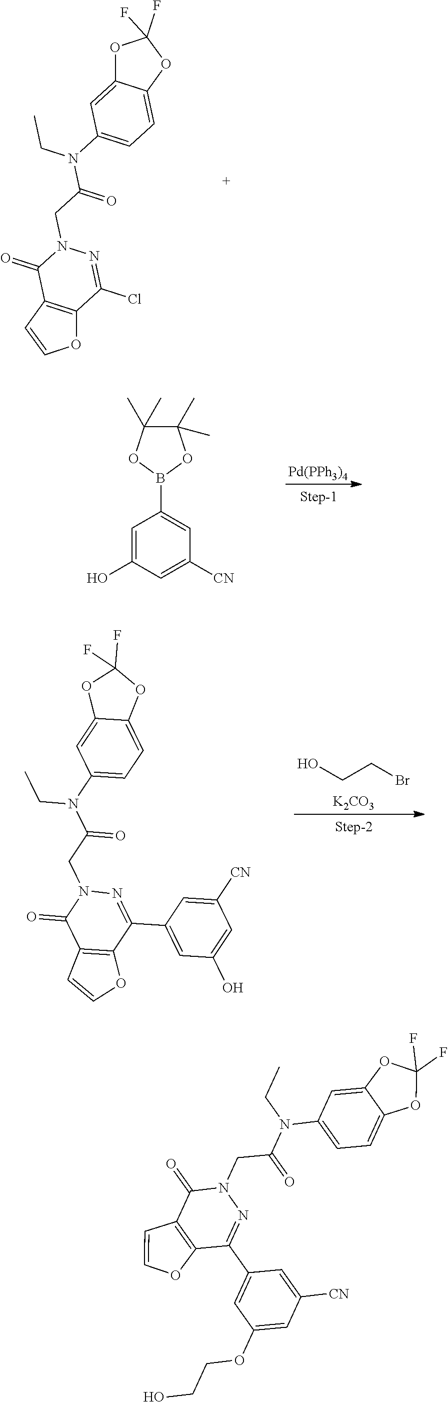

Step-2: A mixture of methyl 2-(7-chloro-4-oxofuro[2,3-d]pyridazin-5(4H)-yl)-N-(2,2-difluorobenzo[d][1,3]dioxol-5-yl)-N-ethylacetamide (0.6 g, 1.46 mmol), 3-hydroxy-5-(4,4,5,5-tetramethyl-1,3,2-dioxaborolan-2-yl)benzonitrile (0.39 g, 1.60 mmol) in ethanol (1.5 mL), toluene (3 mL), water (1 mL), sodium carbonate (0.183 g, 2.19 mmol) was degassed and then Pd(PPh₃)₄ (0.168 g, 2.19 mmol) was added. The resultant reaction was heated 70° C. for 16 h and extracted with ethyl acetate. The combined organic layer was washed with brine, dried over Na₂SO₄, and concentrated in vacuo. The residue was purified by column chromatography (0-20% ethyl acetate in Hexane) to give methyl 2-(7-(3-cyano-5-hydroxyphenyl)-4-oxofuro[2,3-d]pyridazin-5(4H)-yl)-N-(2,2-difluorobenzo[d][1,3]dioxol-5-yl)-N-ethylacetamide (0.75 g, 86%). (MS ESI +Ve 493.2 [M+1]).

Step-2: A mixture of 2-(7-(3-cyano-5-hydroxyphenyl)-4-oxofuro[2,3-d]pyridazin-5(4H)-yl)-N-(2,2-difluorobenzo[d][1,3]dioxol-5-yl)-N-ethylacetamide (0.10 g, 0.20 mmol) in DMF (2 mL), potassium carbonate (0.084 g, 61 mmol), 2-bromoethan-1-ol (0.038 g, 1.5 mmol) was heated 90° C. for 16 h. The mixture was diluted with water, extracted with ethyl acetate. The combined organic layers were washed with brine, dried over sodium sulfate and concentrated in vacuum.

The residue was purified by column chromatography (0-70% ethyl acetate in hexane) to afford 2-(7-(3-cyano-5-(2-hydroxyethoxy)phenyl)-4-oxofuro[2,3-d]pyridazin-5(4H)-yl)-N-(2,2-difluorobenzo[d][1,3]dioxol-5-yl)-N-ethylacetamide (0.03 g, 94%) as off-white solid. (MS ESI +Ve 539.00 [M+1]); ¹H NMR (400 MHz, DMSO-d₆) δ: 1.03 (m, 3H), 3.68 (m, 2H), 3.77 (m, 2H), 4.16 (m, 2H), 4.80 (s, 2H), 4.99 (m, 1H), 7.25 (s, 1H), 7.34 (d, J=8.0 Hz, 1H), 7.55 (d, J=8.0 Hz, 1H), 7.64-7.68 (m, 2H), 7.91 (s, 1H), 8.00 (s, 1H), 8.39 (s, 1H).

### Example 215

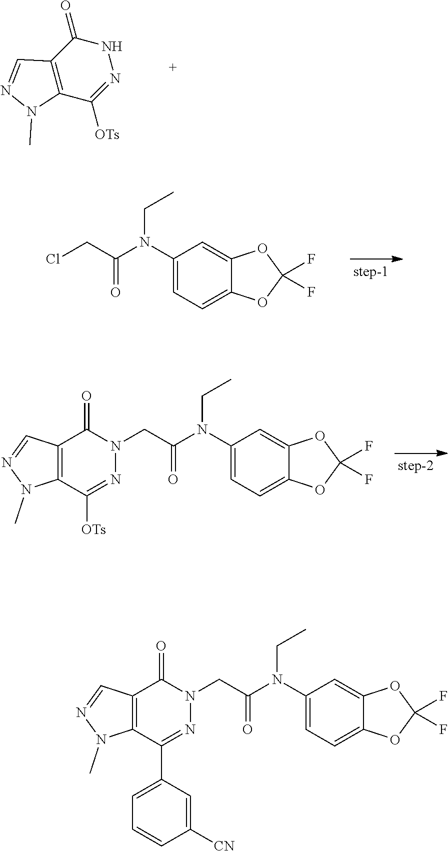 [SMILES: Cc1ccc(S(=O)(=O)Oc2n[nH]c(=O)c3cnn(C)c23)cc1 ; "+" ; CCN(C(=O)CCl)c1ccc2c(c1)OC(F)(F)O2 ; CCN(C(=O)Cn1nc(OS(=O)(=O)c2ccc(C)cc2)c2c(cnn2C)c1=O)c1ccc2c(c1)OC(F)(F)O2 ; CCN(C(=O)Cn1nc(-c2cccc(C#N)c2)c2c(cnn2C)c1=O)c1ccc2c(c1)OC(F)(F)O2]

Step-1: To a solution of 1-methyl-4-oxo-4,5-dihydro-1H-pyrazolo[3,4-d]pyridazin-7-yl 4-methylbenzenesulfonate (1.0 g, 3.1 mmol) in DMF (15 mL) was added LiHMDS (5.67 mL, 4.6 mmol, 1 M in THF) at 0° C. The resultant solution was stirred at 0° C. for 20 min and then a solution of 2-chloro-N-(2,2-difluorobenzo[d][1,3]dioxol-5-yl)-N-ethylacetamide (0.9 g, 3.0 mmol) in DMF (3 mL) was added drop wise. The reaction mixture allowed to warm up to rt and stirred overnight. The reaction was quenched with ice-cold water, extracted with ethyl acetate (3×150 mL). The combined organic layer was washed with brine and dried over sodium sulfate. The solvent was removed under vacuum and the residue was purified by column chromatography (2-3% methanol in dichloromethane) to give 1-methyl-4-oxo-4,5-dihydro-1H-pyrazolo[3,4-d]pyridazin-7-yl 4-methylbenzenesulfonate (0.50 g, 28%). (MS ESI +Ve 562.0 [M+1]).

Step-2: A mixture of 2-(7-chloro-4-oxo-1-(phenylsulfonyl)-1,4-dihydro-5H-pyrrolo[2,3-d]pyridazin-5-yl)-N-(2,2-difluorobenzo[d][1,3]dioxol-5-yl)-N-ethylacetamide (0.10 g, 0.17 mmol) in ethanol (2 mL), toluene (2 mL), water (1 mL), cesium carbonate (0.09 g, 0.089 mmol), (3-cyanophenyl)boronic acid (0.028 g, 0.20 mmol) was degassed and Pd(PPh₃)₄ (0.102 g, 0.089 mmol) was added. The reaction mixture was heated at 90° C. overnight and was concentrated under vacuum. The product was purified by a flash column and then by prep-HPLC to give 2-(7-(3-cyanophenyl)-1-methyl-4-oxo-1,4-dihydro-5H-pyrazolo[3,4-d]pyridazin-5-yl)-N-(2,2-difluorobenzo[d][1,3]dioxol-5-yl)-N-ethylacetamide (0.050 g, 57%) (MS ESI +Ve 493.2.0[M+1]); ¹H NMR: (400 MHz, DMSO) δ:1.02 (t, J=7.2 Hz, 3H), 3.34 (m, 2H), 4.68 (s, 2H), 7.33 (m, 1H), 7.54 (d, J=8.8 Hz, 1H), 7.68 (s, 1H), 7.79 (t, J=8.0 Hz, 1H), 8.00-8.08 (m, 2H), 8.16 (s, 1H), 8.33 (s, 1H).

### Example 216

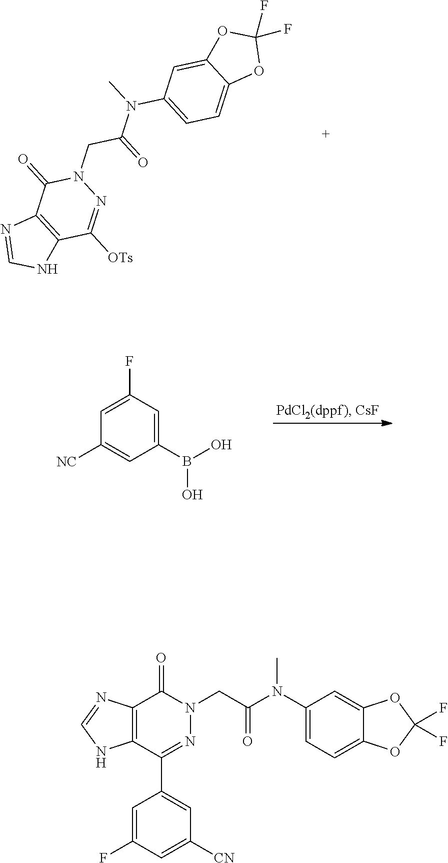 [SMILES: Cc1ccc(S(=O)(=O)Oc2nn(CC(=O)N(C)c3ccc4c(c3)OC(F)(F)O4)c(=O)c3nc[nH]c23)cc1 ; "+" ; N#Cc1cc(F)cc(B(O)O)c1 ; "PdCl2(dppf), CsF" ; CN(C(=O)Cn1nc(-c2cc(F)cc(C#N)c2)c2[nH]cnc2c1=O)c1ccc2c(c1)OC(F)(F)O2]

A mixture of 5-(2-((2,2-difluorobenzo[d][1,3]dioxol-5-yl)(methyl)amino)-2-oxoethyl)-4-oxo-4,5-dihydro-1H-imidazo[4,5-d]pyridazin-7-yl 4-methylbenzenesulfonate (0.10 g, 0.187 mmol) in 1,2-dimethoxyethane (8 mL) and water (1 mL), (3-cyano-5-fluorophenyl)boronic acid (0.037 g, 0.224 mmol), cesium fluoride (0.056 g, 0.374 mmol) was degassed and then PdCl₂(dppf) (0.013 g, 0.0187 mmol) was added. The mixture was degassed again and stirred at 80° C. overnight. The mixture was diluted with water, extracted with ethyl acetate. The organic layer was washed with brine, dried and evaporated. The residue was purified by combiflash (0-62% ethyl acetate in n-hexane) and then subjected to reverse phase preparative HPLC to give 2-(7-(3-cyano-5-fluorophenyl)-4-oxo-1,4-dihydro-5H-imidazo[4,5-d]pyridazin-5-yl)-N-(2,2-difluorobenzo[d][1,3]dioxol-5-yl)-N-methylacetamide (0.025 g, 28%) as off white solid. (MS ESI +Ve 483 [M+1]). ¹H NMR: (400 MHz, DMSO) δ: 3.20 (s, 3H), 4.88 (s, 2H), 7.41 (m, 1H), 7.55 (m, 1H), 7.73 (m, 1H), 8.02 (m, 1H), 8.56 (s, 1H), 8.60 (m, 1H), 8.74 (m, 1H), 14.4 (s, 1H).

In similar fashion, the following examples were synthesized:

| Exam- | | | |
|---|---|---|---|
| ple | | | |
| # | Stucture | m/z | ¹H NMR |
| 217 | 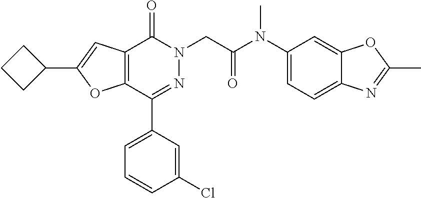 [SMILES: Cc1nc2ccc(N(C)C(=O)Cn3nc(-c4cccc(Cl)c4)c4oc(C5CCC5)cc4c3=O)cc2o1] | 503.8 [M + H] | (400 MHz, DMSO) δ: 1.89- 1.94 (m, 1H), 1.99-2.11 (m, 2H), 2.29-2.33 (m, 1H), 2.37- 2.45 (m, 2H), 3.23 (s, 3H), 4.21 (m, 1H), 4.59 (s, 2H), 7.401 (d, J = 8.0 Hz, 1H), 7.54-7.60 (m, 2H), 7.71-7.78 (m, 3H), 7.91 (s, 1H), 8.64 (s, 1H) |
| 218 | 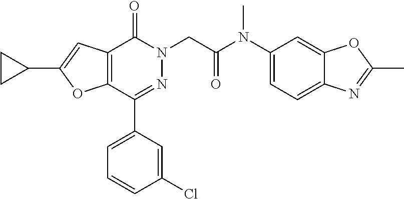 [SMILES: Cc1nc2ccc(N(C)C(=O)Cn3nc(-c4cccc(Cl)c4)c4oc(C5CC5)cc4c3=O)cc2o1] | 489.2 [M + 1] | (400 MHz, DMSO) δ: 1.1 (d, J = 2.8 Hz, 2H), 1.15-1.19 (m, 2H), 2.69 (s, 3H), 2.73 (m, 1H), 3.25 (s, 3H), 4.62 (s, 2H), 7.42 (d, J = 8.0 Hz, 1H), 7.54-7.61 (m, 2H), 7.72- 7.77 (m, 3H), 7.92 (s, 1H), 8.46 (s, 1H) |
| 219 | 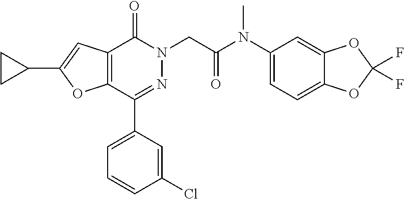 [SMILES: CN(C(=O)Cn1nc(-c2cccc(Cl)c2)c2oc(C3CC3)cc2c1=O)c1ccc2c(c1)OC(F)(F)O2] | 514.0 [M + 1] | (400 MHz, DMSO) δ: 1.11- 1.20 (m, 4H), 2.70 (m, 1H), 3.20 (s, 3H), 4.64 (s, 2H), 7.35 (d, J = 8.0 Hz, 1H), 7.50-7.61 (m, 3H), 7.68 (s, 1H), 7.76-7.79 (m, 2H), 8.47 (s, 1H) |
| 220 | 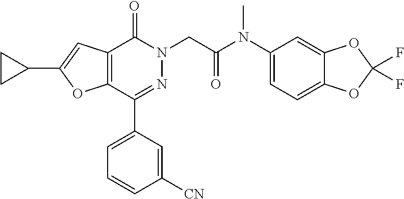 [SMILES: CN(C(=O)Cn1nc(-c2cccc(C#N)c2)c2oc(C3CC3)cc2c1=O)c1ccc2c(c1)OC(F)(F)O2] | 505.2 [M + 1] | (400 MHz, DMSO) δ: 1.12- 1.21 (m, 4H), 2.70 (m, 1H), 3.20 (s, 3H), 4.66 (s, 2H), 7.36 (s, 1H), 7.51 (d, J = 8.0 Hz, 1H), 7.69-7.77 (m, 2H), 8.00 (d, J = 8.0 Hz, 1H), 8.12 (d, J = 8.0 Hz, 1H), 8.18 (s, 1H), 8.57 (s, 1H) |
| 221 | 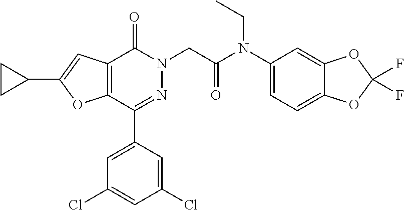 [SMILES: CCN(C(=O)Cn1nc(-c2cc(Cl)cc(Cl)c2)c2oc(C3CC3)cc2c1=O)c1ccc2c(c1)OC(F)(F)O2] | 562.3 [M + H] | (400 MHz, DMSO) δ: 1.03 (t, J = 7.2 Hz, 3H), 1.11 (d, J = 2.4, 2H), 1.20 (d, J = 8.0 Hz, 2H), 2.69 (m, 1H), 3.68 (q, J = 7.2 Hz, 2H), 4.59 (s, 2H), 7.30 (d, J = 7.6 Hz, 1H), 7.52 (d, J = 8.4 Hz, 1H), 7.65 (s, 1H), 7.75-7.80 (m, 3H), 8.53 (s, 1H) |
| 222 | 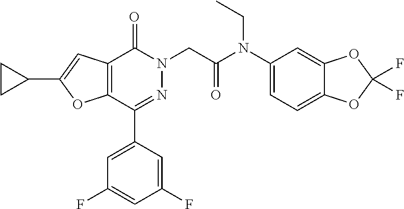 [SMILES: CCN(C(=O)Cn1nc(-c2cc(F)cc(F)c2)c2oc(C3CC3)cc2c1=O)c1ccc2c(c1)OC(F)(F)O2] | 530.2 [M + H] | (400 MHz, DMSO) δ: 1.14- 1.21 (m, 7H), 1.27 (s, 1H,), 2.68 (m, 1H), 3.71 (q, J = 7.2 Hz, 2H), 4.65 (s, 2H), 7.20- 7.31 (m, 2H), 7.40-7.48 (m, 4H), 8.40 (s, 1H) |
| 223 | 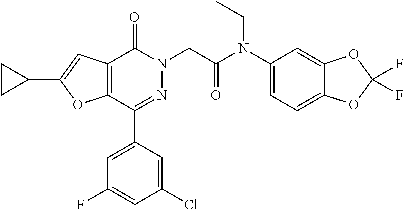 [SMILES: CCN(C(=O)Cn1nc(-c2cc(F)cc(Cl)c2)c2oc(C3CC3)cc2c1=O)c1ccc2c(c1)OC(F)(F)O2] | 546.2 [M + H] | (400 MHz, DMSO) δ: 1.03 (m, 3H), 1.11 (m, 2H,), 1.17- 1.23 (m, 1H), 2.69 (m, 1H), 3.67 (q, J = 6.4 Hz, 2H), 4.58 (s, 1H), 7.29 (d, J = 7.6 Hz, 1H), 7.51 (d, J = 8.0 Hz, 1H), 7.56-7.63 (m, 4H), 8.54 (s, 1H) |
| 224 | 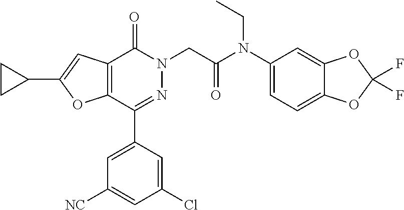 [SMILES: CCN(C(=O)Cn1nc(-c2cc(Cl)cc(C#N)c2)c2oc(C3CC3)cc2c1=O)c1ccc2c(c1)OC(F)(F)O2] | 553.3 [M + H] | (400 MHz, DMSO) δ: 1.03 (t, J = 6.8 Hz, 3H), 1.12 (d, J = 2.4 Hz, 2H), 1.20 (d, J = 8.0 Hz, 2H), 2.69 (m, 1H), 3.67 (q, J = 6.8 Hz, 2H), 4.60 (s, 1H), 7.30 (d, J = 8.4 Hz, 1H), 7.53 (d, J = 8.8 Hz, 1H), 7.65 (s, 1H), 8.09 (s, 1H), 8.16 (s, 1H), 8.22 (s, 1H), 8.63 (s, 1H) |
| 225 | 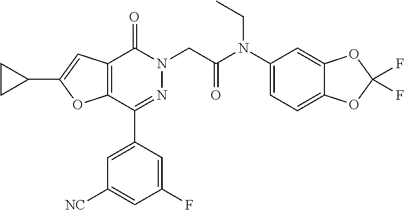 [SMILES: CCN(C(=O)Cn1nc(-c2cc(F)cc(C#N)c2)c2oc(C3CC3)cc2c1=O)c1ccc2c(c1)OC(F)(F)O2] | 537.3 [M + H] | (400 MHz, DMSO) δ: 1.03 (t, J = 6.8 Hz, 3H), 1.12 (d, J = 2.8 Hz, 2H), 1.20 (t, J = 7.6 Hz, 2H), 2.68 (s, 1H), 3.68 (q, J = 6.8 Hz, 2H), 4.60 (s, 2H), 7.30 (d, J = 8.4 Hz, 1H), 7.52 (d, J = 8.4 Hz, 1H), 7.65 (s, 1H), 7.93 (d, J = 9.2 Hz, 1H), 8.04 (m, 2H), 8.63 (s, 1H) |
| 226 | 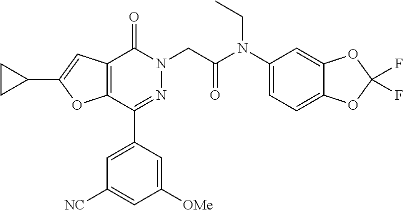 [SMILES: CCN(C(=O)Cn1nc(-c2cc(C#N)cc(OC)c2)c2oc(C3CC3)cc2c1=O)c1ccc2c(c1)OC(F)(F)O2] | 549.2 [M + H] | (400 MHz, DMSO) δ: 1.03 (m, 3H), 1.11 (s, 2H), 1.20 (m, 2H), 2.70 (m, 1H), 3.68 (m, 2H), 3.90 (s, 3H), 4.58 (s, 2H), 7.29 (m, 1H), 7.50- 7.72 (m, 5H), 8.55 (s, 1H) |
| 227 | 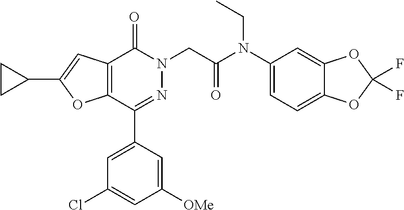 [SMILES: CCN(C(=O)Cn1nc(-c2cc(Cl)cc(OC)c2)c2oc(C3CC3)cc2c1=O)c1ccc2c(c1)OC(F)(F)O2] | 558.4 [M + H] | (400 MHz, DMSO) δ: 1.03 (t, J = 6.8 Hz, 3H), 1.11 (d, J = 2.8 Hz, 2H), 1.19 (d, J = 8.0 Hz, 2H), 2.69 (m, 1H), 3.68 (q, J = 6.8 Hz, 2H), 3.87 (s, 3H), 4.58 (s, 2H), 7.19-7.33 (m, 3H), 7.52 (d, J = 8.4 Hz, 1H), 7.63 (s, 1H), 8.47 (s, 1H) |
| 228 | 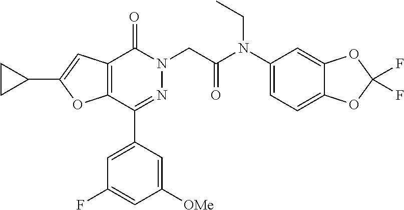 [SMILES: CCN(C(=O)Cn1nc(-c2cc(F)cc(OC)c2)c2oc(C3CC3)cc2c1=O)c1ccc2c(c1)OC(F)(F)O2] | 542.5 [M + H] | (400 MHz, DMSO) δ: 1.03 (t, J = 6.8 Hz, 3H), 1.11 (m, 2H), 1.20 (m, 2H), 2.69 (m, 1H), 3.68 (, J = 6.8 Hz, 2H), 3.87 (s, 3H), 4.58 (s, 2H), 7.01 (d, J = 10.8 Hz, 1H), 7.14 (s, 2H), 7.28 (d, J = −7.6 Hz, 1H), 7.51 (d, J = 8.4 Hz, 1H), 7.62 (s, 1H), 8.48 (s, 1H) |
| 229 | 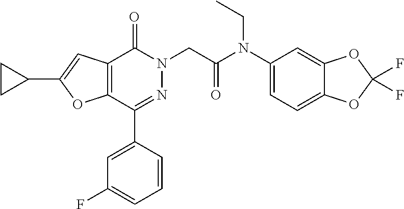 [SMILES: CCN(C(=O)Cn1nc(-c2cccc(F)c2)c2oc(C3CC3)cc2c1=O)c1ccc2c(c1)OC(F)(F)O2] | 512.2 [M + H] | (400 MHz, DMSO) δ: 1.03 (t, J = 6.8 Hz, 3H), 1.11 (d, J = 2.8 Hz, 2H), 1.19 (m, 2H), 2.70 (m, 1H), 3.67 (q, J = 6.8 Hz, 2H), 4.59 (s, 2H), 7.29 (d, J = 6.8 Hz, 1H), 7.38 (m, 1H), 7.50- 7.66 (m, 5H), 8.50 (s, 1H) |
| 230 | 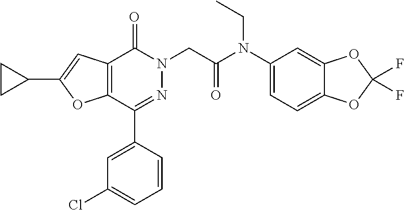 [SMILES: CCN(C(=O)Cn1nc(-c2cccc(Cl)c2)c2oc(C3CC3)cc2c1=O)c1ccc2c(c1)OC(F)(F)O2] | 528.2 [M + H] | (400 MHz, DMSO) δ: 1.03 (t, J = 7.2 Hz, 3H), 1.11 (s, 2H), 1.19 (d, J = 8.4 Hz, 2H), 2.70 (t, J = 8.4 Hz, 1H), 3.68 (q, J = 7.2 Hz, 2H), 4.59 (s, 2H), 7.30 (d, J = 8.4 Hz, 1H), 7.51-7.64 (m, 4H), 7.75-7.79 (m, 2H), 8.48 (s, 1H) |
| 231 | 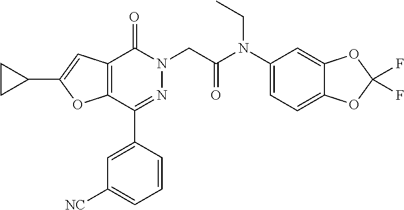 [SMILES: CCN(C(=O)Cn1nc(-c2cccc(C#N)c2)c2oc(C3CC3)cc2c1=O)c1ccc2c(c1)OC(F)(F)O2] | 519.2 [M + H] | (400 MHz, DMSO) δ: 1.02 (t, J = 7.2 Hz, 3H), 1.11 (d, J = 2.8 Hz, 2H), 1.15-1.20 (m, 2H), 2.70 (m, 1H), 3.67 (q, J = 7.2 Hz, 2H), 4.59 (s, 2H), 7.29 (d, J = 7.6 Hz, 1H), 7.51 (d, J = 8.4 Hz, 1H), 7.63 (s, 1H), 7.74 (t, J = 8.0 Hz, 1H), 7.99 (d, J = 7.6 Hz, 1H), 8.10 (d, J = 7.6 Hz, 1H), 8.17 (s, 1H), 8.56 (s, 1H) |
| 232 | 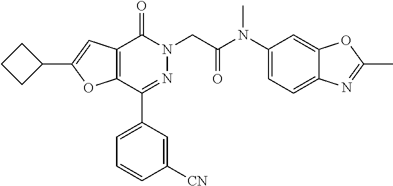 [SMILES: Cc1nc2ccc(N(C)C(=O)Cn3nc(-c4cccc(C#N)c4)c4oc(C5CCC5)cc4c3=O)cc2o1] | 494.2 [M + 1] | (400 MHz, DMSO) δ: 2.05- 2.09 (m, 2H), 2.29-2.47 (m, 4H), 2.69 (s, 3H), 3.25 (s, 3H), 4.23 (m, 1H), 4.61 (s, 2H), 7.41 (d, J = 8.0 Hz, 1H), 7.72-7.77 (m, 2H), 7.90 (s, 1H), 8.00 (d, J = 7.6 Hz, 1H), 8.12 (d, J = 8.0 Hz, 1H), 8.18 (s, 1H), 8.73 (s, 1H) |
| 233 | 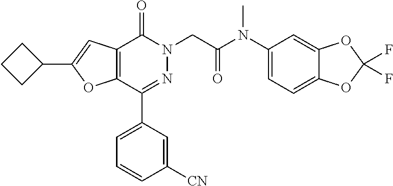 [SMILES: CN(C(=O)Cn1nc(-c2cccc(C#N)c2)c2oc(C3CCC3)cc2c1=O)c1ccc2c(c1)OC(F)(F)O2] | 519.2 [M + 1] | (400 MHz, DMSO) δ: 1.93 (m, 1H), 2.07 (m, 1H), 2.33- 2.47 (m, 4H), 3.18 (s, 3H), 4.22 (m, 1H), 4.64 (s, 2H), 7.34 (d, J = 7.6 Hz, 1H), 7.50 (d, J = 8.0 Hz, 1H), 7.67 (s, 1H), 7.75 (t, J = 8.0 Hz, 1H), 8.00 (d, J = 8.4 Hz, 1H), 8.13 (d, J = 8.0 Hz, 1H), 8.19 (s, 1H), 8.74 (s, 1H) |
| 234 | 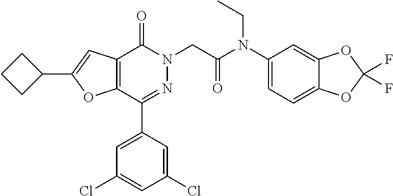 [SMILES: CCN(C(=O)Cn1nc(-c2cc(Cl)cc(Cl)c2)c2oc(C3CCC3)cc2c1=O)c1ccc2c(c1)OC(F)(F)O2] | 578.3 [M + H] | (400 MHz, DMSO-d₆): 1.02 (t, J = 6.8 Hz, 3H), 1.91 (m, 2H), 2.06 (m, 2H), 2.33 (m, 2H), 3.67 (q, J = 6.8 Hz, 2H), 4.22 (m, 1H), 4.58 (s, 2H), 7.29 (d, J = 7.6 Hz, 1H), 7.51 (d, J = 8.4 Hz, 1H), 7.63 (s, 1H), 7.77-7.80 (m, 3H), 8.71 (s, 1H) |
| 235 | 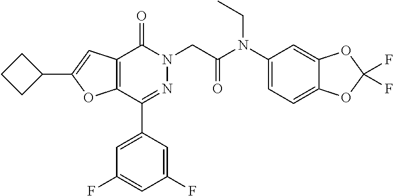 [SMILES: CCN(C(=O)Cn1nc(-c2cc(F)cc(F)c2)c2oc(C3CCC3)cc2c1=O)c1ccc2c(c1)OC(F)(F)O2] | 544.4 [M + H] | (400 MHz, DMSO-d₆): 1.02 (t, J = 6.8 Hz, 3H), 1.92 (m, 1H), 2.07 (m, 1H), 2.32 (m, 2H), 2.44 (m, 2H), 3.67 (q, J = 6.8 Hz, 2H), 4.22 (m, 1H), 4.58 (s, 2H), 7.29 (d, J = 7.6 Hz, 1H), 7.43-7.53 (m, 4H), 7.63 (s, 1H), 8.74 (s, 1H) |
| 236 | 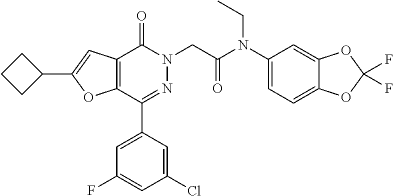 [SMILES: CCN(C(=O)Cn1nc(-c2cc(F)cc(Cl)c2)c2oc(C3CCC3)cc2c1=O)c1ccc2c(c1)OC(F)(F)O2] | 560.4 [M + H] | (400 MHz, DMSO-d₆): 1.03 (t, J = 6.8 Hz, 3H), 1.92 (m, 1H), 2.07 (m, 1H), 2.33 (m, 2H), 2.42 (m, 2H), 3.67 (q, J = 6.8 Hz, 2H), 4.22 (m, 1H), 4.58 (s, 2H), 7.29 (d, J = 7.6 Hz, 1H), 7.52 (d, J = 8.4 Hz, 1H), 7.60-7.66 (m, 4H), 8.73 (s, 1H) |
| 237 | 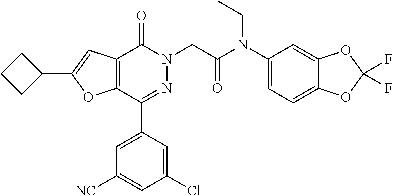 [SMILES: CCN(C(=O)Cn1nc(-c2cc(Cl)cc(C#N)c2)c2oc(C3CCC3)cc2c1=O)c1ccc2c(c1)OC(F)(F)O2] | 567.4 [M + H] | (400 MHz, DMSO-d₆): 1.03 (t, J = 6.8 Hz, 3H), 1.92 (m, 1H), 2.06 (m, 1H), 2.32 (m, 2H), 2.43 (m, 2H), 3.68 (q, J = 6.8 Hz, 2H), 4.23 (m, 1H), 4.60 (s, 2H), 7.30 (d, J = 7.2 Hz, 1H), 7.52 (d, J = 8.4 Hz, 1H), 7.64 (s, 1H), 8.11 (s, 1H), 8.18 (s, 1H), 8.23 (s, 1H), 8.81 (s, 1H) |
| 238 | 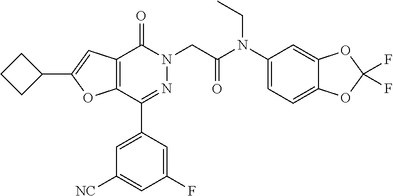 [SMILES: CCN(C(=O)Cn1nc(-c2cc(F)cc(C#N)c2)c2oc(C3CCC3)cc2c1=O)c1ccc2c(c1)OC(F)(F)O2] | 551.4 [M + H] | (400 MHz, DMSO-d₆): 1.02 (m, 3H), 1.92 (m, 1H), 2.06 (m, 1H), 2.32 (m, 2H), 2.41 (m, 2H), 3.66 (m, 2H), 4.22 (m, 1H), 4.58 (s, 2H), 7.29 (m, 1H), 7.51 (m, 1H), 7.63 (s, 1H), 7.94-8.06 (m, 3H), 8.80 (s, 1H) |
| 239 | 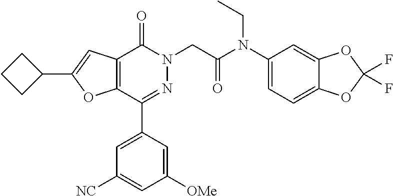 [SMILES: CCN(C(=O)Cn1nc(-c2cc(C#N)cc(OC)c2)c2oc(C3CCC3)cc2c1=O)c1ccc2c(c1)OC(F)(F)O2] | 563.2 [M + H] | (400 MHz, DMSO) δ: 1.02 (t, J = 7.2 Hz, 3H), 1.91 (m, 1H), 2.08 (m, 1H), 2.31 (m, 2H), 2.42 (m, 2H), 3.67 (q, J = 7.2 Hz, 2H), 3.91 (s, 3H), 4.21 (m, 1H), 4.58 (s, 2H), 7.28 (d, J = 9.6 Hz, 1H), 7.51 (d, J = 8.4 Hz, 1H), 7.58-7.62 (m, 1H), 7.61 (s, 2H), 7.74 (s, 1H), 8.73 (s, 1H) |
| 240 | 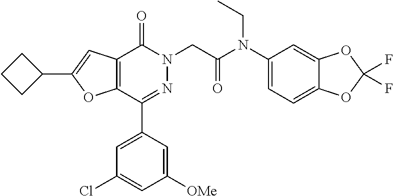 [SMILES: CCN(C(=O)Cn1nc(-c2cc(Cl)cc(OC)c2)c2oc(C3CCC3)cc2c1=O)c1ccc2c(c1)OC(F)(F)O2] | 572.5 [M + H] | (400 MHz, DMSO): 1.02 (t, J = 7.2 Hz, 3H), 1.91 (m, 1H), 2.05 (m, 1H), 2.31 (m, 2H), 2.41 (m, 2H), 3.67 (q, J = 7.2 Hz, 2H), 3.87 (s, 3H), 4.21 (m, 1H), 4.56 (s, 2H), 7.20 (d, J = 2.0 Hz, 1H), 7.28-7.25 (m, 2H), 7.34 (s, 1H), 7.51 (d, J = 8.4 Hz, 1H), 7.61 (s, 1H), 8.64 (s, 1H) |
| 241 | 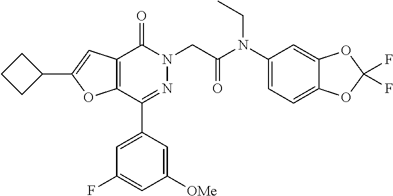 [SMILES: CCN(C(=O)Cn1nc(-c2cc(F)cc(OC)c2)c2oc(C3CCC3)cc2c1=O)c1ccc2c(c1)OC(F)(F)O2] | 556.5 [M + H] | (400 MHz, DMSO) 1.03 (t, J = 6.8 Hz, 3H), 1.94 (m, 1H), 2.07 (m, 1H), 2.32 (m, 2H), 2.44 (m, 2H), 3.68 (q, J = 6.8 Hz, 2H), 3.87 (s, 3H), 4.22 (m, 1H), 4.57 (s, 2H), 7.02 (d, J = 11.2 Hz, 1H), 7.13-7.16 (m, 2H), 7.28 (d, J = 8.0 Hz, 1H), 7.51 (d, J = 8.4 Hz, 1H), 7.61 (s, 1H), 8.66 (s, 1H) |
| 242 | 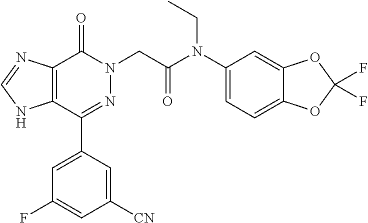 [SMILES: CCN(C(=O)Cn1nc(-c2cc(F)cc(C#N)c2)c2[nH]cnc2c1=O)c1ccc2c(c1)OC(F)(F)O2] | 497.0 [M + H] | (400 MHz, DMSO) δ: 1.05 (t, J = 7.2 Hz, 3H), 3.69 (m, 2H), 4.82 (s, 2H), 7.35 (d, J = 8.8 Hz, 1H), 7.54 (d, J = −8.4 Hz, 1H), 7.67 (s, 1H), 8.02 (d, J = 8.4 Hz, 1H), 8.53 (s, 1H), 8.62 (d, J = 8.4 Hz, 1H), 8.73 (s, 1H), 14.35 (br, 1H) |
| 243 | 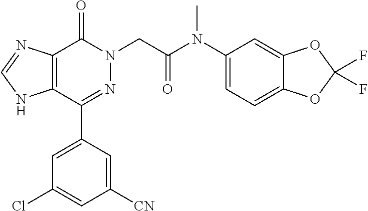 [SMILES: CN(C(=O)Cn1nc(-c2cc(Cl)cc(C#N)c2)c2[nH]cnc2c1=O)c1ccc2c(c1)OC(F)(F)O2] | 499.0 [M + 1] | (400 MHz, DMSO) δ: 3.20 (s, 3H) 4.88 (s, 2H), 7.41 (m, 1H), 7.54 (d, J = 8.4 Hz, 1H), 7.74 (m, 1H), 8.19 (s, 1H), 8.56 (s, 1H), 8.83 (m, 2H), 14.37 (br, 1H) |
| 244 | 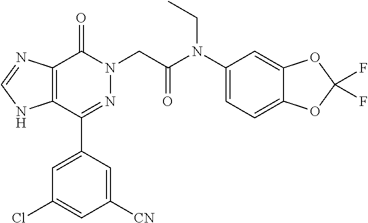 [SMILES: CCN(C(=O)Cn1nc(-c2cc(Cl)cc(C#N)c2)c2[nH]cnc2c1=O)c1ccc2c(c1)OC(F)(F)O2] | 513.0 [M + 1] | (400 MHz DMSO) δ: 1.05 (t, J = 7.2 Hz, 3H), 3.69 (s, 2H), 4.83 (s, 2H), 7.35 (d, J = 9.2 Hz, 1H), 7.54 (d, J = 9.2 Hz, 1H), 7.68 (s, 1H), 8.19 (s, 1H), 8.57 (s, 1H), 8.83 (m, 2H), 14.45 (br, 1H) |
| 245 | 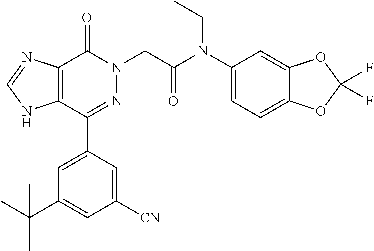 [SMILES: CCN(C(=O)Cn1nc(-c2cc(C#N)cc(C(C)(C)C)c2)c2[nH]cnc2c1=O)c1ccc2c(c1)OC(F)(F)O2] | 535.4 [M + 1] | (400 MHz, DMSO) δ: 1.05 (m, 3H), 1.38 (s, 9H), 3.69 (s, 2H), 4.71 (s, 2H), 7.34 (m, 1H), 7.51-7.53 (d, J = 8.2 Hz, 1H), 7.65 (s, 1H), 7.87-7.91 (m, 2H), 8.79 (s, 1H), 8.99 (s, 1H) |
| 246 | 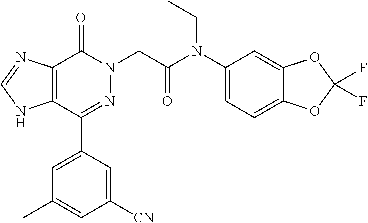 [SMILES: CCN(C(=O)Cn1nc(-c2cc(C)cc(C#N)c2)c2[nH]cnc2c1=O)c1ccc2c(c1)OC(F)(F)O2] | 493.4 [M + H] | (400 MHz, DMSO) δ: 1.05 (t, J = 7.2 Hz, 3H), 2.34 (s, 3H), 3.68-3.70 (q, J = 7.2 Hz, 2H), 4.81 (s, 2H), 7.34- 7.36 (d, J = 7.2 Hz, 1H), 7.54-7.56 (d, J = 8.4 Hz, 1H), 7.68 (s, 1H), 7.81 (s, 1H), 8.54 (m, 2H), 8.65 (m, 1H), 14.28 (br, 1H) |
| 247 | 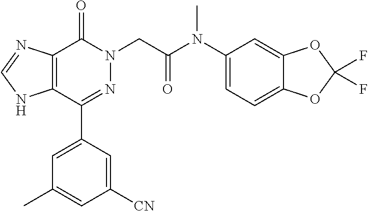 [SMILES: Cc1cc(C#N)cc(-c2nn(CC(=O)N(C)c3ccc4c(c3)OC(F)(F)O4)c(=O)c3nc[nH]c23)c1] | 479.5 [M + H] | (400 MHz, DMSO): 2.47 (s, 3H), 3.20 (s, 3H), 4.86 (s, 2H), 7.41 (m, 1H), 7.52-7.54 (d, J = 7.6 Hz, 1H), 7.73 (m, 1H), 7.80 (m, 1H), 8.55 (m, 2H), 8.66 (m, 1H), 14.29 (br, 1H) |
| 248 | 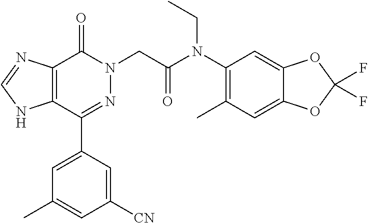 [SMILES: CCN(C(=O)Cn1nc(-c2cc(C)cc(C#N)c2)c2[nH]cnc2c1=O)c1cc2c(cc1C)OC(F)(F)O2] | 507.2 [M + H] | (400 MHz, DMSO): 1.07- 1.09 (m, 3H), 2.40 (s, 3H), 2.47 (s, 3H), 3.19-3.26 (q, J = 7.0 Hz, 1H), 3.98-4.03 (q, J = 7.0 Hz, 1H,), 4.53- 4.59 (m, 1H), 4.76-4.80 (m, 1H), 7.55 (s, 1H), 7.64 (s, 1H), 7.81 (s, 1H), 8.55 (m, 1H), 8.58 (m, 1H), 8.68 (s, 1H), 14.31 (br, 1H) |
| 249 | 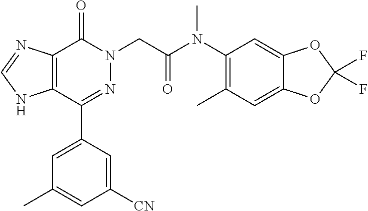 [SMILES: Cc1cc(C#N)cc(-c2nn(CC(=O)N(C)c3cc4c(cc3C)OC(F)(F)O4)c(=O)c3nc[nH]c23)c1] | 493.2 [M + H] | (400 MHz, DMSO): 2.40 (s, 3H), 2.47 (s, 3H), 3.12 (s, 3H), 4.59-4.63 (m, 1H), 4.78- 4.82 (m, 1H), 7.54 (s, 1H), 7.70 (s, 1H), 7.81 (s, 1H), 8.54-8.57 (m, 2H), 8.67 (s, 1H), 14.32 (br, 1H) |
| 250 | 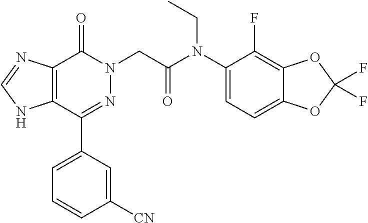 [SMILES: CCN(C(=O)Cn1nc(-c2cccc(C#N)c2)c2[nH]cnc2c1=O)c1ccc2c(c1F)OC(F)(F)O2] | 497.2 [M + H] | (400 MHz, DMSO): 1.04 (t, J = 7.2 Hz, 3H), 3.66 (q, J = 7.2 Hz, 2H), 4.76-4.80 (d, J = 16 Hz, 1H), 4.87-4.91 (d, J = 16 Hz, 1H), 7.76- 7.85 (m, 3H), 7.99 (d, J = 7.2 Hz, 1H), 8.56 (s, 1H), 8.68 (m, 1H), 8.82 (m, 1H), 14.29 (br, 1H) |
| 251 | 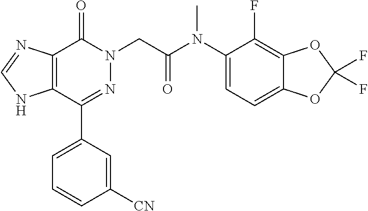 [SMILES: CN(C(=O)Cn1nc(-c2cccc(C#N)c2)c2[nH]cnc2c1=O)c1ccc2c(c1F)OC(F)(F)O2] | 483.0 [M + H] | (400 MHz, DMSO): 3.17 (s, 3H), 4.80-4.85 (d, J = 16.4 Hz, 1H), 4.92-4.96 (d, J = 16.4 Hz, 1H), 7.78-7.81 (m, 2H), 7.90-7.92 (d, J = 6.0 Hz, 1H), 7.97-7.99 (d, J = 7.2 Hz, 1H), 8.53 (s, 1H), 8.69-8.71 (d, J = 7.2 Hz, 1H), 8.83 (s, 1H), 14.31 (br, 1H) |
| 252 | 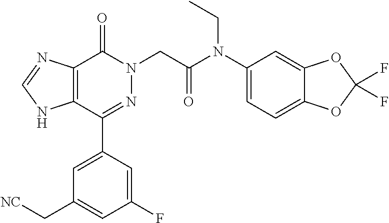 [SMILES: CCN(C(=O)Cn1nc(-c2cc(F)cc(CC#N)c2)c2[nH]cnc2c1=O)c1ccc2c(c1)OC(F)(F)O2] | 511 [M + 1] | (400 MHz, DMSO) δ: 1.03 (t, J = 6.4 Hz, 3H); 3.68 (d, J = 6.4 Hz, 2H); 4.21 (s, 2H); 4.79 (s, 2H); 7.34 (d, J = 8.4 Hz, 2H); 7.53 (d, J = 8.4 Hz, 1H); 7.66 (s, 1H); 8.28 (m, 2H); 8.54 (s, 1H); 14.2 (s, 1H) |
| 253 | 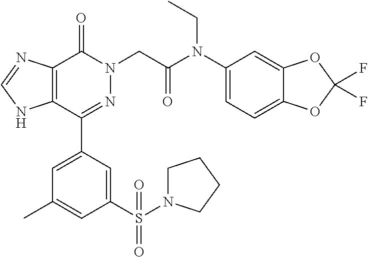 [SMILES: CCN(C(=O)Cn1nc(-c2cc(C)cc(S(=O)(=O)N3CCCC3)c2)c2[nH]cnc2c1=O)c1ccc2c(c1)OC(F)(F)O2] | 601 [M + 1] | (400 MHz, DMSO) δ: 1.04 (t, J = 6.4 Hz, 3H); 1.62- 1.69 (m, 4H); 3.20 (m, 4H), 3.65 (q, J = 6.4 Hz, 2H); 4.80 (s, 2H); 7.34 (d, J = 8.4 Hz, 1H); 7.54 (d, J = 8.0 Hz, 1H); 7.66 (s, 1H); 7.73 (s, 1H); 8.46 (s, 1H); 8.57 (s, 1H); 8.75 (s, 1H), 14.26 (br, 1H) |
| 254 | 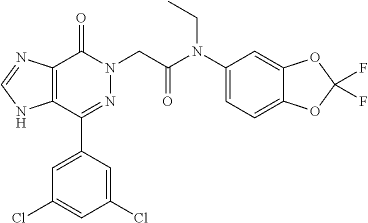 [SMILES: CCN(C(=O)Cn1nc(-c2cc(Cl)cc(Cl)c2)c2[nH]cnc2c1=O)c1ccc2c(c1)OC(F)(F)O2] | 522 [M + 1] | (400 MHz, DMSO) δ: 1.05 (s, 3H); 3.69 (m, 2H); 4.83 (s, 2H); 7.36 (m, 1H); 7.54 (m, 1H); 7.68 (m, 1H); 7.77 (s, 1H); 8.51-8.56 (m, 3H); 14.29 (br, 1H) |
| 255 | 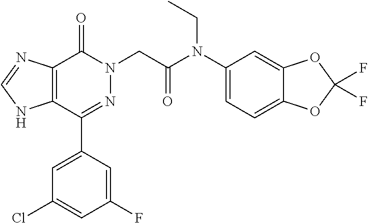 [SMILES: CCN(C(=O)Cn1nc(-c2cc(F)cc(Cl)c2)c2[nH]cnc2c1=O)c1ccc2c(c1)OC(F)(F)O2] | 504 [M − 1] | (400 MHz, DMSO) δ: 1.05 (m, 3H); 3.70 (d, J = 6.8 Hz, 2H); 4.79 (s, 2H); 7.35 (m, 1H), (s, 2H); 7.339- 7.361 (d, J = 8.8, 1H); 7.53- 7.72 (m, 3H); 8.28 (m, 1H), 8.42 (m, 1H), 8.56 (s, 1H), 14.29 (br, 1H) |
| 256 | 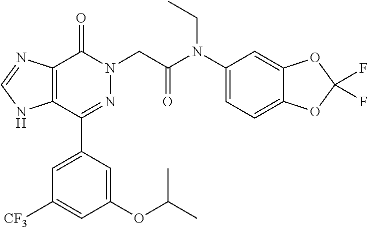 [SMILES: CCN(C(=O)Cn1nc(-c2cc(OC(C)C)cc(C(F)(F)F)c2)c2[nH]cnc2c1=O)c1ccc2c(c1)OC(F)(F)O2] | 580 [M + 1] | (400 MHz, DMSO) δ: 1.03 (t, J = 6.8 Hz, 3H); 1.34 (d, J = 6.0 Hz, 6H); 3.16 (m, 1H); 3.67 (d, J = 6.8 Hz, 2H); 4.78 (m, 2H); 7.32 (m, 2H); 7.51 (d, J = 8.4 Hz, 1H); 7.63 (s, 1H); 8.30 (m, 2H); 8.53 (s, 1H). |
| 257 | 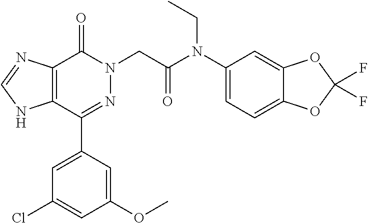 [SMILES: CCN(C(=O)Cn1nc(-c2cc(Cl)cc(OC)c2)c2[nH]cnc2c1=O)c1ccc2c(c1)OC(F)(F)O2] | 518.0 [M + H] | (400 MHz, DMSO-d₆): 1.05 (t, J = 7.2 Hz, 3H), 3.68 (q, J = 7.2 Hz, 2H), 3.86 (s, 3H), 4.78 (s, 2H), 7.15 (s, 1H), 7.33 (d, J = 7.2 Hz, 1H), 7.53 (d, J = 8.4 Hz, 1H), 7.65 (s, 1H), 8.04 (m, 2H), 8.47 (s, 1H), 14.08 (br, 1H) |
| 258 | 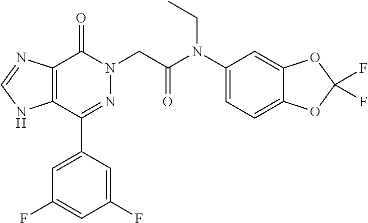 [SMILES: CCN(C(=O)Cn1nc(-c2cc(F)cc(F)c2)c2[nH]cnc2c1=O)c1ccc2c(c1)OC(F)(F)O2] | 490 [M + 1] | (400 MHz, DMSO-d₆): 1.05 (t, J = 6.4 Hz, 3H), 3.69 (q, J = 6.4 Hz, 2H), 4.81 (s, 2H), 7.33-7.43 (m, 2H), 7.54 (m, 1H), 7.68 (s, 1H), 8.17 (m, 2H), 8.53 (s, 1H), 14.00 (br, 1H). |
| 259 | 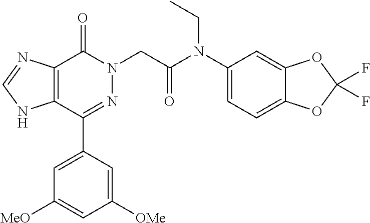 [SMILES: CCN(C(=O)Cn1nc(-c2cc(OC)cc(OC)c2)c2[nH]cnc2c1=O)c1ccc2c(c1)OC(F)(F)O2] | 514 [M + 1] | (400 MHz, DMSO) δ: 1.05 (t, J = 6.4 Hz, 3H); 3.707 (q, J = 6.4 Hz, 2H); 3.84 (s, 6H); 4.79 (s, 2H); 6.65 (s, 1H); 7.34 (d, J = 7.6 Hz, 1H); 7.50-7.59 (m, 4H); 8.51 (s, 1H); 14.18 (br, 1H) |
| 260 | 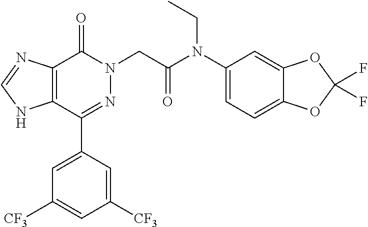 [SMILES: CCN(C(=O)Cn1nc(-c2cc(C(F)(F)F)cc(C(F)(F)F)c2)c2[nH]cnc2c1=O)c1ccc2c(c1)OC(F)(F)O2] | 590 [M + 1] | (400 MHz, DMSO) δ: 1.04 (t, J = 6.8 Hz, 3H); 3.68 (q, J = 6.8 Hz, 2H); 4.86 (s, 2H); 7.32 (d, J = 7.6 Hz, 1H); 7.51 (d, J = 8.4 Hz, 1H); 7.63 (s, 1H); 8.26 (s, 1H); 8.60 (s, 1H); 9.12 (s, 2H) 14.35 (br, 1H) |
| 261 | 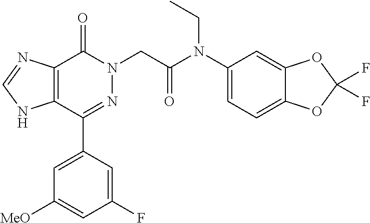 [SMILES: CCN(C(=O)Cn1nc(-c2cc(F)cc(OC)c2)c2[nH]cnc2c1=O)c1ccc2c(c1)OC(F)(F)O2] | 502 [M + 1] | (400 MHz, DMSO) δ: 1.03 (t, J = 6.8 Hz, 3H); 3.68 (q, J = 6.8 Hz, 2H); 3.85 (s, 3H); 4.78 (s, 2H); 6.97 (d, J = 10.8 Hz, 1H); 7.32 (d, J = 8.0 Hz, 1H); 7.512 (d, J = 8.4 Hz, 1H); 7.66 (s, 1H); 7.80 (m, 1H); 7.93 (m, 1H); 8.51 (s, 1H); 14.21 (br, 1H) |
| 262 | 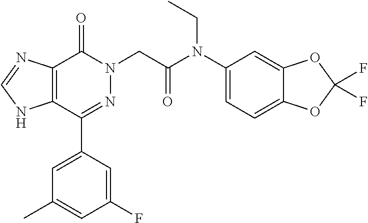 [SMILES: CCN(C(=O)Cn1nc(-c2cc(C)cc(F)c2)c2[nH]cnc2c1=O)c1ccc2c(c1)OC(F)(F)O2] | 486 [M + 1] | (400, DMSO) δ: 1.03 (t, J = 6.8, 3H); 2.33 (s, 3H); 3.66 (q, J = 6.8 Hz, 2H); 4.77 (s, 2H); 7.16 (d, J = 9.6 Hz, 1H); 7.33 (d, J = 8.0 Hz, 1H); 7.53 (d, J = 8.4 Hz, 1H); 7.66 (m, 1H); 8.02 (d, J = 9.6 Hz, 1H); 8.076 (s, 1H); 8.47 (s, 1H) |
| 263 | 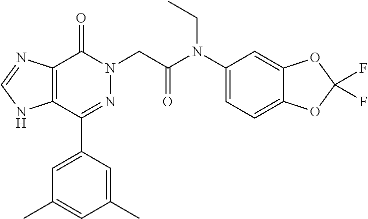 [SMILES: CCN(C(=O)Cn1nc(-c2cc(C)cc(C)c2)c2[nH]cnc2c1=O)c1ccc2c(c1)OC(F)(F)O2] | 482 [M + 1] | (400 MHz, DMSO) δ: 1.03 (t, J = 6.8 Hz, 3H); 2.36 (s, 6H); 3.67 (q, J = 6.8 Hz, 2H); 4.76 (s, 2H); 7.11 (s, 1H); 7.33 (d, J = 8.4 Hz, 1H); 7.54 (d, J = 8.4 Hz, 1H); 7.66 (s, 1H); 7.97 (m, 2H); 8.45 (s, 1H); 14.09 (br, 1H) |
| 264 | 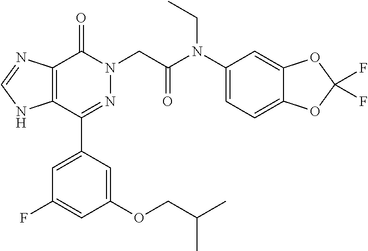 [SMILES: CCN(C(=O)Cn1nc(-c2cc(F)cc(OCC(C)C)c2)c2[nH]cnc2c1=O)c1ccc2c(c1)OC(F)(F)O2] | 544 [M + 1] | (400 MHz, DMSO) δ: 1.00- 1.05 (m, 9H), 2.06 (m, 1H), 3.68 (q, J = 6.8 Hz, 2H); 3.84 (d, J = 6.4 Hz, 2H), 4.79 (s, 2H), 6.96 (m, 1H), 7.32 (d, J = 9.2 Hz, 1H), 7.52 (d, J = 8.4 Hz, 1H), 7.65 (s, 1H), 7.81 (d, J = 10 Hz, 1H), 7.95 (s, 1H), 8.53 (s, 1H), 14.23 (br, 1H) |
| 265 | 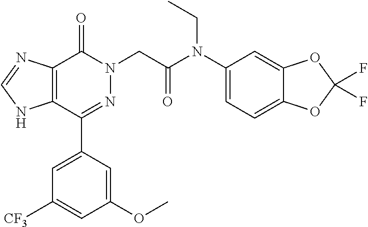 [SMILES: CCN(C(=O)Cn1nc(-c2cc(OC)cc(C(F)(F)F)c2)c2[nH]cnc2c1=O)c1ccc2c(c1)OC(F)(F)O2] | 552 [M + 1] | (400 MHz, DMSO) δ: 1.03 (t, J = 7.2 Hz, 3H); 3.68 (d, J = 7.2 Hz, 2H); 3.93 (s, 3H); 4.81 (s, 2H); 7.32 (d, J = 8.4 Hz, 1H); 7.37 (s, 1H), 7.52 (d, J = 8.4 Hz, 1H); 7.65 (s, 1H); 8.31 (m, 1H); 8.39 (m, 1H); 8.55 (s, 1H); 14.23 (br, 1H) |
| 266 | 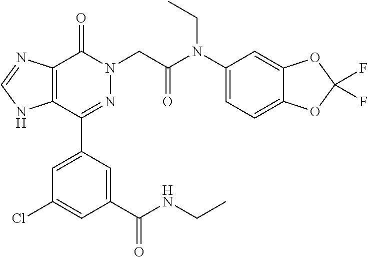 [SMILES: CCNC(=O)c1cc(Cl)cc(-c2nn(CC(=O)N(CC)c3ccc4c(c3)OC(F)(F)O4)c(=O)c3nc[nH]c23)c1] | 559 [M + 1] | (400 MHz, DMSO) δ: 1.05 (t, J = 6.8 Hz, 3H); 1.17 (t, J = 7.2 Hz, 3H); 3.32 (m, 2H); 3.70 (q, J = 6.8 Hz, 2H); 4.82 (s, 2H); 7.37 (d, J = 7.2 Hz, 1H); 7.56 (d, J = 8.8 Hz, 1H); 7.695 (s, 1H); 7.70 (s, 1H); 8.47 (s, 1H); 8.58-8.80 (m, 3H); 14.26 (br, 1H) |
| 267 | 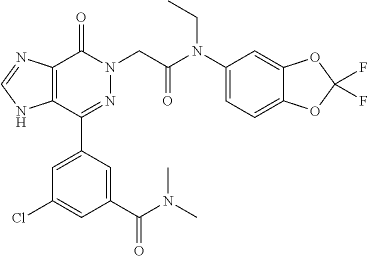 [SMILES: CCN(C(=O)Cn1nc(-c2cc(Cl)cc(C(=O)N(C)C)c2)c2[nH]cnc2c1=O)c1ccc2c(c1)OC(F)(F)O2] | 559 [M + 1] | (400 MHz, DMSO) δ: 1.03 (t, J = 6.8 Hz, 3H); 2.95 (s, 3H); 3.02 (s, 3H); 3.68 (q, J = 6.8 Hz, 2H); 4.80 (s, 2H); 7.34 (d, J = 8.4 Hz, 1H); 7.53 (d, J = 8.8 Hz, 1H); 7.59 (s, 1H); 7.67 (s, 1H); 8.43 (s, 1H); 8.52 (m, 2H); 14.26 (br, 1H) |
| 268 | 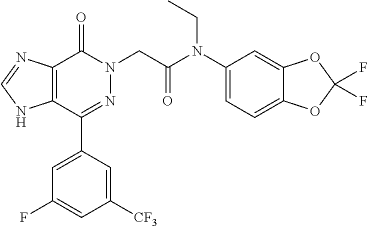 [SMILES: CCN(C(=O)Cn1nc(-c2cc(F)cc(C(F)(F)F)c2)c2[nH]cnc2c1=O)c1ccc2c(c1)OC(F)(F)O2] | 540 [M + 1] | (400 MHz, DMSO) δ: 1.05 (t, J = 6.8 Hz, 3H); 3.69 (q, J = 6.8 Hz, 2H); 4.82 (s, 2H); 7.34 (d, J = 8.4 Hz, 1H); 7.53 (d, J = 8.4 Hz, 1H); 7.66 (s, 1H); 7.83 (d, J = 8.0 Hz, 1H); 8.45 (s, 1H); 8.61 (d, J = 9.6 Hz, 1H); 8.77 (s, 1H) |
| 269 | 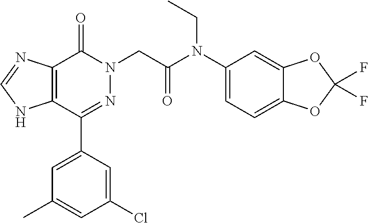 [SMILES: CCN(C(=O)Cn1nc(-c2cc(C)cc(Cl)c2)c2[nH]cnc2c1=O)c1ccc2c(c1)OC(F)(F)O2] | 502.3 [M + H] | (400 MHz, DMSO-d₆): 1.05 (m, 3H), 2.43 (s, 3H), 3.70 (q, J = 6.4 Hz, 2H), 4.81 (s, 2H), 7.36 (m, 1H), 7.41 (s, 1H), 7.54-7.56 (d, J = 7.2 Hz, 1H), 7.68 (m, 1H), 8.22 (m, 1H), 8.33 (s, 1H), 8.53 (s, 1H), 14.24 (bs, 1H) |
| 270 | 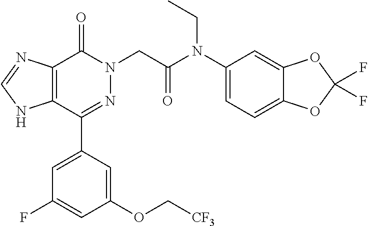 [SMILES: CCN(C(=O)Cn1nc(-c2cc(F)cc(OCC(F)(F)F)c2)c2[nH]cnc2c1=O)c1ccc2c(c1)OC(F)(F)O2] | 570.0 [M + 1] | (400 MHz, DMSO) δ: 1.03 (t, J = 6.8 Hz, 3H), 3.67 (q, J = 6.8 Hz, 2H), 4.77 (s, 2H), 4.88 (q, J = 8.8 Hz, 2H), 7.15 (d, J = 6.8 Hz, 1H), 7.33 (d, J = 8.4 Hz, 1H), 7.52 (d, J = 8.4 Hz, 1H), 7.66 (s, 1H), 7.96 (d, J = 10.0 Hz, 1H), 8.01 (s, 1H), 8.42 (s, 1H) |
| 271 | 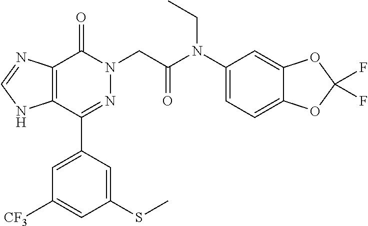 [SMILES: CCN(C(=O)Cn1nc(-c2cc(SC)cc(C(F)(F)F)c2)c2[nH]cnc2c1=O)c1ccc2c(c1)OC(F)(F)O2] | 568.2 [M + 1] | (400 MHz, DMSO) δ: 1.03 (s, J = 7.2 Hz, 3H), 2.67 (s, 3H), 3.68 (q, J = 7.2 Hz, 2H), 4.81 (s, 2H), 7.33 (d, J = 8.4 Hz, 1H), 7.52 (d, J = 8.4 Hz, 1H), 7.65 (s, 2H), 8.54 (s, 2H), 8.61 (m, 1H), 14.23 (br, 1H) |
| 272 | 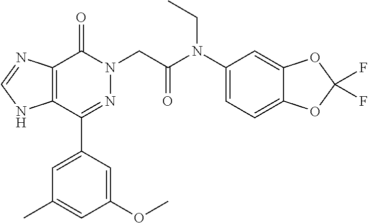 [SMILES: CCN(C(=O)Cn1nc(-c2cc(C)cc(OC)c2)c2[nH]cnc2c1=O)c1ccc2c(c1)OC(F)(F)O2] | 498.0 [M + 1] | (400 MHz, DMSO) δ: 1.03 (m, 3H), 2.37 (s, 3H), 3.67 (q, J = 6.4 Hz, 2H), 3.80 (s, 3H), 4.76 (s, 2H), 6.88 (s, 1H), 7.32 (d, J = 8.0 Hz, 2H), 7.51 (d, J = 8.0 Hz, 1H), 7.64 (s, 1H), 7.45 (m, 1H), 8.47 (s, 1H) |
| 273 | 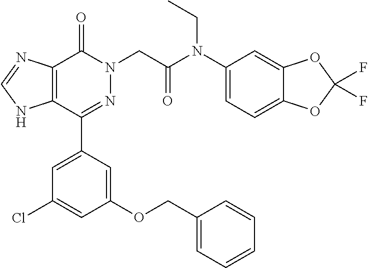 [SMILES: CCN(C(=O)Cn1nc(-c2cc(Cl)cc(OCc3ccccc3)c2)c2[nH]cnc2c1=O)c1ccc2c(c1)OC(F)(F)O2] | 594.2 [M + 1] | (400 MHz, DMSO) δ: 1.05 (m, 3H), 3.69 (m, 2H), 4.80 (s, 2H), 5.22 (s, 2H), 7.27- 7.67 (m, 9H), 8.11 (s, 2H), 8.52 (s, 1H), 14.12 (br, 1H) |
| 274 | 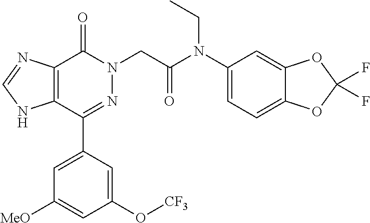 [SMILES: CCN(C(=O)Cn1nc(-c2cc(OC)cc(OC(F)(F)F)c2)c2[nH]cnc2c1=O)c1ccc2c(c1)OC(F)(F)O2] | 568 [M + 1] | (400 MHz, DMSO) δ: 1.03 (t, J = 6.8 Hz, 3H); 3.67 (q, J = 6.8 Hz, 2H); 3.87 (s, 3H); 4.70 (s, 2H); 6.96 (s, 1H); 7.31 (d, J = 7.6 Hz, 1H); 7.51 (d, J = 8.4 Hz, 1H); 7.63 (s, 1H); 7.98 (s, 1H); 8.16 (s, 1H); 8.25 (s, 1H) |
| 275 | 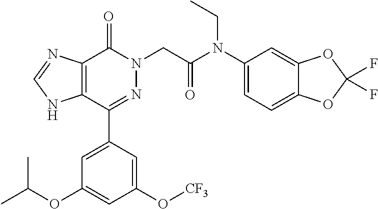 [SMILES: CCN(C(=O)Cn1nc(-c2cc(OC(C)C)cc(OC(F)(F)F)c2)c2[nH]cnc2c1=O)c1ccc2c(c1)OC(F)(F)O2] | 596 [M + 1] | (400 MHz, DMSO) δ: 1.03 (m, 3H); 1.32 (d, J = 5.6 Hz, 6H); 3.67 (q, J = 6.0 Hz, 2H); 4.74 (m, 1H); 4.79 (s, 2H), 7.04 (s, 1H); 7.31 (d, J = 6.4 Hz, 1H); 7.51 (d, J = 8.0 Hz, 1H); 7.63 (s, 1H); 7.94 (s, 1H); 8.08 (s, 1H); 8.51 (s, 1H); 14.18 (br, 1H) |
| 276 | 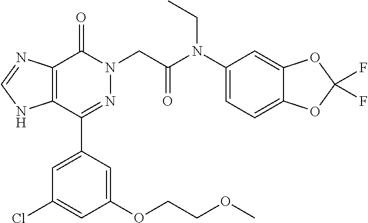 [SMILES: CCN(C(=O)Cn1nc(-c2cc(Cl)cc(OCCOC)c2)c2[nH]cnc2c1=O)c1ccc2c(c1)OC(F)(F)O2] | 562.2 [M + 1] | (400 MHz, DMSO) δ: 1.03 (t, J = 6.8 Hz, 3H); 3.33 (m, 4H); 3.69 (m, 3H); 4.20 (m, 2H); 4.79 (s, 2H); 7.17 (s, 1H); 7.32 (d, J = 7.6 Hz, 1H); 7.52 (d, J = 8.4 Hz, 1H); 7.66 (s, 1H); 8.04 (s, 2H); 8.51 (s, 1H), 14.20 (br, 1H). |
| 277 | 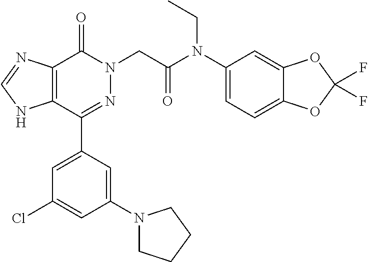 [SMILES: CCN(C(=O)Cn1nc(-c2cc(Cl)cc(N3CCCC3)c2)c2[nH]cnc2c1=O)c1ccc2c(c1)OC(F)(F)O2] | 557.2 [M + 1] | (400 MHz, DMSO) δ: 1.03 (m, 3H), 1.98 (m, 4H), 3.30 (m, 4H), 3.67 (q, J = 5.6 Hz, 2H), 4.77 (s, 2H); 6.61 (s, 1H), 7.32 (d, J = 7.6 Hz, 1H), 751-7.65 (m, 4H), 8.49 (s, 1H), 14.16 (br, 1H) |
| 278 | 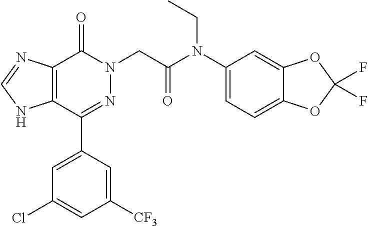 [SMILES: CCN(C(=O)Cn1nc(-c2cc(Cl)cc(C(F)(F)F)c2)c2[nH]cnc2c1=O)c1ccc2c(c1)OC(F)(F)O2] | 556.1 [M + 1] | (400 MHz, DMSO) δ: 1.03 (m, 3H), 3.67 (m, 2H), 4.82 (s, 2H), 7.33 (m, 1H), 7.51 (m, 1H), 7.64 (s, 1H), 7.80 (s, 1H), 8.57 (s, 1H), 8.77 (m, 2H), 14.26 (br, 1H) |
| 279 | 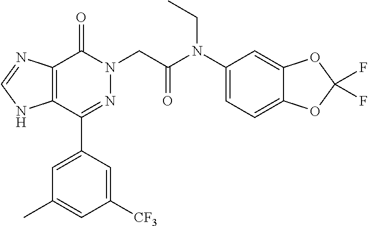 [SMILES: CCN(C(=O)Cn1nc(-c2cc(C)cc(C(F)(F)F)c2)c2[nH]cnc2c1=O)c1ccc2c(c1)OC(F)(F)O2] | 534.3 [M − 1] | (400 MHz, DMSO) δ: 1.03 (t, J = 6.8 Hz, 3H), 2.50 (s, 3H), 3.67 (q, J = 6.8 Hz, 2H), 4.80 (s, 2H), 7.33 (d, J = 7.6 Hz, 1H), 7.52 (d, J = 8.4 Hz, 1H), 7.65 (s, 1H), 7.68 (s, 1H), 8.48 (s, 1H), 8.53 (s, 1H), 8.63 (s, 1H), 14.18 (br, 1H) |
| 280 | 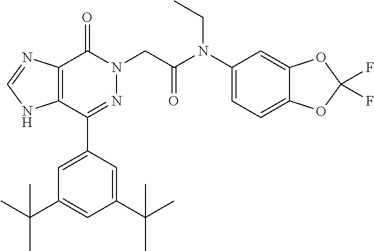 [SMILES: CCN(C(=O)Cn1nc(-c2cc(C(C)(C)C)cc(C(C)(C)C)c2)c2[nH]cnc2c1=O)c1ccc2c(c1)OC(F)(F)O2] | 564 [M − 1] | (400 MHz, DMSO) δ: 1.04 (t, J = 6.8 Hz, 3H), 1.15 (s, 18H), 3.68 (q, J = 6.8 Hz, 2H), 4.78 (s, 2H), 7.30 (d, J = 8.0 Hz, 1H), 7.49-7.52 (m, 2H), 7.60 (s, 1H), 8.17 (m, 2H), 8.50 (s, 1H), 14.07 (br, 1H) |
| 281 | 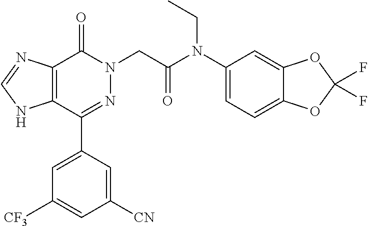 [SMILES: CCN(C(=O)Cn1nc(-c2cc(C#N)cc(C(F)(F)F)c2)c2[nH]cnc2c1=O)c1ccc2c(c1)OC(F)(F)O2] | 547.2 [M + 1] | (400 MHz, DMSO) δ: 1.05 (t, J = 6.8 Hz, 3H), 3.70 (q, J = 6.8 Hz, 2H), 4.87 (s, 2H), 7.36 (d, J = 8.0 Hz, 1H), 7.54 (d, J = 8.4 Hz, 1H), 7.68 (s, 1H), 8.51 (s, 1H), 8.59 (s, 1H), 9.11-9.16 (m, 2H), 14.35 (br, 1H) |
| 282 | 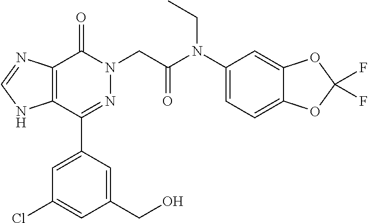 [SMILES: CCN(C(=O)Cn1nc(-c2cc(Cl)cc(CO)c2)c2[nH]cnc2c1=O)c1ccc2c(c1)OC(F)(F)O2] | 516.5 [M − 1] | (400 MHz, DMSO) δ: 1.06 (t, J = 6.4 Hz, 3H), 3.70 (q, J = 6.4 Hz, 2H), 4.62 (d, J = 4.4 Hz, 2H), 4.80 (s, 2H), 5.48 (s, 1H), 7.36 (d, J = 8.0 Hz, 1H), 7.49 (s, 1H), 7.56 (d, J = 8.4 Hz, 1H), 7.68 (s, 1H), 8.32 (s, 1H), 8.39 (s, 1H), 8.53 (s, 1H) |
| 283 | 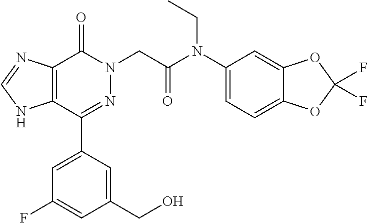 [SMILES: CCN(C(=O)Cn1nc(-c2cc(F)cc(CO)c2)c2[nH]cnc2c1=O)c1ccc2c(c1)OC(F)(F)O2] | 502.4 [M + 1] | (400 MHz, DMSO) δ: 1.05 (t, J = 6.8 Hz, 3H), 3.70 (q, J = 6.8 Hz, 2H), 4.62 (d, J = 5.6 Hz, 2H), 4.80 (s, 2H), 5.48 (t, J = 5.6 Hz, 1H), 7.26 (d, J = 8.8 Hz, 1H), 7.36 (d, J = 7.6 Hz, 1H), 7.55 (d, J = 8.4 Hz, 1H), 7.68 (s, 1H), 8.32- 8.39 (m, 2H), 8.53 (s, 1H), 14.19 (br, 1H) |
| 284 | 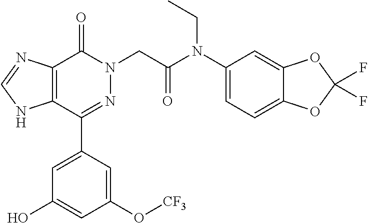 [SMILES: CCN(C(=O)Cn1nc(-c2cc(O)cc(OC(F)(F)F)c2)c2[nH]cnc2c1=O)c1ccc2c(c1)OC(F)(F)O2] | 554 [M + 1] | (400 MHz, DMSO) δ: 1.03 (t, J = 6.8 Hz, 3H); 3.68 (d, J = 6.8 Hz, 2H); 4.77 (s, 2H); 6.81 (s, 1H); 7.31 (d, J = 8 Hz, 1H); 7.52 (d, J = 8.4 Hz, 1H); 7.63 (s, 1H); 7.85 (m, 1H); 7.93 (m, 1H); 8.51 (s, 1H); 10.31 (s, 1H), 14.17 (br, 1H) |
| 285 | 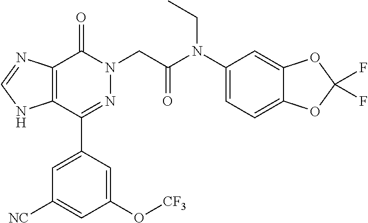 [SMILES: CCN(C(=O)Cn1nc(-c2cc(C#N)cc(OC(F)(F)F)c2)c2[nH]cnc2c1=O)c1ccc2c(c1)OC(F)(F)O2] | 563.4 [M + 1] | (400 MHz, DMSO) δ: 1.03 (t, J = 7.2 Hz, 3H), 3.67 (q, J = 7.2 Hz, 2H), 4.83 (s, 2H), 7.33 (d, J = 7.6 Hz, 1H), 7.52 (d, J = 8.0 Hz, 1H), 7.65 (s, 1H), 8.17 (s, 1H), 8.57 (s, 1H), 8.81 (s, 1H), 8.86 (s, 1H), 14.19 (br, 1H) |
| 286 | 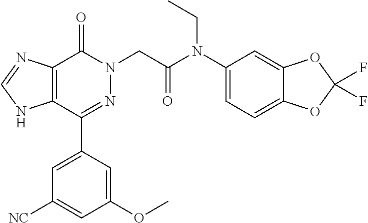 [SMILES: CCN(C(=O)Cn1nc(-c2cc(C#N)cc(OC)c2)c2[nH]cnc2c1=O)c1ccc2c(c1)OC(F)(F)O2] | 507 [M − 1] | (400 MHz, DMSO) δ: 1.03 (t, J = 6.8 Hz, 3H), 3.68 (q, J = 6.8 Hz, 2H); 3.90 (s, 3H); 4.81 (s, 2H); 7.33 (d, J = 7.2 Hz, 1H); 7.52 (d, J = 8.4 Hz, 1H); 7.58 (s, 1H); 7.66 (s, 1H); 8.40 (m, 2H); 8.53 (s, 1H); 14.30 (br, 1H) |
| 287 | 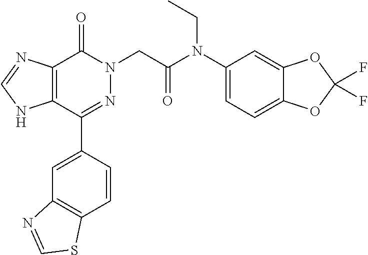 [SMILES: CCN(C(=O)Cn1nc(-c2ccc3scnc3c2)c2[nH]cnc2c1=O)c1ccc2c(c1)OC(F)(F)O2] | 511 [M + 1] | (400 MHz, DMSO) δ: 1.04 (t, J = 7.2 Hz, 3H), 3.69 (q, J = 7.2 Hz, 2H), 4.82 (s, 2H), 7.35 (m, 1H), 7.54 (d, J = 8.4 Hz, 1H), 7.67 (s, 1H), 8.31 (d, J = 8.4 Hz, 1H), 8.44 (m, 1H), 8.54 (s, 1H), 9.27 (br, 1H), 9.48 (s, 1H), 14.15 (br, 1H) |
| 288 | 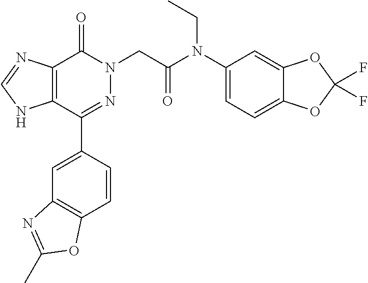 [SMILES: CCN(C(=O)Cn1nc(-c2ccc3oc(C)nc3c2)c2[nH]cnc2c1=O)c1ccc2c(c1)OC(F)(F)O2] | 509 [M + 1] | (400 MHz, DMSO) δ: 1.03 (t, J = 6.8 Hz, 3H); 2.66 (s, 3H); 3.68 (q, J = 6.8 Hz, 2H); 4.78 (s, 2H); 7.33 (d, J = 8.0 Hz, 1H); 7.52 (d, J = 8.4 Hz, 1H); 7.66 (s, 1H); 7.78 (d, J = 8.8 Hz, 1H); 8.36-8.39 (m, 2H); 8.75 (s, 1H); 10.37 (s, 1H) |
| 289 | 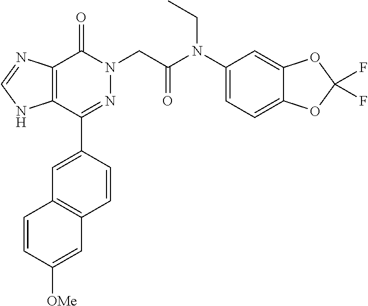 [SMILES: CCN(C(=O)Cn1nc(-c2ccc3cc(OC)ccc3c2)c2[nH]cnc2c1=O)c1ccc2c(c1)OC(F)(F)O2] | 534.2 [M + 1] | (400 MHz, DMSO) δ: 1.06 (t, J = 6.8 Hz, 3H), 3.71 (q, J = 6.8 Hz, 2H), 3.90 (s, 3H), 4.82 (s, 2H), 7.233- 7.24 (d, J = 8.8 Hz, 1H), 7.37 (d, J = 8.0 Hz, 1H), 7.41 (s, 1H), 7.56 (d, J = 8.8 Hz, 1H), 7.70 (s, 1H), 7.93-7.99 (m, 2H), 8.33 (d, J = 7.2 Hz, 1H), 8.52 (s, 1H), 9.10 (s, 1H) |
| 290 | 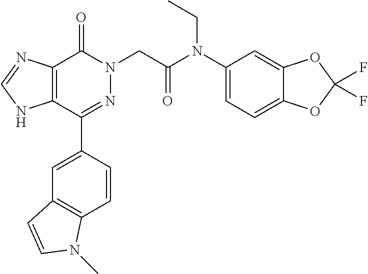 [SMILES: CCN(C(=O)Cn1nc(-c2ccc3c(ccn3C)c2)c2[nH]cnc2c1=O)c1ccc2c(c1)OC(F)(F)O2] | 507.3 [M + 1] | (400 MHz, DMSO) δ: 1.04 (t, J = 6.8 Hz, 3H), 3.69 (q, J = 6.8 Hz, 2H), 3.84 (s, 3H), 4.78 (s, 2H), 6.56 (d, J = 2.4 Hz, 1H), 7.34 (d, J = 8.0 Hz, 1H), 7.39 (d, J = 2.8 Hz, 1H), 7.52-7.56 (m, 2H), 7.66 (s, 1H), 8.08 (m 1H), 8.47 (s, 1H), 8.75 (m, 1H), 14.01 (br, 1H) |
| 291 | 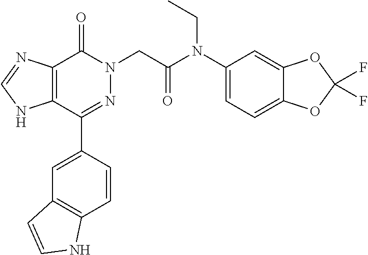 [SMILES: CCN(C(=O)Cn1nc(-c2ccc3[nH]ccc3c2)c2[nH]cnc2c1=O)c1ccc2c(c1)OC(F)(F)O2] | 493 [M + 1] | (400 MHz, DMSO) δ: 1.04 (t, J = 6.8 Hz, 3H); 3.69 (q, J = 6.8 Hz, 2H); 4.77 (s, 2H); 6.56 (s, 1H) 7.33 (d, J = 8.0 Hz, 1H); 7.41 (s, 1H) 7.48-7.54 (m, 2H); 7.65 (s, 1H); 8.04 (m, 1H); 8.47 (s, 1H); 8.79 (s, 1H), 11.27 (s, 1H), 14.06 (br, 1H) |
| 292 | 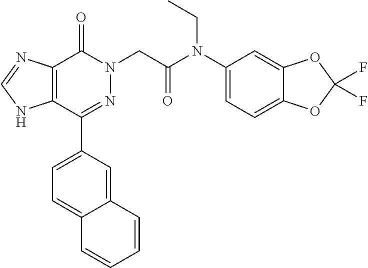 [SMILES: CCN(C(=O)Cn1nc(-c2ccc3ccccc3c2)c2[nH]cnc2c1=O)c1ccc2c(c1)OC(F)(F)O2] | 504 [M + 1] | (400 MHz, DMSO) δ: 1.06 (t, J = 6.8 Hz, 3H); 3.71 (q, J = 6.8 Hz, 2H); 4.84 (s, 2H); 7.38 (d, J = 8.4 Hz, 1H); 7.56 (d, J = 8.4 Hz, 1H), 7.61 (m, 2H); 7.70 (s, 1H); 8.00 (m, 1H); 8.07 (d, J = 8.0 Hz, 2H); 8.355 (m, 1H); 8.57 (s, 1H); 9.19 (br, 1H) |
| 293 | 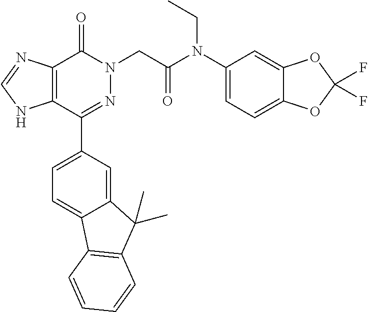 [SMILES: CCN(C(=O)Cn1nc(-c2ccc3c(c2)C(C)(C)c2ccccc2-3)c2[nH]cnc2c1=O)c1ccc2c(c1)OC(F)(F)O2] | 570 [M + 1] | (400 MHz, DMSO) δ: 1.05 (t, J = 6.8 Hz, 3H); 1.51 (s, 6H) 3.69 (q, J = 6.8 Hz, 2H); 4.81 (s, 2H); 7.33- 7.39 (m, 3H); 7.54 (d, J = 8.4 Hz, 1H) 7.61 (d, J = 6.0 Hz, 1H); 7.67 (s, 1H); 7.90 (d, J = 6.0 Hz, 1H); 7.98 (d, J = 7.6 Hz, 1H); 8.42 (m, 1H), 8.50-8.54 (m, 2H), 14.17 (br, 1H) |
| 294 | 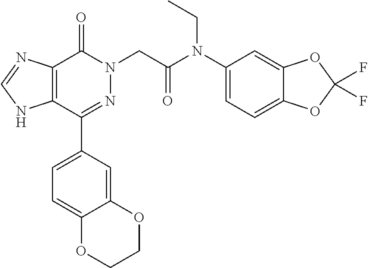 [SMILES: CCN(C(=O)Cn1nc(-c2ccc3c(c2)OCCO3)c2[nH]cnc2c1=O)c1ccc2c(c1)OC(F)(F)O2] | 512 [M + 1] | (400 MHz, DMSO) δ: 1.03 (t, J = 6.8 Hz, 3H); 3.67 (d, J = 6.8 Hz, 2H); 4.30 (s, 4H); 4.74 (s, 2H); 6.99 (d, J = 8.4 Hz, 1H); 7.31 (d, J = 8.4 Hz, 1H); 7.51 (d, J = 8.4 Hz, 1H); 7.64 (s, 1H); 7.90 (m, 1H), 7.99 (m, 1H), 8.46 (s, 1H); 14.13 (s, 1H) |
| 295 | 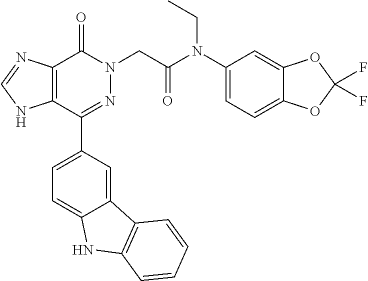 [SMILES: CCN(C(=O)Cn1nc(-c2ccc3[nH]c4ccccc4c3c2)c2[nH]cnc2c1=O)c1ccc2c(c1)OC(F)(F)O2] | 543 [M + 1] | (400 MHz, DMSO) δ: 1.05 (t, J = 6.8 Hz, 3H); 3.69 (q, J = 6.8 Hz, 2H); 4.81 (s, 2H); 7.22 (t, J = 7.6 Hz, 1H); 7.36 (d, J = 8.0 Hz, 1H); 7.43 (t, J = 7.6 Hz, 1H); 7.52-7.62 (m, 3H); 7.69 (s, 1H); 8.15 (d, J = 7.6 Hz, 1H); 8.39 (m, 1H); 8.52 (s, 1H); 9.16 (br, 1H); 11.50 (s, 1H), 13.82 (br, 1H) |
| 296 | 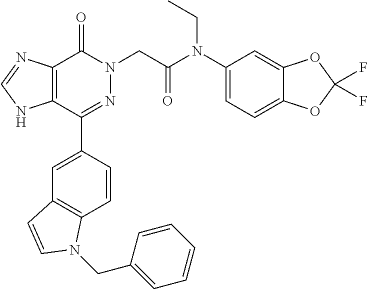 [SMILES: CCN(C(=O)Cn1nc(-c2ccc3c(ccn3Cc3ccccc3)c2)c2[nH]cnc2c1=O)c1ccc2c(c1)OC(F)(F)O2] | 583 [M + 1] | (400 MHz, DMSO) δ: 1.03 (m, 3H); 3.68 (q, J = 6.0 Hz, 2H); 4.76 (s, 2H); 5.48 (s, 2H); 6.63 (s, 1H); 7.21- 7.34 (m, 6H); 7.53 (d, J = 8.4 Hz, 1H); 7.58 (m, 2H); 7.65 (s, 1H); 8.02 (m, 1H); 8.46 (s, 1H); 8.77 (m, 1H); 14.09 (br, 1H) |
| 297 | 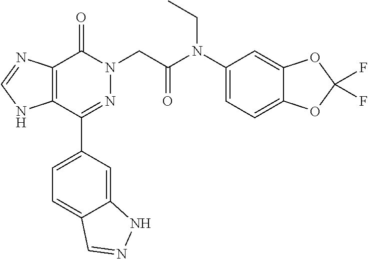 [SMILES: CCN(C(=O)Cn1nc(-c2ccc3cn[nH]c3c2)c2[nH]cnc2c1=O)c1ccc2c(c1)OC(F)(F)O2] | 494.3 [M + 1] | (400 MHz, DMSO) δ: 1.06 (d, J = 7.2 Hz, 3H), 3.71 (q, J = 7.2 Hz, 2H), 4.82 (s, 2H), 7.37 (d, J = 6.8 Hz, 1H), 7.56 (d, J = 8.8 Hz, 1H), 7.69 (s, 1H), 7.89 (d, J = 8.8 Hz, 1H), 8.01 (m, 1H), 8.15 (s, 1H), 8.54 (s, 1H), 8.96 (m, 1H), 13.32 (br, 1H), 14.12 (br, 1H) |
| 298 | 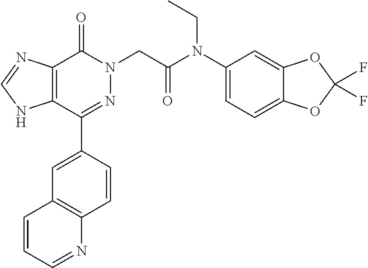 [SMILES: CCN(C(=O)Cn1nc(-c2ccc3ncccc3c2)c2[nH]cnc2c1=O)c1ccc2c(c1)OC(F)(F)O2] | 505.2 [M + 1] | (400 MHz, DMSO) δ: 1.04 (m, 3H), 3.69 (m, 2H), 4.84 (s, 2H), 7.37 (m, 1H) 7.53- 7.69 (m, 3H), 8.15 (d, J = 8.4 Hz, 1H), 8.52 (d, J = 7.6 Hz, 1H), 8.58 (m, 2H), 8.96 (s, 1H), 9.26 (m, 1H), 14.20 (br, 1H) |
| 299 | 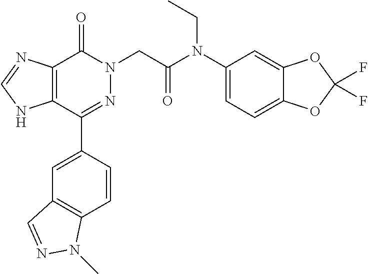 [SMILES: CCN(C(=O)Cn1nc(-c2ccc3c(cnn3C)c2)c2[nH]cnc2c1=O)c1ccc2c(c1)OC(F)(F)O2] | 508.3 [M + H] | (400 MHz, DMSO-d₆): 1.06 (t, J = 6.8 Hz, 3H), 3.71 (q, J = 6.8 Hz, 2H), 4.11 (s, 3H), 4.81 (s, 2H), 7.36 (d, J = 8.0 Hz, 1H), 7.55 (d, J = 7.6 Hz, 1H), 7.69 (s, 1H), 7.79 (d, J = 6.0 Hz, 1H), 8.24 (s, 1H), 8.30 (m, 1H), 8.52 (s, 1H), 9.07 (m, 1H), 14.13 (br, 1H) |
| 300 | 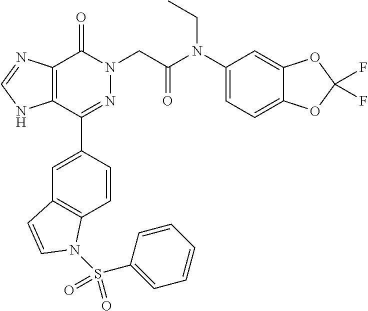 [SMILES: CCN(C(=O)Cn1nc(-c2ccc3c(ccn3S(=O)(=O)c3ccccc3)c2)c2[nH]cnc2c1=O)c1ccc2c(c1)OC(F)(F)O2] | 633.2 [M + H] | (400 MHz, DMSO-d₆): 1.02 (t, J = 7.2 Hz, 3H), 3.67 (q, J = 7.2 Hz, 2H), 4.76 (s, 2H), 7.00 (d, J = 3.6 Hz, 1H), 7.31-7.33 (d, J = 8.4 Hz, 1H), 7.52 (d, J = 8.8 Hz, 1H), 7.58-7.70 (m, 3H), 7.72 (m, 1H), 7.89 (d, J = 3.6 Hz, 1H), 8.03 (d, J = 7.2 Hz, 2H), 8.08 (d, J = 8.8 Hz, 1H), 8.25 (m, 1H), 8.47 (s, 1H), 8.70 (s, 1H), 14.09 (br, 1H) |
| 301 | 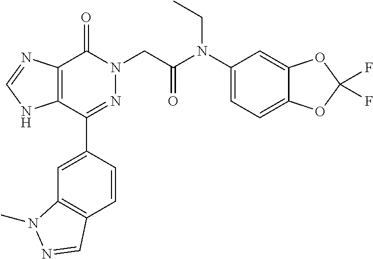 [SMILES: CCN(C(=O)Cn1nc(-c2ccc3cnn(C)c3c2)c2[nH]cnc2c1=O)c1ccc2c(c1)OC(F)(F)O2] | 508.2 [M + H] | (400 MHz, DMSO-d₆): 1.04 (t, J = 6.8 Hz, 3H), 3.69 (q, J = 6.8 Hz, 2H), 4.12 (s, 3H), 4.81 (s, 2H), 7.35 (d, J = 6.8 Hz, 1H), 7.54 (d, J = 8.8 Hz, 1H), 7.67 (s, 1H), 7.88 (d, J = 8.4 Hz, 1H), 8.03 (br, 1H), 8.11 (s, 1H), 8.55 (s, 1H), 8.79 (m, 1H), 14.24 (s, 1H) |
| 302 | 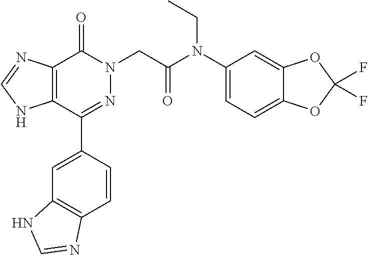 [SMILES: CCN(C(=O)Cn1nc(-c2ccc3nc[nH]c3c2)c2[nH]cnc2c1=O)c1ccc2c(c1)OC(F)(F)O2] | 494.0 [M + 1] | (400 MHz, DMSO) δ: 1.06 (t, J = 6.8 Hz, 3H); 3.70 (q, J = 6.8 Hz, 2H); 4.81 (s, 2H); 7.36 (d, J = 8.8 Hz, 1H); 7.55 (d, J = 8.8 Hz, 1H), 7.68 (m, 1H) 7.77 (d, J = 6.8 Hz, 1H); 8.15 (m, 1H); 8.33 (d, J = 6.8 Hz, 1H); 8.50 (s, 1H); 8.84 (m, 1H), 12.69 (m, 1H), 14.05 (br, 1H) |
| 303 | 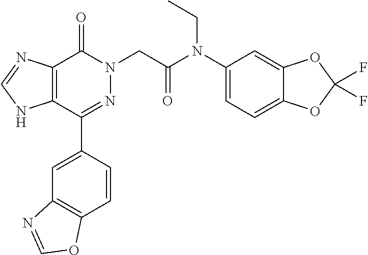 [SMILES: CCN(C(=O)Cn1nc(-c2ccc3ocnc3c2)c2[nH]cnc2c1=O)c1ccc2c(c1)OC(F)(F)O2] | 495.0 [M + H] | (400 MHz, DMSO-d₆): 1.03 (t, J = 6.8 Hz, 3H), 3.68 (q, J = 6.8 Hz, 2H), 4.81 (s, 2H), 7.34 (d, J = 8.8 Hz, 1H), 7.53 (d, J = 8.4 Hz, 1H), 7.67 (s, 1H), 7.93 (d, J = 8.8 Hz, 1H), 8.44 (m, 1H), 8.52 (s, 1H), 8.84 (s, 1H), 9.91 (m, 1H), 14.21 (br, 1H) |
| 304 | 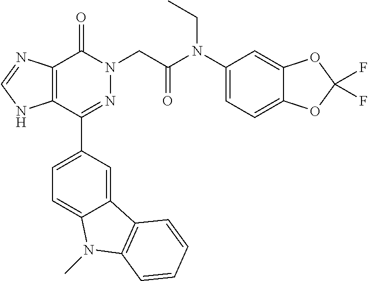 [SMILES: CCN(C(=O)Cn1nc(-c2ccc3c(c2)c2ccccc2n3C)c2[nH]cnc2c1=O)c1ccc2c(c1)OC(F)(F)O2] | 557 [M + 1] | (400 MHz, DMSO) δ: 1.05 (m, 3H); 3.71 (m, 2H); 3.94 (s, 3H); 4.81 (s, 2H); 7.28 (d, J = 7.2 Hz, 1H); 7.37 (d, J = 8.8 Hz, 1H); 7.51-7.57 (m, 2H); 7.642]- 7.75 (m, 3H); 8.19 (d, J = 7.2 Hz, 1H); 8.52 (m, 2H); 9.19 (m, 1H); 14.02 (br, 1H) |
| 305 | 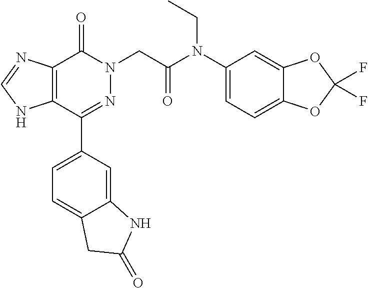 [SMILES: CCN(C(=O)Cn1nc(-c2ccc3c(c2)NC(=O)C3)c2[nH]cnc2c1=O)c1ccc2c(c1)OC(F)(F)O2] | 509.0 [M + H] | (400 MHz, DMSO-d₆): 1.05 (t, J = 6.8 Hz, 3H), 3.58 (s, 2H), 3.70 (q, J = 6.8, 2H), 4.78 (s, 2H), 7.35-7.36 (m, 2H), 7.55 (d, J = 8.4 Hz, 1H), 7.66 (s, 1H), 7.93 (m, 1H), 8.06 (m, 1H), 8.51 (s, 1H), 10.54 (s, 1H), 10.58 (s, 1H), 14.17 (br, 1H) |
| 306 | 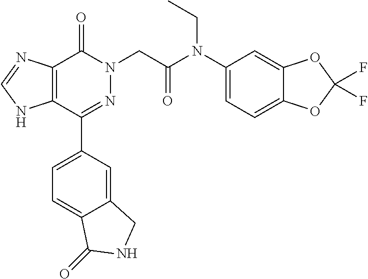 [SMILES: CCN(C(=O)Cn1nc(-c2ccc3c(c2)CNC3=O)c2[nH]cnc2c1=O)c1ccc2c(c1)OC(F)(F)O2] | 509.2 [M + H] | (400 MHz, DMSO-d₆): 1.03 (t, J = 7.2 Hz, 3H), 3.68 (q, J = 7.2, 2H), 4.49 (s, 2H), 4.80 (s, 2H), 7.34 (d, J = 8.4 Hz, 1H), 7.54 (d, J = 8.4 Hz, 1H), 7.68 (s, 1H), 7.81 (d, J = 8.0 Hz, 1H), 8.45 (m, 1H), 8.53 (s, 1H), 8.63 (m, 1H), 8.70 (s, 1H), 14.22 (s, 1H) |
| 307 | 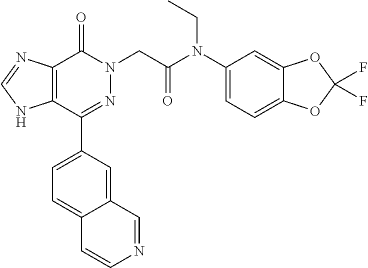 [SMILES: CCN(C(=O)Cn1nc(-c2ccc3ccncc3c2)c2[nH]cnc2c1=O)c1ccc2c(c1)OC(F)(F)O2] | 505.2 [M + H] | (400 MHz, DMSO-d₆): 1.04 (t, J = 7.2 Hz, 3H), 3.69 (q, J = 7.2 Hz, 2H), 4.84 (s, 2H), 7.36 (d, J = 8.4 Hz, 1H), 7.55 (d, J = 8.4 Hz, 1H), 7.69 (s, 1H), 7.91 (d, J = 5.6 Hz, 1H), 8.11 (d, J = 8.8 Hz, 1H), 8.53-8.58 (m, 3H), 9.38 (br, 1H), 9.44 (s, 1H), 14.29 (br, 1H) |
| 308 | 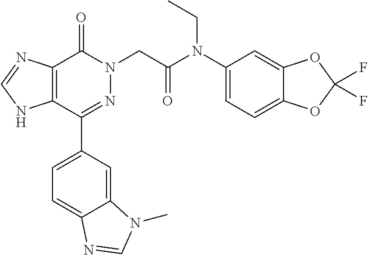 [SMILES: CCN(C(=O)Cn1nc(-c2ccc3ncn(C)c3c2)c2[nH]cnc2c1=O)c1ccc2c(c1)OC(F)(F)O2] | 508 [M + 1] | (400 MHz, DMSO) δ: 1.06 (t, J = 7.2 Hz, 3H); 3.71 (q, J = 7.2 Hz, 2H); 3.93 (s, 3H); 4.82 (s, 2H); 7.36 (d, J = 8 Hz, 1H); 7.55 (d, J = 8.8 Hz, 1H); 7.69 (s, 1H); 7.78 (d, J = 8.8 Hz, 1H); 8.31 (s, 1H); 8.54 (s, 1H); 8.74 (m, 2H) |
| 309 | 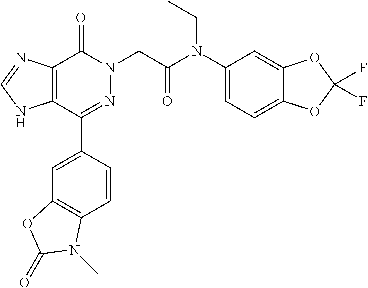 [SMILES: CCN(C(=O)Cn1nc(-c2ccc3c(c2)oc(=O)n3C)c2[nH]cnc2c1=O)c1ccc2c(c1)OC(F)(F)O2] | 525 [M + 1] | (400 MHz, DMSO) δ: 1.03 (t, J = 6.8 Hz, 3H); 3.40 (s, 3H); 3.68 (q, J = 6.8 Hz, 2H); 4.48 (s, 2H); 7.33 (d, J = 7.6 Hz, 1H); 7.41 (d, J = 8.4 Hz, 1H); 7.53 (d, J = 8.4 Hz, 1H) 7.66 (s, 1H); 8.30 (s, 1H); 8.39 (d, J = 6.4 Hz, 1H); 8.50 (s, 1H); 14.15 (br, 1H) |
| 310 | 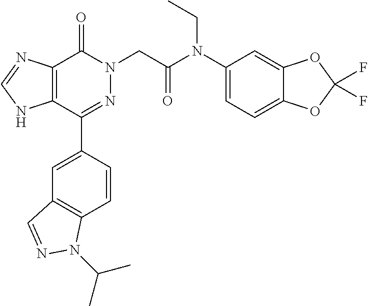 [SMILES: CCN(C(=O)Cn1nc(-c2ccc3c(cnn3C(C)C)c2)c2[nH]cnc2c1=O)c1ccc2c(c1)OC(F)(F)O2] | 536 [M + 1] | (400 MHz, DMSO) δ: 1.04 (t, J = 6.8 Hz, 3H); 1.50 (d, J = 5.6 Hz, 6H); 3.69 (d, J = 6.8 Hz, 2H); 4.78 (s, 2H); 5.02 (m, 1H); 7.33 (d, J = 8.0 Hz, 1H); 7.53 (d, J = 8.4 Hz, 1H); 7.66 (s, 1H); 7.82 (d, J = 8.8 Hz, 1H); 8.23 (s, 1H); 8.27 (m, 1H), 8.51 (s, 1H); 9.05 (s, 1H); 14.15 (br, 1H) |
| 311 | 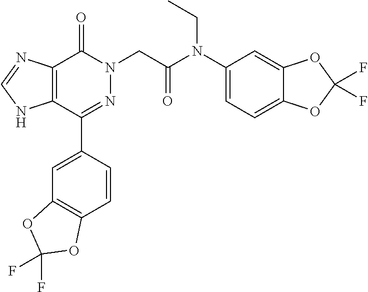 [SMILES: CCN(C(=O)Cn1nc(-c2ccc3c(c2)OC(F)(F)O3)c2[nH]cnc2c1=O)c1ccc2c(c1)OC(F)(F)O2] | 534 [M + 1] | (400 MHz, DMSO) δ: 1.03 (t, J = 6.8 Hz, 3H); 3.66 (d, J = 6.8 Hz, 2H); 4.78 (s, 2H); 7.32 (d, J = 8.0 Hz, 1H); 7.52 (d, J = 8.4 Hz, 1H), −7.69 (d, J = 8.4 Hz, 1H), 7.66 (s, H); 8.30 (s, 1H); 8.35 (m, 1H), 8.52 (s, 1H); 14.23 (br, 1H) |
| 312 | 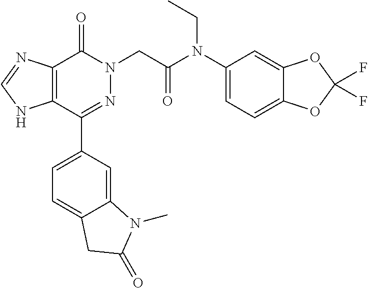 [SMILES: CCN(C(=O)Cn1nc(-c2ccc3c(c2)N(C)C(=O)C3)c2[nH]cnc2c1=O)c1ccc2c(c1)OC(F)(F)O2] | 523 [M + 1] | (400 MHz, DMSO) δ: 1.04 (t, J = 6.8 Hz, 3H); 3.19 (s, 3H); 3.68 (q, J = 6.8 Hz, 2H); 4.69 (s, 2H); 6.09 (s, 1H); 7.34 (d, J = 7.6 Hz, 2H); 7.53 (d, J = 8.4 Hz, 1H); 7.66 (s, 1H); 7.96 (s, 1H), 8.04 (s, 1H); 8.31 (d, J = 8.0 Hz, 1H) |
| 313 | 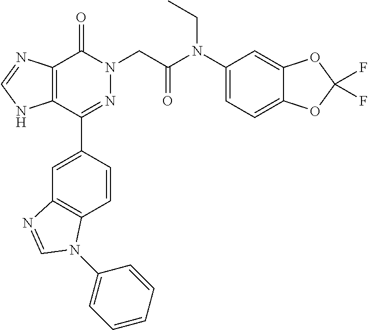 [SMILES: CCN(C(=O)Cn1nc(-c2ccc3c(c2)ncn3-c2ccccc2)c2[nH]cnc2c1=O)c1ccc2c(c1)OC(F)(F)O2] | 570 [M + 1] | (400 MHz, DMSO) δ: 1.04 (t, J = 6.8 Hz, 3H); 3.69 (q, J = 6.9 Hz, 2H); 4.81 (s, 2H); 7.34 (d, J = 8.8 Hz, 1H); 7.52-7.56 (m, 2H); 7.67 (t, J = 7.6 Hz, 3H); 7.73-7.76 (m 3H); 8.35 (d, J = 8.0 Hz, 1H); 8.54 (s, 1H), 8.66 (s, 1H), 9.01 (s, 1H), 14.17 (br, 1H) |
| 314 | 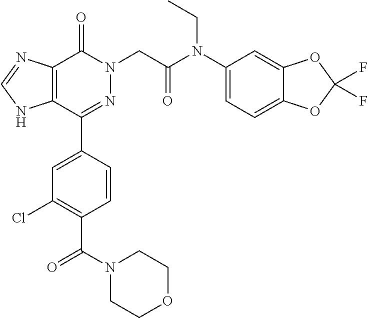 [SMILES: CCN(C(=O)Cn1nc(-c2ccc(C(=O)N3CCOCC3)c(Cl)c2)c2[nH]cnc2c1=O)c1ccc2c(c1)OC(F)(F)O2] | 601.4 [M + 1] | (400 MHz, DMSO): 1.03 (m, 3H), 3.19 (s, 2H); 3.55 (s, 2H), 3.68 (s, 6H), 4.79 (s, 2H), 7.33 (d, J = 8.0 Hz, 2H), 7.51-7.57 (m, 2H), 7.65 (s, 1H), 8.42-8.44 (d, J = 7.2 Hz, 1H), 8.50 (s, 1H), 8.56 (s, 1H), 14.2 (br, 1H) |
| 315 | 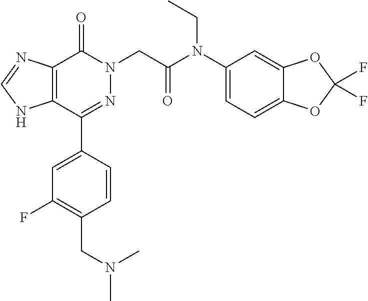 [SMILES: CCN(C(=O)Cn1nc(-c2ccc(CN(C)C)c(F)c2)c2[nH]cnc2c1=O)c1ccc2c(c1)OC(F)(F)O2] | 529 [M + 1] | (400 MHz, DMSO) 1.03 (m, 3H), 2.19 (s, 6H); 3.51 (s, 2H), 3.68 (q, J = 6.8 Hz, 2H), 4.78 (s, 2H), 7.33 (d, J = 8.4 Hz, 1H), 7.51- 7.56 (m, 2H), 7.66 (s, 1H), 8.21 (m, 2H), 8.51 (s, 1H), 13.91 (br, 1H) |
| 316 | 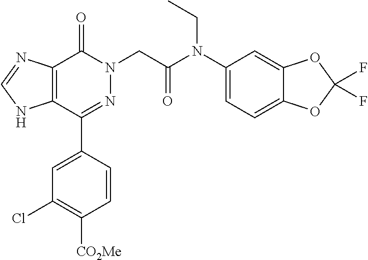 [SMILES: CCN(C(=O)Cn1nc(-c2ccc(C(=O)OC)c(Cl)c2)c2[nH]cnc2c1=O)c1ccc2c(c1)OC(F)(F)O2] | 546 [M + 1] | (400 MHz, DMSO) δ: 1.03 (m, 3H); 3.67 (m, 2H); 3.90 (s, 3H); 4.81 (s, 2H); 7.35 (m, 1H); 7.54 (d, J = 8.4 Hz, 1H); 7.67 (s, 1H); 7.99 (d, J = 7.6 Hz, 1H); 8.51 (m, 2H); 8.69 (s, 1H); 14.26 (br, 1H) |
| 317 | 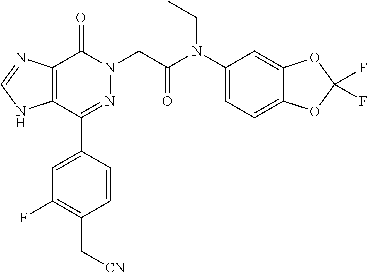 [SMILES: CCN(C(=O)Cn1nc(-c2ccc(CC#N)c(F)c2)c2[nH]cnc2c1=O)c1ccc2c(c1)OC(F)(F)O2] | 511 [M + 1] | (400 MHz, DMSO) δ: 1.03 (t, J = 6.8 Hz, 3H); 3.68 (q, J = 6.8 Hz, 2H); 4.17 (s, 2H); 4.79 (s, 2H); 7.33 (d, J = 7.6 Hz, 1H); 7.53 (d, J = 8.4 Hz, 1H); 7.62 (d, J = 8.4 Hz, 1H); 7.66 (s, 1H), 8.30 (m, 2H); 8.52 (s, 1H); 14.26 (br, 1H) |
| 318 | 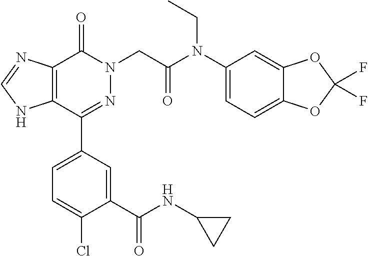 [SMILES: CCN(C(=O)Cn1nc(-c2ccc(Cl)c(C(=O)NC3CC3)c2)c2[nH]cnc2c1=O)c1ccc2c(c1)OC(F)(F)O2] | 571.0 [M + H] | (400 MHz, DMSO-d₆): 0.56 (m, 2H), 0.73 (m, 2H), 1.05 (t, J = 6.8 Hz, 3H), 2.88 (m, 1H), 3.70 (q, J = 6.8 Hz, 2H), 4.80 (s, 2H), 7.36 (d, J = 8.0 Hz, 1H), 7.55 (d, J = 8.4 Hz, 1H), 7.66-7.68 (m, 2H), 8.43 (s, 1H), 8.52 (m, 2H), 8.63 (d, J = 4.4 Hz, 1H), 14.24 (br, 1H) |
| 319 | 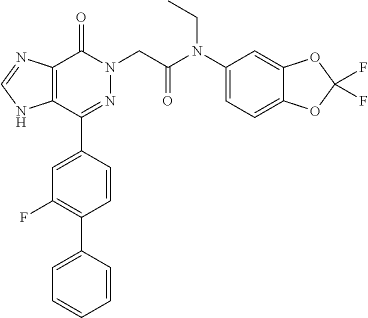 [SMILES: CCN(C(=O)Cn1nc(-c2ccc(-c3ccccc3)c(F)c2)c2[nH]cnc2c1=O)c1ccc2c(c1)OC(F)(F)O2] | 548 [M + 1] | (400 MHz, DMSO) δ: 1.04 (t, J = 6.8 Hz, 3H); 3.67 (q, J = 6.8 Hz, 2H); 4.74 (s, 2H); 7.33 (d, J = 8.4 Hz, 1H); 7.42 (d, J = 7.2 Hz, 1H); 7.52 (t, J = 8.4 Hz, 3H); 7.62-7.68 (m, 4H); 8.11 (s, 1H); 8.44 (d, J = 8.0 Hz, 1H); 8.51 (d, J = 13.6 Hz, 1H) |
| 320 | 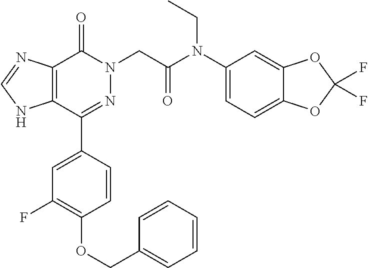 | 556 [M + 1] | (400 MHz, DMSO) δ: 1.05 (t, J = 6.8 Hz, 3H); 3.67 (d, J = 6.8 Hz, 2H); 4.81 (s, 2H); 7.31 (d, J = 8.4 Hz, 1H); 7.51 (d, J = 8.8 Hz, 1H); 7.64 (s, 1H); 7.92 (d, J = 9.6 Hz, 1H); 8.54 (s, 1H); 8.67 (m, 1H); 9.00 (s, 1H); 14.26 (s, 1H) |
| 321 | 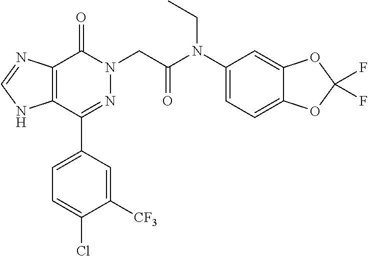 [SMILES: CCN(C(=O)Cn1nc(-c2ccc(Cl)c(C(F)(F)F)c2)c2[nH]cnc2c1=O)c1ccc2c(c1)OC(F)(F)O2] | 578 [M + 1] | (400 MHz, DMSO) δ: 1.03 (t, J = 6.8 Hz, 3H); 3.66 (q, J = 6.8 Hz, 2H); 4.76 (s, 2H); 5.27 (s, 2H); 7.32 (d, J = 8.4 Hz, 1H); 7.37 (d, J = 7.2 Hz, 1H); 7.47-7.45 (m, 3H); 7.49-7.52 (m, 3H); 7.66 (s, 1H); 8.21- 8.26 (m, 2H); 8.48 (s, 1H); 14.14 (br, 1H) |
| 322 | 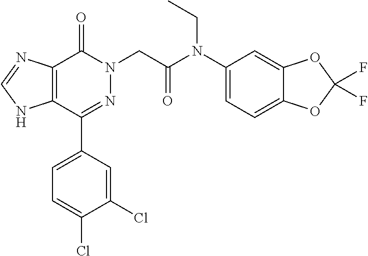 [SMILES: CCN(C(=O)Cn1nc(-c2ccc(Cl)c(Cl)c2)c2[nH]cnc2c1=O)c1ccc2c(c1)OC(F)(F)O2] | 522 [M + 1] | (400 MHz, DMSO) δ: 1.03 (t, J = 6.8 Hz, 3H); 3.67 (q, J = 6.8 Hz, 2H); 4.79 (s, 2H); 7.33 (d, J = 8.0 Hz, 1H); 7.52 (d, J = 8.4 Hz, 1H); 7.66 (s, 1H); 7.81 (d, J = 8.4 Hz, 1H); 8.39 (d, J = 7.2 Hz, 1H); 8.53 (s, 1H); 8.69 (s, 1H); 14.26 (s, 1H) |
| 323 | 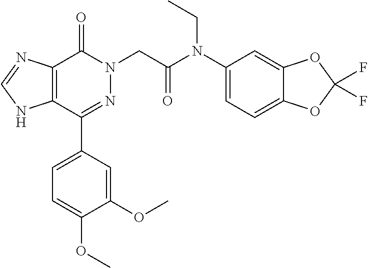 [SMILES: CCN(C(=O)Cn1nc(-c2ccc(OC)c(OC)c2)c2[nH]cnc2c1=O)c1ccc2c(c1)OC(F)(F)O2] | 514.1 [M + H] | (400 MHz, DMSO) δ: 1.03 (t, J = 6.8 Hz, 3H), 3.34- 3.67 (q, J = 6.8 Hz, 2H), 3.83 (s, 3H), 3.84 (s, 3H), 4.76 (s, 2H), 7.11 (d, J = 8.4 Hz, 2H), 7.31 (d, J = 8.4 Hz, 2H), 7.52 (d, J = 8.4 Hz, 2H), 7.63 (s, 1H), 7.06 (m, 1H), 8.07 (m, 1H), 8.48 (s, 1H), 14.08 (br, 1H) |
| 324 | 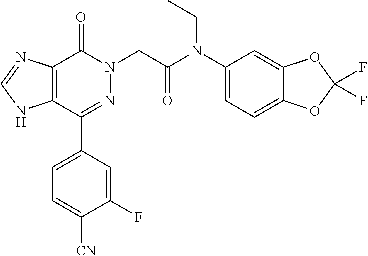 [SMILES: CCN(C(=O)Cn1nc(-c2ccc(C#N)c(F)c2)c2[nH]cnc2c1=O)c1ccc2c(c1)OC(F)(F)O2] | 497 [M + 1] | (400 MHz, DMSO) δ: 1.05 (t, J = 7.2 Hz, 3H); 3.69 (q, J = 7.2 Hz, 2H); 4.84 (s, 2H); 7.35 (d, J = 8.4 Hz, 1H); 7.55 (d, J = 8.4 Hz, 1H); 7.69 (s, 1H); 8.14 (t, J = 7.6 Hz, 1H); 8.53 (m, 2H); 8.59 (s, 1H); 14.35 (br, 1H) |
| 325 | 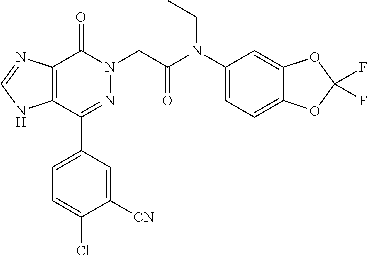 [SMILES: CCN(C(=O)Cn1nc(-c2ccc(Cl)c(C#N)c2)c2[nH]cnc2c1=O)c1ccc2c(c1)OC(F)(F)O2] | 513 [M + 1] | (400 MHz, DMSO) δ: 1.03 (t, J = 7.2 Hz, 3H); 3.68 (q, J = 7.2, 2H); 4.81 (s, 2H); 7.33 (d, J = 7.6 Hz, 1H); 7.53 (d, J = 8.8 Hz, 1H); 7.67 (s, 1H); 7.95 (d, J = 8.4 Hz, 1H); 8.55 (s, 1H); 8.76 (m, 1H); 8.94 (s, 1H) 14.31 (br, 1H) |
| 326 | 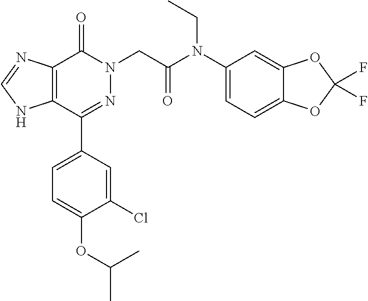 [SMILES: CCN(C(=O)Cn1nc(-c2ccc(OC(C)C)c(Cl)c2)c2[nH]cnc2c1=O)c1ccc2c(c1)OC(F)(F)O2] | 546 [M + 1] | (400 MHz, DMSO) δ: 1.03 (t, J = 6.8 Hz, 3H); 1.34 (d, J = 6.0 Hz, 6H) 3.67 (q, J = 6.8 Hz, 2H); 4.76-4.81 (m, 3H); 7.33 (d, J = 8.8 Hz, 2H); 7.52 (d, J = 8.8 Hz, 1H); 7.65 (s, 1H); 8.36 (s, 1H); 8.50 (s, 2H); 14.17 (br, 1H) |
| 327 | 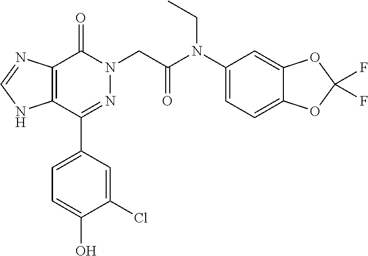 [SMILES: CCN(C(=O)Cn1nc(-c2ccc(O)c(Cl)c2)c2[nH]cnc2c1=O)c1ccc2c(c1)OC(F)(F)O2] | 504 [M + 1] | (400 MHz, DMSO) δ: 1.05 (t, J = 6.8 Hz, 3H); 3.69 (q, J = 6.8 Hz, 2H); 4.77 (s, 2H); 7.12 (d, J = 8.4 Hz, 1H); 7.34 (d, J = 8.0 Hz, 1H); 7.53 (d, J = 8.4 Hz, 1H); 7.66 (s, 1H); 8.25 (s, 1H); 8.50 (s, 2H); 10.67 (br, 1H), 14.17 (br, 1H) |
| 328 | 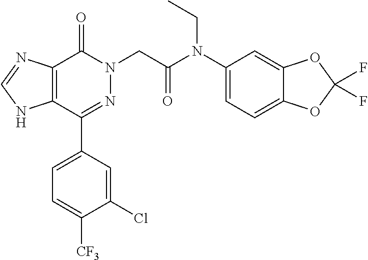 [SMILES: CCN(C(=O)Cn1nc(-c2ccc(C(F)(F)F)c(Cl)c2)c2[nH]cnc2c1=O)c1ccc2c(c1)OC(F)(F)O2] | 556 [M + 1] | (400 MHz, DMSO) δ: 1.03 (t, J = 6.8 Hz, 3H); 3.67 (q, J = 6.8 Hz, 2H); 4.82 (s, 2H); 7.32 (d, J = 7.6 Hz, 1H); 7.52 (d, J = 8.4 Hz, 1H); 7.66 (s, 1H); 8.04 (d, J = 8.4 Hz, 1H); 8.55-8.59 (m, 2H); 8.65 (s, 1H); 14.20 (br, 1H) |
| 329 | 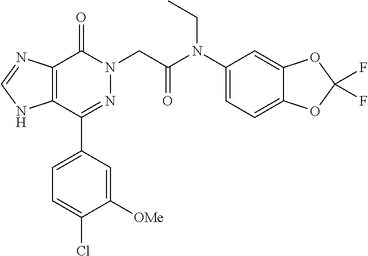 [SMILES: CCN(C(=O)Cn1nc(-c2ccc(Cl)c(OC)c2)c2[nH]cnc2c1=O)c1ccc2c(c1)OC(F)(F)O2] | 518.2 [M + H] | (400 MHz, DMSO-d₆): 1.05 (t, J = 6.8 Hz, 3H), 3.70 (q, J = 6.8 Hz, 2H), 3.97 (s, 3H), 4.80 (s, 2H), 7.34 (d, J = 8.0 Hz, 1H), 7.55 (d, J = 8.4 Hz, 1H), 7.61 (d, J = 8.4 Hz, 1H), 7.67 (s, 1H), 8.03 (m, 1H), 8.21 (s, 1H), 8.52 (s, 1H), 14.14 (br, 1H) |
| 330 | 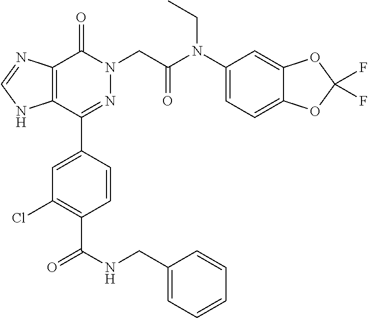 [SMILES: CCN(C(=O)Cn1nc(-c2ccc(C(=O)NCc3ccccc3)c(Cl)c2)c2[nH]cnc2c1=O)c1ccc2c(c1)OC(F)(F)O2] | 621 [M + 1] | (400 MHz, DMSO) δ: 1.03 (m, 3H), 3.68 (q, J = 5.2 Hz, 2H), 4.48 (s, 2H), 4.80 (s, 2H), 7.27 (m, 1H), 7.38 (m, 5H), 7.54 (d, J = 7.6 Hz, 1H), 7.63-7.68 (m, 2H), 8.43 (m, 1H), 8.55 (m, 2H), 9.12 (m, 1H), 14.23 (br, 1H) |
| 331 | 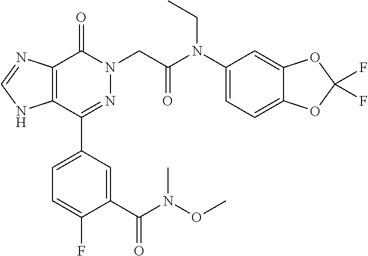 [SMILES: CCN(C(=O)Cn1nc(-c2ccc(F)c(C(=O)N(C)OC)c2)c2[nH]cnc2c1=O)c1ccc2c(c1)OC(F)(F)O2] | 559.2 [M + H] | (400 MHz, DMSO-d₆): 1.03 (t, J = 6.8 Hz, 3H), 3.53 (s, 3H), 3.68 (q, J = 6.8 Hz, 2H), 4.78 (s, 2H), 7.33 (d, J = 8.0 Hz, 1H), 7.46-7.54 (m, 2H), 7.66 (s, 1H), 8.52 (s, 3H), 14.23 (br, 1H) |
| 332 | 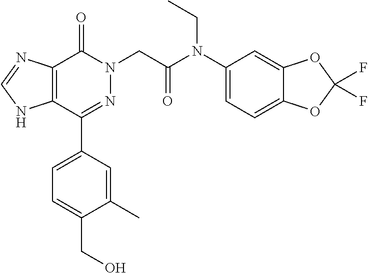 [SMILES: CCN(C(=O)Cn1nc(-c2ccc(CO)c(C)c2)c2[nH]cnc2c1=O)c1ccc2c(c1)OC(F)(F)O2] | 498.2 [M + 1] | (400 MHz, DMSO) δ: 1.05 (t, J = 6.4 Hz, 3H), 2.34 (s, 3H), 3.70 (q, J = 6.4 Hz, 2H), 4.58 (s, 2H), 4.78 (s, 2H), 5.22 (s, 1H), 7.35 (d, J = 8.0 Hz, 1H), 7.51-7.60 (m, 2H), 7.68 (s, 1H), 8.12 (s, 1H), 8.18 (m, 1H), 8.48 (s, 1H), 13.93 (br, 1H) |
| 333 | 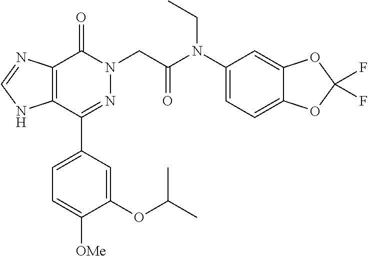 [SMILES: CCN(C(=O)Cn1nc(-c2ccc(OC)c(OC(C)C)c2)c2[nH]cnc2c1=O)c1ccc2c(c1)OC(F)(F)O2] | 542.4 [M + H] | (400 MHz, DMSO-d₆): 1.04 (m, 3H), 1.31 (d, J = 5.6 Hz, 6H), 3.70 (q, J = 5.6 Hz, 2H), 3.85 (s, 3H), 4.60 (sept, J = 5.6 Hz, 1H), 4.78 (s, 2H), 7.13 (d, J = 8.4 Hz, 1H), 7.33 (d, J = 6.4 Hz, 1H), 7.54 (d, J = 8.0 Hz, 1H), 7.64 (s, 1H), 8.03 (m, 2H), 8.49 (s, 1H), 14.08 (br, 1H) |
| 334 | 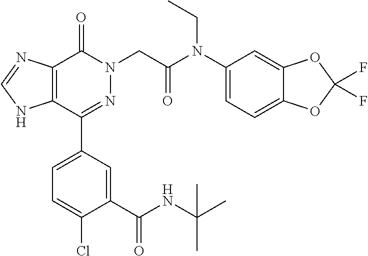 [SMILES: CCN(C(=O)Cn1nc(-c2ccc(Cl)c(C(=O)NC(C)(C)C)c2)c2[nH]cnc2c1=O)c1ccc2c(c1)OC(F)(F)O2] | 587.2 [M + 1] | (400 MHz, DMSO) δ: 1.03 (t, J = 6.8 Hz, 3H), 1.38 (s, 9H), 3.68 (q, J = 6.8 Hz, 2H), 4.79 (s, 2H), 7.34 (d, J = 8.4 Hz, 1H), 7.51-7.56 (m, 2H), 7.67 (s, 1H), 8.15 (s, 1H), 8.37 (m, 1H), 8.53 (s, 2H); 14.22 (br, 1H) |
| 335 | 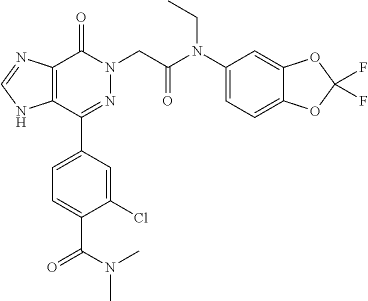 [SMILES: CCN(C(=O)Cn1nc(-c2ccc(C(=O)N(C)C)c(Cl)c2)c2[nH]cnc2c1=O)c1ccc2c(c1)OC(F)(F)O2] | 559.2 [M + 1] | (400 MHz, DMSO) δ: 1.03 (t, J = 6.8 Hz, 3H), 2.82 (s, 3H), 3.04 (s, 3H), 3.68 (q, J = 6.8 Hz, 2H), 4.80 (s, 2H), 7.34 (d, J = 8.4 Hz, 1H), 7.52-7.55 (m, 2H), 7.66 (s, 1H), 8.44 (m, 1H), 8.52-8.56 (m, 2H), 14.22 (br, 1H) |
| 336 | 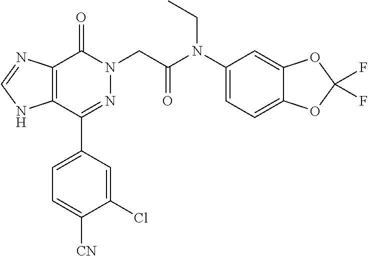 [SMILES: CCN(C(=O)Cn1nc(-c2ccc(C#N)c(Cl)c2)c2[nH]cnc2c1=O)c1ccc2c(c1)OC(F)(F)O2] | 513.3 [M + H] | (400 MHz, DMSO-d₆): 1.06 (t, J = 6.4 Hz, 3H), 3.69 (q, J = 6.4 Hz, 2H), 4.84 (s, 2H), 7.36 (d, J = 8.0 Hz, 1H), 7.55 (d, J = 8.0 Hz, 1H), 7.69 (s, 1H), 8.18 (d, J = 8.4 Hz, 1H), 8.57 (s, 1H), 8.62 (d, J = 7.2 Hz, 1H), 8.80 (s, 1H), 14.30 (br, 1H) |
| 337 | 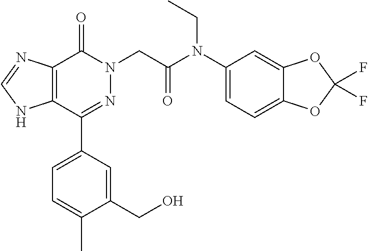 [SMILES: CCN(C(=O)Cn1nc(-c2ccc(C)c(CO)c2)c2[nH]cnc2c1=O)c1ccc2c(c1)OC(F)(F)O2] | 498 [M + 1] | (400 MHz, DMSO) δ: 1.03 (m, 3H); 2.31 (s, 3H); 3.68 (q, J = 6.8 Hz, 2H); 4.56 (d, J = 4.8 Hz, 2H); 4.76 (s, 2H); 5.21 (m, 1H); 7.28 (d, J = 7.6 Hz, 1H); 7.34 (d, J = 7.6 Hz, 1H); 7.54 (d, J = 8.0 Hz, 1H); 7.66 (s, 1H); 8.23 (m, 1H); 8.37 (m, 1H); 8.48 (s, 1H); 14.13 (br, 1H) |
| 338 | 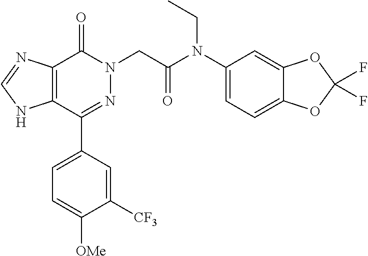 [SMILES: CCN(C(=O)Cn1nc(-c2ccc(OC)c(C(F)(F)F)c2)c2[nH]cnc2c1=O)c1ccc2c(c1)OC(F)(F)O2] | 552.1 [M + 1] | (400 MHz, DMSO) δ: 400; 1.05 (t, J = 6.4 Hz, 3H), 3.68 (q, J = 6.4 Hz, 2H), 3.97 (s, 3H), 4.78 (s, 2H), 7.31 (d, J = 8.0 Hz, 1H), 7.45 (d, J = 8.8 Hz, 1H), 7.51 (d, J = 8.0 Hz, 1H), 7.64 (s, 1H), 8.53 (s, 1H), 8.66 (m, 1H), 8.76 (m, 11H), 14.21 (br, 1H) |
| 339 | 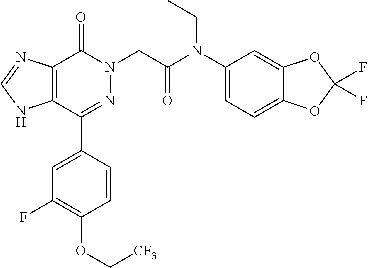 [SMILES: CCN(C(=O)Cn1nc(-c2ccc(OCC(F)(F)F)c(F)c2)c2[nH]cnc2c1=O)c1ccc2c(c1)OC(F)(F)O2] | 570.0 [M + 1] | (400 MHz, DMSO) δ: 1.03 (t, J = 6.8 Hz, 3H), 3.67 (q, J = 6.8 Hz, 2H), 4.77 (s, 2H), 4.96 (q, J = 8.8 Hz, 2H), 7.34 (d, J = 8.4 Hz, 1H), 7.46 (t, J = 8.4 Hz, 1H), 7.52 (d, J = 8.4 Hz, 1H), 7.66 (s, 1H), 8.26- 8.29 (m, 2H), 8.51 (s, 1H), 14.15 (br, 1H) |
| 340 | 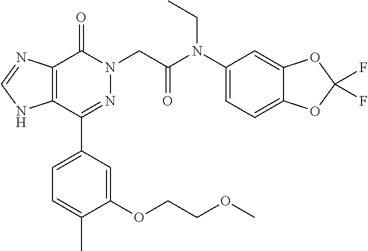 [SMILES: CCN(C(=O)Cn1nc(-c2ccc(C)c(OCCOC)c2)c2[nH]cnc2c1=O)c1ccc2c(c1)OC(F)(F)O2] | 542 [M + 1] | (400 MHz, DMSO) δ: 1.03 (t, J = 6.8 Hz, 3H); 2.23 (s, 3H); 3.68 (q, J = 6.8 Hz, 2H); 3.74 (d, J = 4.4 Hz, 2H); 4.18 (d, J = 4.4 Hz, 2H); 4.76 (s, 2H); 7.30 (m, 2H); 7.52 (d, J = 8.4 Hz, 1H); 7.64 (s, 1H); 7.93 (m, 2H); 8.00 (m, 1H), 8.48 (s, 1H); 14.12 (br, 1H) |
| 341 | 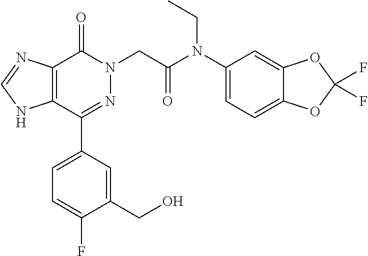 [SMILES: CCN(C(=O)Cn1nc(-c2ccc(F)c(CO)c2)c2[nH]cnc2c1=O)c1ccc2c(c1)OC(F)(F)O2] | 502.2 [M + 1] | (400 MHz, DMSO) δ: 1.03 (t, J = 6.8 Hz, 3H), 3.68 (d, J = 6.8 Hz, 2H), 4.63 (d, J = 4.8 Hz, 2H), 4.77 (s, 2H), 5.41 (t, J = 4.8 Hz, 1H), 7.28-7.33 (m, 2H), 7.53 (d, J = 8.4 Hz, 1H), 7.66 (s, 1H), 8.32 (m, 1H), 8.47 (m, 2H), 14.13 (br, 1H) |
| 342 | 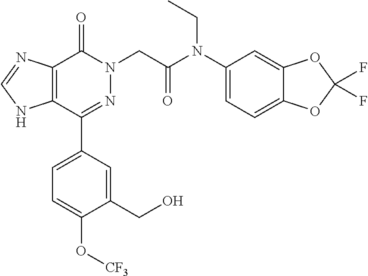 [SMILES: CCN(C(=O)Cn1nc(-c2ccc(OC(F)(F)F)c(CO)c2)c2[nH]cnc2c1=O)c1ccc2c(c1)OC(F)(F)O2] | 568.2 [M + H] | (400 MHz, DMSO) δ: 1.03 (t, J = 6.8 Hz, 3H), 3.68 (q, J = 6.8 Hz, 2H), 4.64 (d, J = 5.6 Hz, 2H), 4.78 (s, 2H), 5.51 (t, J = 5.6 Hz, 1H), 7.33 (d, J = 7.6 Hz, 1H), 7.49 (d, J = 8.8 Hz, 1H), 7.5 (d, J = 8.4 Hz, 1H), 7.66 (s, 1H), 8.42 (m, 1H), 8.52 (s, 1H), 8.61 (m, 1H), 14.20 (br, 1H) |
| 343 | 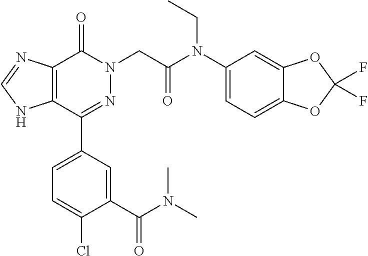 [SMILES: CCN(C(=O)Cn1nc(-c2ccc(Cl)c(C(=O)N(C)C)c2)c2[nH]cnc2c1=O)c1ccc2c(c1)OC(F)(F)O2] | 559.1 [M + 1] | (400 MHz, DMSO) δ: 1.03 (t, J = 6.8 Hz, 3H), 2.83 (s, 3H), 3.04 (s, 3H), 3.68 (q, J = 6.8 Hz, 2H), 4.78 (s, 2H), 7.34 (d, J = 8.0 Hz, 1H), 7.53 (d, J = 8.4 Hz, 1H), 7.66 (s, 1H), 7.70 (d, J = 8.4 Hz, 1H), 8.40 (m, 1H), 8.48-8.51 (m, 2H), 14.20 (br, 1H) |
| 344 | 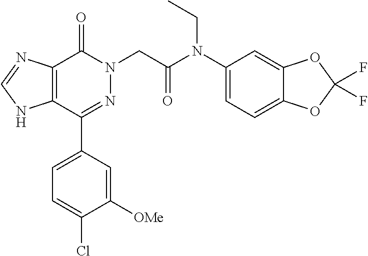 [SMILES: CCN(C(=O)Cn1nc(-c2ccc(Cl)c(OC)c2)c2[nH]cnc2c1=O)c1ccc2c(c1)OC(F)(F)O2] | 552.0 [M + 1] | (400 MHz, DMSO) δ: 1.03 (m, 3H), 3.68 (q, J = 6.8 Hz, 2H), 3.98 (s, 3H), 4.73 (s, 2H), 7.32 (d, J = 8.0 Hz, 1H), 7.52 (d, J = 8.0 Hz, 1H), 7.65 (s, 1H), 7.71 (d, J = 8.0 Hz, 1H), 8.05 (s, 1H), 8.21 (d, J = 7.6 Hz, 1H), 8.51 (s, 1H) |
| 345 | 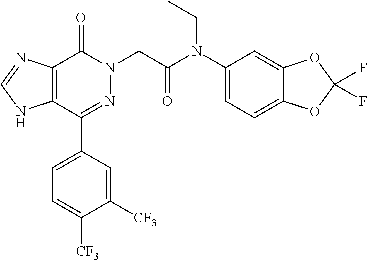 [SMILES: CCN(C(=O)Cn1nc(-c2ccc(C(F)(F)F)c(C(F)(F)F)c2)c2[nH]cnc2c1=O)c1ccc2c(c1)OC(F)(F)O2] | 590 [M + 1] | (400 MHz, DMSO) δ: 1.03 (t, J = 6.8 Hz, 3H); 3.68 (q, J = 6.8 Hz, 2H); 4.85 (s, 2H); 7.32 (d, J = 7.6 Hz, 1H); 7.51 (d, J = 8.4 Hz, 1H); 7.64 (s, 1H); 8.26 (d, J = 8.4 Hz, 1H); 8.59 (s, 1H); 8.91 (d, J = 7.6 Hz, 1H); 9.13 (s, 1H); 14.35 (br, 1H) |
| 346 | 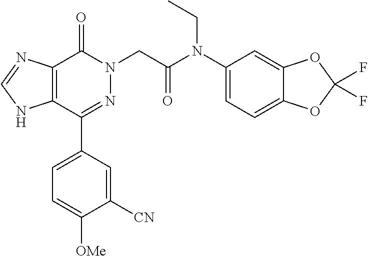 [SMILES: CCN(C(=O)Cn1nc(-c2ccc(OC)c(C#N)c2)c2[nH]cnc2c1=O)c1ccc2c(c1)OC(F)(F)O2] | 509 [M + 1] | (400 MHz, DMSO) δ: 1.05 (t, J = 6.8 Hz, 3H); 3.69 (q, J = 6.8 Hz, 2H); 4.02 (s, 3H); 4.80 (s, 2H); 7.35 (d, J = 8.4 Hz, 1H); 7.47 (d, J = 9.2 Hz, 1H); 7.54 (d, J = 8.4 Hz, 1H); 7.68 (s, 1H); 8.55 (s, 1H); 8.77 (m, 2H); 14.25 (br, 1H) |
| 347 | 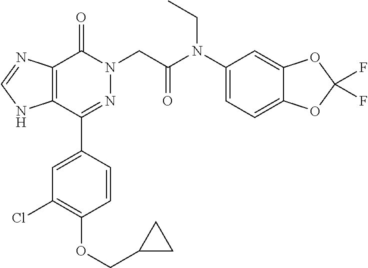 [SMILES: CCN(C(=O)Cn1nc(-c2ccc(OCC3CC3)c(Cl)c2)c2[nH]cnc2c1=O)c1ccc2c(c1)OC(F)(F)O2] | 558 [M + 1] | (400 MHz, DMSO) δ: 0.39 (d, J = 4.0 Hz, 2H); 0.61 (d, J = 6.8 Hz, 2H); 1.03 (t, J = 6.8 Hz, 3H); 1.29 (m, 1H); 3.67 (m, 2H); 4.01 (m, 2H); 4.77 (s, 2H); 7.28 (d, J = 8.8 Hz, 1H), 7.33 (d, J = 8.4 Hz, 1H); 7.52 (d, J = 8.4 Hz, 1H); 7.65 (s, 1H); 8.40 (m, 1H); 8.51 (s, 2H); 14.18 (br, 1H) |
| 348 | 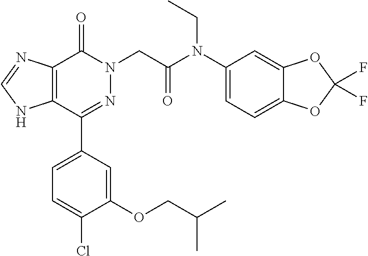 [SMILES: CCN(C(=O)Cn1nc(-c2ccc(Cl)c(OCC(C)C)c2)c2[nH]cnc2c1=O)c1ccc2c(c1)OC(F)(F)O2] | 560 [M + 1] | (400 MHz, DMSO) δ: 1.03- 1.08 (m, 9H); 2.14 (m, 1H); 3.69 (m, 2H); 3.95 (d, J = 6.0 Hz, 2H); 4.80 (s, 2H); 7.34 (d, J = 8.0 Hz, 1H); 7.54 (d, J = 8.4 Hz, 1H); 7.60 (d, J = 8.4 Hz, 1H); 7.66 (s, 1H); 8.01 (m, 1H); 8.19 (m, 1H); 8.53 (s, 1H) 14.20 (br, 1H) |
| 349 | 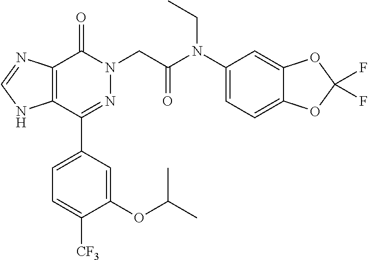 [SMILES: CCN(C(=O)Cn1nc(-c2ccc(C(F)(F)F)c(OC(C)C)c2)c2[nH]cnc2c1=O)c1ccc2c(c1)OC(F)(F)O2] | 580.2 [M + 1] | (400 MHz, DMSO) δ: 1.03 (t, J = 6.8 Hz, 3H), 1.37 (d, J = 6.0 Hz, 6H), 3.68 (q, J = 6.8 Hz, 2H), 4.81-4.85 (m, 3H), 7.32 (d, J = 8.0 Hz, 1H), 7.52 (d, J = 8.4 Hz, 1H), 7.64 (s, 1H), 7.75 (d, J = 8.4 Hz, 1H), 8.01 (d, J = 8.0 Hz, 1H), 8.40 (s, 1H), 8.56 (s, 1H), 14.27 (br, 1H) |
| 350 | 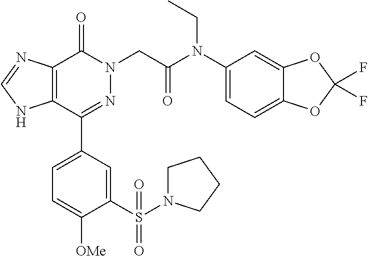 [SMILES: CCN(C(=O)Cn1nc(-c2ccc(OC)c(S(=O)(=O)N3CCCC3)c2)c2[nH]cnc2c1=O)c1ccc2c(c1)OC(F)(F)O2] | 617.2 [M + 1] | (400 MHz, DMSO) δ: 1.03 (t, J = 6.8 Hz, 3H), 1.78 (m, 4H), 3.28-3.34 (m, 4H), 3.68 (q, J = 6.8 Hz, 2H), 3.98 (s, 3H), 4.76 (s, 2H), 7.33 (d, J = 8.0 Hz, 1H), 7.42 (d, J = 9.2 Hz, 1H), 7.53 (d, J = 8.4 Hz, 1H), 7.65 (s, 1H), 8.46 (s, 1H), 8.68 (d, J = 8.0 Hz, 1H), 8.88 (s, 1H) |
| 351 | 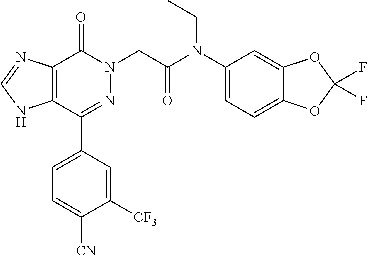 [SMILES: CCN(C(=O)Cn1nc(-c2ccc(C#N)c(C(F)(F)F)c2)c2[nH]cnc2c1=O)c1ccc2c(c1)OC(F)(F)O2] | 547.2 [M + 1] | (400 MHz, DMSO) δ: 1.03 (t, J = 6.8 Hz, 3H), 3.67 (q, J = 6.8 Hz, 2H), 4.84 (s, 2H), 7.32 (d, J = 8.0 Hz, 1H), 7.52 (d, J = 8.4 Hz, 1H), 7.65 (s, 1H), 8.36 (d, J = 8.4 Hz, 1H), 8.61 (s, 1H), 8.90 (d, J = 6.8 Hz, 1H), 9.08 (s, 1H), 14.38 (br, 1H) |
| 352 | 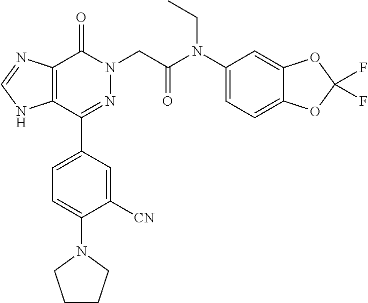 [SMILES: CCN(C(=O)Cn1nc(-c2ccc(N3CCCC3)c(C#N)c2)c2[nH]cnc2c1=O)c1ccc2c(c1)OC(F)(F)O2] | 548.2 [M + 1] | (400 MHz, DMSO) δ: 1.03 (m, 3H), 1.98 (m, 4H), 3.61 (m, 4H), 3.66 (m, 2H), 4.75 (s, 2H), 6.92 (d, J = 8.0 Hz, 1H), 7.33 (m, 1H), 7.51 (m, 1H), 7.65 (s, 1H), 8.36 (m, 1H), 8.49 (s, 1H), 8.75 (m, 1H), 14.15 (br, 1H) |
| 353 | 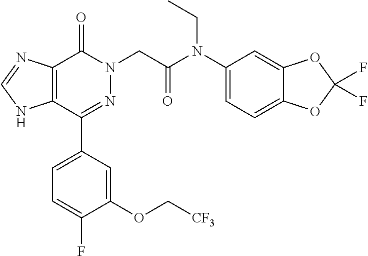 [SMILES: CCN(C(=O)Cn1nc(-c2ccc(F)c(OCC(F)(F)F)c2)c2[nH]cnc2c1=O)c1ccc2c(c1)OC(F)(F)O2] | 570.2 [M + 1] | (400 MHz, DMSO) δ: 1.03 (t, J = 6.8 Hz, 3H), 3.68 (q, J = 6.8 Hz, 2H), 4.78 (s, 2H), 4.93 (q, J = 4.4 Hz, 2H), 7.316-7.33 (m, 1H), 7.44-7.55 (m, 2H), 7.65 (s, 1H), 8.20 (m, 2H), 8.52 (s, 1H), 14.21 (br, 1H) |
| 354 | 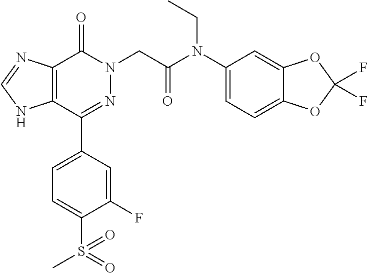 [SMILES: CCN(C(=O)Cn1nc(-c2ccc(S(C)(=O)=O)c(F)c2)c2[nH]cnc2c1=O)c1ccc2c(c1)OC(F)(F)O2] | 550.2 [M + 1] | (400 MHz, DMSO) δ: 1.03 (t, J = 6.8 Hz, 3H), 3.39 (s, 3H), 3.68 (q, J = 6.8 Hz, 2H), 4.82 (s, 2H) 7.34 (d, J = 7.6 Hz, 1H), 7.54 (d, J = 8.8 Hz, 1H), 7.67 (s, 1H), 8.04 (t, J = 8.0 Hz, 1H), 8.52 (m, 2H), 8.59 (s, 1H), 14.35 (br, 1H) |
| 355 | 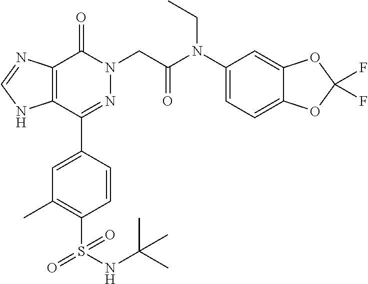 [SMILES: CCN(C(=O)Cn1nc(-c2ccc(S(=O)(=O)NC(C)(C)C)c(C)c2)c2[nH]cnc2c1=O)c1ccc2c(c1)OC(F)(F)O2] | 603.2 [M + 1] | (400 MHz, DMSO) δ: 1.03 (t, J = 6.8 Hz, 3H), 1.12 (s, 9H), 2.68 (s, 3H), 3.68 (q, J = 6.8 Hz, 2H), 4.80 (s, 2H), 7.34 (d, J = 8.0 Hz, 1H), 7.54 (d, J = 8.4 Hz, 1H), 7.61 (s, 1H), 7.66 (s, 1H), 8.02 (d, J = 8.4 Hz, 1H), 8.35-8.37 (m, 2H), 8.54 (s, 1H), 14.2 (br, 1H) |
| 356 | 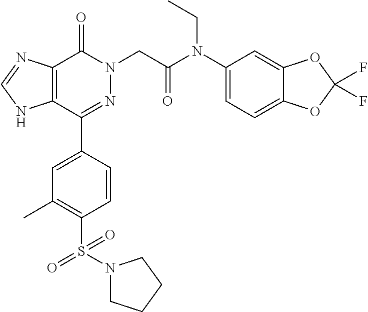 [SMILES: CCN(C(=O)Cn1nc(-c2ccc(S(=O)(=O)N3CCCC3)c(C)c2)c2[nH]cnc2c1=O)c1ccc2c(c1)OC(F)(F)O2] | 601.4 [M + 1] | (400 MHz, DMSO) δ: 1.03 (t, J = 6.8 Hz, 3H), 1.83 (s, 4H), 2.65 (s, 3H), 3.23 (s, 4H), 3.68 (q, J = 6.8 Hz, 2H), 4.79 (s, 2H), 7.33 (d, J = 8.0 Hz, 1H), 7.53 (d, J = 8.4 Hz, 1H), 7.65 (s, 1H), 7.93 (d, J = 8.4 Hz, 1H), 8.40 (s, 2H), 8.49 (s, 1H) |
| 357 | 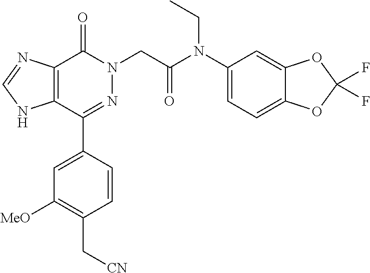 [SMILES: CCN(C(=O)Cn1nc(-c2ccc(CC#N)c(OC)c2)c2[nH]cnc2c1=O)c1ccc2c(c1)OC(F)(F)O2] | 523.2 [M + 1] | (400 MHz, DMSO) δ: 1.03 (m, 3H), 3.68 (m, 2H), 3.93 (m, 5H), 4.78 (s, 2H), 7.33 (m, 1H), 7.51-7.53 (m, 2H), 7.64 (s, 1H), 7.99- 8.06 (m, 2H), 8.51 (s, 1H), 14.18 (br, 1H) |
| 358 | 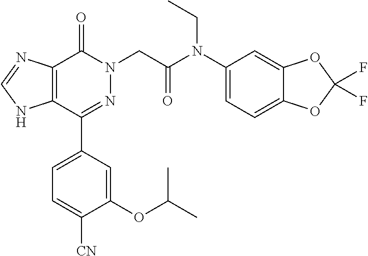 [SMILES: CCN(C(=O)Cn1nc(-c2ccc(C#N)c(OC(C)C)c2)c2[nH]cnc2c1=O)c1ccc2c(c1)OC(F)(F)O2] | 537.2 [M + 1] | (400 MHz, DMSO) δ: 1.03 (t, J = 6.8 Hz, 3H), 1.41 (d, J = 6.0 Hz, 6H), 3.68 (q, J = 6.8 Hz, 2H), 4.81-8.84 (m, 3H), 7.32 (d, J = 8.0 Hz, 1H), 7.52 (d, J = 8.4 Hz, 1H), 7.64 (s, 1H), 7.87 (d, J = 8.4 Hz, 1H), 8.00 (m, 1H), 8.39 (m, 1H), 8.55 (s, 1H), 14.27 (br, 1H) |
| 359 | 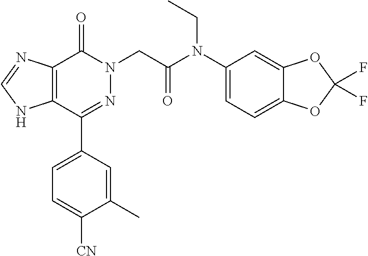 [SMILES: CCN(C(=O)Cn1nc(-c2ccc(C#N)c(C)c2)c2[nH]cnc2c1=O)c1ccc2c(c1)OC(F)(F)O2] | 493.2 [M + H] | (400 MHz, DMSO-d₆): 1.03 (t, J = 6.8 Hz, 3H), 2.67 (s, 3H), 3.68 (q, J = 6.8 Hz, 2H), 4.80 (s, 2H), 7.33 (d, J = 8.0 Hz, 1H), 7.53 (d, J = 8.4 Hz, 1H), 7.66 (s, 1H), 7.94 (d, J = 8.0 Hz, 1H), 8.44 (m, 2H), 8.54 (s, 1H), 14.27 (br, 1H) |
| 360 | 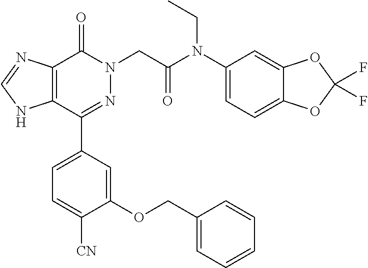 [SMILES: CCN(C(=O)Cn1nc(-c2ccc(C#N)c(OCc3ccccc3)c2)c2[nH]cnc2c1=O)c1ccc2c(c1)OC(F)(F)O2] | 585.2 [M + 1] | (400 MHz, DMSO) δ: 1.03 (t, J = 6.8 Hz, 3H), 3.69 (q, J = 6.8 Hz, 2H), 4.81 (s, 2H), 5.38 (s, 2H), 7.31- 7.37 (m, 2H), 7.42-7.45 (m, 2H), 7.52-7.57 (m, 3H), 7.66 (s, 1H), 7.91 (d, J = 8.0 Hz, 1H), 8.41 (s, 1H), 8.55 (s, 1H), 14.24 (br, 1H) |
| 361 | 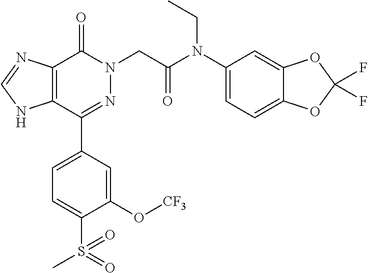 [SMILES: CCN(C(=O)Cn1nc(-c2ccc(S(C)(=O)=O)c(OC(F)(F)F)c2)c2[nH]cnc2c1=O)c1ccc2c(c1)OC(F)(F)O2] | 616.0 [M + 1] | (400 MHz, DMSO) δ: 1.06 (m, 3H), 3.39 (s, 3H), 3.70 (m, 2H), 4.85 (s, 2H), 7.34 (d, J = 5.6 Hz, 1H), 7.54 (d, J = 8.0 Hz, 1H), 7.64 (s, 1H), 8.21 (d, J = 8.0 Hz, 1H), 8.62 (m, 2H), 8.84 (s, 1H), 14.36 (s, 1H) |
| 362 | 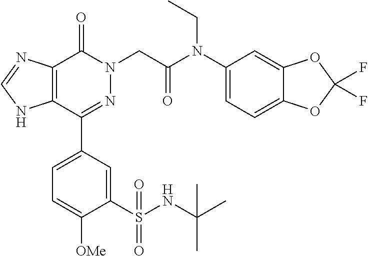 [SMILES: CCN(C(=O)Cn1nc(-c2ccc(OC)c(S(=O)(=O)NC(C)(C)C)c2)c2[nH]cnc2c1=O)c1ccc2c(c1)OC(F)(F)O2] | 619.4 [M + H] | (400 MHz, DMSO-d₆): 1.05 (t, J = 6.8 Hz, 3H), 1.09 (s, 9H), 3.68 (q, J = 6.8 Hz, 2H), 3.99 (s, 3H), 4.76 (s, 2H), 7.16 (s, 1H), 7.33- 7.38 (m, 2H), 7.53 (d, J = 8.0 Hz, 1H), 7.65 (s, 1H), 8.52 (s, 1H), 8.64 (m, 1H), 8.87 (m, 1H), 14.17 (br, 1H) |
| 363 | 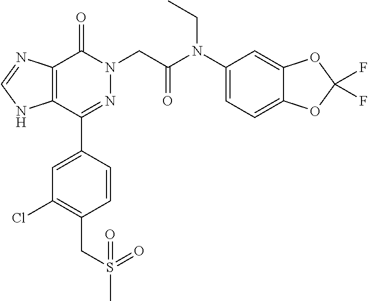 [SMILES: CCN(C(=O)Cn1nc(-c2ccc(CS(C)(=O)=O)c(Cl)c2)c2[nH]cnc2c1=O)c1ccc2c(c1)OC(F)(F)O2] | 580.0 [M + H] | (400 MHz, DMSO-d₆): 1.03 (t, J = 6.8 Hz, 3H), 3.06 (s, 3H), 3.68 (q, J = 6.8 Hz, 2H), 4.74 (s, 2H), 4.80 (s, 2H), 7.34 (d, J = 8.0 Hz, 1H), 7.53 (d, J = 8.4 Hz, 1H), 7.66 (s, 1H), 7.71 (d, J = 8.0 Hz, 1H), 8.38 (m, 1H), 8.53 (s, 1H), 8.59 (m, 1H), 14.22 (br, 1H) |
| 364 | 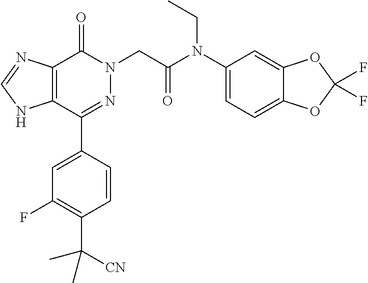 [SMILES: CCN(C(=O)Cn1nc(-c2ccc(C(C)(C)C#N)c(F)c2)c2[nH]cnc2c1=O)c1ccc2c(c1)OC(F)(F)O2] | 539.2 [M + H] | (400 MHz, DMSO) δ: 1.03 (m, 3H), 1.78 (s, 6H), 3.67 (q, J = 6.4 Hz, 2H), 4.79 (s, 2H), 7.31 (d, J = 7.6 Hz, 1H), 7.51 (d, J = 8.0 Hz, 1H), 7.64 (s, 2H), 8.29 (m, 2H), 8.53 (s, 1H), 14.24 (br, 1H) |
| 365 | 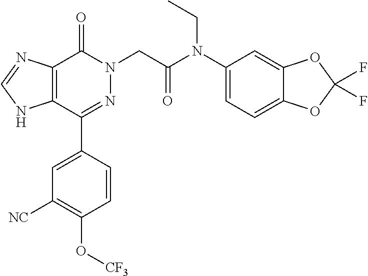 [SMILES: CCN(C(=O)Cn1nc(-c2ccc(OC(F)(F)F)c(C#N)c2)c2[nH]cnc2c1=O)c1ccc2c(c1)OC(F)(F)O2] | 563.1 [M + H] | (400 MHz, DMSO-d₆): 1.03 (m, 3H), 3.67 (m, 2H), 4.81 (s, 2H), 7.33 (d, J = 8.4 Hz, 1H), 7.52 (s, 1H), 7.67 (s, 1H), 7.91 (d, J = 8.4 Hz, 1H), 8.58 (s, 1H), 8.87 (m, 1H), 8.96 (m, 1H), 14.34 (br, 1H) |
| 366 | 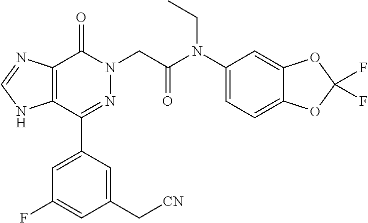 [SMILES: CCN(C(=O)Cn1nc(-c2cc(F)cc(CC#N)c2)c2[nH]cnc2c1=O)c1ccc2c(c1)OC(F)(F)O2] | 521 [M − 1] | (400 MHz, DMSO) δ: 0.69 (s, 2H); 0.93 (s, 2H); 3.33 (m, 1H); 4.18 (m, 2H); 5.21 (s, 2H); 7.16 (d, J = 7.6 Hz, 1H); 7.32 (d, J = 8.0 Hz, 2H); 7.42 (m, 2H); 8.22- 8.30 (m, 2H); 8.46 (d, J = 7.6 Hz, 1H) |
| 367 | 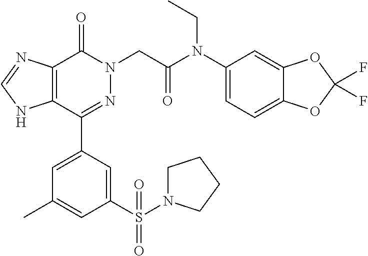 | 613.0 [M + 1] | (400 MHz, DMSO) δ: 0.68 (s, 2H), 0.91 (s, 2H), 1.74 (s, 4H), 2.51 (s, 3H), 3.26 (s, 4H), 3.32 (s, 1H), 5.21 (s, 2H), 7.15-7.17 (d, J = 7.6 Hz, 1H), 7.40-7.42 (d, J = 8.0 Hz, 2H), 7.72 (s, 1H), 8.49 (m, 2H), 8.76 (s, 1H), 14.04 (s, 1H) |
| 368 | 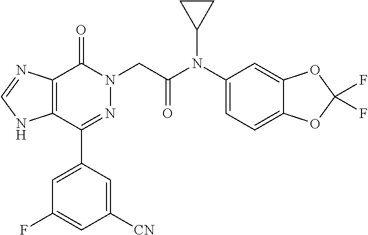 [SMILES: N#Cc1cc(F)cc(-c2nn(CC(=O)N(c3ccc4c(c3)OC(F)(F)O4)C3CC3)c(=O)c3nc[nH]c23)c1] | 509.3 [M + 1] | (400 MHz, DMSO) 0.60- 0.86 (m, 4H), 5.42 (s, 2H), 7.17 (m, 1H), 7.46 (m, 2H), 7.97 (d, J = 7.2 Hz, 1H), 8.33 (m, 1H), 8.72 (m, 1H), 8.81 (s, 1H) |
| 369 | 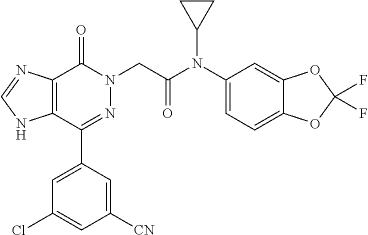 [SMILES: N#Cc1cc(Cl)cc(-c2nn(CC(=O)N(c3ccc4c(c3)OC(F)(F)O4)C3CC3)c(=O)c3nc[nH]c23)c1] | 523.5 [M − H] | (400 MHz, DMSO-d₆): 0.67 (s, 2H), 0.91 (d, J = 5.6 Hz, 2H), 3.31 (m, 1H), 5.21 (s, 2H), 7.13-7.16 (m, 1H), 7.39 (m, 2H), 8.05 (s, 1H), 8.45 (s, 1H), 8.80 (m, 2H) |
| 370 | 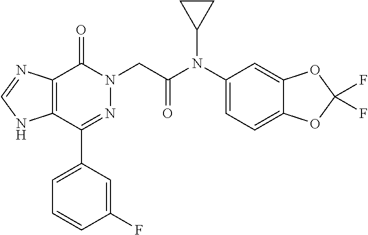 [SMILES: O=C(Cn1nc(-c2cccc(F)c2)c2[nH]cnc2c1=O)N(c1ccc2c(c1)OC(F)(F)O2)C1CC1] | 484.1 [M + 1] | (400 MHz, DMSO) δ: 0.63 (m, 2H), 0.90 (m, 2H), 5.13 (s, 2H), 7.15 (m, 1H), 7.21 (m, 1H), 7.37 (m, 2H), 7.52 (m, 1H), 8.08 (s, 1H), (8.34 (m, 2H) |
| 371 | 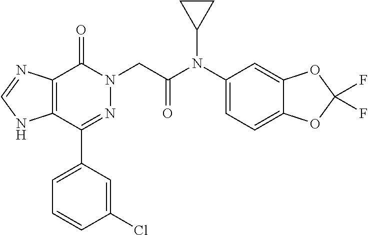 [SMILES: O=C(Cn1nc(-c2cccc(Cl)c2)c2[nH]cnc2c1=O)N(c1ccc2c(c1)OC(F)(F)O2)C1CC1] | 500. [M + 1] | (400 MHz, DMSO) δ: 0.67 (s, 2H), 0.90 (m, 2H), 3.30 (m, 1H), 5.18 (s, 2H), 7.14 (dd, J = 8.8, 2.0 Hz, 1H), 7.37-7.39 (m, 2H), 7.50- 7.57 (m, 2H), 8.37-8.39 (m, 2H), 8.47 (s, 1H), 13.60 (br, 1H) |
| 372 | 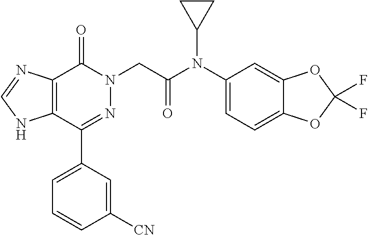 [SMILES: N#Cc1cccc(-c2nn(CC(=O)N(c3ccc4c(c3)OC(F)(F)O4)C3CC3)c(=O)c3nc[nH]c23)c1] | 491.2 [M + H] | (400 MHz, DMSO-d₆): 0.67 (s, 2H), 0.90 (d, J = 5.6 Hz, 2H), 3.30 (m, 1H), 5.20 (s, 2H), 7.13-7.16 (m, 1H), 7.37-7.39 (m, 2H), 7.73- 7.77 (m, 1H), 7.89-7.91 (d, J = 7.6 Hz, 1H), 8.41 (s, 1H), 8.70-8.72 (d, J = 8.0 Hz, 1H), 8.80 (s, 1H), 13.69 (br, 1H) |
| 373 | 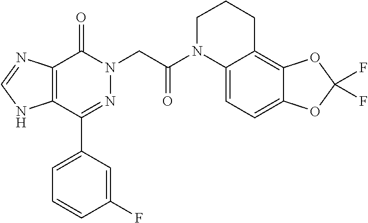 [SMILES: O=C(Cn1nc(-c2cccc(F)c2)c2[nH]cnc2c1=O)N1CCCc2c1ccc1c2OC(F)(F)O1] | 484.5 [M + H] | (400 MHz, DMSO): 2.00 (m, 2H), 2.82 (m, 2H), 3.83 (m, 2H), 5.35 (s, 2H), 7.22- 7.24 (d, J = 8.8 Hz, 1H), 7.36 (m, 1H), 7.51 (m, 1H), 7.57-7.63 (m, 1H), 8.24- 8.30 (m, 2H), 8.53 (s, 1H), 14.28 (br, 1H) |
| 374 | 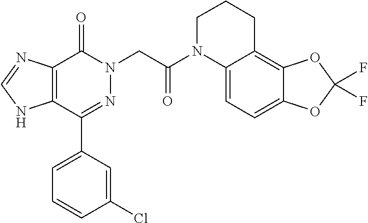 [SMILES: O=C(Cn1nc(-c2cccc(Cl)c2)c2[nH]cnc2c1=O)N1CCCc2c1ccc1c2OC(F)(F)O1] | 500.4 [M + H] | (400 MHz, DMSO): 2.00 (m, 2H), 2.80-2.83 (t, J = 6.4 Hz, 2H), 3.83 (m, 2H), 5.36 (s, 2H), 7.22- 7.24 (d, J = 9.2 Hz, 1H), 7.52-7.61 (m, 3H), 8.42 (m, 1H), 8.51-8.54 (m, 2H), 14.28 (br, 1H) |
| 375 | 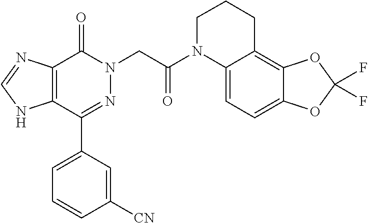 [SMILES: N#Cc1cccc(-c2nn(CC(=O)N3CCCc4c3ccc3c4OC(F)(F)O3)c(=O)c3nc[nH]c23)c1] | 491.2 [M + H] | (400 MHz, DMSO): 2.00 (m, 2H), 2.81-2.84 (t, J = 6.4 Hz, 2H), 3.83 (m, 2H), 5.37 (s, 2H), 7.23- 7.25 (d, J = 8.0 Hz, 1H), 7.52-7.53 (d, J = 6.0 Hz, 1H), 7.77-7.81 (m, 1H), 7.97-7.99 (d, J = 8.0 Hz, 1H), 8.55 (s, 1H), 8.76 (m, 1H), 8.86 (m, 1H), 14.33 (br, 1H) |
| 376 | 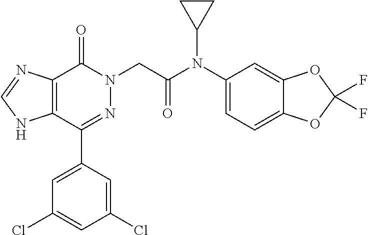 [SMILES: O=C(Cn1nc(-c2cc(Cl)cc(Cl)c2)c2[nH]cnc2c1=O)N(c1ccc2c(c1)OC(F)(F)O2)C1CC1] | 535 [M + 1] | (400 MHz, DMSO) δ: 0.69 (m, 2H); 0.93 (m, 2H); 3.32 (m, 1H); 5.22 (s, 2H); 7.156-7.177 (d, J = 8.4 Hz, 1H); 7.40-7.43 (m, 2H); 7.68 (s, 1H); 8.46-8.51 (m, 2H), 14.03 (br, 1H) |
| 377 | 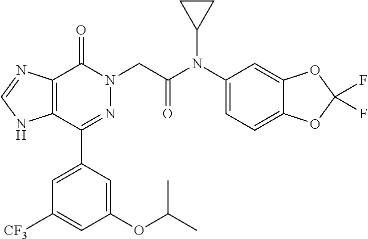 [SMILES: CC(C)Oc1cc(-c2nn(CC(=O)N(c3ccc4c(c3)OC(F)(F)O4)C3CC3)c(=O)c3nc[nH]c23)cc(C(F)(F)F)c1] | 592.2 [M + 1] | (400 MHz, DMSO) δ: 0.67 (s, 2H), 0.91 (s, 2H), 1.37-1.39 (d, J = 6.0 Hz, 6H), 3.31 (m, 1H), 4.82 (sept, J = 6.0 Hz, 1H), 5.21 (s, 2H), 7.15 (d, J = 8.4 Hz, 1H), 7.30 (s, 1H), 7.41 (m, 2H), 8.34 (m, 2H), 8.43 (s, 1H) |
| 378 | 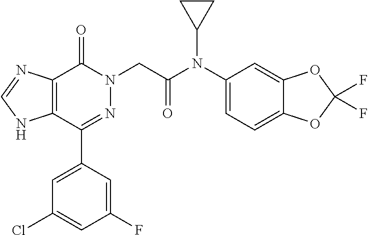 [SMILES: O=C(Cn1nc(-c2cc(F)cc(Cl)c2)c2[nH]cnc2c1=O)N(c1ccc2c(c1)OC(F)(F)O2)C1CC1] | 518 [M + 1] | (400 MHz, DMSO) δ: 0.69 (m, 2H); 0.92 (m, 2H); 3.32 (m, 1H); 5.22 (s, 2H); 7.17 (d, J = 8.4 Hz, 1H); 7.40- 7.52 (m, 3H); 8.26 (d, J = 10.0 Hz, 1H); 8.40 (m, 1H), 8.49 (m, 1H) 14.06 (br, 1H) |
| 379 | 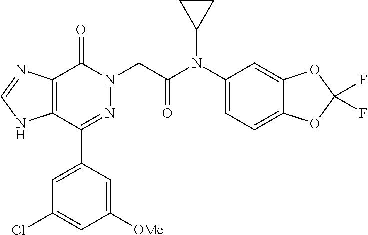 [SMILES: COc1cc(Cl)cc(-c2nn(CC(=O)N(c3ccc4c(c3)OC(F)(F)O4)C3CC3)c(=O)c3nc[nH]c23)c1] | 530 [M + 1] | (400 MHz, DMSO) δ: 0.68 (m, 2H); 0.92 (m, 2H); 3.31 (m, 1H); 3.90 (s, 3H); 5.20 (s, 2H); 7.13-7.18 (m, 2H); 7.40-7.43 (m, 2H); 8.03 (s, 1H); 8.06 (s, 1H), 8.16 (s, 1H). 8.44 (s, 1H), 13.94 (br, 1H) |
| 380 | 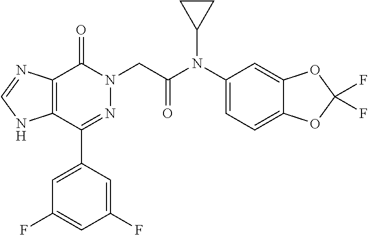 [SMILES: O=C(Cn1nc(-c2cc(F)cc(F)c2)c2[nH]cnc2c1=O)N(c1ccc2c(c1)OC(F)(F)O2)C1CC1] | 502 [M + 1] | (400 MHz, DMSO) δ: 0.68 (s, 2H); 0.92 (d, J = 5.6 Hz, 2H); 3.32 (m, 1H); 5.21 (s, 2H); 7.16 (dd, J = 8.8, 2.0 Hz, 1H); 7.30 (t, J = 8.8 Hz, 1H); 7.40-7.43 (m, 2H); 8.16 (s, 1H), 8.17 (s, 1H), 8.48 (s, 1H), 14.05 (br, 1H) |
| 381 | 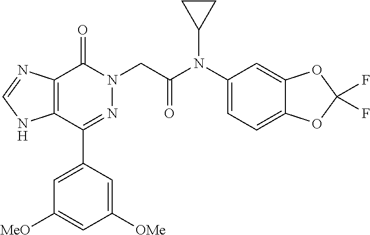 [SMILES: COc1cc(OC)cc(-c2nn(CC(=O)N(c3ccc4c(c3)OC(F)(F)O4)C3CC3)c(=O)c3nc[nH]c23)c1] | 526 [M + 1] | (400 MHz, DMSO) δ: 0.68 (m, 2H); 0.92 (m, 2H); 3.31 (m, 1H); 3.86 (s, 6H); 5.18 (s, 2H); 6.65 (t, J = 2.4 Hz, 1H); 7.15 (m, 1H); 7.40 (m, 2H); 7.60 (m, 2H). 8.40 (s, 1H) 13.84 (br, 1H) |
| 382 | 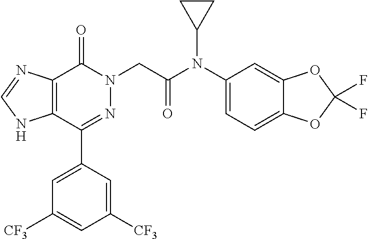 [SMILES: O=C(Cn1nc(-c2cc(C(F)(F)F)cc(C(F)(F)F)c2)c2[nH]cnc2c1=O)N(c1ccc2c(c1)OC(F)(F)O2)C1CC1] | 602.0 [M + 1] | (400 MHz, DMSO) δ: 0.69 (s, 2H), 0.92 (s, 2H), 3.33 (s, 1H), 5.25 (s, 2H), 7.15- 7.17 (t, J = 8.4 Hz, 1H), 7.40-7.41 (m, 2H), 8.18 (d, J = 9.2 Hz, 1H), 8.55 (d, J = 10.0 Hz, 1H), 9.14 (d, J = 8.4 Hz, 2H), 14.10 (br, 1H) |
| 383 | 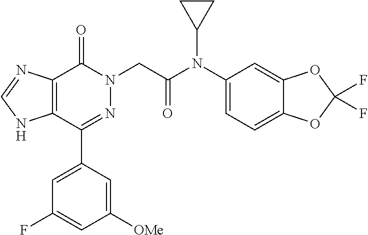 [SMILES: COc1cc(F)cc(-c2nn(CC(=O)N(c3ccc4c(c3)OC(F)(F)O4)C3CC3)c(=O)c3nc[nH]c23)c1] | 514.0 [M + 1] | (400 MHz, DMSO) δ: 0.67 (s, 2H), 0.90 (m, 2H), 3.30 (m, 1H), 3.89 (s, 3H), 5.19 (s, 2H), 6.93 (d, J = 10.8 Hz, 1H), 7.16 (d, J = 8.4 Hz, 1H), 7.40-7.41 (m, 2H), 7.79 (m, 1H), 7.93 (m, 1H), 8.44 (s, 1H), 13.93 (br, 1H) |
| 384 | 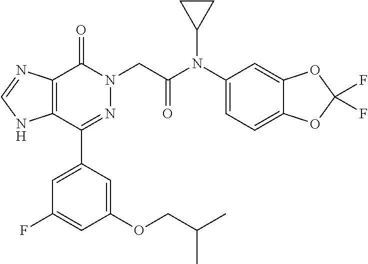 [SMILES: CC(C)COc1cc(F)cc(-c2nn(CC(=O)N(c3ccc4c(c3)OC(F)(F)O4)C3CC3)c(=O)c3nc[nH]c23)c1] | 554 [M − 1] | (400 MHz, DMSO) δ: 0.68 (s, 2H); 0.92 (d, J = 6.0 Hz, 2H); 1.04 (d, J = 6.8 Hz, 6H); 2.10 (m, 1H); 3.31 (m, 1H); 3.88 (d, J = 6.4 Hz, 2H); 5.20 (s, 2H); 6.92 (d, J = 10.8 Hz, 1H); 7.16 (dd, J = 8.4, 2.0 Hz, 1H); 7.39-7.42 (m, 2H); 7.79 (d, J = 9.6 Hz, 1H). 7.92 (m, 1H), 8.44 (s, 1H), 13.95 (br, 1H) |
| 385 | 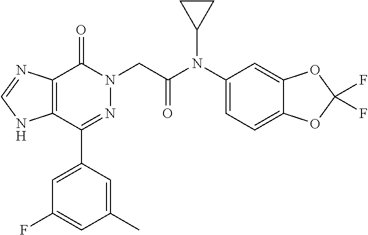 [SMILES: Cc1cc(F)cc(-c2nn(CC(=O)N(c3ccc4c(c3)OC(F)(F)O4)C3CC3)c(=O)c3nc[nH]c23)c1] | 498 [M + 1] | (400 MHz, DMSO) δ: 0.68 (s, 2H); 0.92 (d, J = 5.6 Hz, 2H); 2.45 (s, 3H); 3.31 (m, 1H); 5.19 (s, 2H); 7.12-7.18 (m, 2H); 7.40-7.43 (m, 2H); 8.02 (d, J = 8.4 Hz, 1H), 8.08 (s, 1H), 8.43 (s, 1H) 13.91 (br, 1H) |
| 386 | 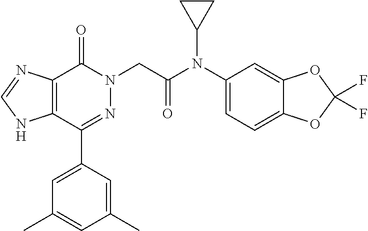 [SMILES: Cc1cc(C)cc(-c2nn(CC(=O)N(c3ccc4c(c3)OC(F)(F)O4)C3CC3)c(=O)c3nc[nH]c23)c1] | 494.2 [M + 1] | (400 MHz, DMSO) δ: 0.68 (s, 2H), 0.91 (m, 2H), 2.39 (s, 6H), 3.31 (s, 1H), 5.17 (s, 2H) 7.13 (m, 2H), 7.41 (m, 2H), 7.95 (s, 2H), 8.39 (s, 1H), 13.80 (br, 1H) |
| 387 | 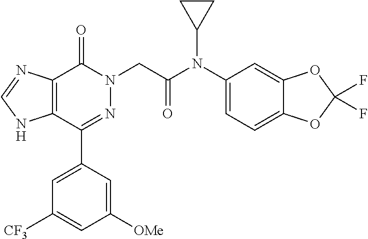 [SMILES: COc1cc(-c2nn(CC(=O)N(c3ccc4c(c3)OC(F)(F)O4)C3CC3)c(=O)c3nc[nH]c23)cc(C(F)(F)F)c1] | 564.0 [M + 1] | (400 MHz, DMSO) δ: 0.68 (s, 2H), 0.92 (d, J = 5.6 Hz, 2H), 3.32 (m, 1H), 3.96 (s, 3H), 5.21 (s, 2H), 7.16 (d, J = 8.4 Hz, 1H), 7.34-7.42 (m, 3H), 8.34-8.44 (m, 3H), 13.85 (br, 1H) |
| 388 | 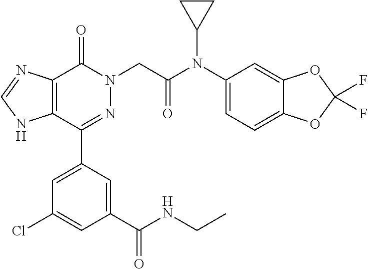 [SMILES: CCNC(=O)c1cc(Cl)cc(-c2nn(CC(=O)N(c3ccc4c(c3)OC(F)(F)O4)C3CC3)c(=O)c3nc[nH]c23)c1] | 571.2 [M + 1] | (400 MHz, DMSO) δ: 0.68 (s, 2H), 0.92 (d, J = 5.6 Hz, 2H), 1.20 (t, J = 6.8 Hz, 3H), 3.32-3.39 (m, 3H), 5.22 (s, 2H), 7.17 (dd, J = 8.4, 2.0 Hz, 1H), 7.40-7.42 (m, 2H), 7.96 (s, 1H), 8.47 (m, 2H), 8.63 (s, 1H), 8.75 (s, 1H), 13.94 (s, 1H) |
| 389 | 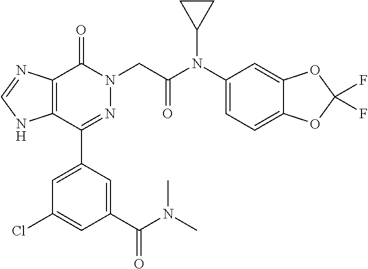 [SMILES: CN(C)C(=O)c1cc(Cl)cc(-c2nn(CC(=O)N(c3ccc4c(c3)OC(F)(F)O4)C3CC3)c(=O)c3nc[nH]c23)c1] | 571.2 [M + 1] | (400 MHz, DMSO) δ: 0.68 (s, 2H), 0.92 (d, J = 6.0 Hz, 2H), 3.02 (s, 6H), 3.32 (m, 1H), 5.21 (s, 2H), 7.16 (dd, J = 8.8, 1.6 Hz, 1H), 7.41 (m, 2H), 7.56 (s, 1H), 8.43-8.46 (m, 2H), 8.55 (s, 1H) |
| 390 | 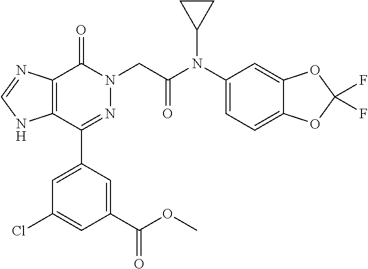 [SMILES: COC(=O)c1cc(Cl)cc(-c2nn(CC(=O)N(c3ccc4c(c3)OC(F)(F)O4)C3CC3)c(=O)c3nc[nH]c23)c1] | 558 [M + 1] | (400 MHz, DMSO) δ: 0.69 (s, 2H); 0.92 (d, J = 6.0 Hz, 2H); 3.32 (m, 1H); 3.96 (s, 3H); 5.21 (s, 2H); 7.17 (d, J = 8.4 Hz, 1H); 7.40-7.43 (m, 2H); 8.00 (s, 1H); 8.36 (s, 1H); 8.83 (s, 1H); 9.03 (s, 1H) |
| 391 | 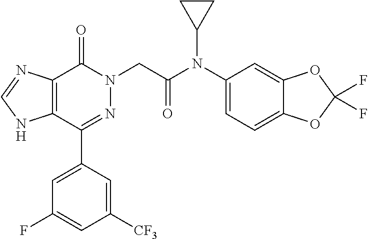 [SMILES: O=C(Cn1nc(-c2cc(F)cc(C(F)(F)F)c2)c2[nH]cnc2c1=O)N(c1ccc2c(c1)OC(F)(F)O2)C1CC1] | 552 [M + 1] | (400 MHz, DMSO) 0.68 (s, 2H), 0.92 (d, J = 4.8 Hz, 2H), 3.32 (m, 1H), 5.23 (s, 2H), 7.16 (dd, J = 8.4, 1.6 Hz, 1H), 7.39-7.42 (m, 2H), 7.73 (d, J = 8.4 Hz, 1H), 8.49 (s, 1H), 8.57 (d, J = 10.0 Hz, 1H), 8.73 (s, 1H), 14.02 (br, 1H) |
| 392 | 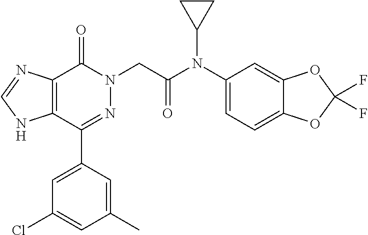 [SMILES: Cc1cc(Cl)cc(-c2nn(CC(=O)N(c3ccc4c(c3)OC(F)(F)O4)C3CC3)c(=O)c3nc[nH]c23)c1] | 514.0 [M + 1] | (400 MHz, DMSO) δ: 0.68 (s, 2H), 0.92 (d, J = 5.6 Hz, 2H), 2.44 (s, 3H), 3.32 (m, 1H), 5.19 (s, 2H), 7.16 (dd, J = 8.4, 2.0 Hz, 1H), 7.40- 7.43 (m, 3H), 8.18 (m, 1H), 8.27 (m, 1H), 8.44 (s, 1H), 13.93 (br, 1H) |
| 393 | 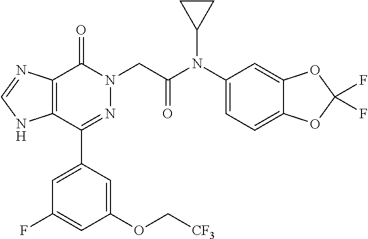 [SMILES: O=C(Cn1nc(-c2cc(F)cc(OCC(F)(F)F)c2)c2[nH]cnc2c1=O)N(c1ccc2c(c1)OC(F)(F)O2)C1CC1] | 582.2 [M + 1] | (400 MHz, DMSO) δ: 0.68 (s, 2H), 0.92 (d, J = 6.0 Hz, 2H), 3.32 (m, 1H), 4.85 (q, J = 8.8 Hz, 2H), 5.20 (s, 2H), 7.12-7.18 (m, 2H), 7.39-7.42 (m, 2H), 7.95 (d, J = 10.0 Hz, 1H), 8.01 (s, 1H), 8.45 (s, 1H), 13.95 (s, 1H) |
| 394 | 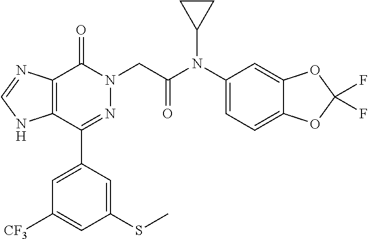 [SMILES: CSc1cc(-c2nn(CC(=O)N(c3ccc4c(c3)OC(F)(F)O4)C3CC3)c(=O)c3nc[nH]c23)cc(C(F)(F)F)c1] | 580.2 [M + H] | (400 MHz, DMSO) δ: 0.68 (s, 2H), 0.92 (d, J = 6.4 Hz, 2H), 2.65 (s, 3H), 3.32 (s, 1H), 5.22 (s, 2H), 7.16 (d, J = 9.2 Hz, 1H), 7.40-7.42 (m, 2H), 7.66 (s, 1H), 8.48 (s, 1H), 8.54 (m, 1H), 8.65 (s, 1H), 14.02 (br, 1H) |
| 395 | 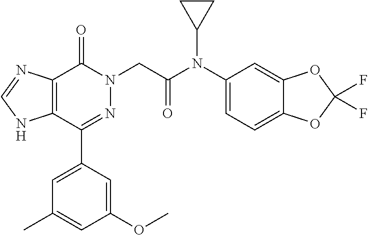 [SMILES: COc1cc(C)cc(-c2nn(CC(=O)N(c3ccc4c(c3)OC(F)(F)O4)C3CC3)c(=O)c3nc[nH]c23)c1] | 510 [M + 1] | (400 MHz, DMSO): 0.67 (s, 2H), 0.92 (d, J = 6.0 Hz, 2H), 2.40 (s, 3H), 3.31 (m, 1H), 3.84 (s, 3H), 5.18 (s, 2H), 6.89 (s, 1H), 7.16 (d, J = 8.4 Hz, 1H), 7.39-7.41 (m, 2H), 7.76-7.78 (m, 2H), 8.40 (s, 1H), 13.71 (br, 1H) |
| 396 | 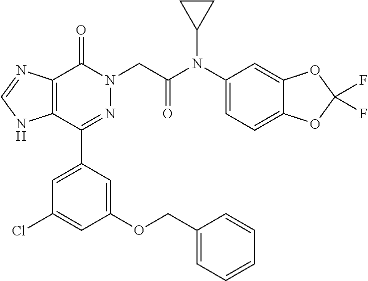 [SMILES: O=C(Cn1nc(-c2cc(Cl)cc(OCc3ccccc3)c2)c2[nH]cnc2c1=O)N(c1ccc2c(c1)OC(F)(F)O2)C1CC1] | 606.2 [M + H] | (400 MHz, DMSO-d₆): 0.68 (s, 2H), 0.92 (d, J = 6.0 Hz, 2H), 3.32 (m, 1H), 5.20 (s, 2H), 5.24 (s, 2H), 7.16 (d, J = 8.8 Hz, 1H), 7.23 (s, 1H), 7.36-7.44 (m, 5H), 7.50 (s, 1H), 7.52 (s, 1H), 8.13 (m, 2H), 8.45 (s, 1H), 14.02 (br, 1H) |
| 397 | 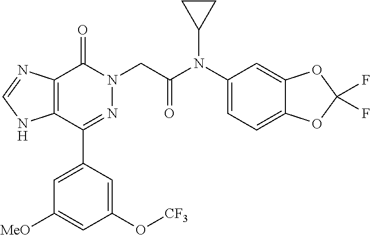 [SMILES: COc1cc(OC(F)(F)F)cc(-c2nn(CC(=O)N(c3ccc4c(c3)OC(F)(F)O4)C3CC3)c(=O)c3nc[nH]c23)c1] | 580.29 [M + H] | (400 MHz, DMSO-d₆): 0.67 (m, 2H), 0.92 (m, 2H), 3.32 (m, 1H), 3.92 (s, 3H), 5.19 (s, 2H), 7.03 (s, 1H), 7.16 (dd, J = 8.8, 1.6 Hz, 1H), 7.40 (s, 1H), 7.41 (d, J = 8.0 Hz, 1H), 8.02 (m, 1H), 8.10 (m, 1H), 8.46 (s, 1H), 13.99 (br, 1H) |
| 398 | 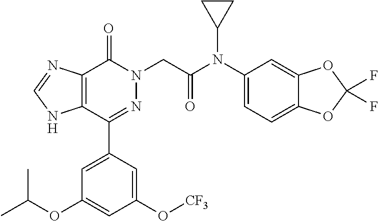 [SMILES: CC(C)Oc1cc(OC(F)(F)F)cc(-c2nn(CC(=O)N(c3ccc4c(c3)OC(F)(F)O4)C3CC3)c(=O)c3nc[nH]c23)c1] | 608.4 [M + H] | (400 MHz, DMSO-d₆): 0.68 (s, 2H), 0.92 (d, J = 5.6 Hz, 2H), 1.37 (d, J = 6.0 Hz, 6H), 3.31 (m, 1H), 4.75 (m, 1H), 5.20 (s, 2H), 7.00 (s, 1H), 7.15 (d, J = 8.0 Hz, 1H), 7.39 (s, 1H), 7.41 (d, J = 8.0 Hz, 1H), 7.96 (m, 1H), 8.09 (m, 1H), 8.46 (s, 1H), 13.94 (br, 1H) |
| 399 | 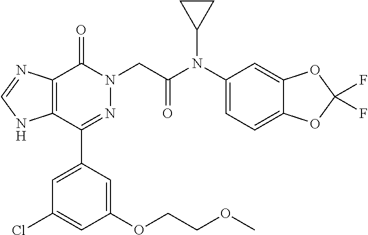 [SMILES: COCCOc1cc(Cl)cc(-c2nn(CC(=O)N(c3ccc4c(c3)OC(F)(F)O4)C3CC3)c(=O)c3nc[nH]c23)c1] | 574.2 [M + H] | (400 MHz, DMSO-d₆): 0.68 (s, 2H), 0.92 (d, J = 5.2 Hz, 2H), 3.32 (m, 1H), 3.37 (s, 3H), 3.74 (s, 2H), 4.24 (s, 2H), 5.202 (s, 2H), 7.15- 7.16 (m, 2H), 7.39-7.41 (m, 2H), 8.05 (m, 2H), 8.44 (s, 1H), 13.82 (br, 1H) |
| 400 | 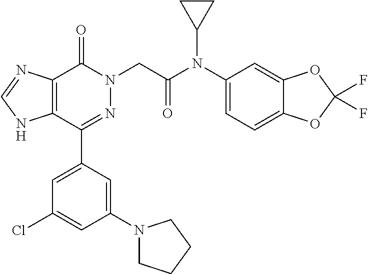 [SMILES: O=C(Cn1nc(-c2cc(Cl)cc(N3CCCC3)c2)c2[nH]cnc2c1=O)N(c1ccc2c(c1)OC(F)(F)O2)C1CC1] | 569 [M + 1] | (400 MHz, DMSO) δ: 0.68 (s, 2H); 0.92 (d, J = 5.6 Hz, 2H); 2.03 (s, 4H); 3.08 (s, 4H); 5.18 (s, 2H); 6.64 (s, 1H); 7.15 (d, J = 7.6 Hz, 1H); 7.39-7.42 (m, 2H); 7.58 (s, 1H); 7.66 (m, 1H), 8.41 (s, 1H); 13.80 (br, 1H) |
| 401 | 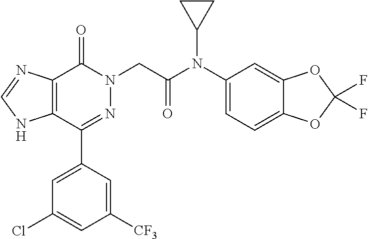 [SMILES: O=C(Cn1nc(-c2cc(Cl)cc(C(F)(F)F)c2)c2[nH]cnc2c1=O)N(c1ccc2c(c1)OC(F)(F)O2)C1CC1] | 568 [M + 1] | (400 MHz, DMSO) δ: 0.68 (s, 2H); 0.92 (d, J = 5.6 Hz, 2H); 3.32 (m, 1H); 5.24 (s, 2H); 7.17 (d, J = 8.4 Hz, 1H); 7.40-7.43 (m, 2H); 7.94 (s, 1H); 8.52 (s, 1H); 8.79-8.82 (m, 2H); 14.09 (br, 1H) |
| 402 | 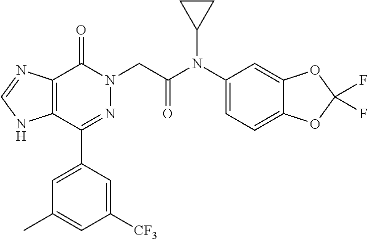 [SMILES: Cc1cc(-c2nn(CC(=O)N(c3ccc4c(c3)OC(F)(F)O4)C3CC3)c(=O)c3nc[nH]c23)cc(C(F)(F)F)c1] | 548 [M + 1] | (400 MHz, DMSO) δ: 0.68 (s, 2H); 0.92 (d, J = 5.6 Hz, 2H); 3.32 (m, 2H); 5.21 (s, 2H); 7.16 (d, J = 7.6 Hz, 1H); 7.40-7.42 (m, 2H); 7.65 (s, 1H); 8.45 (s, 1H), 8.50 (s, 1H); 8.61 (s, 2H); 13.93 (br, 1H) |
| 403 | 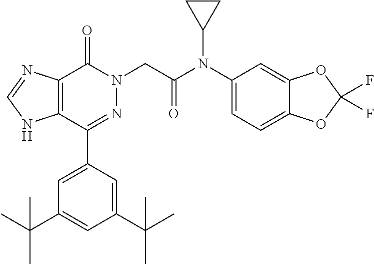 [SMILES: CC(C)(C)c1cc(-c2nn(CC(=O)N(c3ccc4c(c3)OC(F)(F)O4)C3CC3)c(=O)c3nc[nH]c23)cc(C(C)(C)C)c1] | 578 [M + 1] | (400 MHz, DMSO) δ: 0.68 (s, 2H); 0.92 (d, J = 6.0 Hz, 2H); 1.39 (s, 18H); 3.31 (m, 1H); 5.18 (s, 2H); 7.14 (d, J = 7.2 Hz, 1H); 7.37- 7.42 (m, 2H); 7.55 (s, 1H); 8.24 (m, 2H); 8.43 (s, 1H); 13.83 (br, 1H) |
| 404 | 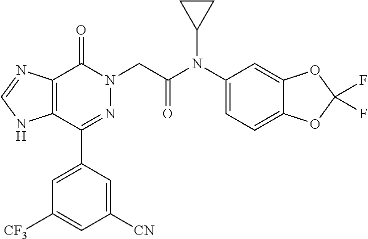 [SMILES: N#Cc1cc(-c2nn(CC(=O)N(c3ccc4c(c3)OC(F)(F)O4)C3CC3)c(=O)c3nc[nH]c23)cc(C(F)(F)F)c1] | 559 [M + 1] | (400 MHz, DMSO) δ: 0.69 (s, 2H); 0.92 (d, J = 6.0 Hz, 2H); 3.33 (m, 1H); 5.25 (s, 2H); 7.17 (dd, J = 8.8, 2.0 Hz, 1H); 7.39-7.42 (m, 2H); 8.38 (s, 1H); 8.53 (s, 1H); 9.09-9.13 (m, 2H); 14.14 (br, 1H) |
| 405 | 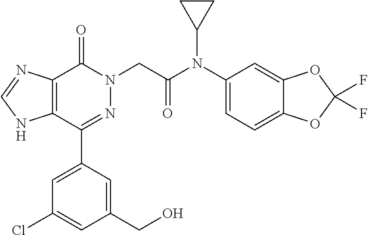 [SMILES: O=C(Cn1nc(-c2cc(Cl)cc(CO)c2)c2[nH]cnc2c1=O)N(c1ccc2c(c1)OC(F)(F)O2)C1CC1] | 530 [M + 1] | (400 MHz, DMSO) δ: 0.68 (s, 2H); 0.91 (d, J = 6.4 Hz, 2H); 3.31 (m, 1H); 4.61 (s, 2H); 5.20 (s, 2H); 7.15- 7.18 (m, 1H); 7.40-7.42 (m, 2H); 7.49 (s, 1H); 8.31 (s, 1H); 8.36 (m, 1H), 8.45 (s, 1H), 13.96 (br, 1H) |
| 406 | 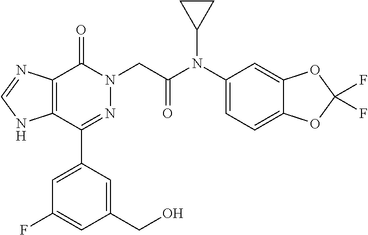 [SMILES: O=C(Cn1nc(-c2cc(F)cc(CO)c2)c2[nH]cnc2c1=O)N(c1ccc2c(c1)OC(F)(F)O2)C1CC1] | 514 [M + 1] | (400 MHz, DMSO) δ: 0.68 (s, 2H); 0.92 (d, J = 5.2 Hz, 2H); 3.32 (m, 1H); 4.64 (d, J = 4.0 Hz, 2H); 5.20 (m, 3H); 7.16 (d, J = 8.0 Hz, 1H); 7.25 (d, J = 8.8 Hz, 1H); 7.42 (s, 2H); 8.10 (m, 1H); 8.21 (m, 1H); 8.45 (s, 1H); 13.95 (br, 1H) |
| 407 | 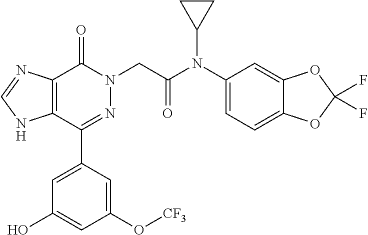 [SMILES: O=C(Cn1nc(-c2cc(O)cc(OC(F)(F)F)c2)c2[nH]cnc2c1=O)N(c1ccc2c(c1)OC(F)(F)O2)C1CC1] | 566 [M + 1] | (400 MHz, DMSO) δ: 0.67 (s, 2H); 0.92 (d, J = 5.6 Hz, 2H); 3.31 (m, 1H); 5.19 (s, 2H); 6.82 (s, 1H); 7.15 (d, J = 8.4 Hz, 1H); 7.40 (s, 1H), 7.41 (d, J = 8.0 Hz, 1H); 7.85 (s, 1H), 7.94 (s, 1H), 8.44 (s, 1H); 10.03 (s, 1H); 13.97 (s, 1H) |
| 408 | 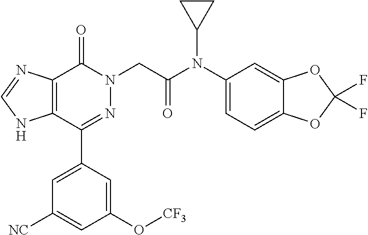 [SMILES: N#Cc1cc(OC(F)(F)F)cc(-c2nn(CC(=O)N(c3ccc4c(c3)OC(F)(F)O4)C3CC3)c(=O)c3nc[nH]c23)c1] | 575 [M + 1] | (400 MHz, DMSO) δ: 0.68 (s, 2H); 0.92 (d, J = 6.0 Hz, 2H); 3.32 (m, 1H); 5.24 (s, 2H); 7.16 (d, J = 7.2 Hz, 1H), 7.40-7.42 (m, 2H); 8.05 (s, 1H); 8.52 (s, 1H); 8.80 (m, 1H); 8.87 (s, 1H); 14.05 (br, 1H) |
| 409 | 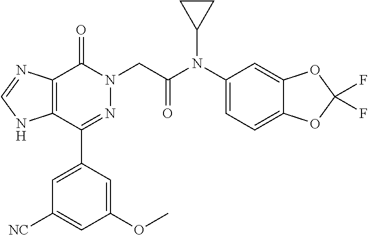 [SMILES: COc1cc(C#N)cc(-c2nn(CC(=O)N(c3ccc4c(c3)OC(F)(F)O4)C3CC3)c(=O)c3nc[nH]c23)c1] | 521 [M + 1] | (400 MHz, DMSO) δ: 0.68 (s, 2H); 0.92 (d, J = 6.0 Hz, 2H); 3.32 (m, 1H); 3.94 (s, 3H); 5.21 (s, 2H); 7.16 (d, J = 7.2 Hz, 1H); 7.41 (m, 2H), 7.53 (s, 1H); 8.36-8.39 (m, 2H); 8.48 (s, 1H); 14.06 (br, 1H) |
| 410 | 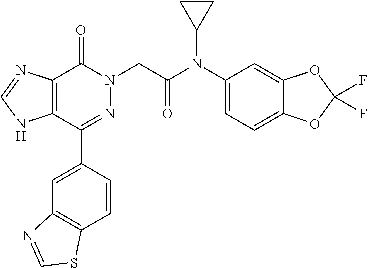 [SMILES: O=C(Cn1nc(-c2ccc3scnc3c2)c2[nH]cnc2c1=O)N(c1ccc2c(c1)OC(F)(F)O2)C1CC1] | 523.0 [M + 1] | (400 MHz, DMSO) δ: 0.69 (s, 2H), 0.93 (d, J = 5.6 Hz, 2H), 3.33 (m, 1H), 5.23 (s, 2H), 7.16-7.18 (d, J = 8.4 Hz, 1H), 7.39-7.42 (m, 2H), 8.29 (d, J = 8.4 Hz, 2H), 8.45-8.48 (m, 2H), 9.24 (s, 1H), 9.44 (s, 1H) |
| 411 | 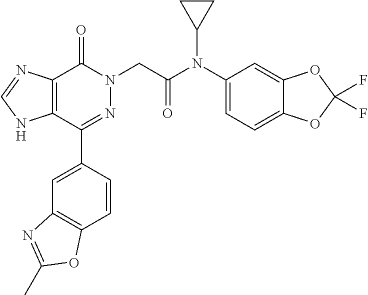 [SMILES: Cc1nc2cc(-c3nn(CC(=O)N(c4ccc5c(c4)OC(F)(F)O5)C4CC4)c(=O)c4nc[nH]c34)ccc2o1] | 521.2 [M + 1] | (400 MHz, DMSO) δ: 0.68 (s, 2H), 0.92 (d, J = 5.2 Hz, 2H), 2.67 (s, 3H), 3.32 (m, 1H), 5.20 (s, 2H), 7.17 (d, J = 7.6 Hz, 1H), 7.39-7.42 (m, 2H), 7.76 (d, J = 8.4 Hz, 1H), 8.38 (m, 2H), 8.73 (s, 1H), 13.82 (br, 1H) |
| 412 | 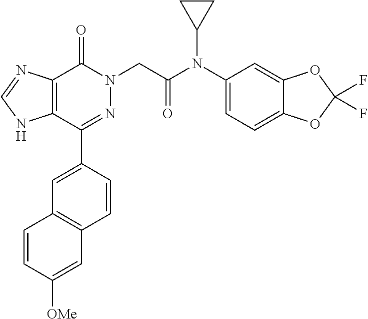 [SMILES: COc1ccc2cc(-c3nn(CC(=O)N(c4ccc5c(c4)OC(F)(F)O5)C4CC4)c(=O)c4nc[nH]c34)ccc2c1] | 546.3 [M + 1] | (400 MHz, DMSO) δ: 0.69 (s, 2H), 0.93 (d, J = 6.0 Hz, 2H), 3.33 (m, 1H), 3.95 (s, 3H), 5.22 (s, 2H), 7.18 (d, J = 7.2 Hz, 1H), 7.24 (dd, J = 8.8, 2.0 Hz, 1H), 7.39- 7.43 (m, 3H), 7.92-7.97 (m, 2H), 8.35 (d, J = 8.0 Hz, 1H), 8.45 (s, 1H), 9.03 (s, 1H), 13.90 (br, 1H) |
| 413 | 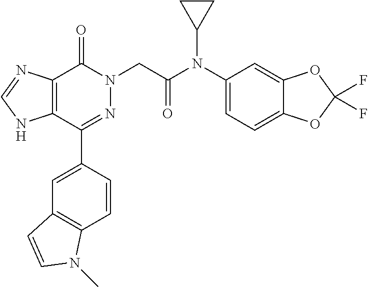 [SMILES: Cn1ccc2cc(-c3nn(CC(=O)N(c4ccc5c(c4)OC(F)(F)O5)C4CC4)c(=O)c4nc[nH]c34)ccc21] | 519.3 [M + 1] | (400 MHz, DMSO) δ: 0.68 (s, 2H), 0.92 (d, J = 6.0 Hz, 2H), 3.32 (m, 1H), 3.86 (s, 3H), 5.19 (s, 2H), 6.57 (d, J = 2.4 Hz, 1H), 7.16 (d, J = 8.4 Hz, 1H), 7.35-7.42 (m, 3H), 7.53-7.56 (m, 1H), 8.13 (d, J = 7.6 Hz, 1H), 8.39 (s, 1H), 8.71 (s, 1H), 13.75 (br, 1H) |
| 414 | 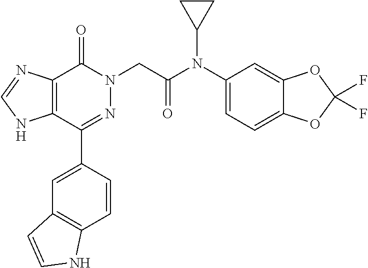 [SMILES: O=C(Cn1nc(-c2ccc3[nH]ccc3c2)c2[nH]cnc2c1=O)N(c1ccc2c(c1)OC(F)(F)O2)C1CC1] | 505 [M + 1] | (400 MHz, DMSO) δ: 0.68 (s, 2H); 0.92 (d, J = 5.6 Hz, 2H); 3.33 (m, 1H); 5.19 (s, 2H); 6.57 (s, 1H) 7.16 (d, J = 8.4 Hz, 2H); 7.38-7.42 (m, 2H); 7.52 (d, J = 8.4 Hz, 1H), 8.05 (m, 1H), 8.38 (s, 1H); 11.06 (s, 1H) 13.65 (br, 1H) |
| 415 | 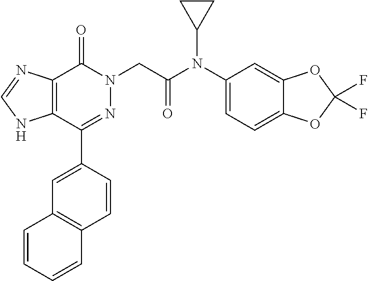 [SMILES: O=C(Cn1nc(-c2ccc3ccccc3c2)c2[nH]cnc2c1=O)N(c1ccc2c(c1)OC(F)(F)O2)C1CC1] | 516 [M + 1] | (400 MHz, DMSO) δ: 0.70 (s, 2H); 0.93 (d, J = 5.6 Hz, 2H); 3.34 (m, 1H); 5.24 (s, 2H); 7.18 (d, J = 8.4 Hz, 1H); 7.40-7.43 (m, 2H); 7.58-7.61 (m, 2H). 7.97- 8.06 (m, 3H); 8.39 (d, J = 8.0 Hz, 1H); 8.48 (s, 1H); 9.12 (s, 1H) 13.93 (br, 1H) |
| 416 | 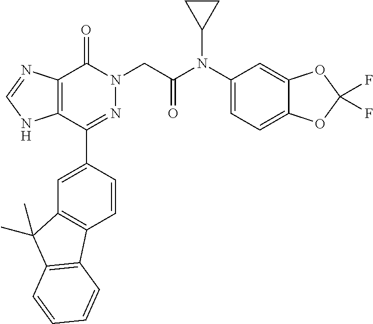 [SMILES: CC1(C)c2ccccc2-c2ccc(-c3nn(CC(=O)N(c4ccc5c(c4)OC(F)(F)O5)C4CC4)c(=O)c4nc[nH]c34)cc21] | 582 [M + 1] | (400 MHz, DMSO): 0.69 (s, 2H), 0.93 (s, 2H), 1.54 (s, 3H), 1.58 (s, 3H), 3.33 (s, 1H), 5.21 (s, 2H), 7.16- 7.18 (d, J = 8.4 Hz, 1H), 7.40-7.42 (m, 3H), 7.60 (s, H), 7.89-7.96 (m, 2H) 8.41-8.49 (m, 3H), 13.88 (br, 1H) |
| 417 | 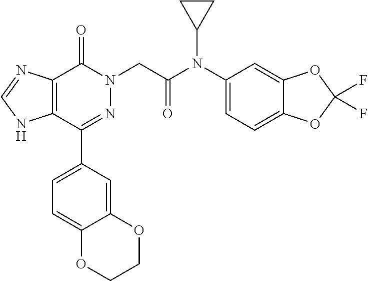 [SMILES: O=C(Cn1nc(-c2ccc3c(c2)OCCO3)c2[nH]cnc2c1=O)N(c1ccc2c(c1)OC(F)(F)O2)C1CC1] | 524.0 [M + H] | (400 MHz, DMSO-d₆): 0.66 (s, 2H), 0.90 (d, J = 6.0 Hz, 2H), 3.29 (m, 1H), 4.31 (s, 4H), 5.10 (s, 2H), 6.93 (d, J = 8.4 Hz, 1H), 7.14 (d, J = 8.4 Hz, 1H), 7.39 (m, 2H), 7.98 (d, J = 8.4 Hz, 1H), 8.04 (s, 1H), 8.09 (s, 1H) |
| 418 | 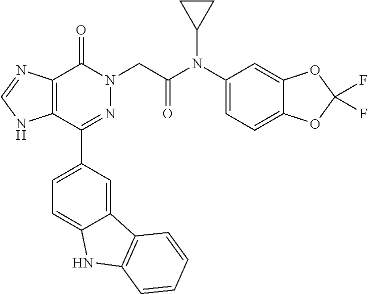 [SMILES: O=C(Cn1nc(-c2ccc3[nH]c4ccccc4c3c2)c2[nH]cnc2c1=O)N(c1ccc2c(c1)OC(F)(F)O2)C1CC1] | 555.3 [M + H] | (400 MHz, DMSO-d₆): 0.70 (s, 2H), 0.93 (d, J = 6.0 Hz, 2H), 3.34 (m, 1H), 5.22 (s, 2H), 7.17-7.26 (m, 2H), 7.40-7.46 (m, 3H), 7.55 (d, J = 8.0 Hz, 1H), 7.62 (d, J = 8.4 Hz, 1H), 8.15 (d, J = 7.6 Hz, 1H), 8.40 (m, 1H), 8.45 (s, 1H), 9.14 (m, 1H), 11.26 (s, 1H), 13.60 (br, 1H) |
| 419 | 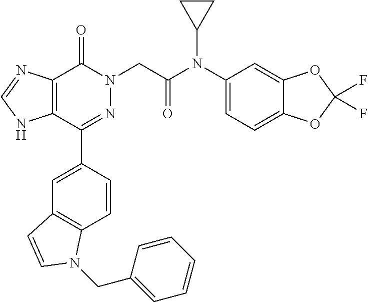 [SMILES: O=C(Cn1nc(-c2ccc3c(ccn3Cc3ccccc3)c2)c2[nH]cnc2c1=O)N(c1ccc2c(c1)OC(F)(F)O2)C1CC1] | 595.2 [M + 1] | (400 MHz, DMSO): 0.67 (s, 2H), 0.92 (d, J = 5.6 Hz, 2H), 3.32 (s, 1H), 5.17 (s, 2H), 5.48 (s, 2H), 6.64 (d, J = 2.8 Hz, 1H), 7.16 (d, J = 2.8 Hz, 1H), 7.24-7.29 (m, 3H), 7.32-7.36 (m, 2H), 7.39-7.42 (m, 2H), 7.51 (d, J = 3.3 Hz, 1H), 7.56 (d, J = 8.4 Hz, 1H), 8.05 (d, J = 8.8 Hz, 1H), 8.38 (s, 1H), 8.70 (s, 1H), 13.74 (br, 1H) |
| 420 | 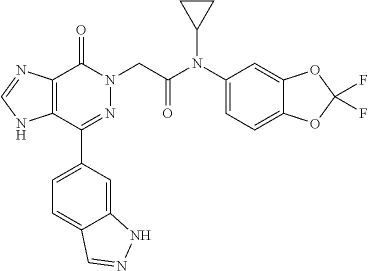 [SMILES: O=C(Cn1nc(-c2ccc3cn[nH]c3c2)c2[nH]cnc2c1=O)N(c1ccc2c(c1)OC(F)(F)O2)C1CC1] | 506 [M + 1] | (400 MHz, DMSO) δ: 0.69 (s, 2H); 0.93 (d, J = 6.0 Hz, 2H); 3.332 (s, 1H); 3.33 (m, 1H), 5.221 (s, 2H); 7.17 (d, J = 8.8 Hz, 1H); 7.40-7.43 (m, 2H); 7.87 (d, J = 8.4 Hz, 1H); 8.04 (s, J = 8.0 Hz, 1H); 8.12 (s, 1H); 8.46 (s, 1H); 8.87 (s, 1H); 13.11 (s, 1H); 13.92 (br, 1H) |
| 421 | 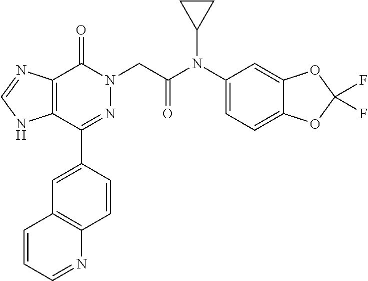 [SMILES: O=C(Cn1nc(-c2ccc3ncccc3c2)c2[nH]cnc2c1=O)N(c1ccc2c(c1)OC(F)(F)O2)C1CC1] | 515 [M − 1] | (400 MHz, DMSO) δ: 0.70 (s, 2H); 0.93 (m, 2H); 3.34 (m, 1H); 5.24 (s, 2H); 7.18 (dd, J = 8.4, 2.0 Hz, 1H); 7.37-7.44 (m, 2H); 7.60 (dd, J = 8.0, 4.0 Hz, 1H); 8.16 (d, J = 8.8 Hz, 1H); 8.47-8.49 (m, 2H); 8.64 (d, J = 8.4 Hz, 1H); 8.97 (m, 1H); 9.17 (s, 1H); 13.86 (br, 1H) |
| 422 | 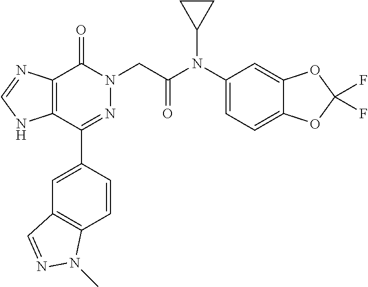 [SMILES: Cn1ncc2cc(-c3nn(CC(=O)N(c4ccc5c(c4)OC(F)(F)O5)C4CC4)c(=O)c4nc[nH]c34)ccc21] | 520.7 [M + 1] | (400 MHz, DMSO): 0.69 (s, 2H); 0.93 (d, J = 6.4 Hz, 2H); 3.33 (m, 1H) 4.11 (s, 3H), 5.21 (s, 2H) 7.11-7.17 (dd, J = 8.4, 2.0 Hz, 1H), 7.41 (m, 2H), 7.73 (d, J = 8.8 Hz, 1H), 8.19 (s, 1H), 8.34 (d, J = 9.2 Hz, 1H), 8.43 (s, 1H), 8.99 (s, 1H), 13.82 (br, 1H) |
| 423 | 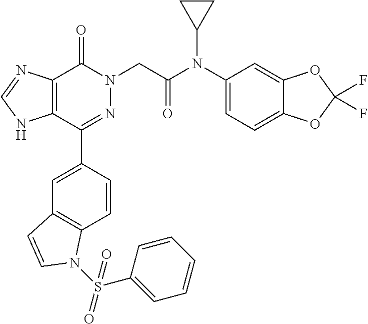 [SMILES: O=C(Cn1nc(-c2ccc3c(ccn3S(=O)(=O)c3ccccc3)c2)c2[nH]cnc2c1=O)N(c1ccc2c(c1)OC(F)(F)O2)C1CC1] | 645 [M + 1] | (400 MHz, DMSO) δ: 0.67 (s, 2H); 0.92 (d, J = 5.6 Hz, 2H); 3.32 (m, 1H); 5.18 (s, 2H); 6.99 (d, J = 3.2 Hz, 1H); 7.15 (d, J = 7.2 Hz, 1H); 7.39-7.41 (m, 2H); 7.59-7.64 (m, 2H); 7.71 (m, 1H); 7.83 (d, J = 3.6 Hz, 1H); 7.98-8.01 (m, 2H); 8.08 (d, J = 8.8 Hz, 1H); 8.29 (m, 1H); 8.41 (s, 1H); 8.70 (m, 1H); 13.82 (br, 1H) |
| 424 | 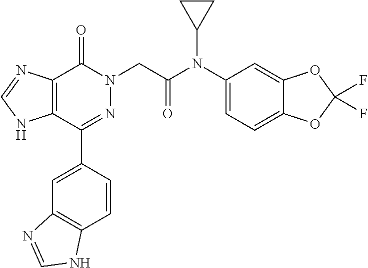 [SMILES: O=C(Cn1nc(-c2ccc3[nH]cnc3c2)c2[nH]cnc2c1=O)N(c1ccc2c(c1)OC(F)(F)O2)C1CC1] | 506.2 [M + 1] | (400 MHz, DMSO): 0.68 (s, 2H), 0.92 (m, 2H), .32 (m, 1H), 5.21 (s, 2H), 7.17 (d, J = 8.0 Hz, 1H), 7.40- 7.42 (m, 2H), 7.71 (s, 1H), 8.19 (m, 1H), 8.25 (s, 1H), 8.41 (s, 1H), 8.78 (s, 1H), 12.48 (br, 1H), 13.77 (br, 1H) |
| 425 | 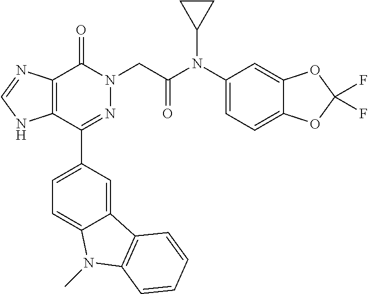 [SMILES: Cn1c2ccccc2c2cc(-c3nn(CC(=O)N(c4ccc5c(c4)OC(F)(F)O5)C4CC4)c(=O)c4nc[nH]c34)ccc21] | 569 [M + 1] | (400 MHz, DMSO) δ: 0.70 (s, 2H); 0.94 (d, J = 5.6 Hz, 2H); 3.34 (m, 1H); 3.96 (s, 3H); 5.23 (s, 2H); 7.18 (d, J = 8.4 Hz, 1H); 7.29 (t, J = 7.2 Hz, 1H); 7.41 (d, J = 8.8 Hz, 1H), 7.43 (s, 1H); 7.53 (t, J = 7.2 Hz, 1H); 7.64 (d, J = 8.0 Hz, 1H); 7.72 (d, J = 8.4 Hz, 1H); 8.20 (d, J = 7.6 Hz, 1H); 8.45 (s, 1H), 8.49 (m, 2H); 9.18 (s, 1H); 13.67 (br, 1H) |
| 426 | 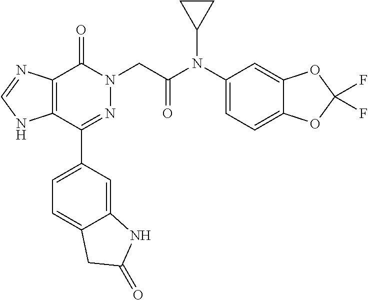 [SMILES: O=C1Cc2ccc(-c3nn(CC(=O)N(c4ccc5c(c4)OC(F)(F)O5)C4CC4)c(=O)c4nc[nH]c34)cc2N1] | 521 [M + 1] | (400 MHz, DMSO) δ: 0.65 (s, 2H); 0.91 (d, J = 5.2 Hz, 2H); 3.31 (m, 1H); 3.55 (s, 2H); 5.18 (s, 2H); 7.165 (dd, J = 10.4, 2.0 Hz, 1H); 7.35 (d, J = 7.2 Hz, 1H); 7.39 (s, 1H), 7.41 (d, 8.8 Hz, 1H), 7.89 (s, 1H); 8.01 (m, 1H); 8.40 (s, 1H); 10.31 (s, 1H); 13.84 (br, 1H) |
| 427 | 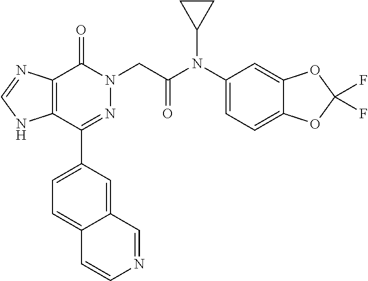 [SMILES: O=C(Cn1nc(-c2ccc3ccncc3c2)c2[nH]cnc2c1=O)N(c1ccc2c(c1)OC(F)(F)O2)C1CC1] | 517 [M + 1] | (400 MHz, DMSO) δ: 0.69 (m, 2H); 0.92 (m, 2H); 3.34 (m, 1H); 5.25 (m, 2H); 7.13-7.20 (m, 1H); 7.39-7.44 (m, 2H); 7.88 (d, J = 5.6 Hz, 1H); 8.11 (d, J = 8.4 Hz, 1H); 8.51 (s, 1H); 8.58 (d, J = 5.6 Hz, 1H); 8.65 (d, J = 8.4 Hz, 1H); 9.31 (s, 1H); 9.43 (s, 1H); 13.86 (br, 1H) |
| 428 | 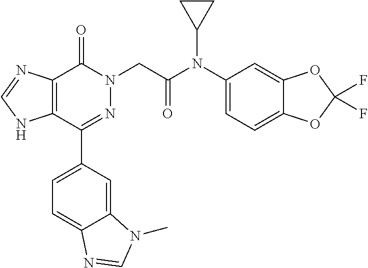 [SMILES: Cn1cnc2ccc(-c3nn(CC(=O)N(c4ccc5c(c4)OC(F)(F)O5)C4CC4)c(=O)c4nc[nH]c34)cc21] | 520 [M + 1] | (400 MHz, DMSO) δ: 0.69 (s, 2H); 0.93 (d, J = 5.6 Hz, 2H); 3.32 (m, 1H); 3.94 (s, 3H); 5.211 (s, 2H); 7.17 (d, J = 8.4 Hz, 1H); 7.41 (m, 2H); 7.77 (d, J = 8.4 Hz, 1H); 8.24 (m, 2H); 8.44 (s, 1H); 8.64 (m, 1H); 13.84 (br, 1H) |
| 429 | 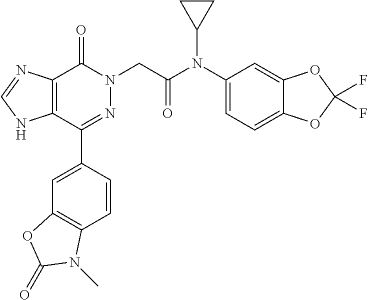 [SMILES: Cn1c(=O)oc2cc(-c3nn(CC(=O)N(c4ccc5c(c4)OC(F)(F)O5)C4CC4)c(=O)c4nc[nH]c34)ccc21] | 537 [M + 1] | (400 MHz, DMSO) δ: 0.68 (s, 2H); 0.92 (d, J = 5.2 Hz, 2H); 3.32 (m, 1H); 3.43 (s, 3H); 5.19 (s, 2H); 7.16 (d, J = 8.8 Hz, 1H); 7.39-7.42 (m, 3H); 8.32 (s, 1H); 8.41- 8.44 (m, 2H); 13.91 (br, 1H) |
| 430 | 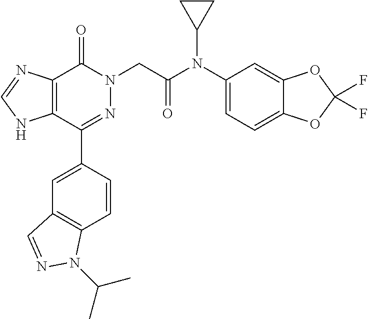 [SMILES: CC(C)n1ncc2cc(-c3nn(CC(=O)N(c4ccc5c(c4)OC(F)(F)O5)C4CC4)c(=O)c4nc[nH]c34)ccc21] | 548 [M + 1] | (400 MHz, DMSO) δ: 0.68 (s, 2H); 0.92 (d, J = 5.6 Hz, 2H); 1.56 (d, J = 6.4 Hz, 6H); 3.33 (m, 1H); 5.04 (m, 1H); 5.20 (s, 2H); 7.17 (d, J = 8.8 Hz, 1H); 7.41 (m, 2H); 7.79 (d, J = 8.8 Hz, 1H); 8.20 (s, 1H); 8.30 (m, 1H); 8.43 (s, 1H); 8.97 (m, 1H); 13.86 (br, 1H) |
| 431 | 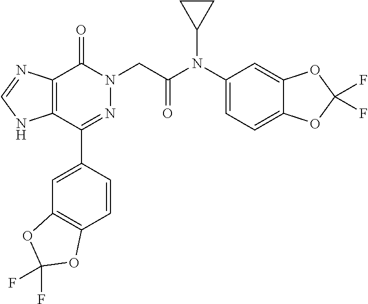 [SMILES: O=C(Cn1nc(-c2ccc3c(c2)OC(F)(F)O3)c2[nH]cnc2c1=O)N(c1ccc2c(c1)OC(F)(F)O2)C1CC1] | 546 [M + 1] | (400 MHz, DMSO) δ: 0.8 (s, 2H); 0.92 (d, J = 6.4 Hz, 2H); 3.32 (m, 1H); 5.19 (s, 2H); 7.15 (d, J = 8.4 Hz, 1H); 7.39-7.41 (m, 2H); 7.55 (d, J = 8.8 Hz, 1H); 8.34-8.46 (m, 3H); 14.03 (br, 1H) |
| 432 | 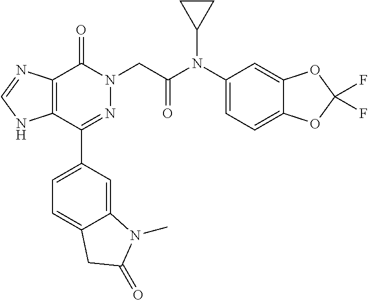 [SMILES: CN1C(=O)Cc2ccc(-c3nn(CC(=O)N(c4ccc5c(c4)OC(F)(F)O5)C4CC4)c(=O)c4nc[nH]c34)cc21] | 535 [M + 1] | (400 MHz, DMSO) δ: 0.68 (s, 2H); 0.92 (d, J = 5.6 Hz, 2H); 3.31 (m, 1H); 4.00 (s, 3H); 5.19 (s, 2H); 7.15 (d, J = 7.2 Hz, 1H); 7.39-7.46 (m, 3H); 8.44 (s, 1H); 8.67 (d, J = 8.8 Hz, 1H); 8.74 (s, 1H), 13.92 (br, 1H) |
| 433 | 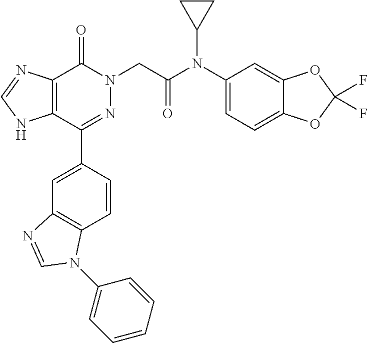 [SMILES: O=C(Cn1nc(-c2ccc3c(c2)ncn3-c2ccccc2)c2[nH]cnc2c1=O)N(c1ccc2c(c1)OC(F)(F)O2)C1CC1] | 582 [M + 1] | (400 MHz, DMSO) δ: 0.69 (s, 2H); 0.93 (m, 2H); 3.33 (m, 1H); 5.22 (s, 2H); 7.17 (d, J = 8.4 Hz, 1H); 7.40- 7.46 (m, 2H); 7.56 (m, 1H); 7.66-7.75 (m, 5H); 8.35 (m, 1H); 8.44 (s, 1H); 8.58 (s, 1H), 8.94 (s, 1H); 13.61 (br, 1H) |
| 434 | 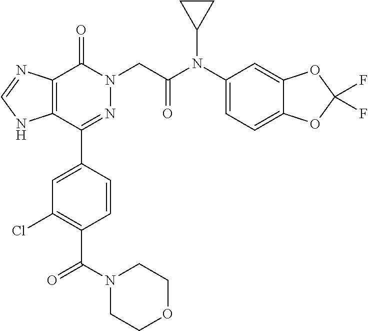 [SMILES: O=C(c1ccc(-c2nn(CC(=O)N(c3ccc4c(c3)OC(F)(F)O4)C3CC3)c(=O)c3nc[nH]c23)cc1Cl)N1CCOCC1] | 613 [M + 1] | (400 MHz, DMSO) δ: 0.68 (s, 2H); 0.92 (d, J = 5.6 Hz, 2H); 3.24 (m, 2H); 3.32 (m, 1H); 3.60-3.72 (m, 6H), 5.21 (s, 2H); 7.16 (d, J = 7.6 Hz, 1H); 7.40-7.42 (m, 2H); 7.55 (d, J = 8.0 Hz, 1H); 8.45 (m, 2H); 8.58 (s, 1H); 13.96 (br, 1H) |
| 435 | 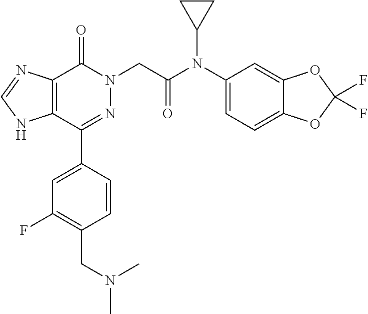 [SMILES: CN(C)Cc1ccc(-c2nn(CC(=O)N(c3ccc4c(c3)OC(F)(F)O4)C3CC3)c(=O)c3nc[nH]c23)cc1F] | 541 [M + 1] | (400 MHz, DMSO) δ: 0.68 (s, 2H); 0.92 (d, J = 5.2 Hz, 2H); 2.26 (s, 6H); 3.32 (m, 1H); 3.58 (s, 2H); 5.20 (s, 2H); 7.16 (d, J = 8.4 Hz, 1H); 7.40-7.42 (m, 2H); 7.56 (dd, J = 8.4, 7.2 Hz, 1H); 8.23 (m, 2H); 8.45 (s, 1H) |
| 436 | 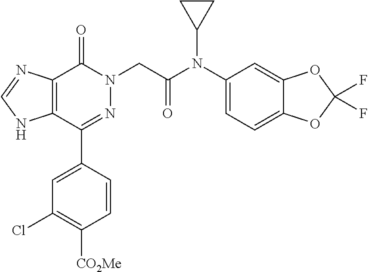 [SMILES: COC(=O)c1ccc(-c2nn(CC(=O)N(c3ccc4c(c3)OC(F)(F)O4)C3CC3)c(=O)c3nc[nH]c23)cc1Cl] | 558.2 [M + 1] | (400 MHz, DMSO) δ: 0.68 (s, 2H), 0.92 (d, J = 6.0 Hz, 2H), 3.32 (m, 1H), 3.93 (s, 3H), 5.22 (s, 2H), 7.16 (d, J = 8.4 Hz, 1H), 7.36-7.39 (m, 2H), 7.97 (d, J = 8.4 Hz, 1H), 8.44 (m, 2H), 8.61 (s, 1H) |
| 437 | 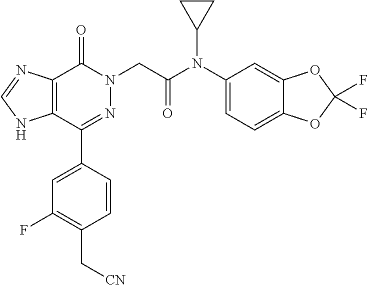 [SMILES: N#CCc1ccc(-c2nn(CC(=O)N(c3ccc4c(c3)OC(F)(F)O4)C3CC3)c(=O)c3nc[nH]c23)cc1F] | 523.2 [M + 1] | (400 MHz, DMSO) δ: 0.68 (s, 2H), 0.92 (d, J = 5.6 Hz, 2H), 3.32 (m, 1H), 4.12 (s, 2H), 5.20 (s, 2H), 7.16 (d, J = 6.8 Hz, 1H), 7.40-7.42 (m, 2H), 7.64 (t, J = 8.0 Hz, 1H), 8.30-8.33 (m, 2H), 8.43 (s, 1H), 13.91 (br, 1H) |
| 438 | 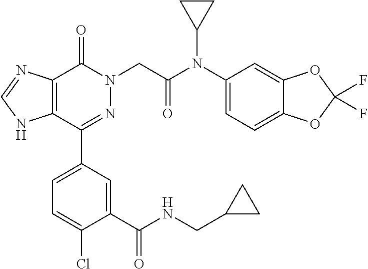 [SMILES: O=C(NCC1CC1)c1cc(-c2nn(CC(=O)N(c3ccc4c(c3)OC(F)(F)O4)C3CC3)c(=O)c3nc[nH]c23)ccc1Cl] | 583.2 [M + 1] | (400 MHz, DMSO) δ: 0.59 (s, 2H), 0.67 (s, 2H), 0.74 (m, 2H), 0.92 (m, 2H), 2.88 (m 1H), 3.31 (m, 1H), 5.19 (s, 2H), 7.13 (d, J = 8.0 Hz, 1H), 7.38 (m, 2H), 7.62 (d, J = 8.4 Hz, 1H), 8.36-8.48 (m, 4H) |
| 439 | 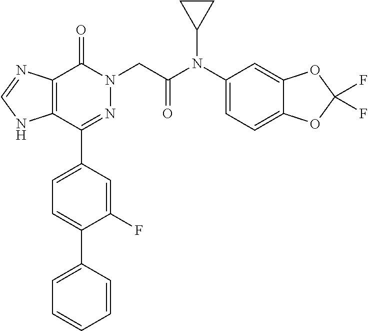 [SMILES: O=C(Cn1nc(-c2ccc(-c3ccccc3)c(F)c2)c2[nH]cnc2c1=O)N(c1ccc2c(c1)OC(F)(F)O2)C1CC1] | 560.3 [M + 1] | (400 MHz, DMSO) δ: 0.69 (s, 2H), 0.93 (d, J = 5.6 Hz, 2H), 3.33 (m, 1H), 5.22 (s, 2H), 7.17 (d, J = 8.4 Hz, 1H), 7.40-7.45 (m, 3H), 7.52-7.55 (m, 2H), 7.64- 7.68 (m, 3H), 8.35-8.44 (m, 3H), 13.93 (br, 1H) |
| 440 | 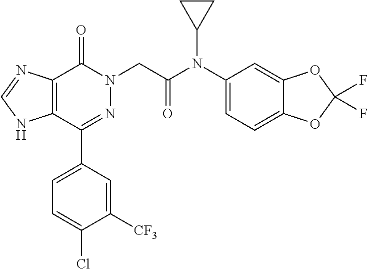 [SMILES: O=C(Cn1nc(-c2ccc(Cl)c(C(F)(F)F)c2)c2[nH]cnc2c1=O)N(c1ccc2c(c1)OC(F)(F)O2)C1CC1] | 568.0 [M + 1] | (400 MHz, DMSO) δ: 0.68 (s, 2H), 0.92 (d, J = 5.6 Hz, 2H), 3.31 (m, 1H), 5.21 (s, 2H), 7.16 (d, J = 8.4 Hz, 1H), 7.40-7.41 (m, 2H), 7.90 (d, J = 8.4 Hz, 1H), 8.49 (s, 1H), 8.70 (d, J = 8.0 Hz, 1H), 8.98 (s, 1H), 14.04 (br, 1H) |
| 441 | 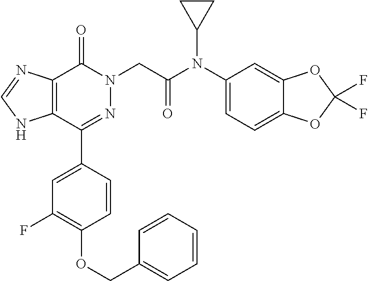 [SMILES: O=C(Cn1nc(-c2ccc(OCc3ccccc3)c(F)c2)c2[nH]cnc2c1=O)N(c1ccc2c(c1)OC(F)(F)O2)C1CC1] | 590.2 [M + 1] | (400 MHz, DMSO) δ: 0.67 (s, 2H), 0.90 (m, 2H), 3.3 (m, 1H), 5.17 (s, 2H), 5.29 (s, 2H), 7.16 (d, J = 8.4 Hz, 1H), 7.41-7.46 (m, 6H), 7.50-7.52 (m, 2H), 8.24- 8.27 (m, 2H), 8.42 (s, 1H), 13.89 (br, 1H) |
| 442 | 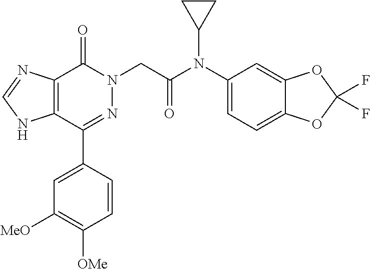 [SMILES: COc1ccc(-c2nn(CC(=O)N(c3ccc4c(c3)OC(F)(F)O4)C3CC3)c(=O)c3nc[nH]c23)cc1OC] | 526.2 [M + 1] | (400 MHz, DMSO) δ: 0.67 (s, 2H), 0.92 (d, J = 5.2 Hz, 2H), 3.31 (m, 1H), 3.88 (s, 6H), 5.16 (s, 2H), 7.10-7.16 (m, 2H), 7.40-7.42 (m, 2H), 8.01-8.06 (m, 2H), 8.38 (s, 1H) |
| 443 | 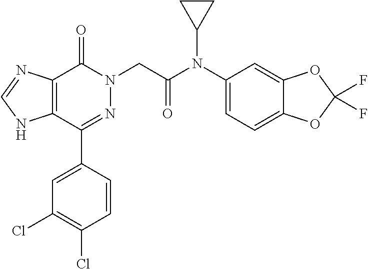 [SMILES: O=C(Cn1nc(-c2ccc(Cl)c(Cl)c2)c2[nH]cnc2c1=O)N(c1ccc2c(c1)OC(F)(F)O2)C1CC1] | 534 [M + 1] | (400 MHz, DMSO) δ: 0.68 (s, 2H); 0.912 (d, J = 5.6 Hz, 2H); 3.32 (m, 1H); 5.21 (s, 2H); 7.16 (d, J = 8.8 Hz, 1H); 7.40-7.42 (m, 2H); 7.80 (d, J = 8.8 Hz, 1H), 8.43 (d, J = 8.8 Hz, 1H), 8.46 (s, H); 8.70 (s, 1H), 14.00 (br, 1H) |
| 444 | 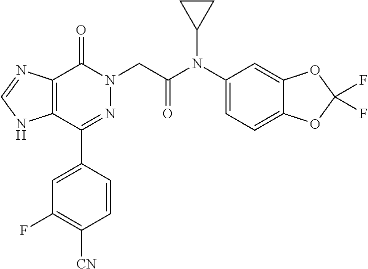 [SMILES: N#Cc1ccc(-c2nn(CC(=O)N(c3ccc4c(c3)OC(F)(F)O4)C3CC3)c(=O)c3nc[nH]c23)cc1F] | 509 [M + 1] | (400 MHz, DMSO) δ: 0.68 (s, 2H); 0.92 (d, J = 5.6 Hz, 2H); 3.32 (m, 1H); 5.23 (s, 2H); 7.16 (d, J = 8.4 Hz, 1H); 7.40-7.43 (m, 2H); 8.07 (t, J = 7.6 Hz, 1H), 8.50-8.56 (m, 3H); 14.12 (br, 1H) |
| 445 | 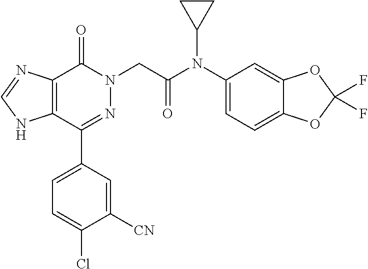 [SMILES: N#Cc1cc(-c2nn(CC(=O)N(c3ccc4c(c3)OC(F)(F)O4)C3CC3)c(=O)c3nc[nH]c23)ccc1Cl] | 525 [M + 1] | (400 MHz, DMSO) δ: 0.68 (s, 2H); 0.92 (d, J = 5.6 Hz, 2H); 3.32 (m, 1H); 5.21 (s, 2H); 7.16 (d, J = 8.0 Hz, 1H); 7.40-7.42 (m, 2H); 7.92 (d, J = 8.8 Hz, 1H); 8.46 (s, 1H); 8.76 (d, J = 8.4 Hz, 1H); 8.95 (s, 1H) |
| 446 | 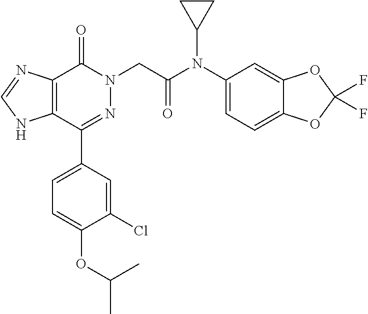 [SMILES: CC(C)Oc1ccc(-c2nn(CC(=O)N(c3ccc4c(c3)OC(F)(F)O4)C3CC3)c(=O)c3nc[nH]c23)cc1Cl] | 558 [M + 1] | (400 MHz, DMSO) δ: 0.68 (s, 2H), 0.92 (d, J = 6.0 Hz, 2H); 1.38 (d, J = 6.0 Hz, 6H), 3.32 (m, 1H); 4.77 (sept, J = 6.0 Hz, 1H); 5.18 (s, 2H); 7.16 (dd, J = 8.8, 2.0 Hz, 1H); 7.31 (d, J = 8.8 Hz, 1H); 7.39-7.42 (m, 2H), 8.36 (d, J = 8.4 Hz, 1H); 8.41 (s, 1H), 8.49 (s, 1H), 13.81 (br, 1H) |
| 447 | 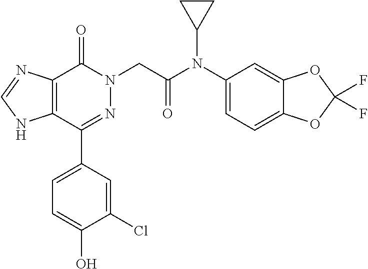 [SMILES: O=C(Cn1nc(-c2ccc(O)c(Cl)c2)c2[nH]cnc2c1=O)N(c1ccc2c(c1)OC(F)(F)O2)C1CC1] | 516.2 [M + H] | (400 MHz, DMSO-d₆): 0.67 (s, 2H), 0.91 (d, J = 5.6 Hz, 2H), 3.31 (m, 1H), 5.16 (s, 2H), 7.11-7.17 (m, 2H), 7.40 (s, 2H), 8.22 (d, J = 7.2 Hz, 1H), 8.40-8.44 (m, 2H), 10.21 (br, 1H), 13.78 (br, 1H) |
| 448 | 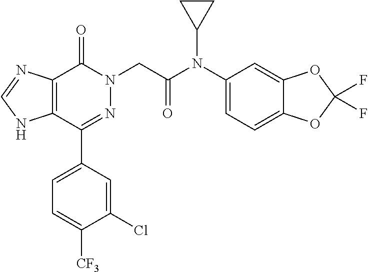 [SMILES: O=C(Cn1nc(-c2ccc(C(F)(F)F)c(Cl)c2)c2[nH]cnc2c1=O)N(c1ccc2c(c1)OC(F)(F)O2)C1CC1] | 568.2 [M + H] | (400 MHz, DMSO-d₆): 0.68 (s, 2H), 0.92 (d, J = 6.4 Hz, 2H), 3.32 (m, 1H), 5.23 (s, 2H), 7.16 (d, J = 8.4 Hz, 1H), 7.40-7.42 (m, 2H), 8.03 (d, J = 8.4 Hz, 1H), 8.50 (s, 1H), 8.60 (d, J = 8.0 Hz, 1H), 8.75 (s, 1H), 13.99 (br, 1H) |
| 449 | 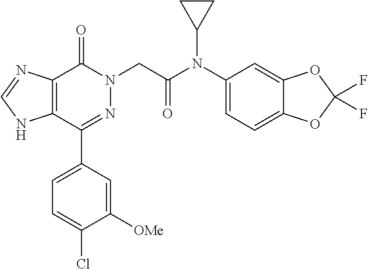 [SMILES: COc1cc(-c2nn(CC(=O)N(c3ccc4c(c3)OC(F)(F)O4)C3CC3)c(=O)c3nc[nH]c23)ccc1Cl] | 530.2 [M + H] | (400 MHz, DMSO-d₆): 0.68 (s, 2H), 0.92 (d, J = 6.0 Hz, 2H), 3.32 (s, 1H), 3.99 (s, 3H), 5.19 (s, 2H), 7.15 (dd, J = 8.4, 2.0 Hz, 1H), 7.39- 7.42 (m, 2H), 7.57 (d, J = 8.4 Hz, 1H), 8.01 (d, J = 9.2 Hz, 1H), 8.23 (s, 1H), 8.44 (s, 1H), 13.90 (br, 1H) |
| 450 | 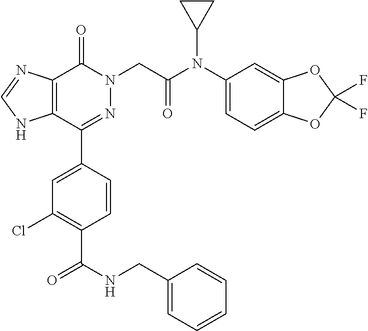 [SMILES: O=C(NCc1ccccc1)c1ccc(-c2nn(CC(=O)N(c3ccc4c(c3)OC(F)(F)O4)C3CC3)c(=O)c3nc[nH]c23)cc1Cl] | 633.2 [M + 1] | (400 MHz, DMSO) 0.68 (s, 2H), 0.91 (s, 2H), 3.32 (s, 1H), 4.53 (d, J = 4.8 Hz, 2H), 5.21 (s, 2H), 7.16 (d, J = 6.8 Hz, 1H), 7.29 (d, J = 6.0 Hz, 1H), 7.37-7.47 (m, 5H), 7.64 (d, J = 8.0 Hz, 1H), 8.45 (m, 2H), 8.57 (s, 1H), 8.82 (s, 1H), 14.02 (br, 1H) |
| 451 | 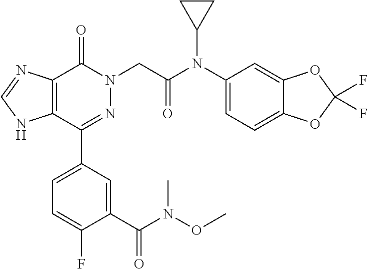 [SMILES: CON(C)C(=O)c1cc(-c2nn(CC(=O)N(c3ccc4c(c3)OC(F)(F)O4)C3CC3)c(=O)c3nc[nH]c23)ccc1F] | 571.2 [M + H] | (400 MHz, DMSO-d₆): 0.68 (s, 2H), 0.92 (d, J = 5.6 Hz, 2H), 3.33 (s, 4H), 3.59 (s, 3H), 5.20 (s, 2H), 7.16 (d, J = 8.0 Hz, 1H), 7.47-7.48 (m, 3H), 8.45 (s, 1H), 8.53 (m, 2H), 13.95 (br, 1H) |
| 452 | 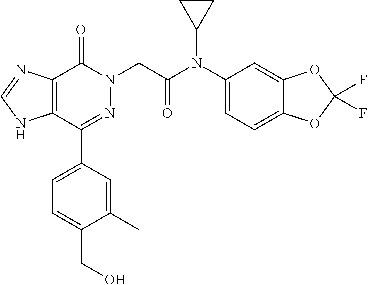 [SMILES: Cc1cc(-c2nn(CC(=O)N(c3ccc4c(c3)OC(F)(F)O4)C3CC3)c(=O)c3nc[nH]c23)ccc1CO] | 510.2 [M + H] | (400 MHz, DMSO-d₆): 0.68 (s, 2H), 0.92 (d, J = 5.6 Hz, 2H), 2.37 (s, 3H), 3.32 (m, 1H), 4.60 (d, J = 4.8 Hz, 2H), 4.92 (m, 1H), 5.18 (s, 2H), 7.16 (d, J = 8.4 Hz, 1H), 7.39-7.42 (m, 2H), 7.52 (d, J = 7.6 Hz, 1H), 8.12-8.16 (m, 2H), 8.40 (s, 1H), 13.83 (br, 1H) |
| 453 | 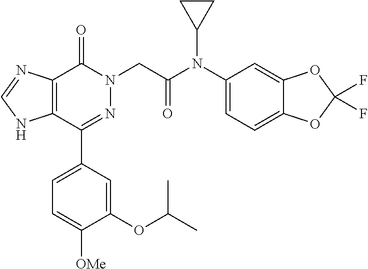 [SMILES: COc1ccc(-c2nn(CC(=O)N(c3ccc4c(c3)OC(F)(F)O4)C3CC3)c(=O)c3nc[nH]c23)cc1OC(C)C] | 554.2 [M + H] | (400 MHz, DMSO-d₆): 0.67 (s, 2H), 0.92 (d, J = 5.6 Hz, 2H), 1.34 (d, J = 6.0 Hz, 6H), 3.31 (m, 1H), 3.87 (s, 3H), 4.58 (sept, J = 6.0 Hz, 1H), 5.16 (s, 2H), 7.11-7.16 (m, 2H), 7.39-7.42 (m, 2H), 8.03 (m, 2H), 8.40 (s, 1H), 13.84 (br, 1H) |
| 454 | 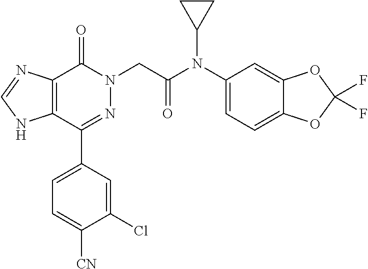 [SMILES: N#Cc1ccc(-c2nn(CC(=O)N(c3ccc4c(c3)OC(F)(F)O4)C3CC3)c(=O)c3nc[nH]c23)cc1Cl] | 525.2 [M + H] | (400 MHz, DMSO-d₆): 0.68 (s, 2H), 0.92 (d, J = 6.0 Hz, 2H), 3.32 (m, 1H), 5.23 (s, 2H), 7.17 (d, J = 8.8 Hz, 1H), 7.40-7.43 (m, 2H), 8.12 (d, J = 8.4 Hz, 1H), 8.49 (s, 1H), 8.62 (d, J = 7.6 Hz, 1H), 8.79 (s, 1H), 14.19 (br, 1H) |
| 455 | 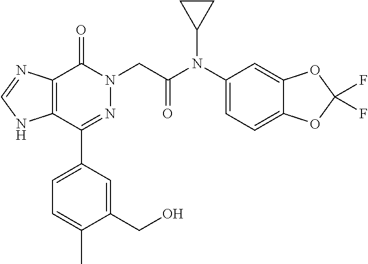 [SMILES: Cc1ccc(-c2nn(CC(=O)N(c3ccc4c(c3)OC(F)(F)O4)C3CC3)c(=O)c3nc[nH]c23)cc1CO] | 510.2 [M + H] | (400 MHz, DMSO-d₆): 0.68 (s, 2H), 0.92 (d, J = 6.0 Hz, 2H), 2.36 (s, 3H), 3.32 (m, 1H), 4.61 (d, J = 4.0 Hz, 2H), 4.91 (s, 1H), 5.17 (s, 2H), 7.16 (dd, J = 8.4, 1.6 Hz, 1H), 7.29 (d, J = 8.0 Hz, 1H), 7.39-7.42 (m, 2H), 8.19 (d, J = 8.0 Hz, 1H), 8.33-8.38 (m, 2H), 13.76 (br, 1H) |
| 456 | 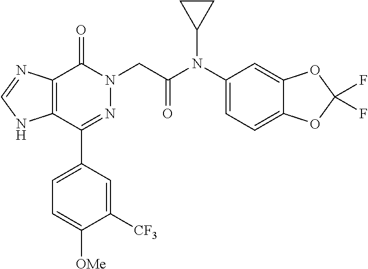 [SMILES: COc1ccc(-c2nn(CC(=O)N(c3ccc4c(c3)OC(F)(F)O4)C3CC3)c(=O)c3nc[nH]c23)cc1C(F)(F)F] | 562 [M − 1] | (400 MHz, DMSO) δ: 0.68 (s, 2H); 0.92 (d, J = 6.0 Hz, 2H); 3.22 (s, 3H); 3.32 (m, 1H); 3.63 (s, 1H); 5.19 (s, 2H); 7.16 (dd, J = 8.8, 2.0 Hz, 1H); 7.40-7.43 (m, 3H); 7.88 (s, 1H); 8.11 (d, J = 6.8 Hz, 1H); 8.421 (s, 1H); 13.83 (br, 1H) |
| 457 | 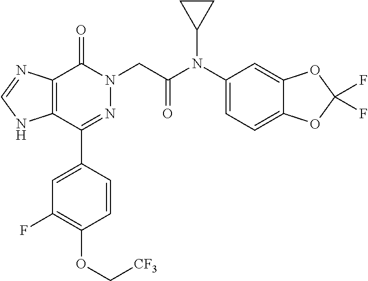 [SMILES: O=C(Cn1nc(-c2ccc(OCC(F)(F)F)c(F)c2)c2[nH]cnc2c1=O)N(c1ccc2c(c1)OC(F)(F)O2)C1CC1] | 582 [M + 1] | (400 MHz, DMSO) δ: 0.68 (s, 2H); 0.92 (d, J = 6.0 Hz, 2H); 3.32 (m, 1H); 4.91 (q, J = 8.8 Hz, 2H); 5.19 (s, 2H); 7.16 (d, J = 8.4 Hz, 1H); 7.39-7.49 (m, 3H); 8.28-8.32 (m, 2H); 8.44 (s, 1H); 13.93 (br, 1H) |
| 458 | 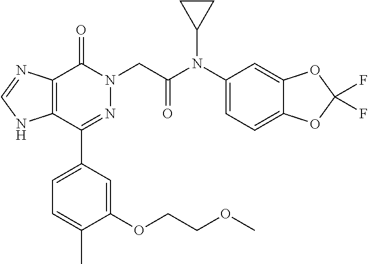 [SMILES: COCCOc1cc(-c2nn(CC(=O)N(c3ccc4c(c3)OC(F)(F)O4)C3CC3)c(=O)c3nc[nH]c23)ccc1C] | 554 [M + 1] | (400 MHz, DMSO) δ: 0.68 (s, 2H), 0.92 (d, J = 5.6 Hz, 2H); 2.27 (s, 3H), 3.32 (m, 1H); 3.40 (s, 3H); 3.77 (t, J = 4.8 Hz, 2H); 4.23 (t, J = 4.8 Hz, 2H), 5.18 (s, 2H), 7.16 (d, J = 8.4 Hz, 1H); 7.30 (d, J = 8.0 Hz, 1H); 7.39-7.42 (m, 2H); 7.90 (m, 1H); 8.00 (s, 1H); 8.41 (s, 1H); 13.83 (br, 1H) |
| 459 | 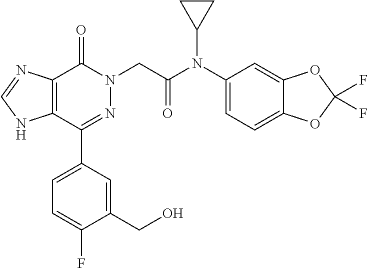 [SMILES: O=C(Cn1nc(-c2ccc(F)c(CO)c2)c2[nH]cnc2c1=O)N(c1ccc2c(c1)OC(F)(F)O2)C1CC1] | 514 [M + 1] | (400 MHz, DMSO) δ: 0.68 (s, 2H); 0.92 (d, J = 5.6 Hz, 2H); 3.32 (m, 1H); 4.67 (s, 2H); 5.19 (s, 3H); 7.17 (d, J = 8.4 Hz, 1H); 7.29 (t, J = 5.6 Hz, 1H); 7.40-7.43 (m, 2H); 8.36 (m, 1H); 8.43 (s, 1H); 8.50 (d, J = 6.0 Hz, 1H); 13.63 (br, 1H) |
| 460 | 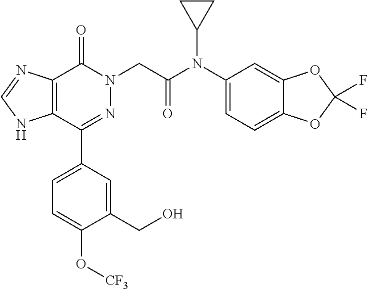 [SMILES: O=C(Cn1nc(-c2ccc(OC(F)(F)F)c(CO)c2)c2[nH]cnc2c1=O)N(c1ccc2c(c1)OC(F)(F)O2)C1CC1] | 580.2 [M + 1] | (400 MHz, DMSO): 0.68 (s, 2H); 0.92 (d, J = 6.0 Hz, 2H); 3.32 (m, 1H), 4.68 (s, 2H), 5.20-5.25 (m, 3H) 7.16 (d, J = 8.0 Hz, 1H), 7.41- 7.48 (m, 3H), 8.44 (m, 2H), 8.60 (s, 1H), 13.72 (br, 1H) |
| 461 | 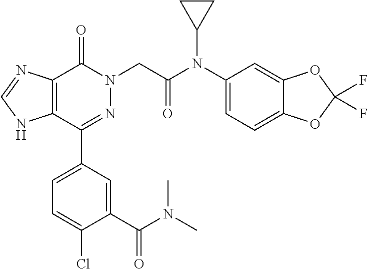 [SMILES: CN(C)C(=O)c1cc(-c2nn(CC(=O)N(c3ccc4c(c3)OC(F)(F)O4)C3CC3)c(=O)c3nc[nH]c23)ccc1Cl] | 569 [M − 1] | (400 MHz, DMSO) δ: 0.68 (s, 2H); 0.91 (m, 2H); 2.87 (s, 3H); 2.87 (s, 3H), 3.32 (m, 1H); 5.21 (s, 2H); 7.16 (d, J = 7.6 Hz, 1H); 7.42 (m, 2H); 7.69 (d, J = 8.8 Hz, 1H); 8.41-8.50 (m, 2H); 13.94 (br, 1H) |
| 462 | 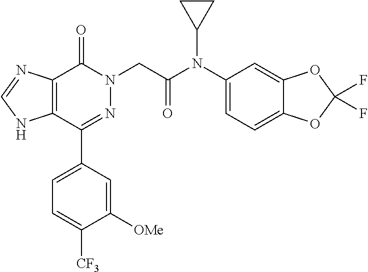 [SMILES: COc1cc(-c2nn(CC(=O)N(c3ccc4c(c3)OC(F)(F)O4)C3CC3)c(=O)c3nc[nH]c23)ccc1C(F)(F)F] | 564 [M + 1] | (400 MHz, DMSO) δ: 0.68 (s, 2H); 0.92 (d, J = 5.2 Hz, 2H); 3.32 (m, 1H); 4.02 (s, 3H); 5.21 (s, 2H); 7.16 (d, J = 8.4 Hz, 1H); 7.40-7.43 (m, 2H); 7.77 (d, J = 8.4 Hz, 1H); 8.12 (d, J = 8.0 Hz, 1H); 8.32 (s, 1H); 8.47 (s, 1H); 14.00 (br, 1H) |
| 463 | 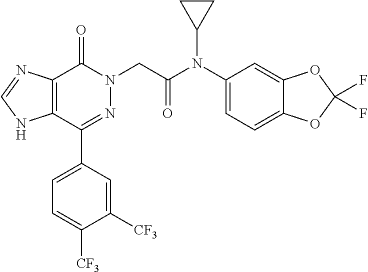 [SMILES: O=C(Cn1nc(-c2ccc(C(F)(F)F)c(C(F)(F)F)c2)c2[nH]cnc2c1=O)N(c1ccc2c(c1)OC(F)(F)O2)C1CC1] | 602 [M + 1] | (400 MHz, DMSO) δ: 0.68 (s, 2H); 0.92 (d, J = 6.0 Hz, 2H); 3.32 (m, 1H); 5.25 (s, 2H); 7.16 (d, J = 7.2 Hz, 1H); 7.40-7.42 (m, 2H); 8.23 (d, J = 8.4 Hz, 1H); 8.53 (s, 1H); 8.94 (d, J = 7.6 Hz, 1H); 9.13 (s, 1H); 14.13 (br, 1H) |
| 464 | 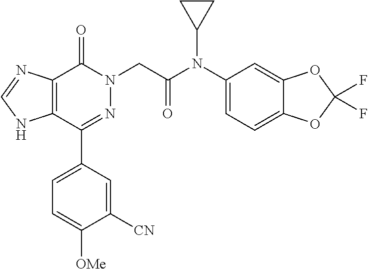 [SMILES: COc1ccc(-c2nn(CC(=O)N(c3ccc4c(c3)OC(F)(F)O4)C3CC3)c(=O)c3nc[nH]c23)cc1C#N] | 521 [M + 1] | (400 MHz, DMSO) δ: 0.68 (s, 2H); 0.92 (d, J = 6.0 Hz, 2H); 3.32 (m, 1H); 4.04 (s, 3H); 5.20 (s, 2H); 7.16 (d, J = 8.8 Hz, 1H); 7.39-7.45 (m, 3H); 8.46 (s, 1H); 8.73 (m, 2H); 13.98 (br, 1H) |
| 465 | 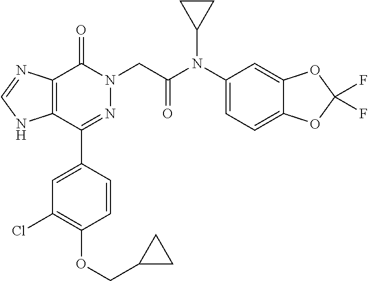 [SMILES: O=C(Cn1nc(-c2ccc(OCC3CC3)c(Cl)c2)c2[nH]cnc2c1=O)N(c1ccc2c(c1)OC(F)(F)O2)C1CC1] | 570 [M + 1] | (400 MHz, DMSO) δ: 0.419-0.431 (d, J = 4.8 Hz, 2H); 0.62-0.68 (m, 4H); 0.92 (d, J = 6.4 Hz, 2H); 1.30 (m, 1H); 3.32 (m, 1H); 4.06 (d, J = 6.8 Hz, 2H); 5.18 (s, 2H); 7.16 (d, J = 8.3 Hz, 1H); 7.29 (d, J = 8.8 Hz, 1H); 7.40-7.42 (m, 2H); 8.38 (d, J = 8.4 Hz, 1H); 8.42 (s, 1H); 8.49 (s, 1H); 13.92 (br, 1H) |
| 466 | 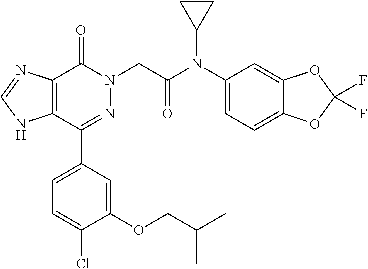 [SMILES: CC(C)COc1cc(-c2nn(CC(=O)N(c3ccc4c(c3)OC(F)(F)O4)C3CC3)c(=O)c3nc[nH]c23)ccc1Cl] | 572 [M + 1] | (400 MHz, DMSO) δ: 0.68 (s, 2H); 0.92 (d, J = 6.0 Hz, 2H); 1.08 (d, J = 6.8 Hz, 6H); 2.17 (m, 1H); 3.31 (m, 1H); 3.97 (d, J = 6.4 Hz, 2H); 5.19 (s, 2H); 7.15 (d, J = 8.4 Hz, 1H); 7.39- 7.42 (m, 2H); 7.57 (d, J = 8.4 Hz, 1H); 7.99 (d, J = 7.6 Hz, 1H); 8.20 (s, 1H); 8.44 (s, 1H); 13.94 (br, 1H) |
| 467 | 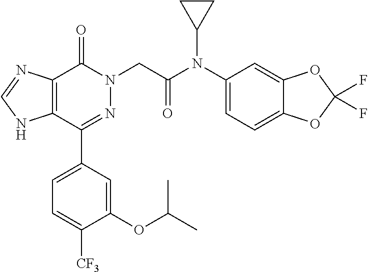 [SMILES: CC(C)Oc1cc(-c2nn(CC(=O)N(c3ccc4c(c3)OC(F)(F)O4)C3CC3)c(=O)c3nc[nH]c23)ccc1C(F)(F)F] | 592 [M + 1] | (400 MHz, DMSO) δ: 0.68 (s, 2H); 0.92 (d, J = 5.6 Hz, 2H); 1.41 (d, J = 5.6 Hz, 6H); 3.32 (m, 1H); 4.85 (sept, J = 5.6 Hz, 1H); 5.21 (s, 2H); 7.11 (d, J = 8.8 Hz, 1H); 7.34-7.38 (m, 2H); 7.72 (d, J = 8.4 Hz, 1H); 8.02 (m, 1H); 8.38 (m, 1H); 8.43 (s, 1H); 14.02 (br, 1H) |
| 468 | 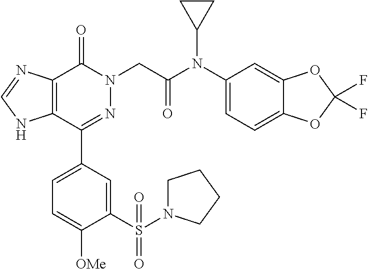 [SMILES: COc1ccc(-c2nn(CC(=O)N(c3ccc4c(c3)OC(F)(F)O4)C3CC3)c(=O)c3nc[nH]c23)cc1S(=O)(=O)N1CCCC1] | 629.2 [M + 1] | (400 MHz, DMSO) δ: 0.68 (s, 2H), 0.92 (d, J = 5.2 Hz, 2H), 1.82 (s, 4H), 3.09 (d, J = 6.0 Hz, 4H), 3.33 (m, 5H), 4.01 (s, 3H), 5.19 (s, 2H), 7.15 (d, J = 8.8 Hz, 1H), 7.35-7.43 (m, 3H), 8.45 (s, 1H), 8.67 (d, J = 9.2 Hz, 1H), 8.88 (s, 1H), 13.91 (br, 1H) |
| 469 | 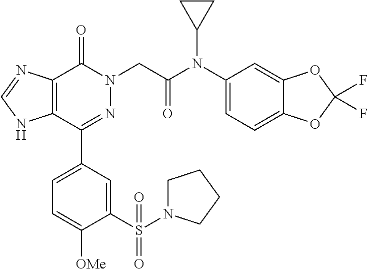 [SMILES: COc1ccc(-c2nn(CC(=O)N(c3ccc4c(c3)OC(F)(F)O4)C3CC3)c(=O)c3nc[nH]c23)cc1S(=O)(=O)N1CCCC1] | 559.2 [M + 1] | (400 MHz, DMSO) δ: 0.68 (s, 2H), 0.92 (d, J = 5.6 Hz, 2H), 3.32 (m, 1H), 5.25 (s, 2H), 7.16 (d, J = 8.4 Hz, 1H), 7.41 (m, 2H), 8.32 (d, J = 8.0 Hz, 1H), 8.53 (s, 1H), 8.93 (d, J = 7.2 Hz, 1H), 9.09 (s, 1H), 14.16 (br, 1H) |
| 470 | 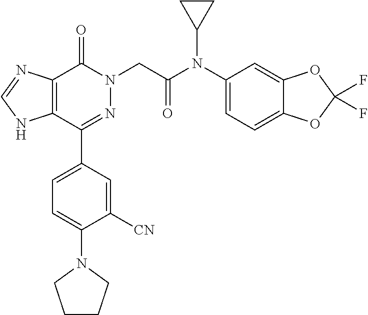 [SMILES: N#Cc1cc(-c2nn(CC(=O)N(c3ccc4c(c3)OC(F)(F)O4)C3CC3)c(=O)c3nc[nH]c23)ccc1N1CCCC1] | 560.2 [M + 1] | (400 MHz, DMSO) δ: 0.67 (s, 2H), 0.91 (d, J = 5.6 Hz, 2H), 2.02 (s, 4H), 3.31 (m, 1H), 3.66 (s, 4H), 5.16 (s, 2H), 6.93 (d, J = 9.2 Hz, 1H), 7.15 (d, J = 8.0 Hz, 1H), 7.40-7.42 (m, 2H), 8.38-8.41 (m, 2H), 8.68 (s, 1H), 13.86 (br, 1H) |
| 471 | 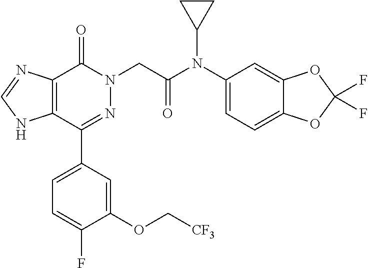 [SMILES: O=C(Cn1nc(-c2ccc(F)c(OCC(F)(F)F)c2)c2[nH]cnc2c1=O)N(c1ccc2c(c1)OC(F)(F)O2)C1CC1] | 582 [M + 1] | (400 MHz, DMSO) δ: 0.68 (s, 2H); 0.92 (d, J = 6.0 Hz, 2H); 3.32 (m, 1H); 4.88 (q, J = 8.8 Hz, 2H); 5.19 (s, 2H); 7.16 (d, J = 8.4 Hz, 1H); 7.40-7.47 (m, 3H); 8.18 (m, 1H); 8.27 (m, 1H); 8.45 (s, 1H); 13.94 (br, 1H) |
| 472 | 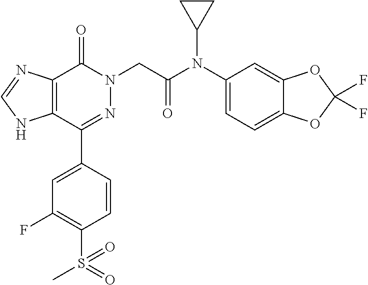 [SMILES: CS(=O)(=O)c1ccc(-c2nn(CC(=O)N(c3ccc4c(c3)OC(F)(F)O4)C3CC3)c(=O)c3nc[nH]c23)cc1F] | 562.2 [M + 1] | (400 MHz, DMSO) δ: 0.68 (s, 2H), 0.92 (d, J = 6.0 Hz, 2H), 3.32 (m, 1H), 3.38 (s, 3H), 5.23 (s, 2H), 7.16 (d, J = 8.4 Hz, 1H), 7.40-7.43 (m, 2H), 8.05 (t, J = 7.6 Hz, 1H), 8.50-8.53 (m, 3H), 14.06 (br, 1H) |
| 473 | 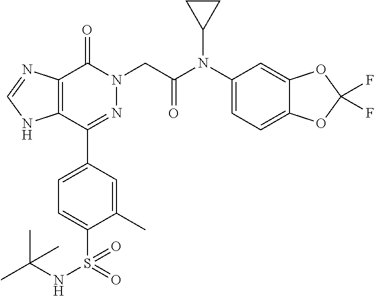 [SMILES: Cc1cc(-c2nn(CC(=O)N(c3ccc4c(c3)OC(F)(F)O4)C3CC3)c(=O)c3nc[nH]c23)ccc1S(=O)(=O)NC(C)(C)C] | 615 [M + 1] | (400 MHz, DMSO) δ: 0.68 (s, 2H); 0.90-0.92 (d, J = 6.0 Hz, 2H); 1.19 (s, 9H); 2.73 (s, 3H); 3.32 (m, 1H), 5.21 (s, 2H); 7.17 (d, J = 8.4 Hz, 1H); 7.30 (s, 1H); 7.40-7.43 (m, 2H); 8.04 (d, J = 8.4 Hz, 1H); 8.40 (m, 1H); 8.46 (s, 1H); 14.02 (br, 1H) |
| 474 | 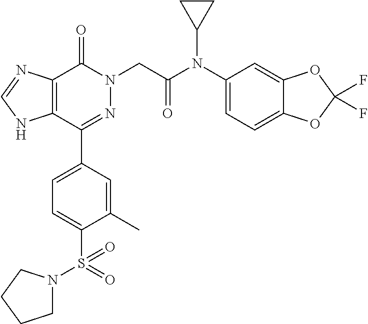 [SMILES: Cc1cc(-c2nn(CC(=O)N(c3ccc4c(c3)OC(F)(F)O4)C3CC3)c(=O)c3nc[nH]c23)ccc1S(=O)(=O)N1CCCC1] | 613 [M + 1] | (400 MHz, DMSO) δ: 0.68 (s, 2H); 0.92 (d, J = 5.6 Hz, 2H); 1.88 (t, J = 6.4 Hz, 4H); 2.70 (s, 3H); 3.28 (t, J = 6.4 Hz, 4H); 3.32 (m, 1H), 5.21 (s, 2H); 7.16 (d, J = 8.8 Hz, 1H); 7.40-7.43 (m, 2H); 7.96 (d, J = 8.4 Hz, 1H); 8.41 (m, 2H); 8.46 (s, 1H); 14.00 (br, 1H) |
| 475 | 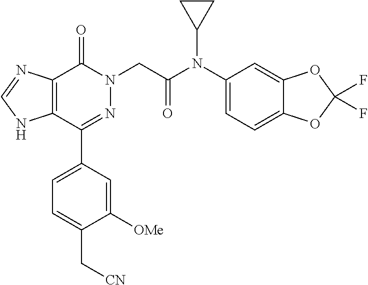 [SMILES: COc1cc(-c2nn(CC(=O)N(c3ccc4c(c3)OC(F)(F)O4)C3CC3)c(=O)c3nc[nH]c23)ccc1CC#N] | 535.2 [M + H] | (400 MHz, DMSO-d₆): 0.68 (s, 2H), 0.92 (d, J = 5.6 Hz, 2H), 3.32 (m, 1H), 3.92 (s, 2H), 3.98 (s, 3H), 5.19 (s, 2H), 7.16 (d, J = 8.4 Hz, 1H), 7.41-7.42 (m, 2H), 7.50 (d, J = 8.0 Hz, 1H), 8.02 (m, 1H), 8.11 (m, 1H), 8.44 (s, 1H), 13.92 (br, 1H) |
| 476 | 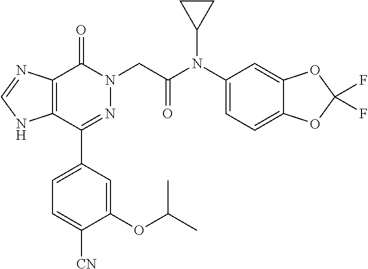 [SMILES: CC(C)Oc1cc(-c2nn(CC(=O)N(c3ccc4c(c3)OC(F)(F)O4)C3CC3)c(=O)c3nc[nH]c23)ccc1C#N] | 549.2 [M + H] | (400 MHz, DMSO-d₆): 0.68 (s, 2H), 0.92 (d, J = 5.6 Hz, 2H), 1.44 (d, J = 6.0 Hz, 6H), 3.32 (m, 1H), 4.86 (sept, J = 6.0 Hz, 1H), 5.22 (s, 2H), 7.15 (d, J = 7.2 Hz, 1H), 7.40-7.43 (m, 2H), 7.84 (d, J = 8.4 Hz, 1H), 8.06 (d, J = 8.0 Hz, 1H), 8.39 (s, 1H), 8.48 (s, 1H), 14.04 (br, 1H) |
| 477 | 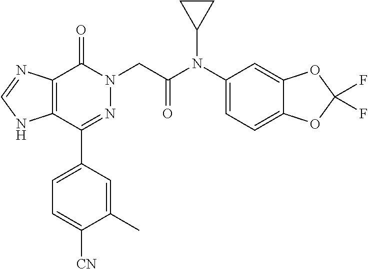 [SMILES: Cc1cc(-c2nn(CC(=O)N(c3ccc4c(c3)OC(F)(F)O4)C3CC3)c(=O)c3nc[nH]c23)ccc1C#N] | 505.2 [M + H] | (400 MHz, DMSO-d₆): 0.68 (s, 2H), 0.92 (d, J = 5.6 Hz, 2H), 2.68 (s, 3H), 3.32 (m, 1H), 5.22 (s, 2H), 7.16 (d, J = 8.4 Hz, 1H), 7.40-7.43 (m, 2H), 7.91 (d, J = 8.0 Hz, 1H), 8.41 (d, J = 8.0 Hz, 1H), 8.47 (s, 2H), 13.97 (br, 1H) |
| 478 | 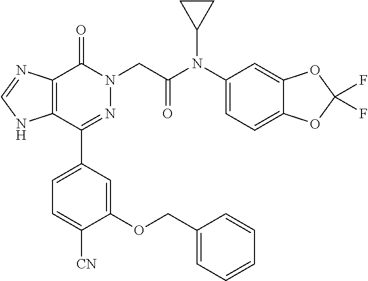 [SMILES: N#Cc1ccc(-c2nn(CC(=O)N(c3ccc4c(c3)OC(F)(F)O4)C3CC3)c(=O)c3nc[nH]c23)cc1OCc1ccccc1] | 597.2 [M + H] | (400 MHz, DMSO-d₆): 0.68- 0.73 (m, 2H), 0.92 (d, J = 5.6 Hz, 2H), 3.32 (m, 1H), 5.21 (s, 2H), 5.40 (s, 2H), 7.16 (d, J = 8.8 Hz, 1H), 7.36-7.47 (m, 5H), 7.56- 7.58 (m, 2H), 7.87 (d, J = 8.0 Hz, 1H), 8.17 (d, J = 8.0 Hz, 1H), 8.46 (d, J = 9.2 Hz, 2H), 14.01 (br, 1H) |
| 479 | 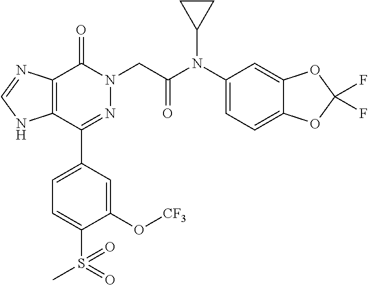 [SMILES: CS(=O)(=O)c1ccc(-c2nn(CC(=O)N(c3ccc4c(c3)OC(F)(F)O4)C3CC3)c(=O)c3nc[nH]c23)cc1OC(F)(F)F] | 628.2 [M + H] | (400 MHz, DMSO-d₆): 0.68 (s, 2H), 0.92 (d, J = 5.6 Hz, 2H), 3.32 (m, 1H), 3.36 (s, 3H), 5.24 (s, 2H), 7.16 (d, J = 8.8 Hz, 1H), 7.42 (m, 2H), 8.19 (d, J = 8.0 Hz, 1H), 8.53 (s, 1H), 8.64 (d, J = 8.4 Hz, 1H), 8.80 (s, 1H), 14.15 (br, 1H) |
| 480 | 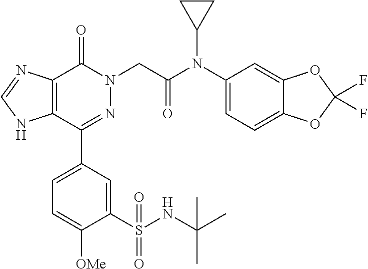 [SMILES: COc1ccc(-c2nn(CC(=O)N(c3ccc4c(c3)OC(F)(F)O4)C3CC3)c(=O)c3nc[nH]c23)cc1S(=O)(=O)NC(C)(C)C] | 631.3 [M + H] | (400 MHz, DMSO-d₆): 0.68 (s, 2H), 0.92 (d, J = 5.6 Hz, 2H), 1.17 (s, 9H), 3.32 (m, 1H), 4.03 (s, 3H), 5.19 (s, 2H), 6.72 (s, 1H), 7.16 (d, J = 8.8 Hz, 1H), 7.36-7.42 (m, 3H), 8.44 (s, 1H), 8.65 (d, J = 8.8 Hz, 1H), 8.89 (s, 1H), 14.03 (br, 1H) |
| 481 | 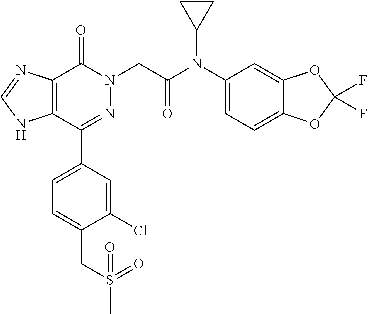 [SMILES: CS(=O)(=O)Cc1ccc(-c2nn(CC(=O)N(c3ccc4c(c3)OC(F)(F)O4)C3CC3)c(=O)c3nc[nH]c23)cc1Cl] | 592.2 [M + H] | (400 MHz, DMSO-d₆): 0.68 (s, 2H), 0.92 (d, J = 4.8 Hz, 2H), 3.04 (s, 3H), 3.32 (m, 1H), 4.73 (s, 2H), 5.21 (s, 2H), 7.17 (d, J = 8.4 Hz, 1H), 7.40-7.43 (m, 2H), 7.74 (d, J = 8.0 Hz, 1H), 8.42 (m, 1H), 8.46 (s, 1H), 8.60 (m, 1H), 14.01 (br, 1H) |
| 482 | 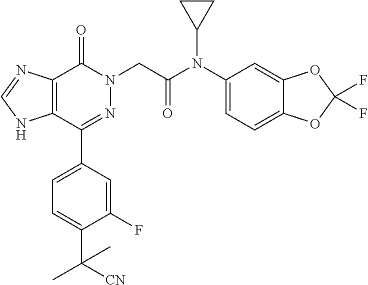 [SMILES: CC(C)(C#N)c1ccc(-c2nn(CC(=O)N(c3ccc4c(c3)OC(F)(F)O4)C3CC3)c(=O)c3nc[nH]c23)cc1F] | 551.2 [M + H] | (400 MHz, DMSO-d₆): 0.68 (s, 2H), 0.92 (d, J = 6.0 Hz, 2H), 1.83 (s, 6H), 3.32 (m, 1H), 5.20 (s, 2H), 7.16 (d, J = 8.4 Hz, 1H), 7.41 (m, 2H), 7.67 (t, J = 8.4 Hz, 1H), 8.32 (m, 2H), 8.46 (s, 1H), 14.02 (bs, 1H) |
| 483 | 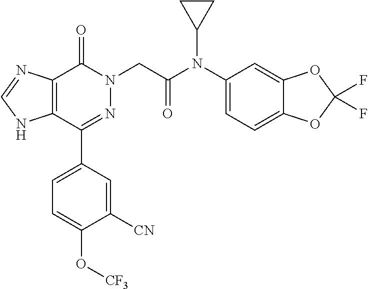 [SMILES: N#Cc1cc(-c2nn(CC(=O)N(c3ccc4c(c3)OC(F)(F)O4)C3CC3)c(=O)c3nc[nH]c23)ccc1OC(F)(F)F] | 575.3 [M + H⁺] | (400 MHz, DMSO-d₆): 0.69 (s, 2H), 0.92 (d, J = 5.6 Hz, 2H), 3.32 (m, 1H), 5.22 (s, 2H), 7.15 (d, J = 8.4 Hz, 1H), 7.38-7.40 (m, 2H), 7.83 (d, J = 9.2 Hz, 1H), 8.46 (s, 1H), 8.87 (m, 1H), 8.95 (s, 1H), 14.00 (br, 1H) |
| 484 | 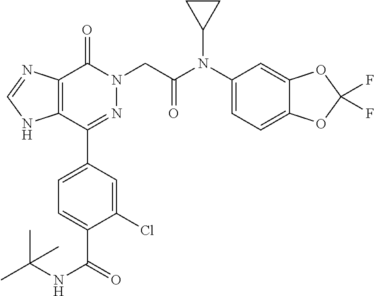 [SMILES: CC(C)(C)NC(=O)c1ccc(-c2nn(CC(=O)N(c3ccc4c(c3)OC(F)(F)O4)C3CC3)c(=O)c3nc[nH]c23)cc1Cl] | 599.2 [M + H] | (400 MHz, DMSO-d₆): 0.68 (m, 2H), 0.92 (d, J = 5.6 Hz, 2H), 1.42 (s, 9H), 3.32 (m, 1H), 5.21 (s, 2H), 7.16 (d, J = 8.8 Hz, 1H), 7.41-7.43 (m, 2H), 7.53 (d, J = 8.0 Hz, 1H), 7.82 (s, 1H), 8.39 (d, J = 7.6 Hz, 1H), 8.45 (s, 1H), 8.52 (s, 1H), 13.97 (br, 1H) |
| 485 | 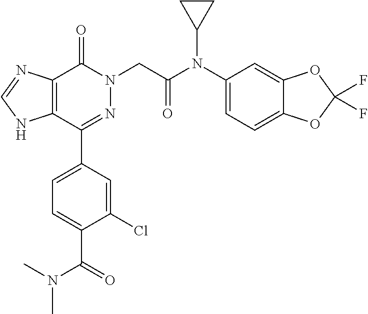 [SMILES: CN(C)C(=O)c1ccc(-c2nn(CC(=O)N(c3ccc4c(c3)OC(F)(F)O4)C3CC3)c(=O)c3nc[nH]c23)cc1Cl] | 571.2 [M + H] | (400 MHz, DMSO-d₆): 0.68 (m, 2H), 0.92 (d, J = 5.6 Hz, 2H), 2.86 (s, 3H), 3.08 (s, 3H), 3.32 (m, 1H), 5.21 (s, 2H), 7.16 (d, J = 8.4 Hz, 1H), 7.40-7.43 (m, 2H), 7.52 (d, J = 8.0 Hz, 1H), 8.44-8.47 (m, 2H), 8.56 (s, 1H), 13.86 (br, 1H) |
| 486 | 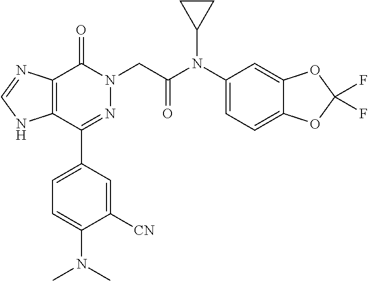 [SMILES: CN(C)c1ccc(-c2nn(CC(=O)N(c3ccc4c(c3)OC(F)(F)O4)C3CC3)c(=O)c3nc[nH]c23)cc1C#N] | 534.0 [M + H] | (400 MHz, DMSO-d6): 0.67 (s, 2H), 0.91 (d, J = 6.0 Hz, 2H), 3.15 (s, 6H), 3.30 (m, 1H), 5.16 (s, 2H), 7.13-7.15 (m, 2H), 7.37-7.39 (m, 2H), 8.39 (s, 1H), 8.46 (d, J = 8.4 Hz, 1H), 8.67 (s, 1H), 13.85 (br, 1H) |
| 487 | 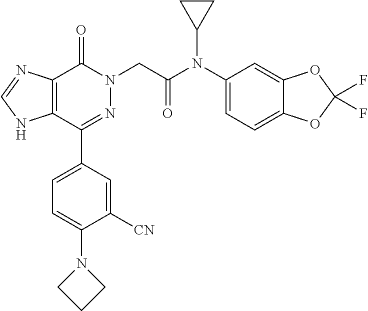 [SMILES: N#Cc1cc(-c2nn(CC(=O)N(c3ccc4c(c3)OC(F)(F)O4)C3CC3)c(=O)c3nc[nH]c23)ccc1N1CCC1] | 546.1 [M + H] | (400 MHz, DMSO-d6): 0.66 (s, 2H), 0.90 (d, J = 5.6 Hz, 2H), 2.33 (m, 2H), 3.29 (m, 1H), 4.25 (t, J = 7.2 Hz, 4H), 5.13 (s, 2H), 6.67 (d, J = 9.2 Hz, 1H), 7.13 (dd, J = 8.4, 2.0 Hz, 1H), 7.36- 7.39 (m, 2H), |
| 488 | 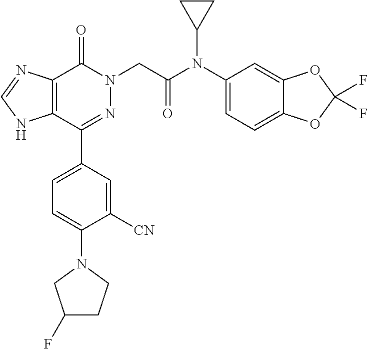 [SMILES: N#Cc1cc(-c2nn(CC(=O)N(c3ccc4c(c3)OC(F)(F)O4)C3CC3)c(=O)c3nc[nH]c23)ccc1N1CCC(F)C1] | 576.1 [M − H] | (500 MHz, DMSO-d6): 0.66-0.94 (m, 4H), 2.28- 2.36 (m, 2H), 3.73-3.85 (m, 4H), 3.99 (m, 1H), 5.45 (s, 1H), 5.55 (s, 1H), 6.97 (d, J = 7.6 Hz, 1H), 7.16 (m, 1H), 7.43-7.45 (m, 2H), 8.42 (d, J = 10.0 Hz, 1H), 8.48 (s, 1H), 8.74 (s, 1H), 14.1 (br, 1H) |
| 489 | 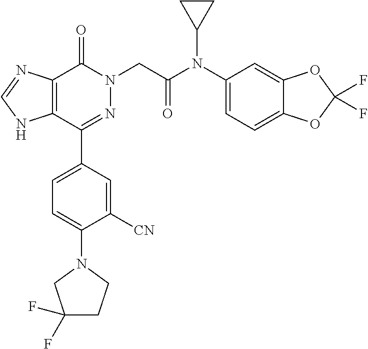 [SMILES: N#Cc1cc(-c2nn(CC(=O)N(c3ccc4c(c3)OC(F)(F)O4)C3CC3)c(=O)c3nc[nH]c23)ccc1N1CCC(F)(F)C1] | 594.2 [M − H] | (500 MHz, DMSO-d6): 0.67 (m, 2H), 0.92 (m, 2H), 2.58- 2.64 (m, 2H), 3.88 (t, J = 6.0 Hz, 2H), 4.08 (t, J = 6.4 Hz, 2H), 5.45 (br, 1H), 7.01 (d, J = 7.6 Hz, 1H), 7.16 (m, 1H), 7.43-7.45 (m, 2H), 8.42 (s, 1H), 8.48 (d, J = 6.4 Hz, 1H), 8.77 (s, 1H), 14.1 (br, 1H) |
| 490 | 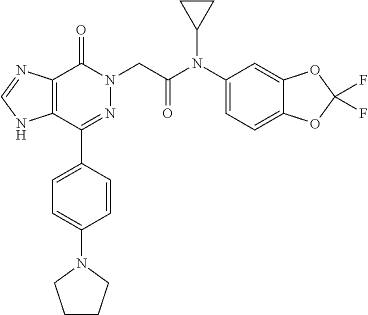 [SMILES: O=C(Cn1nc(-c2ccc(N3CCCC3)cc2)c2[nH]cnc2c1=O)N(c1ccc2c(c1)OC(F)(F)O2)C1CC1] | 533.1 [M − H] | (500 MHz, DMSO-d6): 0.66 (m, 2H), 0.93 (m, 2H), 1.97- 2.00 (m, 4H), 4.85 (s, 1H), 5.45 (s, 1H), 6.64 (d, J = 7.2 Hz, 1H), 7.15 (s, 1H), 7.43- 7.45 (m, 2H), 8.29-8.31 (m, 2H), 8.44 (s, 1H), 14.03, (s, 1H) |
| 491 | 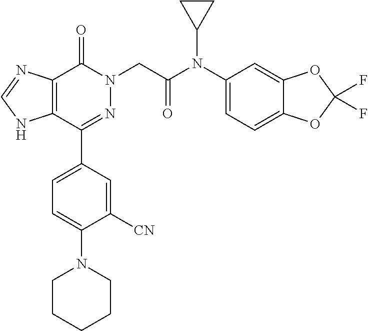 [SMILES: N#Cc1cc(-c2nn(CC(=O)N(c3ccc4c(c3)OC(F)(F)O4)C3CC3)c(=O)c3nc[nH]c23)ccc1N1CCCCC1] | 574.0 [M + H] | (400 MHz, DMSO-d6): 0.67 (s, 2H), 0.90 (d, J = 5.6 Hz, 2H), 1.65 (m, 2H), 1.74 (m, 4H), 3.31 (m, 5H), 5.17 (s, 2H), 7.14 (d, J = 7.6 Hz, 1H), 7.26 (d, J = 8.8 Hz, 1H), 7.37-7.39 (m, 2H), 8.41 (s, 1H), 8.53 (d, J = 8.0 Hz, 1H), 8.68 (s, 1H), 13.83 (br, 1H) |

### Example 492

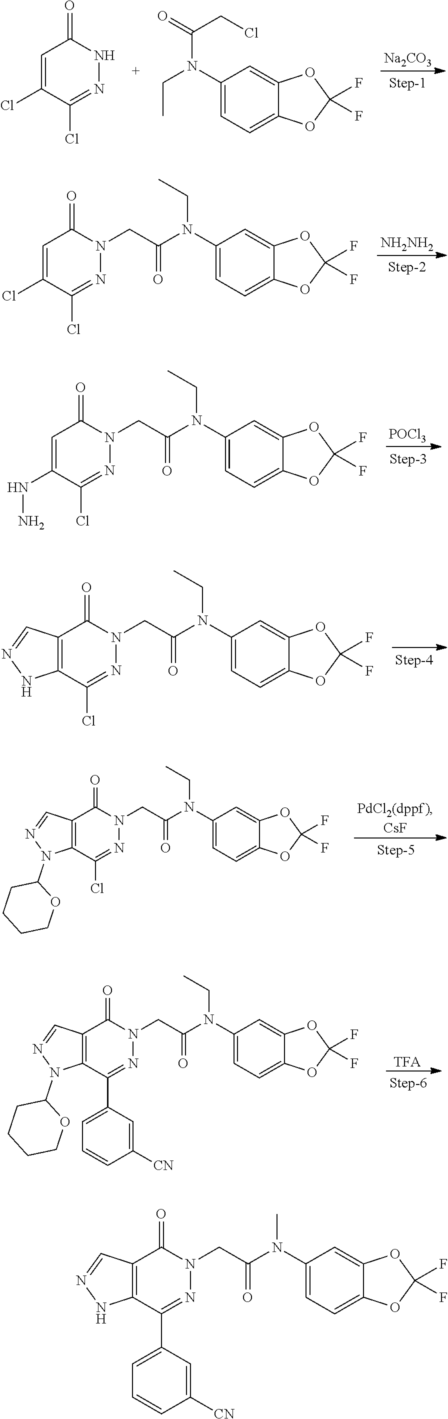

Step-1: A mixture of 5,6-dichloropyridazin-3(2H)-one (4.0 g, 24.3 mmol) in DMF (30 mL), sodium carbonate (15.5 g, 146 mmol), 2-chloro-N-(2,2-difluorobenzo[d][1,3]dioxol-5-yl)-N-ethylacetamide (6.75 g, 24.3 mmol) was stirred at room temperature for 12 h. The reaction mixture was quenched with ice water and extracted with ethyl acetate. The organic layer was dried and concentrated. The product was purified by column chromatography (15-20% ethyl acetate in hexane) to give 2-(3,4-dichloro-6-oxopyridazin-1(6H)-yl)-N-(2,2-difluorobenzo[d][1,3]dioxol-5-yl)-N-ethylacetamide (9.2 g, 93%). (MS ESI +ve, 406.0 [M+1]).

Step-2: To a solution of 2-(3,4-dichloro-6-oxopyridazin-1(6H)-yl)-N-(2,2-difluorobenzo[d][1,3]dioxol-5-yl)-N-ethylacetamide (9.2 g, 22.6 mmol) in ethanol (20 mL) was added hydrazine hydrate (2.26 g, 45.3 mmol). The reaction mixture was stirred at 80° C. for 1 h and the solvent was evaporated to give 2-(3-chloro-4-hydrazineyl-6-oxopyridazin-1(6H)-yl)-N-(2,2-difluorobenzo[d][1,3]dioxol-5-yl)-N-ethylacetamide (8.4 g, 92%). (MS ESI +ve, 402.0 [M+1]).

Step-3: To a solution of DMF (5.6 mL), POCl₃ (4.0 mL) was added 2-(3-chloro-4-hydrazineyl-6-oxopyridazin-1(6H)-yl)-N-(2,2-difluorobenzo[d][1,3]dioxol-5-yl)-N-ethylacetamide (8.0 g, 20.0 mmol) in DMF (6 mL)) dropwise at room temperature. The reaction mixture was stirred at 80° C. for 12 h and poured into ice water. The solid was filtered and purified by combiflash (40% of ethyl acetate in dichloromethane) to give 2-(7-chloro-4-oxo-1,4-dihydro-5H-pyrazolo[3,4-d]pyridazin-5-yl)-N-(2,2-difluorobenzo[d][1,3]dioxol-5-yl)-N-ethylacetamide (0.20 g, 2.4%). (MS ESI +ve, 412.2 [M+1]).

Step-4: To a stirred solution of 2-(7-chloro-4-oxo-1,4-dihydro-5H-pyrazolo[3,4-d]pyridazin-5-yl)-N-(2,2-difluorobenzo[d][1,3]dioxol-5-yl)-N-ethylacetamide (0.20 g, 0.48 mmol) in THF (5 mL) was added 2,3 dihydropyran (0.082 g, 5.35 mol), PTSA (catalytic amount). The mixture was stirred at room temperature for 3 h and solvent was evaporated. The product was purified by column (20% ethyl acetate in dichloromethane) to give 2-(7-chloro-4-oxo-1-(tetrahydro-2H-pyran-2-yl)-1,4-dihydro-5H-pyrazolo[3,4-d]pyridazin-5-yl)-N-(2,2-difluorobenzo[d][1,3]dioxol-5-yl)-N-ethylacetamide (0.09 g, 37%). (MS ESI +ve, 496.3 [M+1]).

Step-5: A mixture of 2-(7-chloro-4-oxo-1-(tetrahydro-2H-pyran-2-yl)-1,4-dihydro-5H-pyrazolo[3,4-d]pyridazin-5-yl)-N-(2,2-difluorobenzo[d][1,3]dioxol-5-yl)-N-ethylacetamide (0.09 g, 0.18 mmol), (3-cyanophenyl) boronic acid (0.032 g, 0.22 mmol) in dimethoxyethane (3 mL), cesium fluoride (0.055 g, 0.36 mmol), water (0.5 mL) was degassed. PdCl₂(dppf) (0.002 g, 0.018 mmol) was added and the mixture was degassed again. The mixture was heated at 85° C. for 4 h and the solvent was evaporated. The residue was purified by column (50% ethyl acetate in hexane) to give 2-(7-(3-cyanophenyl)-4-oxo-1-(tetrahydro-2H-pyran-2-yl)-1,4-dihydro-5H-pyrazolo[3,4-d]pyridazin-5-yl)-N-(2,2-difluorobenzo[d][1,3]dioxol-5-yl)-N-ethylacetamide (0.05 g, 48%). (MS ESI +ve, 563.0[M+1]).

Step-6: A solution of 2-(7-(3-cyanophenyl)-4-oxo-1-(tetrahydro-2H-pyran-2-yl)-1,4-dihydro-5H-pyrazolo[3,4-d]pyridazin-5-yl)-N-(2,2-difluorobenzo[d][1,3]dioxol-5-yl)-N-ethylacetamide (0.050 g, 0.088 mmol) in DCM (1 mL) was treated with TFA (3 mL) at 0° C. The mixture was stirred at room temperature for 12 h and solvent was evaporated. The residue was purified by preparative HPLC to give 2-(7-(3-cyanophenyl)-4-oxo-1,4-dihydro-5H-pyrazolo[3,4-d]pyridazin-5-yl)-N-(2,2-difluorobenzo[d][1,3]dioxol-5-yl)-N-ethylacetamide (0.003 g, 7.0%). (MS ESI +ve, 479 [M+2]). ¹H NMR: (400 MHz, DMSO) δ: 1.02 (t, d=6.8 Hz, 3H), 3.67 (q, J=6.8 Hz, 2H), 4.75 (s, 2H), 7.32 (d, J=8.0 Hz, 1H), 7.52 (d, J=8.4 Hz, 1H), 7.66 (s, 1H), 7.76 (t, J=8.0 Hz, 1H), 8.00 (d, J=7.6 Hz, 1H), 8.23 (d, J=8.0 Hz, 1H), 8.28 (s, 1H), 8.66 (s, 1H), 14.90 (br, 1H).

### Example 493

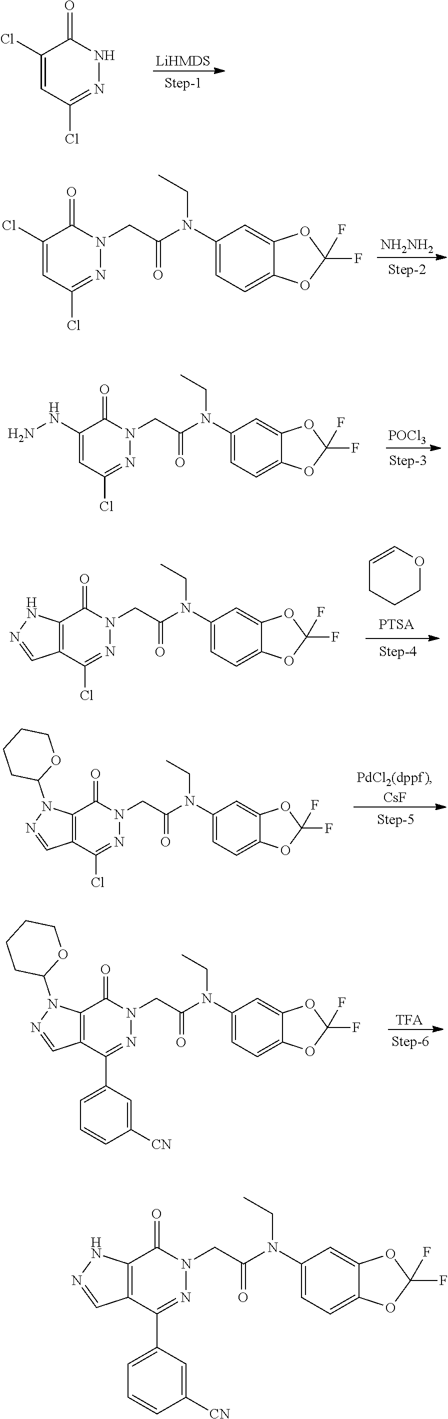

2-(4-(3-cyanophenyl)-7-oxo-1,7-dihydro-6H-pyrazolo[3,4-d]pyridazin-6-yl)-N-(2,2-difluorobenzo[d][1,3]dioxol-5-yl)-N-ethylacetamide was synthesized in a similar fashion as showed in the scheme. (MS ESI +ve, 479 [M+1]). ¹H NMR: (400 MHz, DMSO) δ: 1.04 (t, J=6.8 Hz, 3H), 3.68 (q, J=6.8 Hz, 2H), 4.73 (s, 2H), 7.33 (d, J=8.0 Hz, 1H), 7.53 (d, J=8.4 Hz, 1H), 7.66 (s, 1H), 7.79 (t, J=8.0 Hz, 1H), 8.00 (d, J=7.6 Hz, 1H), 8.60 (m, 1H), 8.68 (m, 1H), 8.84 (m, 1H), 14.75 (br, 1H).

In a similar fashion, the following examples were synthesized:

| Example # | Structure | m/z | ¹H NMR |
|---|---|---|---|
| 494 | 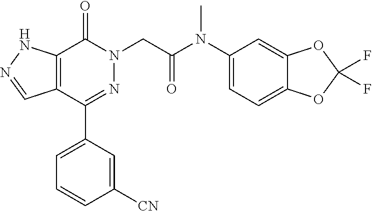 [SMILES: CN(C(=O)Cn1nc(-c2cccc(C#N)c2)c2cn[nH]c2c1=O)c1ccc2c(c1)OC(F)(F)O2] | 465 [M + 1] | (400 MHz, DMSO) δ: 3.18 (s, 3H), 4.74 (s, 2H), 7.36 (m, 1H), 7.50 (d, J = 8.0 Hz, 1H), 7.69 (s, 1H), 7.40 (t, J = 8.0 Hz, 1H), 7.93 (d, J = 7.6 Hz, 1H), 8.54 (s, 1H), 8.70 (d, J = 8.0 Hz, 1H), 8.84 (s, 1H) |
| 495 | 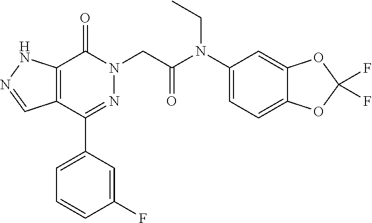 [SMILES: CCN(C(=O)Cn1nc(-c2cccc(F)c2)c2cn[nH]c2c1=O)c1ccc2c(c1)OC(F)(F)O2] | 472.0 [M + 1] | (400 MHz, DMSO) δ: 1.02 (t, J = 6.8 Hz, 3H), 3.67 (q, J = 6.8 Hz, 2H), 4.70 (s, 2H), 7.29- 7.35 (m, 2H), 7.51 (d, J = 8.4 Hz, 1H), 7.58- 7.64 (m, 2H), 8.15 (m, 1H), 8.20 (d, J = 7.2 Hz, 1H), 8.86 (s, 1H), 14.75 (br, 1H) |
| 496 | 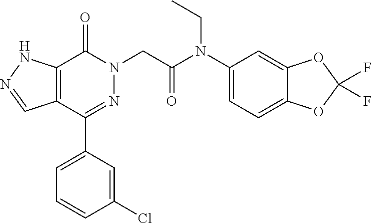 [SMILES: CCN(C(=O)Cn1nc(-c2cccc(Cl)c2)c2cn[nH]c2c1=O)c1ccc2c(c1)OC(F)(F)O2] | 488 [M + 1] | (400 MHz, DMSO) δ: 1.02 (t, J = 5.6 Hz, 3H), 3.67 (q, J = 5.6 Hz, 2H), 4.70 (s, 2H), 7.31 (d, J = 8.4 Hz, 1H), 7.51 (d, J = 8.4 Hz, 1H), 7.56- 7.582 (m, 2H), 7.64 (s, 1H), 8.31 (m, 1H), 8.37 (m, 1H), 8.86 (s, 1H), 14.75 (br, 1H) |
| 497 | 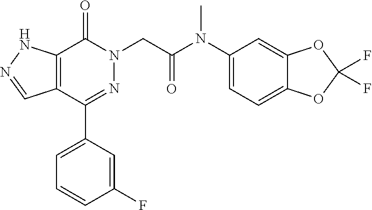 [SMILES: CN(C(=O)Cn1nc(-c2cccc(F)c2)c2cn[nH]c2c1=O)c1ccc2c(c1)OC(F)(F)O2] | 458 [M + 1] | (400 MHz, DMSO) δ: 3.18 (s, 3H), 4.74 (s, 2H), 7.31-7.36 (m, 2H), 7.50 (d, J = 8.0 Hz, 1H), 7.58 (td, J = 8.0, 6.4 Hz, 1H), 7.69 (s, 1H), 8.12 (d, J = 10.4 Hz, 1H), 8.17 (d, J = 8.0 Hz, 1H), 8.71 (s, 11H), 10.14 (br, 1H) |
| 498 | 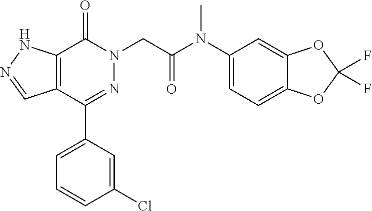 [SMILES: CN(C(=O)Cn1nc(-c2cccc(Cl)c2)c2cn[nH]c2c1=O)c1ccc2c(c1)OC(F)(F)O2] | 474.0 [M + 1] | (400 MHz, DMSO) δ: 3.18 (s, 3H), 4.76 (s, 2H), 7.38 (m, 1H), 7.51 (d, J = 8.4 Hz, 1H), 7.57 (m, 2H), 7.70 (s, 1H), 8.31 (m, 1H), 8.38 )s, 1H), 8.88 (s, 1H), 14.76 (br, 1H) |
| 499 | 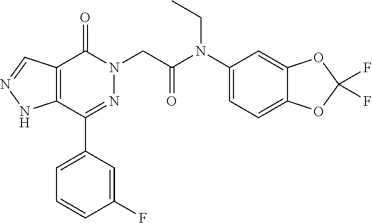 [SMILES: CCN(C(=O)Cn1nc(-c2cccc(F)c2)c2[nH]ncc2c1=O)c1ccc2c(c1)OC(F)(F)O2] | 472.0 [M + 1] | (400 MHz, DMSO) δ: 1.02 (t, J = 5.6 Hz, 3H), 3.67 (q, J = 5.6 Hz, 2H), 4.70 (s, 2H), 7.29- 7.37 (m, 2H), 7.51 (d, J = 8.4 Hz, 1H), 7.56- 7.64 (m, 2H), 8.07 (s, 1H), 8.14 (s, 1H), 8.79 (s, 1H), 14.17 (br, 1H) |
| 500 | 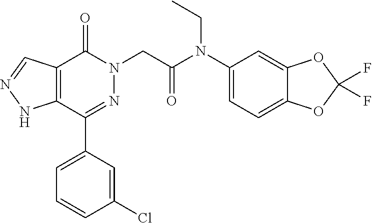 [SMILES: CCN(C(=O)Cn1nc(-c2cccc(Cl)c2)c2[nH]ncc2c1=O)c1ccc2c(c1)OC(F)(F)O2] | 488.0 [M + 1] | (400 MHz, DMSO) δ: 1.04 (t, J = 6.8 Hz, 3H), 3.68 (q, J = 6.8 Hz, 2H), 4.71 (s, 2H), 7.31 (d, J = 8.8 Hz, 1H), 7.52 (d, J = 8.8 Hz, 1H), 7.57- 7.62 (m, 2H), 7.65 (s, 1H), 8.28 (m, 1H), 8.36 (s, 1H), 8.76 (s, 1H), 12.14 (br, 1H) |

### Example 501

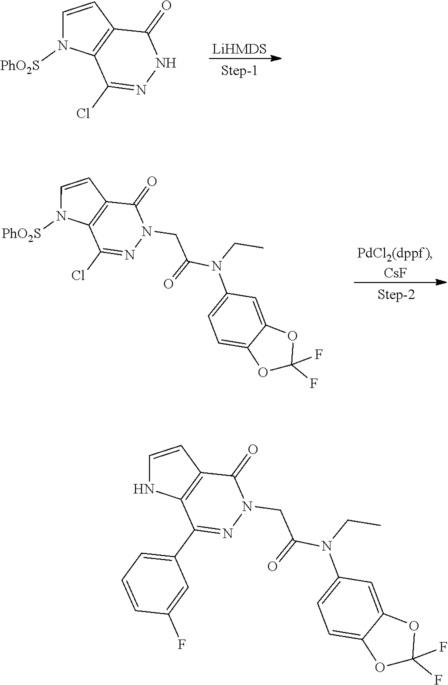

Step-1: To a solution of 7-chloro-1-(phenylsulfonyl)-1,5-dihydro-4H-pyrrolo[2,3-d]pyridazin-4-one (0.50 g, 1.61 mmol) in DMF (20 mL) was added lithium bis(trimethylsilyl)amide (2.4 mL, 2.42 mmol) at 0° C. The mixture was stirred for 15 min followed by addition of 2-chloro-N-(2,2-difluorobenzo[d][1,3]dioxol-5-yl)-N-ethylacetamide (0.49 g, 1.78 mmol). The reaction mixture was stirred overnight at room temperature and quenched with water, extracted with ethyl acetate. The combined organic layer was washed with brine, dried over sodium sulphate and concentrated under reduced pressure to afford 2-(7-chloro-4-oxo-1-(phenylsulfonyl)-1,4-dihydro-5H-pyrrolo[2,3-d]pyridazin-5-yl)-N-(2,2-difluorobenzo[d][1,3]dioxol-5-yl)-N-ethylacetamide (0.85 g, 30%). (MS ESI +Ve 411.25[M+1]).

Step-2: A mixture of 2-(7-chloro-4-oxo-1-(phenylsulfonyl)-1,4-dihydro-5H-pyrrolo[2,3-d]pyridazin-5-yl)-N-(2,2-difluorobenzo[d][1,3]dioxol-5-yl)-N-ethylacetamide (0.85 g, 2.07 mmol) in 1,2-Dimethoxyethane (6 ml), water (1.5 ml), caesium fluoride (0.94 g, 6.21 mmol), 3-fluorophenyl boronic acid (0.72 g, 5.17 mmol) and PdCl₂(dppf) (151 mg, 0.207 mmol) was degassed and stirred at 90° C. for 16 h. Solvent was removed under vacuum and the residue was purified by basic alumina column chromatography (0-10% Methanol in dichloromethane) to give N-(2,2-difluorobenzo[d][1,3]dioxol-5-yl)-N-ethyl-2-(7-(3-fluorophenyl)-4-oxo-1,4-dihydro-5H-pyrrolo[2,3-d]pyridazin-5-yl)acetamide (0.035 g, 15%). (MS ESI +Ve 471.2[M+1]); ¹H NMR: (400 MHz, DMSO) δ:1.02 (t, J=6.4 Hz, 3H), 3.67 (q, J=6.4 Hz, 2H), 4.73 (s, 2H), 6.70 (d, J=2.4 Hz, 1H), 7.30-7.36 (m, 2H), 7.50-7.61 (m, 4H), 7.64-7.69 (m, 2H), 12.84 (br, 1H).

In a similar fashion, the following examples were synthesized:

| Example # | Stucture | m/z | ¹H NMR |
|---|---|---|---|
| 502 | 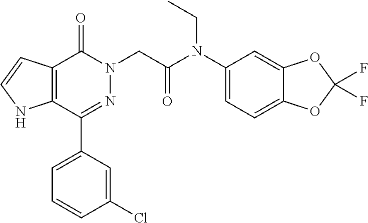 [SMILES: CCN(C(=O)Cn1nc(-c2cccc(Cl)c2)c2[nH]ccc2c1=O)c1ccc2c(c1)OC(F)(F)O2] | 487.0 [M + 1] | (400 MHz, DMSO) δ: 1.04 (m, 3H), 3.68 (q, J = 5.6 Hz, 2H), 4.75 (s, 2H), 6.68 (s, 1H), 7.32 (d, J = 7.2 Hz, 1H), 7.50-7.65 (m, 5H), 7.79 (m, 2H), 12.87 (br, 1H) |
| 503 | 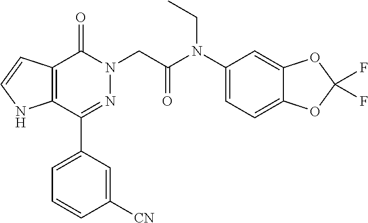 [SMILES: CCN(C(=O)Cn1nc(-c2cccc(C#N)c2)c2[nH]ccc2c1=O)c1ccc2c(c1)OC(F)(F)O2] | 478.2 [M + 1] | (400 MHz, DMSO) δ: 1.04 (t, J = 6.8 Hz, 3H), 3.69 (q, J = 6.8 Hz, 2H), 4.77 (s, 2H), 6.78 (d, J = 2.4 Hz, 1H), 7.33 (d, J = 8.4 Hz, 1H), 7.53 (d, J = 8.4 Hz, 1H), 7.60 (m, 1H), 7.67 (s, 1H), 7.77 (t, J = 7.6 Hz, 1H), 7.99 (d, J = 7.2 Hz, 1H), 8.16-8.20 (m, 2H), 12.91 (br, 1H) |
| 504 | 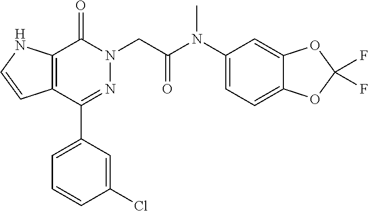 [SMILES: CN(C(=O)Cn1nc(-c2cccc(Cl)c2)c2cc[nH]c2c1=O)c1ccc2c(c1)OC(F)(F)O2] | 473.0 [M + 1] | (400 MHz, DMSO) δ: 3.20 (s, 1H), 4.76 (s, 2H), 6.75 (s, 1H), 7.37 (m, 1H), 7.48-7.76 (m, 6H), 12.24 (s, 1H) |
| 505 | 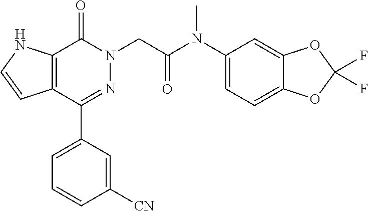 [SMILES: CN(C(=O)Cn1nc(-c2cccc(C#N)c2)c2cc[nH]c2c1=O)c1ccc2c(c1)OC(F)(F)O2] | 464.0 [M + 1] | (400 MHz, DMSO) δ: 3.20 (s, 1H), 4.77 (s, 2H), 6.77 (s, 1H), 7.37 (s, 1H), 7.51-7.53 (m, 2H), 7.78 (t, J = 8.0 Hz, 1H), 8.02 (d, J = 7.6 Hz, 1H), 8.07 (d, J = 7.6 Hz, 1H), 8.19 (s, 1H), 12.29 (s, 1H) |
| 506 | 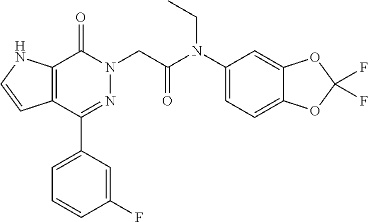 [SMILES: CCN(C(=O)Cn1nc(-c2cccc(F)c2)c2cc[nH]c2c1=O)c1ccc2c(c1)OC(F)(F)O2] | 471.2 [M + 1] | (400 MHz, DMSO) δ: 1.03 (t, J = 6.8 Hz, 3H), 3.67 (q, J = 6.8 Hz, 2H), 4.70 (s, 2H), 6.74 (m, 1H), 7.31 (d, J = 8.0 Hz, 1H), 7.37 (td, J = 6.8, 2.0 Hz, 1H), |

Assays for Detecting and Measuring the Effect of Compounds on dF508-CFTR Channels CFRT-YFP High Throughput Assay:

The following protocol is designed to selectively screen small molecule compounds for F508del CFTR corrector activities in the HTS YFP flux assay. In this protocol, the cells are incubated with test compounds for 24 hours, washed with PBS, stimulated with forskolin and a standard potentiator, and read on a 384-well HTS plate reader, such as the Hamamatsu FDDD-6000.

YFP fluorescence intensity is acquired at high speed before and after iodide buffer is injected to the assay cells. Iodide enters the cells via active CFTR channels in the plasma membrane, and quenches the YFP fluorescence. The rate of fluorescence quenching is proportionally related to the total CFTR activities in the cell membrane. dF508-CFTR corrector accelerates YFP quenching by increasing the number of CFTR molecules in the testing cell plasma membrane.

This method was initially developed for bench top plate readers (Galietta et al., 2001), and was adapted to the HTS format (Sui et al. Assay Drug Dev. Technol. 2010).

Fisher Rat Thyroid (FRT) cells stably expressing both human ΔF508-CFTR and a halide-sensitive yellow fluorescent protein (YFP-H148Q/I152L 25, 22) (Galietta et al., Am. J. Physiol Cell Physiol 281(5), C1734, 2001) were cultured on plastic surface in Coon's modified Ham's F12 medium supplemented with FBS 10%, L-glutamine 2 mM, penicillin 100 U/mL, and streptomycin 100 μg/mL. G418 (0.75-1.0 mg/mL) and zeocin (3.2 ug/mL) were used for selection of FRT cells expressing ΔF508-CFTR and YFP. For primary screening, FRT cells were plated into 384-well black wall, transparent bottom microtiter plates (Costar; Corning Inc.) at a cell density of 20,000-40,000 per well. Test compounds were applied to the cells at varying concentrations. Cells were incubated in a cell culture incubator at 37° C. with 5% CO₂ for 24-26 hr. Assay plates were washed with DPBS media (Thermo, cat #SH30028.02) to remove unbound cells and compound. Stimulation media (25 μL) containing 20 μM Forskolin & 30 μM P3 [6-(Ethyl-phenyl-sulfonyl)-4-oxo-1,4-dihydro-quinoline-3-carboxylic acid 2-methoxy-benzylamide] in Hams F-12 Coon's modified media was added to the plate wells and incubated at room temperature for 60-120 min. 25 μL of HEPES-PBS-I buffer (10 mM HEPES, 1 mM MgCl₂, 3 mM KCl, 1 mM CaCl₂), 150 mM NaI) was then added and fluorescence quench curves (Excitation 500 nm/Emission 540 nm; exposure 136 ms) were immediately recorded on an FDSS-6000 plate reader (Hamamatsu). Quench rates were derived from least squares fitting of the data as described by Sui et al., (2010).

### REFERENCES

Galietta, L. J., Jayaraman, S., and Verkman, A. S. Cell-based assay for high-throughput quantitative screening of CFTR chloride transport agonists. Am. J. Physiol Cell Physiol 281(5), C1734, 2001.
Sui J, Cotard S, Andersen J, Zhu P, Staunton J, Lee M, Lin S. (2010) Optimization of a Yellow fluorescent protein-based iodide influx high-throughput screening assay for cystic fibrosis transmembrane conductance regulator (CFTR) modulators. Assay Drug Dev Technol. 2010 December; 8(6):656-68.
Alejandro A. Pezzulo, Xiao Xiao Tang, Mark J. Hoegger, Mahmoud H. Abou Alaiwa, Shyam Ramachandran, Thomas O. Moninger, Phillip H. Karp, Christine L. Wohlford-Lenane, Henk P. Haagsman, Martin van Eijk, Botond Bánfi, Alexander R. Horswill, David A. Stoltz, Paul B. McCray Jr, Michael J. Welsh, Joseph Zabner. Reduced airway surface pH impairs bacterial killing in the porcine cystic fibrosis lung. Nature 487, 109-115 (2012).

### Determination of Activity in Primary CF Cell:

### Cell Culture:

Primary CF airway epithelial cells were obtained from the UNC Cystic Fibrosis Tissue Procurement and Cell Culture Core. The cells are grown at 37° C. in a Heracell 150i incubator using growth media (BEGM, Fischer). Cells were then transferred to differentiation media (airway liquid interface media (ALI) media; Lechner J F and LaVeck M A, J. Tissue Culture Methods 1985, 9: 43-48) for a minimum of 4 weeks on coated Costar transwells. Two days before the assay the mucus on the apical surface of the cells was aspirated after incubating with 200 μL of differentiation media for at least thirty (30) minutes. One day before the assay test compounds were added to the basolateral surface of the cells at various test concentrations dissolved in DMSO. Duplicate wells were prepared giving a n=4 or n=6 protocol.

### Electrophysiological Procedures

Cells were treated for 24 hours with various combinations and concentrations of the test articles, reference standard (3 μM VX809, positive control). Compounds stock solutions were prepared in DMSO and diluted 1/1000 into ALI media to their final assay concentration. Cells were treated with combination solutions (2 mL of each dilution) and incubated at 37° C. for 24 h.

### Equivalent Current Assay

For the equivalent current assay, cells on six Transwell (24-well) plates were treated. Each Transwell plate was filled with 200 μl of HBS on the apical surface and 2 ml on the basolateral surface. Plates were placed horizontally in a heated mount at 37° C. and equilibrated for 30 minutes. Resting current was measured for 15 min and then blocked by the apical addition of 5 μM benzamil. After 20 min, CFTR activator (10 μM forskolin) and potentiator (either 1 μM VX-770 or 3 μM FDL176) were added to both the apical and basolateral side to stimulate CFTR. An increase in chloride current is seen as an upward deflection of the trace. After another 60 min, CFTR-172 (a CFTR inhibitor, 20 μM) and/or bumetanide (20 uM) was added to block CFTR mediated chloride current.

### Data Collection and Analysis Methods

The raw data, voltage vs. time and resistance vs. time for the equivalent current assay (sampling interval: 6 minutes) were transferred to Excel (Microsoft Office Professional, version 14.0.7106.5003) for analysis. CFTR specific current was computed from I=V/R (Ohms Law), the average amplitude of the increase in current elicited upon addition forskolin and ending upon addition of the CFTR channel specific blocker CFTR-172. This average is equivalent to the sum of the average forskolin activated and the average VX770-potentiated currents. The average current measured in vehicle (0.1% DMSO) treated cells, I_{V}, was subtracted from the current for the test article, I_{TA}, or from the corrector reference standard VX809 (3 uM, I_{STD}). For replicate measurements, the averaged vehicle subtracted response for the test article, was normalized to the averaged vehicle subtracted response of the reference corrector VX809 (3 μM).

I_{NSC}=(I_{TA}−I_{V})₍ₐᵥₑ₎/(I_{STD}−I_{V})₍ₐᵥₑ₎ (Equation 1)

A second endpoint, for the equivalent current assay, evaluated was NAUC, the normalized area under the curve (AUC) measuring the response after addition of forskolin and potentiator to the time point right before the addition of the CFTR inhibitor. The AUC is effectively the average response multiplied by the duration of the response. The AUC of the test article, AUC_{TA} was then corrected by subtracting the average vehicle response, AUC_{V,ave} over the same time range, and normalized as for the inhibitor-sensitive current to the difference of the corrector reference standard VX809 (3 μM _{VX809r,ave} and the vehicle response:

NAUC_{TA}=[AUC_{TA}−AUC_{V,ave}]/[AUC_{Vx809r,ave}−AUC_{V,ave}] (Equation 2).

The normalized value for DMSO is 0.0 and for VX-809 alone is 1.0 for both of these equations.

Experiments were run with a minimum of n=4 replicates per concentration. Since the distribution for the ratio of two normal distributions is a Cauchy distribution, the median value must be used for the average and the average deviation must be used for the error of all normalized data. Potency (EC₅₀) and efficacy (maximum response) were determined by fitting dose response data to a sigmoid dose response model (GraphPad Prism 5.04, Manufacturer) using Equation 3:

E=Eₘᵢₙ+(Eₘₐₓ−Eₘᵢₙ)/(1+10\hat{ }((Log EC₅₀−S)*n_{H})) (Equation 3)

where E is the recorded response, and S is the concentration of test compound. Since there were at most 8 points in the dose response curve only the Hill slope, n_{H}, was fixed equal to 1.

Statistical comparisons (t-test and Mann Whitney) and calculation of averages and errors were performed in Excel.

The table below provides the results of the equivalent current assays in Primary CF airway epithelial cells (Corrector Activity) for exemplary compounds of the invention and the results of the HTS YFP flux assay (Potentiator Activity) for a subset of these compounds. NAUC “+++” refers to an observed NAUC>70% of positive control; NAUC “++” refers to an observed NAUC 70-40% of positive control; NAUC “+” refers to an observed NAUC<40% of positive control and “O” refers to no positive response.

It was noticed that many of the test compounds had potentiator activity in addition to corrector activity. These compounds have been denoted as CoPos by FDL and others. To measure both the corrector and potentiator activity of a compound the following changes were made to the test conditions. Cells were treated as usual with the test compounds for at least 24 hours before the experiment. 30 minutes prior to the experiment the test compound is added back to the testing solution to act as a potentiator and no VX-770 (the standard CFTR potentiator) was used with the test compound. The negative control was no longer just DMSO (no corrector) but changed to VX-809 (the standard corrector) plus VX-770 treatment for at least 24 hours. 30 minutes prior to the experiment both VX-809 and VX-770 were added back to the testing solution. This treatment led to a smaller response than the positive control of correcting F508del CFTR with VX-809 alone for at least 24 hours and adding VX-770 acutely with the forskolin addition (just as in the corrector assay).

A CoPo reading of zero means that the test compound has the same forskolin stimulated response as VX-809+VX-770 treatment for 24 hours. A CoPo reading of 1.0 means the test compound has the same forskolin response as cell treated with VX-809 and stimulated with forskolin+VX-770. CoPo activity “+++” refers to an observed CoPo>70% of positive control; CoPo activity “++” refers to an observed CoPo 70-40% of positive control; CoPo activity “+” refers to an observed CoPo<40% of positive control and “O” refers to no positive response.

| example | Corr | CoPo |
|---|---|---|
| # | Activity | Activity |
| 1 | +++ | |
| 2 | o | |
| 3 | +++ | |
| 4 | +++ | |
| 5 | +++ | |
| 6 | +++ | |
| 7 | +++ | |
| 8 | +++ | ++ |
| 9 | +++ | +++ |
| 10 | +++ | +++ |
| 11 | +++ | +++ |
| 12 | +++ | +++ |
| 18 | ++ | |
| 19 | +++ | |
| 20 | +++ | |
| 21 | + | O |
| 22 | ++ | O |
| 23 | + | + |
| 24 | +++ | +++ |
| 25 | +++ | + |
| 26 | +++ | |
| 27 | | o |
| 28 | | o |
| 32 | +++ | |
| 33 | +++ | |
| 39 | +++ | |
| 40 | ++ | |
| 41 | +++ | |
| 42 | +++ | |
| 43 | +++ | |
| 44 | +++ | |
| 45 | +++ | |
| 46 | ++ | |
| 47 | +++ | |
| 48 | ++ | |
| 49 | ++ | |
| 50 | +++ | |
| 51 | ++ | |
| 52 | ++ | |
| 53 | +++ | |
| 54 | +++ | |
| 55 | +++ | |
| 56 | + | |
| 57 | + | |
| 58 | + | |
| 59 | +++ | |
| 60 | +++ | |
| 61 | + | |
| 62 | + | |
| 63 | ++ | |
| 64 | ++ | |
| 65 | + | |
| 69 | +++ | |
| 70 | +++ | |
| 71 | +++ | |
| 72 | +++ | |
| 73 | +++ | |
| 74 | +++ | |
| 75 | +++ | |
| 76 | +++ | |
| 77 | +++ | |
| 78 | +++ | |
| 79 | +++ | |
| 83 | +++ | |
| 84 | +++ | |
| 85 | +++ | |
| 86 | +++ | |
| 87 | + | |
| 88 | ++ | |
| 89 | ++ | |
| 90 | ++ | |
| 91 | +++ | |
| 92 | + | |
| 93 | + | |
| 94 | ++ | |
| 95 | ++ | |
| 96 | +++ | |
| 97 | + | |
| 98 | ++ | |
| 99 | +++ | |
| 100 | ++ | |
| 101 | +++ | |
| 102 | ++ | |
| 103 | ++ | |
| 104 | +++ | |
| 105 | +++ | |
| 106 | o | |
| 107 | o | |
| 108 | +++ | |
| 109 | +++ | |
| 110 | +++ | |
| 111 | +++ | |
| 112 | +++ | |
| 113 | +++ | |
| 114 | +++ | |
| 115 | +++ | |
| 116 | ++ | |
| 117 | + | |
| 119 | +++ | |
| 120 | +++ | |
| 121 | +++ | |
| 122 | ++ | |
| 123 | + | |
| 124 | ++ | |
| 125 | ++ | |
| 126 | +++ | |
| 127 | ++ | |
| 128 | ++ | |
| 129 | ++ | |
| 130 | +++ | |
| 131 | +++ | |
| 132 | +++ | |
| 133 | +++ | |
| 134 | ++ | |
| 135 | +++ | |
| 136 | +++ | |
| 137 | +++ | |
| 138 | +++ | |
| 139 | +++ | |
| 140 | ++ | |
| 141 | +++ | |
| 142 | ++ | |
| 143 | ++ | |
| 144 | +++ | |
| 145 | ++ | |
| 146 | +++ | |
| 147 | +++ | |
| 148 | +++ | |
| 149 | +++ | |
| 150 | +++ | |
| 151 | +++ | |
| 152 | +++ | |
| 153 | +++ | |
| 154 | +++ | |
| 155 | ++ | o |
| 156 | ++ | o |
| 157 | ++ | |
| 158 | +++ | |
| 159 | +++ | |
| 160 | ++ | + |
| 161 | +++ | ++ |
| 162 | +++ | |
| 163 | +++ | |
| 164 | +++ | |
| 165 | +++ | |
| 166 | +++ | |
| 167 | +++ | |
| 168 | +++ | |
| 169 | +++ | |
| 170 | ++ | |
| 171 | +++ | |
| 172 | +++ | |
| 173 | +++ | |
| 174 | ++ | |
| 175 | +++ | |
| 176 | +++ | |
| 177 | +++ | |
| 178 | + | |
| 179 | ++ | |
| 180 | +++ | |
| 181 | +++ | |
| 182 | +++ | |
| 183 | +++ | |
| 184 | +++ | |
| 185 | +++ | |
| 186 | +++ | |
| 187 | +++ | |
| 188 | +++ | |
| 189 | +++ | |
| 190 | +++ | |
| 191 | +++ | |
| 192 | +++ | |
| 193 | +++ | |
| 194 | +++ | |
| 195 | ++ | |
| 196 | ++ | |
| 197 | +++ | |
| 198 | ++ | |
| 199 | + | |
| 200 | ++ | |
| 201 | +++ | |
| 202 | +++ | |
| 203 | +++ | |
| 204 | +++ | |
| 205 | +++ | |
| 206 | ++ | |
| 207 | +++ | |
| 208 | +++ | |
| 210 | ++ | |
| 211 | ++ | |
| 212 | + | |
| 213 | + | |
| 214 | ++ | |
| 215 | ++ | |
| 216 | +++ | |
| 218 | +++ | |
| 219 | +++ | |
| 220 | ++ | |
| 221 | +++ | |
| 222 | +++ | |
| 223 | +++ | |
| 224 | +++ | |
| 225 | +++ | |
| 226 | +++ | |
| 227 | +++ | |
| 228 | +++ | |
| 229 | +++ | |
| 230 | +++ | |
| 231 | +++ | |
| 232 | ++ | |
| 233 | ++ | |
| 234 | ++ | |
| 235 | ++ | |
| 236 | +++ | |
| 237 | ++ | |
| 238 | ++ | |
| 239 | + | |
| 240 | + | |
| 241 | +++ | |
| 242 | +++ | |
| 243 | +++ | |
| 244 | +++ | |
| 245 | +++ | |
| 246 | +++ | |
| 247 | +++ | |
| 248 | +++ | |
| 249 | +++ | |
| 250 | +++ | |
| 251 | +++ | |
| 252 | ++ | |
| 253 | o | |
| 254 | +++ | |
| 255 | +++ | |
| 256 | o | |
| 257 | +++ | |
| 258 | +++ | |
| 259 | o | |
| 260 | + | |
| 261 | +++ | |
| 262 | +++ | |
| 264 | ++ | |
| 265 | + | |
| 266 | o | |
| 267 | + | |
| 268 | ++ | |
| 269 | +++ | |
| 270 | ++ | |
| 271 | + | |
| 272 | + | |
| 273 | +++ | |
| 274 | + | |
| 275 | o | |
| 276 | +++ | |
| 277 | +++ | |
| 278 | +++ | |
| 279 | ++ | |
| 280 | + | |
| 281 | +++ | |
| 282 | + | |
| 283 | o | |
| 284 | + | |
| 285 | +++ | |
| 286 | +++ | |
| 287 | o | |
| 288 | o | |
| 289 | +++ | |
| 290 | + | |
| 291 | o | |
| 292 | + | |
| 293 | + | |
| 294 | + | |
| 295 | + | |
| 296 | + | |
| 297 | o | |
| 298 | o | |
| 299 | o | |
| 300 | o | |
| 301 | o | |
| 302 | o | |
| 303 | + | |
| 304 | o | |
| 305 | o | |
| 306 | o | |
| 307 | o | |
| 308 | o | |
| 309 | o | |
| 310 | + | |
| 311 | + | |
| 312 | o | |
| 313 | ++ | |
| 314 | o | |
| 315 | o | |
| 316 | ++ | |
| 317 | + | |
| 318 | o | |
| 319 | + | |
| 320 | ++ | |
| 321 | + | |
| 322 | +++ | |
| 323 | o | |
| 324 | o | |
| 325 | +++ | |
| 326 | +++ | |
| 327 | + | |
| 328 | + | |
| 329 | + | |
| 330 | + | |
| 331 | o | |
| 332 | o | |
| 333 | o | |
| 334 | o | |
| 335 | o | |
| 336 | ++ | |
| 337 | + | |
| 338 | ++ | |
| 339 | ++ | |
| 340 | o | |
| 341 | o | |
| 342 | o | |
| 343 | o | |
| 344 | o | |
| 345 | + | |
| 346 | +++ | |
| 347 | +++ | |
| 348 | o | |
| 349 | o | |
| 350 | + | |
| 351 | + | |
| 352 | +++ | |
| 353 | o | |
| 354 | o | |
| 355 | + | |
| 356 | o | |
| 357 | + | |
| 358 | o | |
| 359 | o | |
| 360 | o | |
| 361 | o | |
| 362 | o | |
| 363 | + | |
| 364 | o | |
| 365 | +++ | |
| 366 | + | |
| 367 | o | |
| 368 | +++ | |
| 369 | +++ | |
| 370 | + | |
| 371 | ++ | |
| 372 | +++ | |
| 373 | +++ | |
| 374 | +++ | |
| 375 | +++ | |
| 376 | ++ | |
| 377 | + | |
| 378 | ++ | |
| 379 | ++ | |
| 380 | ++ | |
| 381 | + | |
| 382 | + | |
| 383 | ++ | |
| 384 | ++ | |
| 385 | ++ | |
| 387 | + | |
| 388 | o | |
| 389 | o | |
| 390 | + | |
| 391 | ++ | |
| 392 | +++ | |
| 393 | ++ | |
| 395 | + | |
| 396 | +++ | |
| 397 | + | |
| 398 | o | |
| 399 | ++ | |
| 400 | +++ | |
| 401 | ++ | |
| 402 | ++ | |
| 403 | o | |
| 404 | ++ | |
| 405 | + | |
| 406 | o | |
| 407 | + | |
| 408 | ++ | |
| 409 | ++ | |
| 410 | o | |
| 411 | o | |
| 412 | ++ | |
| 413 | o | |
| 414 | o | |
| 415 | o | |
| 417 | o | |
| 418 | o | |
| 420 | o | |
| 422 | o | |
| 423 | + | |
| 424 | o | |
| 425 | + | |
| 426 | + | |
| 427 | o | |
| 428 | o | |
| 429 | o | |
| 430 | + | |
| 431 | o | |
| 432 | + | |
| 433 | + | |
| 438 | o | |
| 439 | o | |
| 445 | +++ | |
| 446 | +++ | |
| 447 | o | |
| 448 | + | |
| 449 | o | |
| 451 | o | |
| 452 | o | |
| 453 | o | |
| 455 | o | |
| 457 | + | |
| 464 | +++ | |
| 465 | ++ | |
| 469 | + | |
| 470 | +++ | |
| 483 | ++ | |
| 484 | o | |
| 485 | o | |
| 486 | +++ | |
| 487 | ++ | |
| 488 | +++ | |
| 489 | +++ | |
| 490 | + | |
| 491 | ++ | |
| 492 | ++ | |
| 493 | ++ | |
| 494 | ++ | |
| 495 | ++ | |
| 496 | ++ | |
| 497 | + | |
| 498 | + | |
| 499 | + | |
| 500 | ++ | |
| 501 | +++ | |
| 502 | +++ | |
| 503 | +++ | |
| 504 | o | |
| 505 | o | |
| 506 | o | |

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

1. A compound of Formula (I) m 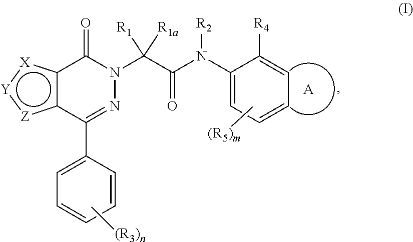
or a pharmaceutically acceptable salt thereof, wherein:
Ring A is a 4- to 6-membered optionally substituted carbocyclic or heterocyclic;
X is O, S or NR₇; Y is N or CR₆; and Z is N or CR₆; or
X is N or CR₆; Y is N or CR₆ and Z is O, S or NR₇; or
X is N or CR₆; Y is O, S or NR₇ and Z is N or CR₆;
n is 0, 1, 2 or 3;
m is 0, 1 or 2;
R₁ and R₁ₐ are independently hydrogen, optionally substituted alkyl, or halogen;
R₂ is hydrogen, optionally substituted alkyl, optionally substituted cycloalkyl or optionally substituted heterocyclyl;
each R₃ is independently CN, hydroxyl, halogen, optionally substituted C₁-C₆-alkyl, optionally substituted C₁-C₆-alkoxy, optionally substituted aryl, R₈OC(O)—, (R₈)(R₉)NC(O)—, or (R₈)(R₉)NS(O)₂—;
alternatively, two adjacent R₃ groups, together with the carbon atoms to which they are attached, form an optionally substituted carbocyclic or heterocyclic;
R₄ is hydrogen or substituted or unsubstituted C₁-C₆-alkyl;
or R₂ and R₄ together form optionally substituted C₂-C₃-alkylene;
each R₅ is independently optionally substituted C₁-C₄-alkyl;
each R₆ is hydrogen, halogen, hydroxyl, optionally substituted C₁-C₆-alkyl, optionally substituted C₁-C₆-alkoxy, optionally substituted C₃-C₆-cycloalkyl, (R₈)(R₉)NC(O)—, and R₁₀C(O)NH—;
each R₇ is hydrogen or optionally substituted C₁-C₄-alkyl;
R₈ and R₉ are each independently hydrogen, optionally substituted C₁-C₆-alkyl, or optionally substituted C₃-C₆-cycloalkyl; alternatively, R₈ and R₉, together with the nitrogen atom to which they are attached, form an optionally substituted C₃-C₈-heterocyclyl; and
R₁₀ is optionally substituted C₁-C₆-alkyl.

2. The compound of claim 1, wherein 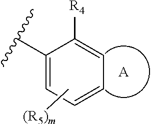
is selected from the group consisting of

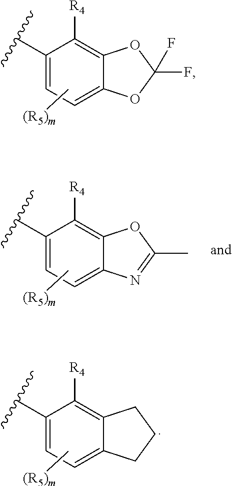

3. The compound of claim 1 represented by one of Formulas (IIa), (IIb), (IIc), (IId), (IIe), (IIf), (IIg), (IIh), (IIi), (IIj), (IIIa), (IIIb), (IIIc), (IIId), (IIIe), (IIIf), (IIIg), (IIIh), (IIIi) and (IIIj), or pharmaceutically acceptable salt thereof,

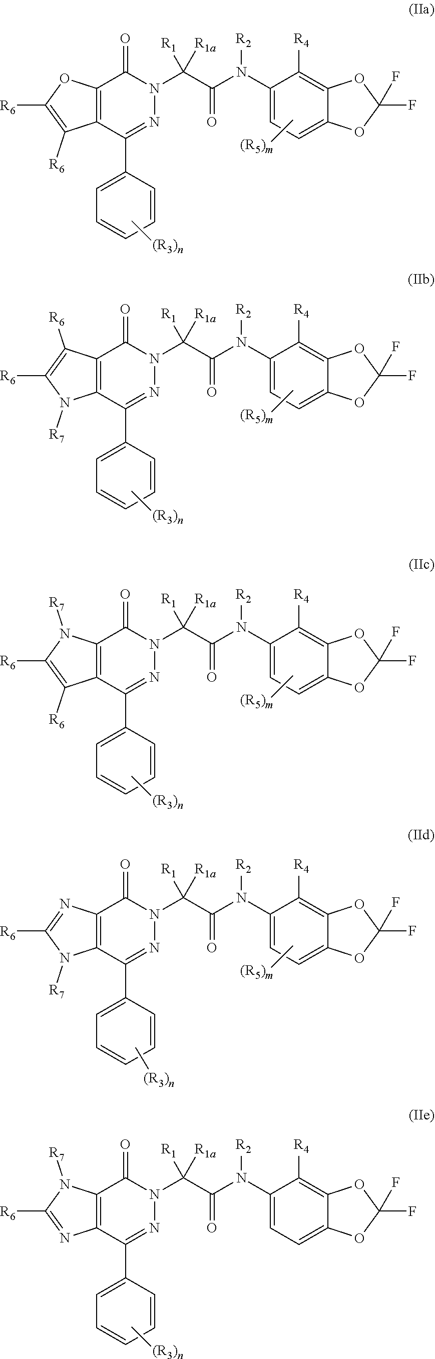

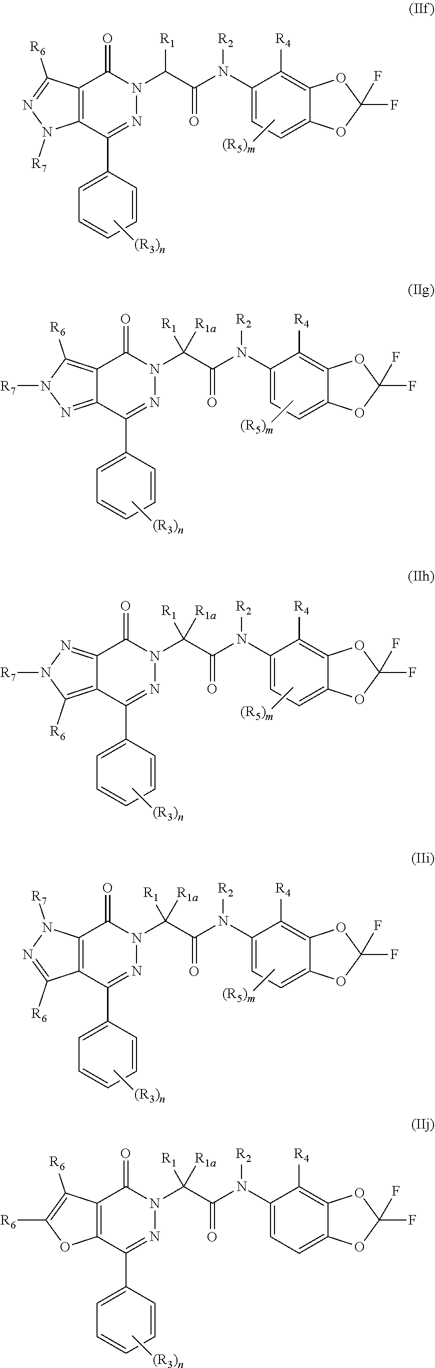

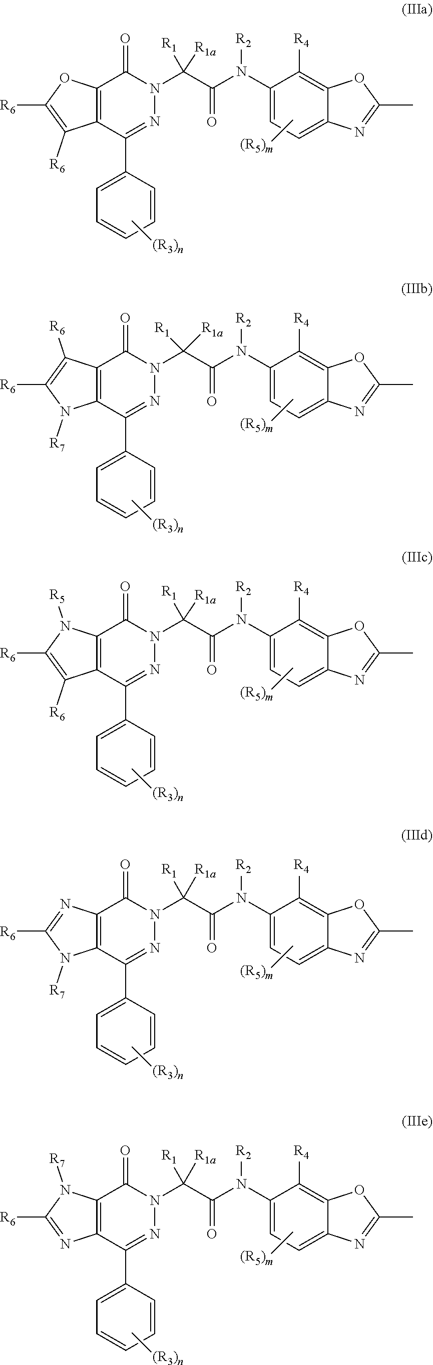

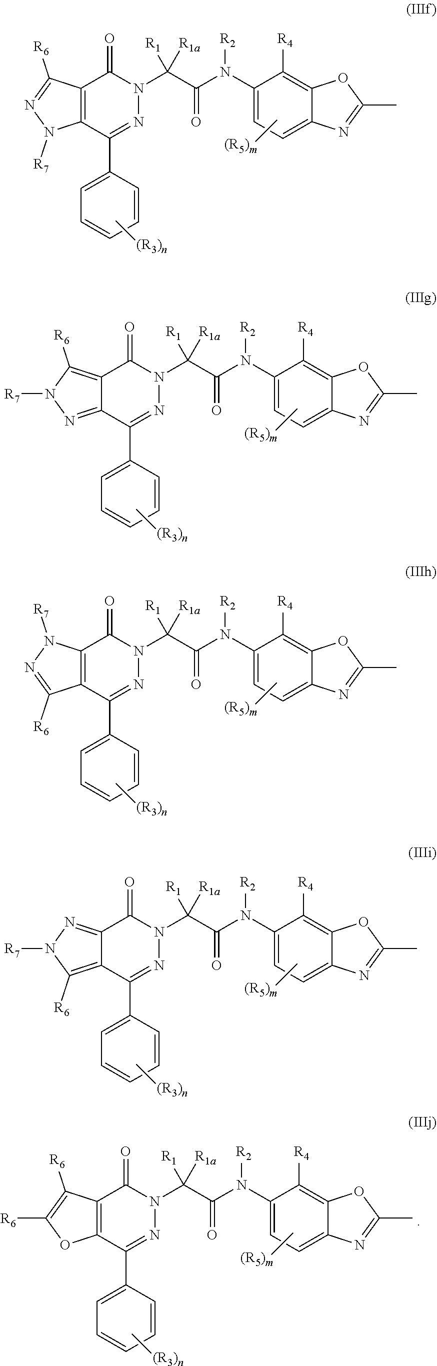

4. (canceled)

5. The compound of claim 1, wherein R₂ is C₁-C₄-alkyl, halo-C₁-C₄-alkyl, C₃-C₆-cycloalkyl or saturated 5- or 6-membered heterocyclyl.

6. The compound of claim 5, wherein R₂ is methyl, ethyl or cyclopropyl.

7. The compound of claim 1, wherein each R₃ is independently CN, F, Cl, hydroxyl, C₁-C₄-alkyl, halo-C₁-C₄-alkyl, C₁-C₄-alkoxy, or halo-C₁-C₄-alkoxy.

8. The compound of claim 7, wherein each R₃ is independently CN, F, Cl, methyl, t-butyl, trifluoromethyl, difluoromethyl, methoxy, t-butoxy or trifluoromethoxy.

9. The compound of claim 1, wherein each R₆ is hydrogen.

10. The compound of claim 1, wherein two of X, Y and Z are CR₆ and at least one R₆ is hydrogen.

11. The compound of claim 1, wherein two of X, Y and Z are CR₆ and at least one R₆ is selected from C₁-C₄-alkyl; fluoro-substituted C₁-C₄-alkyl; hydroxyl-C₁-C₄-alkyl; optionally substituted C₃- or C₄-cycloalkyl; R₈R₉NC(O)— where R₈, R₉ and the nitrogen atom together form a 5- or 6-membered heterocyclyl; and R₈OC(O)—, where R₈ is hydrogen or optionally substituted C₁-C₄-alkyl.

12. The compound of claim 1, wherein 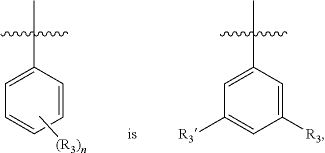
wherein R₃′ is R₃ or hydrogen.

13. The compound of claim 12, wherein R₃′ is hydrogen, and R₃ is selected from CN, F, Cl, methyl, t-butyl, trifluoromethyl, difluoromethyl, methoxy, t-butoxy and trifluoromethoxy.

14. A pharmaceutical composition comprising a compound of claim 1 and a pharmaceutically acceptable carrier or excipient.

15. A method of treating a disease or disorder mediated by cystic fibrosis transmembrane conductance regulator (CFTR) in a subject in need thereof, comprising administering to the subject a therapeutically effective amount of a compound of claim 1.

16. The method according to claim 15, wherein said disease or disorder is selected from the group consisting of cystic fibrosis, hereditary emphysema, hereditary hemochromatosis, coagulation-fibrinolysis deficiencies, type 1 hereditary angioedema, lipid processing deficiencies, such as familial hypercholesterolemia, type 1 chylomicronemia, abetalipoproteinemia, lysosomal storage diseases, such as I-cell disease/pseudo Hurler, mucopolysaccharidoses, Sandhof/Tay-Sachs, Crigler-Najjar type II, polyendocrinopathy/hyperinsulemia, diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, diabetes insipidus (DI), neurophyseal DI, neprogenic DI, Charcot-Marie tooth syndrome, Perlizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington's disease, Spinocerebullar ataxia type I, Spinal and bulbar muscular atrophy, Dentororubal pallidoluysian, and Myotonic dystrophy, as well as, spongiform encephalopathies such as Hereditary Creutzfeldt-Jakob disease, Fabry disease, Straussler-Scheinker disease, secretory diarrhea, polycystic kidney disease, chronic obstructive pulmonary disease (COPD), dry eye disease, or Sjogren's Syndrome, congenital bilateral absence of vas deferens (CBAVD), disseminated bronchiectasis, allergic pulmonary aspergillosis, chronic sinusitis, protein C deficiency, A-lipoproteinemia, mild pulmonary disease, lipid processing deficiencies, coagulation fibrinolyis, CFTR-related metabolic syndrome, chronic bronchitis, constipation, pancreatic insufficiency, melanoma, glycanosis CDG type 1, ACT deficiency, cholestatic liver disease.

17. A method for treating cystic fibrosis in a subject in need thereof, comprising the step of administering to the subject a therapeutically effective amount of a compound according to claim 1.

18. The method of claim 17, further comprising the step of administering to the subject a therapeutically effective amount of a compound which is a CFTR modulator, a mucolytic or an antibiotic.
